SEQUENCE LISTING (634 sequences)

SEQ ID NO: 1 - protein, 15 aa, synthetic construct
CAIAGPGQGARGYTF

SEQ ID NO: 2 - protein, 12 aa, synthetic construct
CAISEGSPEAFF

SEQ ID NO: 3 - protein, 13 aa, synthetic construct
CASGLRLNEKLFF

SEQ ID NO: 4 - protein, 12 aa, synthetic construct
CASGLVDYELFF

SEQ ID NO: 5 - protein, 17 aa, synthetic construct
CASIHLVGGTGRQPQHF

SEQ ID NO: 6 - protein, 17 aa, synthetic construct
CASKEATAASTNEKLFF

SEQ ID NO: 7 - protein, 11 aa, synthetic construct
CASKQGNEQFF

SEQ ID NO: 8 - protein, 16 aa, synthetic construct
CASLLDAGAANTEAFF

SEQ ID NO: 9 - protein, 14 aa, synthetic construct
CASLSRGLNEKLFF

SEQ ID NO: 10 - protein, 15 aa, synthetic construct
CASMLQGALNQPQHF

SEQ ID NO: 11 - protein, 14 aa, synthetic construct
CASNFGQGRYGYTF

SEQ ID NO: 12 - protein, 16 aa, synthetic construct
CASNPDNALDNSPLHF

SEQ ID NO: 13 - protein, 14 aa, synthetic construct
CASRDSYSNQPQHF

SEQ ID NO: 14 - protein, 16 aa, synthetic construct
CASRDTQGGGADTQYF

SEQ ID NO: 15 - protein, 15 aa, synthetic construct
CASREQGQGTGELFF

SEQ ID NO: 16 - protein, 15 aa, synthetic construct
CASRGDSGFNYGYTF

SEQ ID NO: 17 - protein, 15 aa, synthetic construct
CASRGQGAATDTQYF

SEQ ID NO: 18 - protein, 15 aa, synthetic construct
CASRGQRRINYGYTF

SEQ ID NO: 19 - protein, 14 aa, synthetic construct
CASRGTGVNQPQHF

SEQ ID NO: 20 - protein, 12 aa, synthetic construct
CASRKDTGELFF

SEQ ID NO: 21 - protein, 12 aa, synthetic construct
CASRLDRSEAFF

SEQ ID NO: 22 - protein, 14 aa, synthetic construct
CASRPGQGYEKLFF

SEQ ID NO: 23 - protein, 12 aa, synthetic construct
CASRRNGLYYTF

SEQ ID NO: 24 - protein, 15 aa, synthetic construct
CASRSGTGGSGELFF

SEQ ID NO: 25 - protein, 15 aa, synthetic construct
CASRSPWTGANVLTF

SEQ ID NO: 26 - protein, 15 aa, synthetic construct
CASRTGGNLDDTQYF

SEQ ID NO: 27 - protein, 14 aa, synthetic construct
CASRTLSGGDTQYF

SEQ ID NO: 28 - protein, 11 aa, synthetic construct
CASSAFYEQYF

SEQ ID NO: 29 - protein, 14 aa, synthetic construct
CASSALGSGNTIYF

SEQ ID NO: 30 - protein, 15 aa, synthetic construct
CASSAQGTSYNEQFF

SEQ ID NO: 31 - protein, 13 aa, synthetic construct
CASSASFYQPQHF

SEQ ID NO: 32 - protein, 14 aa, synthetic construct
CASSATGGNSPLHF

SEQ ID NO: 33 - protein, 14 aa, synthetic construct
CASSEGQGDYGYTF

SEQ ID NO: 34 - protein, 14 aa, synthetic construct
CASSENRRREPQHF

SEQ ID NO: 35 - protein, 16 aa, synthetic construct
CASSEQSGLTNSPLHF

SEQ ID NO: 36 - protein, 15 aa, synthetic construct
CASSFAAGLGYEQYF

SEQ ID NO: 37 - protein, 15 aa, synthetic construct
CASSFAGVYTGELFF

SEQ ID NO: 38 - protein, 16 aa, synthetic construct
CASSFPTGGLSSEQFF

SEQ ID NO: 39 - protein, 11 aa, synthetic construct
CASSFQETQYF

SEQ ID NO: 40 - protein, 15 aa, synthetic construct
CASSFRGGLQETQYF

SEQ ID NO: 41 - protein, 13 aa, synthetic construct
CASSFTDRRKDTF

SEQ ID NO: 42 - protein, 17 aa, synthetic construct
CASSFYGTGGEGKPQHF

SEQ ID NO: 43 - protein, 13 aa, synthetic construct
CASSGTDFYEQYF

SEQ ID NO: 44 - protein, 14 aa, synthetic construct
CASSGTGDSGEAFF

SEQ ID NO: 45 - protein, 17 aa, synthetic construct
CASSGTGGAISNQPQHF

SEQ ID NO: 46 - protein, 14 aa, synthetic construct
CASSIGGTTGELFF

SEQ ID NO: 47 - protein, 14 aa, synthetic construct
CASSISTNTGELFF

SEQ ID NO: 48 - protein, 15 aa, synthetic construct
CASSITGSSGQPQHF

SEQ ID NO: 49 - protein, 16 aa, synthetic construct
CASSKDRGLALETQYF

SEQ ID NO: 50 - protein, 16 aa, synthetic construct
CASSKESANRYNEQFF

SEQ ID NO: 51 - protein, 13 aa, synthetic construct
CASSLASNQPQHF

SEQ ID NO: 52 - protein, 14 aa, synthetic construct
CASSLDQTSNEQFF

SEQ ID NO: 53 - protein, 16 aa, synthetic construct
CASSLDRGLGNSPLHF

SEQ ID NO: 54 - protein, 16 aa, synthetic construct
CASSLDSGTNTGELFF

SEQ ID NO: 55 - protein, 15 aa, synthetic construct
CASSLDSLATDTQYF

SEQ ID NO: 56 - protein, 16 aa, synthetic construct
CASSLEGGLAKNIQYF

SEQ ID NO: 57 - protein, 17 aa, synthetic construct
CASSLEGRGPTNEKLFF

SEQ ID NO: 58 - protein, 14 aa, synthetic construct
CASSLESGNSPLHF

SEQ ID NO: 59 - protein, 13 aa, synthetic construct
CASSLETGVEQFF

SEQ ID NO: 60 - protein, 15 aa, synthetic construct
CASSLFGGGGEKLFF

SEQ ID NO: 61 - protein, 14 aa, synthetic construct
CASSLFLGSYEQYF

SEQ ID NO: 62 - protein, 14 aa, synthetic construct
CASSLGGGNQPQHF

SEQ ID NO: 63 - protein, 14 aa, synthetic construct
CASSLGGNTGELFF

SEQ ID NO: 64 - protein, 18 aa, synthetic construct
CASSLGGSGSFYHNEQFF

SEQ ID NO: 65 - protein, 15 aa, synthetic construct
CASSLGLRPINEQFF

SEQ ID NO: 66 - protein, 15 aa, synthetic construct
CASSLGRGPTDTQYF

SEQ ID NO: 67 - protein, 13 aa, synthetic construct
CASSLGVNTEAFF

SEQ ID NO: 68 - protein, 13 aa, synthetic construct
CASSLGYRGEQYF

SEQ ID NO: 69 - protein, 15 aa, synthetic construct
CASSLLDRGDSPLHF

SEQ ID NO: 70 - protein, 12 aa, synthetic construct
CASSLLTGGQYF

SEQ ID NO: 71 - protein, 13 aa, synthetic construct
CASSLNRGYEQYV

SEQ ID NO: 72 - protein, 11 aa, synthetic construct
CASSLNTEAFF

SEQ ID NO: 73 - protein, 13 aa, synthetic construct
CASSLQGRTEAFF

SEQ ID NO: 74 - protein, 13 aa, synthetic construct
CASSLQGSYGYTF

SEQ ID NO: 75 - protein, 11 aa, synthetic construct
CASSLRGEAFF

SEQ ID NO: 76 - protein, 12 aa, synthetic construct
CASSLRGNEQFF

SEQ ID NO: 77 - protein, 15 aa, synthetic construct
CASSLRTGGRMPQHF

SEQ ID NO: 78 - protein, 15 aa, synthetic construct
CASSLRTNTGEKLFF

SEQ ID NO: 79 - protein, 16 aa, synthetic construct
CASSLSPGKSNQPQHF

SEQ ID NO: 80 - protein, 11 aa, synthetic construct
CASSLSYEQYF

SEQ ID NO: 81 - protein, 15 aa, synthetic construct
CASSLTEGVRTEAFF

SEQ ID NO: 82 - protein, 11 aa, synthetic construct
CASSLVETQYF

SEQ ID NO: 83 - protein, 13 aa, synthetic construct
CASSLVGGNTIYF

SEQ ID NO: 84 - protein, 13 aa, synthetic construct
CASSLVGNTEAFF

SEQ ID NO: 85 - protein, 13 aa, synthetic construct
CASSLVSTAEQYF

SEQ ID NO: 86 - protein, 13 aa, synthetic construct
CASSLVVTGELFF

SEQ ID NO: 87 - protein, 14 aa, synthetic construct
CASSLWGATDTQYF

SEQ ID NO: 88 - protein, 16 aa, synthetic construct
CASSPDSSFGNQPQHF

SEQ ID NO: 89 - protein, 13 aa, synthetic construct
CASSPDSYNEQFF

SEQ ID NO: 90 - protein, 11 aa, synthetic construct
CASSPEETQYF

SEQ ID NO: 91 - protein, 15 aa, synthetic construct
CASSPGQAANSPLHF

SEQ ID NO: 92 - protein, 11 aa, synthetic construct
CASSPGRVAFF

SEQ ID NO: 93 - protein, 13 aa, synthetic construct
CASSPGTDQPQHF

SEQ ID NO: 94 - protein, 11 aa, synthetic construct
CASSPGTEAFF

SEQ ID NO: 95 - protein, 15 aa, synthetic construct
CASSPMGTGNTEAFF

SEQ ID NO: 96 - protein, 15 aa, synthetic construct
CASSPPDRGRHEQFF

SEQ ID NO: 97 - protein, 14 aa, synthetic construct
CASSPPSGSGELFF

SEQ ID NO: 98 - protein, 16 aa, synthetic construct
CASSPPVLVSGNTIYF

SEQ ID NO: 99 - protein, 17 aa, synthetic construct
CASSPQDRGQGNTEAFF

SEQ ID NO: 100 - protein, 16 aa, synthetic construct
CASSPRDRGLYQPQHF

SEQ ID NO: 101 - protein, 14 aa, synthetic construct
CASSPRGAGNTIYF

SEQ ID NO: 102 - protein, 15 aa, synthetic construct
CASSPRTGGNQPQHF

SEQ ID NO: 103 - protein, 15 aa, synthetic construct
CASSPSAGAGYEQYF

SEQ ID NO: 104 - protein, 18 aa, synthetic construct
CASSPSQGIDSGANVLTF

SEQ ID NO: 105 - protein, 15 aa, synthetic construct
CASSPSRDRSYEQYF

SEQ ID NO: 106 - protein, 13 aa, synthetic construct
CASSPTDRIRAFF

SEQ ID NO: 107 - protein, 13 aa, synthetic construct
CASSPYRGLNHSF

SEQ ID NO: 108 - protein, 15 aa, synthetic construct
CASSQDGGGTDTQYF

SEQ ID NO: 109 - protein, 15 aa, synthetic construct
CASSQDGVATDTQYF

SEQ ID NO: 110 - protein, 15 aa, synthetic construct
CASSQDKGRDQPQHF

SEQ ID NO: 111 - protein, 15 aa, synthetic construct
CASSQDRPSFTEAFF

SEQ ID NO: 112 - protein, 16 aa, synthetic construct
CASSQDRQKLSGELFF

SEQ ID NO: 113 - protein, 16 aa, synthetic construct
CASSQDRTSTRDEQFF

SEQ ID NO: 114 - protein, 16 aa, synthetic construct
CASSQDWVVGNQPQHF

SEQ ID NO: 115 - protein, 15 aa, synthetic construct
CASSQEDRGNQPQHF

SEQ ID NO: 116 - protein, 14 aa, synthetic construct
CASSQGGVGETQYF

SEQ ID NO: 117 - protein, 15 aa, synthetic construct
CASSQGRGGYQPQHF

SEQ ID NO: 118 - protein, 15 aa, synthetic construct
CASSQGTGGMRGYTF

SEQ ID NO: 119 - protein, 12 aa, synthetic construct
CASSQQGSEQYV

SEQ ID NO: 120 - protein, 13 aa, synthetic construct
CASSQSEVGGQFF

SEQ ID NO: 121 - protein, 16 aa, synthetic construct
CASSRDSGRAGDTQYF

SEQ ID NO: 122 - protein, 12 aa, synthetic construct
CASSREGYGYTF

SEQ ID NO: 123 - protein, 14 aa, synthetic construct
CASSRQSSGNTIYF

SEQ ID NO: 124 - protein, 16 aa, synthetic construct
CASSRSGLFNTEGAFF

SEQ ID NO: 125 - protein, 15 aa, synthetic construct
CASSRTALAANVLTF

SEQ ID NO: 126 - protein, 14 aa, synthetic construct
CASSRTGDNSPLHF

SEQ ID NO: 127 - protein, 14 aa, synthetic construct
CASSRTGGGRGYTF

SEQ ID NO: 128 - protein, 12 aa, synthetic construct
CASSSENSPLHF

SEQ ID NO: 129 - protein, 14 aa, synthetic construct
CASSSGGLNTEAFF

SEQ ID NO: 130 - protein, 12 aa, synthetic construct
CASSSGNSPLHF

SEQ ID NO: 131 - protein, 16 aa, synthetic construct
CASSSLQVNSGNTIYF

SEQ ID NO: 132 - protein, 14 aa, synthetic construct
CASSSLTPGYGYTF

SEQ ID NO: 133 - protein, 16 aa, synthetic construct
CASSSPTLSTNEKLFF

SEQ ID NO: 134 - protein, 14 aa, synthetic construct
CASSSRTGYYEQYF

SEQ ID NO: 135 - protein, 15 aa, synthetic construct
CASSSSQRTMDGYTF

SEQ ID NO: 136 - protein, 12 aa, synthetic construct
CASSSTGTGPFF

SEQ ID NO: 137 - protein, 15 aa, synthetic construct
CASSSVWGQGGEQYF

SEQ ID NO: 138 - protein, 16 aa, synthetic construct
CASSTGGWGPNSPLHF

SEQ ID NO: 139 - protein, 13 aa, synthetic construct
CASSTGPQETQYF

SEQ ID NO: 140 - protein, 13 aa, synthetic construct
CASSTQENEKLFF

SEQ ID NO: 141 - protein, 15 aa, synthetic construct
CASSTRTNQHEKLFF

SEQ ID NO: 142 - protein, 14 aa, synthetic construct
CASSTTAAGNTIYF

SEQ ID NO: 143 - protein, 16 aa, synthetic construct
CASSVGGLASSYEQYF

SEQ ID NO: 144 - protein, 16 aa, synthetic construct
CASSVGLAGSQETQYF

SEQ ID NO: 145 - protein, 14 aa, synthetic construct
CASSWGMPNEKLFF

SEQ ID NO: 146 - protein, 13 aa, synthetic construct
CASSWGSNQPQHF

SEQ ID NO: 147 - protein, 14 aa, synthetic construct
CASSWTPAGETQYF

SEQ ID NO: 148 - protein, 16 aa, synthetic construct
CASSYPSGAFGNEQFF

SEQ ID NO: 149 - protein, 14 aa, synthetic construct
CASSYRGAGQPQHF

SEQ ID NO: 150 - protein, 11 aa, synthetic construct
CASSYSYEQYF

SEQ ID NO: 151 - protein, 11 aa, synthetic construct
CASSYTTEAFF

SEQ ID NO: 152 - protein, 14 aa, synthetic construct
CASTPGSGANVLTF

SEQ ID NO: 153 - protein, 15 aa, synthetic construct
CASTPSQGHNSPLHF

SEQ ID NO: 154 - protein, 12 aa, synthetic construct
CASRRNGLYYTF

SEQ ID NO: 155 - protein, 15 aa, synthetic construct
CATSDDSGQGAEAFF

SEQ ID NO: 156 - protein, 15 aa, synthetic construct
CATSDMGLADNEQFF

SEQ ID NO: 157 - protein, 16 aa, synthetic construct
CATSDPSGPNYNEQFF

SEQ ID NO: 158 - protein, 15 aa, synthetic construct
CATSEGGQGGYGYTF

SEQ ID NO: 159 - protein, 13 aa, synthetic construct
CATSGGGAYEQYF

SEQ ID NO: 160 - protein, 16 aa, synthetic construct
CATSQERRQVGSPLHF

SEQ ID NO: 161 - protein, 16 aa, synthetic construct
CAWSALAGSWAGELFF

SEQ ID NO: 162 - protein, 13 aa, synthetic construct
CSAAGTGNTEAFF

SEQ ID NO: 163 - protein, 14 aa, synthetic construct
CSAARQRTNYGYTF

SEQ ID NO: 164 - protein, 14 aa, synthetic construct
CSADRTSAKNIQYF

SEQ ID NO: 165 - protein, 17 aa, synthetic construct
CSAFRLAAQGGSYEQYF

SEQ ID NO: 166 - protein, 15 aa, synthetic construct
CSAIRPGVGDYEQYF

SEQ ID NO: 167 - protein, 14 aa, synthetic construct
CSAKSTGYDYEQYF

SEQ ID NO: 168 - protein, 15 aa, synthetic construct
CSALSQSGGTNIQYF

SEQ ID NO: 169 - protein, 13 aa, synthetic construct
CSALWSGDGEQFF

SEQ ID NO: 170 - protein, 15 aa, synthetic construct
CSAMTREGGNQPQHF

SEQ ID NO: 171 - protein, 14 aa, synthetic construct
CSANPLAGGGEQYF

SEQ ID NO: 172 - protein, 14 aa, synthetic construct
CSAPGPAAAGELFF

SEQ ID NO: 173 - protein, 15 aa, synthetic construct
CSAPGTSAGANVLTF

SEQ ID NO: 174 - protein, 15 aa, synthetic construct
CSAPKLVGSGNTIYF

SEQ ID NO: 175 - protein, 13 aa, synthetic construct
CSAPQDRNNEQFF

SEQ ID NO: 176 - protein, 14 aa, synthetic construct
CSAPSTDRVRGYTF

SEQ ID NO: 177 - protein, 16 aa, synthetic construct
CSARDHTSGSGNEQFF

SEQ ID NO: 178 - protein, 16 aa, synthetic construct
CSARDPDRGSGNEQYF

SEQ ID NO: 179 - protein, 16 aa, synthetic construct
CSARDQGALLNSPLHF

SEQ ID NO: 180 - protein, 14 aa, synthetic construct
CSARDRGGNTEAFF

SEQ ID NO: 181 - protein, 13 aa, synthetic construct
CSARDRVGGEQFF

SEQ ID NO: 182 - protein, 14 aa, synthetic construct
CSARDVRLNTEAFF

SEQ ID NO: 183 - protein, 21 aa, synthetic construct
CSARDVWGTGNSQASGNEQFF

SEQ ID NO: 184 - protein, 14 aa, synthetic construct
CSARGLAGADTQYF

SEQ ID NO: 185 - protein, 14 aa, synthetic construct
CSARGPGGNTEAFF

SEQ ID NO: 186 - protein, 13 aa, synthetic construct
CSARGPGTDTQYF

SEQ ID NO: 187 - protein, 13 aa, synthetic construct
CSARGRQDQPQHF

SEQ ID NO: 188 - protein, 13 aa, synthetic construct
CSARTITGSGYTF

SEQ ID NO: 189 - protein, 15 aa, synthetic construct
CSARTSGRGYNEQFF

SEQ ID NO: 190 - protein, 16 aa, synthetic construct
CSARVLGAGPNNEQFF

SEQ ID NO: 191 - protein, 15 aa, synthetic construct
CSARVSAVSTDTQYF

SEQ ID NO: 192 - protein, 15 aa, synthetic construct
CSASPLAGGSYEQYF

SEQ ID NO: 193 - protein, 15 aa, synthetic construct
CSASPLKAGANVLTF

SEQ ID NO: 194 - protein, 13 aa, synthetic construct
CSASRDSNQPQHF

SEQ ID NO: 195 - protein, 15 aa, synthetic construct
CSASSDRGGNQPQHF

SEQ ID NO: 196 - protein, 13 aa, synthetic construct
CSASSGTVGGYTF

SEQ ID NO: 197 - protein, 15 aa, synthetic construct
CSASSGVSSYNEQFF

SEQ ID NO: 198 - protein, 16 aa, synthetic construct
CSATRFGQANTGELFF

SEQ ID NO: 199 - protein, 15 aa, synthetic construct
CSATTWTGGNTEAFF

SEQ ID NO: 200 - protein, 16 aa, synthetic construct
CSAVDWTSGSSYEQYV

SEQ ID NO: 201 - protein, 16 aa, synthetic construct
CSAVLGLAGVRDTQYF

SEQ ID NO: 202 - protein, 15 aa, synthetic construct
CSISPDRGGNQPQHF

SEQ ID NO: 203 - protein, 15 aa, synthetic construct
CSLVPDRGGNQPQHF

SEQ ID NO: 204 - protein, 12 aa, synthetic construct
CSRGGREGEQFF

SEQ ID NO: 205 - protein, 13 aa, synthetic construct
CSVEGQATYEQYF

SEQ ID NO: 206 - protein, 13 aa, synthetic construct
CSVEGQGNYGYTF

SEQ ID NO: 207 - protein, 16 aa, synthetic construct
CSVLGQGAPRSYEQYF

SEQ ID NO: 208 - protein, 12 aa, synthetic construct
CSVRGRANEQYF

SEQ ID NO: 209 - protein, 13 aa, synthetic construct
CASARTGQETQYF

SEQ ID NO: 210 - protein, 13 aa, synthetic construct
CASNLPRSGELFF

SEQ ID NO: 211 - protein, 14 aa, synthetic construct
CASRPELDSYEQYF

SEQ ID NO: 212 - protein, 15 aa, synthetic construct
CASRRGGISNQPQHF

SEQ ID NO: 213 - protein, 14 aa, synthetic construct
CASSEHGGNYGYTF

SEQ ID NO: 214 - protein, 15 aa, synthetic construct
CASSIFTLSNQPQHF

SEQ ID NO: 215 - protein, 13 aa, synthetic construct
CASSKQGATEAFF

SEQ ID NO: 216 - protein, 13 aa, synthetic construct
CASSLGANYGYTF

SEQ ID NO: 217 - protein, 13 aa, synthetic construct
CASSLTDLYEQYF

SEQ ID NO: 218 - protein, 12 aa, synthetic construct
CASSPDRVEQYF

SEQ ID NO: 219 - protein, 16 aa, synthetic construct
CASSPPGGTEVYEQYF

SEQ ID NO: 220 - protein, 17 aa, synthetic construct
CASSPPPGRAETGELFF

SEQ ID NO: 221 - protein, 12 aa, synthetic construct
CASSRGAGELFF

SEQ ID NO: 222 - protein, 13 aa, synthetic construct
CASSVGGDYGYTF

SEQ ID NO: 223 - protein, 14 aa, synthetic construct
CASSYGTANTEAFF

SEQ ID NO: 224 - protein, 18 aa, synthetic construct
CASSYSTLAGGHSYEQYF

SEQ ID NO: 225 - protein, 11 aa, synthetic construct
CASSYSYEQYF

SEQ ID NO: 226 - protein, 13 aa, synthetic construct
CSVAGQGNSPLHF

SEQ ID NO: 227 - protein, 12 aa, synthetic construct
CASGGTTDTQYF

SEQ ID NO: 228 - protein, 13 aa, synthetic construct
CASRRGNTGELFF

SEQ ID NO: 229 - protein, 14 aa, synthetic construct
CASRSTGTGEKLFF

SEQ ID NO: 230 - protein, 16 aa, synthetic construct
CASSFWAGVSTDTQYF

SEQ ID NO: 231 - protein, 14 aa, synthetic construct
CASSGGRKLDTQYF

SEQ ID NO: 232 - protein, 18 aa, synthetic construct
CASSLNLLDRASLETQYF

SEQ ID NO: 233 - protein, 14 aa, synthetic construct
CASSLTGYNSPLHF

SEQ ID NO: 234 - protein, 13 aa, synthetic construct
CASSLVLEHEQFF

SEQ ID NO: 235 - protein, 16 aa, synthetic construct
CASSQDRITSGYGYTF

SEQ ID NO: 236 - protein, 15 aa, synthetic construct
CASSTGGRSNQPQHF

SEQ ID NO: 237 - protein, 15 aa, synthetic construct
CASSVVPGAGGEQFF

SEQ ID NO: 238 - protein, 16 aa, synthetic construct
CASSVVPGGPGGELFF

SEQ ID NO: 239 - protein, 14 aa, synthetic construct
CASSWAPHTDEQFF

SEQ ID NO: 240 - protein, 14 aa, synthetic construct
CASSWTGNTGELFF

SEQ ID NO: 241 - protein, 12 aa, synthetic construct
CASSYTQETQYF

SEQ ID NO: 242 - protein, 16 aa, synthetic construct
CASSYTTSGGTYEQYF

SEQ ID NO: 243 - protein, 14 aa, synthetic construct
CASTPSGGTQPQHF

SEQ ID NO: 244 - protein, 13 aa, synthetic construct
CATSDPGTREQFF

SEQ ID NO: 245 - protein, 16 aa, synthetic construct
CATSYRAGGGYNEQFF

SEQ ID NO: 246 - protein, 10 aa, synthetic construct
CSARGNEQFF

SEQ ID NO: 247 - protein, 11 aa, synthetic construct
CSASSSGTQYF

SEQ ID NO: 248 - protein, 13 aa, synthetic construct
CAAEDLAKNIQYF

SEQ ID NO: 249 - protein, 14 aa, synthetic construct
CAGRRRLGDSPLHF

SEQ ID NO: 250 - protein, 13 aa, synthetic construct
CASKQDLNTEAFF

SEQ ID NO: 251 - protein, 13 aa, synthetic construct
CASLSGPGYEQYF

SEQ ID NO: 252 - protein, 14 aa, synthetic construct
CASNAGYTSGELFF

SEQ ID NO: 253 - protein, 13 aa, synthetic construct
CASQDRTALEQYF

SEQ ID NO: 254 - protein, 15 aa, synthetic construct
CASRGGSSGANVLTF

SEQ ID NO: 255 - protein, 15 aa, synthetic construct
CASRGTSGRTYEQYF

SEQ ID NO: 256 - protein, 16 aa, synthetic construct
CASRLAGQEANYGYTF

SEQ ID NO: 257 - protein, 13 aa, synthetic construct
CASRPSLLRELFF

SEQ ID NO: 258 - protein, 13 aa, synthetic construct
CASRRGNTGELFF

SEQ ID NO: 259 - protein, 14 aa, synthetic construct
CASRRNSGANVLTF

SEQ ID NO: 260 - protein, 14 aa, synthetic construct
CASSARDRYYGYTF

SEQ ID NO: 261 - protein, 13 aa, synthetic construct
CASSDRDTDTQYF

SEQ ID NO: 262 - protein, 15 aa, synthetic construct
CASSEGGTRHETQYF

SEQ ID NO: 263 - protein, 14 aa, synthetic construct
CASSEGQGADTQYF

SEQ ID NO: 264 - protein, 14 aa, synthetic construct
CASSEVSGNQPQHF

SEQ ID NO: 265 - protein, 14 aa, synthetic construct
CASSFGLTNEKLFF

SEQ ID NO: 266 - protein, 14 aa, synthetic construct
CASSFGTGVYGYTF

SEQ ID NO: 267 - protein, 15 aa, synthetic construct
CASSFMDRDNSPLHF

SEQ ID NO: 268 - protein, 13 aa, synthetic construct
CASSFSGDNEQFF

SEQ ID NO: 269 - protein, 14 aa, synthetic construct
CASSGQGGGYGYTF

SEQ ID NO: 270 - protein, 13 aa, synthetic construct
CASSITRKETQYF

SEQ ID NO: 271 - protein, 12 aa, synthetic construct
CASSLAHYEQYF

SEQ ID NO: 272 - protein, 14 aa, synthetic construct
CASSLAPHTDEQFF

SEQ ID NO: 273 - protein, 15 aa, synthetic construct
CASSLDEQGQNEQFF

SEQ ID NO: 274 - protein, 13 aa, synthetic construct
CASSLEADYEQYF

SEQ ID NO: 275 - protein, 13 aa, synthetic construct
CASSLEDNQPQHF

SEQ ID NO: 276 - protein, 14 aa, synthetic construct
CASSLGGQVYGYTF

SEQ ID NO: 277 - protein, 15 aa, synthetic construct
CASSLGQGLNEKLFF

SEQ ID NO: 278 - protein, 13 aa, synthetic construct
CASSLGRNYGYTF

SEQ ID NO: 279 - protein, 15 aa, synthetic construct
CASSLGTSAYNEQFF

SEQ ID NO: 280 - protein, 15 aa, synthetic construct
CASSLMQAANSPLHF

SEQ ID NO: 281 - protein, 15 aa, synthetic construct
CASSLMSATNYGYTF

SEQ ID NO: 282 - protein, 14 aa, synthetic construct
CASSLQGAREKLFF

SEQ ID NO: 283 - protein, 13 aa, synthetic construct
CASSLQGGTEAFF

SEQ ID NO: 284 - protein, 15 aa, synthetic construct
CASSLSGSSYNEQFF

SEQ ID NO: 285 - protein, 18 aa, synthetic construct
CASSLSGTGNGRNQPQHF

SEQ ID NO: 286 - protein, 15 aa, synthetic construct
CASSLSRDAVGGYTF

SEQ ID NO: 287 - protein, 15 aa, synthetic construct
CASSLTGTGGYEQYF

SEQ ID NO: 288 - protein, 14 aa, synthetic construct
CASSLVAGGYEQYF

SEQ ID NO: 289 - protein, 11 aa, synthetic construct
CASSLYNEQFF

SEQ ID NO: 290 - protein, 14 aa, synthetic construct
CASSPPFGSYEQYF

SEQ ID NO: 291 - protein, 11 aa, synthetic construct
CASSQADTQYF

SEQ ID NO: 292 - protein, 14 aa, synthetic construct
CASSQGQEFGKLFF

SEQ ID NO: 293 - protein, 15 aa, synthetic construct
CASSQTSGSYNEQFF

SEQ ID NO: 294 - protein, 11 aa, synthetic construct
CASSRTYEQYF

SEQ ID NO: 295 - protein, 12 aa, synthetic construct
CASSSANYGYTF

SEQ ID NO: 296 - protein, 15 aa, synthetic construct
CASSSGEGEAGELFF

SEQ ID NO: 297 - protein, 14 aa, synthetic construct
CASSSGGLNTEAFF

SEQ ID NO: 298 - protein, 15 aa, synthetic construct
CASSSGGRAWDTQYF

SEQ ID NO: 299 - protein, 14 aa, synthetic construct
CASSSGLAAYEQYF

SEQ ID NO: 300 - protein, 14 aa, synthetic construct
CASSSQGSQETQYF

SEQ ID NO: 301 - protein, 14 aa, synthetic construct
CASSSTGGNQPQHF

SEQ ID NO: 302 - protein, 15 aa, synthetic construct
CASSVRQGSAGELFF

SEQ ID NO: 303 - protein, 14 aa, synthetic construct
CASSWGLADETQYF

SEQ ID NO: 304 - protein, 13 aa, synthetic construct
CASSYDSRYGYTF

SEQ ID NO: 305 - protein, 12 aa, synthetic construct
CASSYSAGEQYF

SEQ ID NO: 306 - protein, 15 aa, synthetic construct
CASSYSPSTKNIQYF

SEQ ID NO: 307 - protein, 12 aa, synthetic construct
CATEGRGNTIYF

SEQ ID NO: 308 - protein, 16 aa, synthetic construct
CATPPGGLANTGELFF

SEQ ID NO: 309 - protein, 16 aa, synthetic construct
CATSDSSGRYYNEQFF

SEQ ID NO: 310 - protein, 12 aa, synthetic construct
CAWSGMNTEAFF

SEQ ID NO: 311 - protein, 13 aa, synthetic construct
CSARGGHSFEQYF

SEQ ID NO: 312 - protein, 12 aa, synthetic construct
CSARNGDTEAFF

SEQ ID NO: 313 - protein, 14 aa, synthetic construct
CASSLVAGGYEQYF

SEQ ID NO: 314 - protein, 11 aa, synthetic construct
CSVRVNTEAFF

SEQ ID NO: 315 - protein, 12 aa, synthetic construct
CAFILNNNDMRF

SEQ ID NO: 316 - protein, 15 aa, synthetic construct
CALSEAVGGANNLFF

SEQ ID NO: 317 - protein, 16 aa, synthetic construct
CAFMKQSGGSQGNLIF

SEQ ID NO: 318 - protein, 16 aa, synthetic construct
CAVRSYSGAGSYQLTF

SEQ ID NO: 319 - protein, 15 aa, synthetic construct
CAACDRGSTLGRLYF

SEQ ID NO: 320 - protein, 11 aa, synthetic construct
CAAGNNARLMF

SEQ ID NO: 321 - protein, 12 aa, synthetic construct
CAGLSNDYKLSF

SEQ ID NO: 322 - protein, 11 aa, synthetic construct
CIVKSWGKLQF

SEQ ID NO: 323 - protein, 11 aa, synthetic construct
CATDAGNDMRF

SEQ ID NO: 324 - protein, 15 aa, synthetic construct
CAVGAGGTSYGKLTF

SEQ ID NO: 325 - protein, 13 aa, synthetic construct
CAGHSNTGNQFYF

SEQ ID NO: 326 - protein, 15 aa, synthetic construct
CVVSRVKAAGNKLTF

SEQ ID NO: 327 - protein, 17 aa, synthetic construct
CAAPYPTGGTSYGKLTF

SEQ ID NO: 328 - protein, 13 aa, synthetic construct
CAVSERGFQKLVF

SEQ ID NO: 329 - protein, 12 aa, synthetic construct
CALSESWGKLQF

SEQ ID NO: 330 - protein, 15 aa, synthetic construct
CAVGASGAAGNKLTF

SEQ ID NO: 331 - protein, 14 aa, synthetic construct
CAVSDNQGAQKLVF

SEQ ID NO: 332 - protein, 15 aa, synthetic construct
CAAPTSGGGADGLTF

SEQ ID NO: 333 - protein, 14 aa, synthetic construct
CAGQGYFGNEKLTF

SEQ ID NO: 334 - protein, 13 aa, synthetic construct
CALSYNQGGKLIF

SEQ ID NO: 335 - protein, 17 aa, synthetic construct
CAVSEPGSGGSNYKLTF

SEQ ID NO: 336 - protein, 13 aa, synthetic construct
CVVSDVNRNQFYF

SEQ ID NO: 337 - protein, 14 aa, synthetic construct
CAVNAPFGNEKLTF

SEQ ID NO: 338 - protein, 14 aa, synthetic construct
CVVNWGGGYNKLIF

SEQ ID NO: 339 - protein, 16 aa, synthetic construct
CAEITRYGGSQGNLIF

SEQ ID NO: 340 - protein, 13 aa, synthetic construct
CALSTSGTYKYIF

SEQ ID NO: 341 - protein, 17 aa, synthetic construct
CVVGVRGGGTSYGKLTF

SEQ ID NO: 342 - protein, 15 aa, synthetic construct
CALRAQNSGYSTLTF

SEQ ID NO: 343 - protein, 16 aa, synthetic construct
CAFMKHMSGNNRKLIW

SEQ ID NO: 344 - protein, 11 aa, synthetic construct
CAASGTYKYIF

SEQ ID NO: 345 - protein, 11 aa, synthetic construct
CAEIAGNQFYF

SEQ ID NO: 346 - protein, 12 aa, synthetic construct
CAVSDSFQKLVF

SEQ ID NO: 347 - protein, 13 aa, synthetic construct
CAVSSTYGNKLVF

SEQ ID NO: 348 - protein, 14 aa, synthetic construct
CAAIGSSNTGKLIF

SEQ ID NO: 349 - protein, 14 aa, synthetic construct
CAVSEDTGGFKTIF

SEQ ID NO: 350 - protein, 13 aa, synthetic construct
CAVLGTSGSRLTF

SEQ ID NO: 351 - protein, 15 aa, synthetic construct
CAVRDKRSNDYKLSF

SEQ ID NO: 352 - protein, 16 aa, synthetic construct
CAASNVLTGGGNKLTF

SEQ ID NO: 353 - protein, 15 aa, synthetic construct
CAAISRTGSARQLTF

SEQ ID NO: 354 - protein, 14 aa, synthetic construct
CATDATGANSKLTF

SEQ ID NO: 355 - protein, 15 aa, synthetic construct
CALSDSGGGADGLTF

SEQ ID NO: 356 - protein, 15 aa, synthetic construct
CALSEAGNAGNMLTF

SEQ ID NO: 357 - protein, 14 aa, synthetic construct
CAVPGGTSYGKLTF

SEQ ID NO: 358 - protein, 14 aa, synthetic construct
CAVGARQAGTALIF

SEQ ID NO: 359 - protein, 18 aa, synthetic construct
CAFMTINAGGTSYGKLTF

SEQ ID NO: 360 - protein, 13 aa, synthetic construct
CVVRVGFGNVLHC

SEQ ID NO: 361 - protein, 14 aa, synthetic construct
CAATRRSNDYKLSF

SEQ ID NO: 362 - protein, 14 aa, synthetic construct
CIAPNMDSNYQLIW

SEQ ID NO: 363 - protein, 15 aa, synthetic construct
CATPKIYNQGGKLIF

SEQ ID NO: 364 - protein, 14 aa, synthetic construct
CAVRPPGANSKLTF

SEQ ID NO: 365 - protein, 13 aa, synthetic construct
CAVEDDNNARLMF

SEQ ID NO: 366 - protein, 12 aa, synthetic construct
CAASPRFNKFYF

SEQ ID NO: 367 - protein, 11 aa, synthetic construct
CAVNSNYQLIW

SEQ ID NO: 368 - protein, 15 aa, synthetic construct
CAVKGPAGNNRKLIW

SEQ ID NO: 369 - protein, 13 aa, synthetic construct
CALSVGGAQKLVF

SEQ ID NO: 370 - protein, 15 aa, synthetic construct
CVVLERNTGGFKTIF

SEQ ID NO: 371 - protein, 13 aa, synthetic construct
CATDRDSNYQLIW

SEQ ID NO: 372 - protein, 13 aa, synthetic construct
CAMREGSDYKLSF

SEQ ID NO: 373 - protein, 13 aa, synthetic construct
CAASGGGADGLTF

SEQ ID NO: 374 - protein, 13 aa, synthetic construct
CAASLPGNTPLVF

SEQ ID NO: 375 - protein, 13 aa, synthetic construct
CAYRSGATNKLIF

SEQ ID NO: 376 - protein, 15 aa, synthetic construct
CAVPPPNFGNEKLTF

SEQ ID NO: 377 - protein, 14 aa, synthetic construct
CATDSSNTGNQFYF

SEQ ID NO: 378 - protein, 15 aa, synthetic construct
CIAHDRGSTLGRLYF

SEQ ID NO: 379 - protein, 15 aa, synthetic construct
CALSPQGTGGFKTIF

SEQ ID NO: 380 - protein, 13 aa, synthetic construct
CAALAGPGYALNF

SEQ ID NO: 381 - protein, 14 aa, synthetic construct
CAVRGAGNNRKLIW

SEQ ID NO: 382 - protein, 14 aa, synthetic construct
CADSYGGATNKLIF

SEQ ID NO: 383 - protein, 17 aa, synthetic construct
CILSNVYSGAGSYQLTF

SEQ ID NO: 384 - protein, 10 aa, synthetic construct
CAFDNNDMRF

SEQ ID NO: 385 - protein, 17 aa, synthetic construct
CAFIGLLGIQGAQKLVF

SEQ ID NO: 386 - protein, 15 aa, synthetic construct
CAASIRSGGSYIPTF

SEQ ID NO: 387 - protein, 13 aa, synthetic construct
CAANAGNNRKLIW

SEQ ID NO: 388 - protein, 14 aa, synthetic construct
CAAEFTGTASKLTF

SEQ ID NO: 389 - protein, 14 aa, synthetic construct
CAEGRLTGGFKTIF

SEQ ID NO: 390 - protein, 13 aa, synthetic construct
CAVRGGGGFKTIF

SEQ ID NO: 391 - protein, 14 aa, synthetic construct
CGTEEMNRDDKIIF

SEQ ID NO: 392 - protein, 12 aa, synthetic construct
CAASNSGYALNF

SEQ ID NO: 393 - protein, 12 aa, synthetic construct
CAAASGYSTLTF

SEQ ID NO: 394 - protein, 15 aa, synthetic construct
CVVSLLTGGGNKLTF

SEQ ID NO: 395 - protein, 14 aa, synthetic construct
CAVRSSLGNNRLAF

SEQ ID NO: 396 - protein, 13 aa, synthetic construct
CAAMGNRDDKIIF

SEQ ID NO: 397 - protein, 18 aa, synthetic construct
CAVDRARNSGGSNYKLTF

SEQ ID NO: 398 - protein, 12 aa, synthetic construct
CALGEYGNKLVF

SEQ ID NO: 399 - protein, 14 aa, synthetic construct
CAASGANSGYALNF

SEQ ID NO: 400 - protein, 15 aa, synthetic construct
CAGPTNSGGYQKVTF

SEQ ID NO: 401 - protein, 16 aa, synthetic construct
CIVRVAYNNAGNMLTF

SEQ ID NO: 402 - protein, 13 aa, synthetic construct
CAAGDTGRRALTF

SEQ ID NO: 403 - protein, 13 aa, synthetic construct
CASGRGSQGNLIF

SEQ ID NO: 404 - protein, 15 aa, synthetic construct
CALIDRGSTLGRLYF

SEQ ID NO: 405 - protein, 15 aa, synthetic construct
CAAPPGGTSYGKLTF

SEQ ID NO: 406 - protein, 14 aa, synthetic construct
CAVQAAGGYQKVTF

SEQ ID NO: 407 - protein, 12 aa, synthetic construct
CAERQGNTPLVF

SEQ ID NO: 408 - protein, 13 aa, synthetic construct
CAASGDRDDKIIF

SEQ ID NO: 409 - protein, 12 aa, synthetic construct
CATDPGANNLFF

SEQ ID NO: 410 - protein, 16 aa, synthetic construct
CALPPGGGTSYGKLTF

SEQ ID NO: 411 - protein, 14 aa, synthetic construct
CAVRDAGGYNKLIF

SEQ ID NO: 412 - protein, 14 aa, synthetic construct
CAMRLGAAGNKLTF

SEQ ID NO: 413 - protein, 14 aa, synthetic construct
CALSDYNQGGKLIF

SEQ ID NO: 414 - protein, 14 aa, synthetic construct
CALSPRGGYQKVTF

SEQ ID NO: 415 - protein, 13 aa, synthetic construct
CAVRGRGYSTLTF

SEQ ID NO: 416 - protein, 15 aa, synthetic construct
CAGDFGGTSYGKLTF

SEQ ID NO: 417 - protein, 12 aa, synthetic construct
CAASRGNNRLAF

SEQ ID NO: 418 - protein, 14 aa, synthetic construct
CAVRDGGGYNKLIF

SEQ ID NO: 419 - protein, 16 aa, synthetic construct
CAVQGYLGGATNKLIF

SEQ ID NO: 420 - protein, 12 aa, synthetic construct
CAVYSNTGKLIF

SEQ ID NO: 421 - protein, 12 aa, synthetic construct
CAMRVNNARLMF

SEQ ID NO: 422 - protein, 15 aa, synthetic construct
CALSDRTGANSKLTF

SEQ ID NO: 423 - protein, 14 aa, synthetic construct
CALSDRGSARQLTF

SEQ ID NO: 424 - protein, 13 aa, synthetic construct
CAAKGNTGNQFYF

SEQ ID NO: 425 - protein, 16 aa, synthetic construct
CAALRDRGSTLGRLYF

SEQ ID NO: 426 - protein, 13 aa, synthetic construct
CAVSETGFQKLVF

SEQ ID NO: 427 - protein, 13 aa, synthetic construct
CATDATSGSRLTF

SEQ ID NO: 428 - protein, 12 aa, synthetic construct
CAERNNNARLMF

SEQ ID NO: 429 - protein, 11 aa, synthetic construct
CAASTGNQFYF

SEQ ID NO: 430 - protein, 14 aa, synthetic construct
CVVTLNNAGNMLTF

SEQ ID NO: 431 - protein, 16 aa, synthetic construct
CAASGGEGGGADGLTF

SEQ ID NO: 432 - protein, 13 aa, synthetic construct
CAVGPWGDYKLSF

SEQ ID NO: 433 - protein, 12 aa, synthetic construct
CAASWGNTPLVF

SEQ ID NO: 434 - protein, 15 aa, synthetic construct
CAVRRRGDSNYQLIW

SEQ ID NO: 435 - protein, 15 aa, synthetic construct
CAVSEKGAGGFKTIF

SEQ ID NO: 436 - protein, 14 aa, synthetic construct
CAVSQMDSSYKLIF

SEQ ID NO: 437 - protein, 13 aa, synthetic construct
CAVSGLNNARLMF

SEQ ID NO: 438 - protein, 14 aa, synthetic construct
CALSGGQAGTALIF

SEQ ID NO: 439 - protein, 12 aa, synthetic construct
CAVRRQGGKLIF

SEQ ID NO: 440 - protein, 16 aa, synthetic construct
CAVRPESNFGNEKLTF

SEQ ID NO: 441 - protein, 16 aa, synthetic construct
CIVRVAGGTSYGKLTF

SEQ ID NO: 442 - protein, 13 aa, synthetic construct
CAEMNNAGNMLTF

SEQ ID NO: 443 - protein, 14 aa, synthetic construct
CAASIVGSQGNLIF

SEQ ID NO: 444 - protein, 18 aa, synthetic construct
CIVRVGGISNFGNEKLTF

SEQ ID NO: 445 - protein, 12 aa, synthetic construct
CAVETSGSRLTF

SEQ ID NO: 446 - protein, 15 aa, synthetic construct
CALRAGGTSYGKLTF

SEQ ID NO: 447 - protein, 13 aa, synthetic construct
CAASTGGYNKLIF

SEQ ID NO: 448 - protein, 12 aa, synthetic construct
CAYSGDGYALNF

SEQ ID NO: 449 - protein, 16 aa, synthetic construct
CATDARGDFGNEKLTF

SEQ ID NO: 450 - protein, 13 aa, synthetic construct
CATDKGSNYQLIW

SEQ ID NO: 451 - protein, 11 aa, synthetic construct
CAASTGNQFYF

SEQ ID NO: 452 - protein, 15 aa, synthetic construct
CLVGVDQTGANNLFF

SEQ ID NO: 453 - protein, 14 aa, synthetic construct
CAVSVPGSNYQLIW

SEQ ID NO: 454 - protein, 13 aa, synthetic construct
CAANNNAGNMLTF

SEQ ID NO: 455 - protein, 12 aa, synthetic construct
CAGRNSGYALNF

SEQ ID NO: 456 - protein, 16 aa, synthetic construct
CAVPYLSGAGSYQLTF

SEQ ID NO: 457 - protein, 15 aa, synthetic construct
CAVSPNSGGYQKVTF

SEQ ID NO: 458 - protein, 14 aa, synthetic construct
CAASTPNNNARLMF

SEQ ID NO: 459 - protein, 14 aa, synthetic construct
CVVEPGNYGQNFVF

SEQ ID NO: 460 - protein, 14 aa, synthetic construct
CAMSASTGGFKTIF

SEQ ID NO: 461 - protein, 14 aa, synthetic construct
CAENMMDSSYKLIF

SEQ ID NO: 462 - protein, 14 aa, synthetic construct
CAASGNFGNEKLTF

SEQ ID NO: 463 - protein, 17 aa, synthetic construct
CAVPSNAGGTSYGKLTF

SEQ ID NO: 464 - protein, 14 aa, synthetic construct
CAVRDGAGSYQLTF

SEQ ID NO: 465 - protein, 15 aa, synthetic construct
CALTGMMDSSYKLIF

SEQ ID NO: 466 - protein, 12 aa, synthetic construct
CAYRTPPNDMRF

SEQ ID NO: 467 - protein, 14 aa, synthetic construct
CAASATDSSYKLIF

SEQ ID NO: 468 - protein, 14 aa, synthetic construct
CAVNAPFGNEKLTF

SEQ ID NO: 469 - protein, 13 aa, synthetic construct
CAASRTGRRALTF

SEQ ID NO: 470 - protein, 14 aa, synthetic construct
CAHTSSGGSYIPTF

SEQ ID NO: 471 - protein, 12 aa, synthetic construct
CAASDSNYQLIW

SEQ ID NO: 472 - protein, 13 aa, synthetic construct
CAVGAYNNNDMRF

SEQ ID NO: 473 - protein, 12 aa, synthetic construct
CAGFNSGYALNF

SEQ ID NO: 474 - protein, 14 aa, synthetic construct
CAVSSTGANSKLTF

SEQ ID NO: 475 - protein, 16 aa, synthetic construct
CAFILPSGAGSYQLTF

SEQ ID NO: 476 - protein, 13 aa, synthetic construct
CAMSEPNGQNFVF

SEQ ID NO: 477 - protein, 13 aa, synthetic construct
CAPRDSGYSTLTF

SEQ ID NO: 478 - protein, 13 aa, synthetic construct
CAVRRNAGNMLTF

SEQ ID NO: 479 - protein, 12 aa, synthetic construct
CAESHNTDKLIF

SEQ ID NO: 480 - protein, 13 aa, synthetic construct
CVVGTGTASKLTF

SEQ ID NO: 481 - protein, 13 aa, synthetic construct
CAMKTGGGNKLTF

SEQ ID NO: 482 - protein, 14 aa, synthetic construct
CVVKLNSSASKIIF

SEQ ID NO: 483 - protein, 12 aa, synthetic construct
CAASLNFNKFYF

SEQ ID NO: 484 - protein, 14 aa, synthetic construct
CAVRDGGGYSTLTF

SEQ ID NO: 485 - protein, 14 aa, synthetic construct
CASRFSGGYNKLIF

SEQ ID NO: 486 - protein, 14 aa, synthetic construct
CAVSDPGGYNKLIF

SEQ ID NO: 487 - protein, 14 aa, synthetic construct
CAVSEPGGYQKVTF

SEQ ID NO: 488 - protein, 12 aa, synthetic construct
CAFRSNNNDMRF

SEQ ID NO: 489 - protein, 15 aa, synthetic construct
CAVWGVNQAGTALIF

SEQ ID NO: 490 - protein, 13 aa, synthetic construct
CGTPSGGYQKVTF

SEQ ID NO: 491 - protein, 13 aa, synthetic construct
CAASQAAGNKLTF

SEQ ID NO: 492 - protein, 13 aa, synthetic construct
CAASMGTGNQFYF

SEQ ID NO: 493 - protein, 13 aa, synthetic construct
CALSEVYNNDMRF

SEQ ID NO: 494 - protein, 13 aa, synthetic construct
CALHWRGAQKLVF

SEQ ID NO: 495 - protein, 13 aa, synthetic construct
CAVRDQAGTALIF

SEQ ID NO: 496 - protein, 13 aa, synthetic construct
CAASMAAGNQFYF

SEQ ID NO: 497 - protein, 13 aa, synthetic construct
CAASTGAGNMLTF

SEQ ID NO: 498 - protein, 12 aa, synthetic construct
CAVRDTGNQFYF

SEQ ID NO: 499 - protein, 13 aa, synthetic construct
CAVKGSNTGKLIF

SEQ ID NO: 500 - protein, 13 aa, synthetic construct
CATHPNSGYALNF

SEQ ID NO: 501 - protein, 12 aa, synthetic construct
CAGLNTGNQFYF

SEQ ID NO: 502 - protein, 12 aa, synthetic construct
CALSDQNARLMF

SEQ ID NO: 503 - protein, 15 aa, synthetic construct
CIVRVSNSGNTPLVF

SEQ ID NO: 504 - protein, 12 aa, synthetic construct
CALSDPQAALTF

SEQ ID NO: 505 - protein, 15 aa, synthetic construct
CVVSDRPGGGNKLTF

SEQ ID NO: 506 - protein, 13 aa, synthetic construct
CAMRTGGGNKLTF

SEQ ID NO: 507 - protein, 14 aa, synthetic construct
CAVSEPGGYNKLIF

SEQ ID NO: 508 - protein, 12 aa, synthetic construct
CIVKNTGTALIF

SEQ ID NO: 509 - protein, 15 aa, synthetic construct
CAVGGRGSTLGRLYF

SEQ ID NO: 510 - protein, 12 aa, synthetic construct
CAMSNNFNKFYF

SEQ ID NO: 511 - protein, 15 aa, synthetic construct
CAPPRGTGGYNKLIF

SEQ ID NO: 512 - protein, 14 aa, synthetic construct
CAVSEPGGYQKVTF

SEQ ID NO: 513 - protein, 14 aa, synthetic construct
CAVRDSGGYNKLIF

SEQ ID NO: 514 - protein, 14 aa, synthetic construct
CAVSESGGYQKVTF

SEQ ID NO: 515 - protein, 14 aa, synthetic construct
CAVRGFSDGQKLLF

SEQ ID NO: 516 - protein, 15 aa, synthetic construct
CARRGSSGSARQLTF

SEQ ID NO: 517 - protein, 14 aa, synthetic construct
CAVPYLTNAGKSTF

SEQ ID NO: 518 - protein, 13 aa, synthetic construct
CAVEETSGSRLTF

SEQ ID NO: 519 - protein, 11 aa, synthetic construct
CAGQAAYKYIF

SEQ ID NO: 520 - protein, 13 aa, synthetic construct
CAVSQAWGGKLIF

SEQ ID NO: 521 - protein, 11 aa, synthetic construct
CAGTNTDKLIF

SEQ ID NO: 522 - protein, 12 aa, synthetic construct
CAESIGTDKLIF

SEQ ID NO: 523 - protein, 12 aa, synthetic construct
CAVLFGNEKLTF

SEQ ID NO: 524 - protein, 15 aa, synthetic construct
CAVDDSGGGADGLTF

SEQ ID NO: 525 - protein, 15 aa, synthetic construct
CAASWGGTSYGKLTF

SEQ ID NO: 526 - protein, 13 aa, synthetic construct
CAPILQGAQKLVF

SEQ ID NO: 527 - protein, 13 aa, synthetic construct
CILRDNYGQNFVF

SEQ ID NO: 528 - protein, 12 aa, synthetic construct
CAVAGTASKLTF

SEQ ID NO: 529 - protein, 12 aa, synthetic construct
CAVYSNTGKLIF

SEQ ID NO: 530 - protein, 11 aa, synthetic construct
CAVNTGNQFYF

SEQ ID NO: 531 - protein, 14 aa, synthetic construct
CAVLLTGGGNKLTF

SEQ ID NO: 532 - protein, 13 aa, synthetic construct
CIVGNTGGFKTIF

SEQ ID NO: 533 - protein, 14 aa, synthetic construct
CAASVWGGSEKLVF

SEQ ID NO: 534 - protein, 13 aa, synthetic construct
CALSDRGGNKLVF

SEQ ID NO: 535 - protein, 14 aa, synthetic construct
CAARETYNTDKLIF

SEQ ID NO: 536 - protein, 13 aa, synthetic construct
CGSPGAGSYQLTF

SEQ ID NO: 537 - protein, 14 aa, synthetic construct
CAGGNAGNNRKLIW

SEQ ID NO: 538 - protein, 16 aa, synthetic construct
CAFMMLTGGGADGLTF

SEQ ID NO: 539 - protein, 14 aa, synthetic construct
CAVRDGAGSYQLTF

SEQ ID NO: 540 - protein, 12 aa, synthetic construct
CIVRVEAGKSTF

SEQ ID NO: 541 - protein, 13 aa, synthetic construct
CATIQTGANNLFF

SEQ ID NO: 542 - protein, 13 aa, synthetic construct
CAGYNSGTYKYIF

SEQ ID NO: 543 - protein, 14 aa, synthetic construct
CAVQAVNNNARLMF

SEQ ID NO: 544 - protein, 11 aa, synthetic construct
CAFMNTDKLIF

SEQ ID NO: 545 - protein, 15 aa, synthetic construct
CAMREGSGGYNKLIF

SEQ ID NO: 546 - protein, 15 aa, synthetic construct
CAMRPRSSNTGKLIF

SEQ ID NO: 547 - protein, 13 aa, synthetic construct
CATDFFGNEKLTF

SEQ ID NO: 548 - protein, 12 aa, synthetic construct
CAVSHTGNQFYF

SEQ ID NO: 549 - protein, 14 aa, synthetic construct
CAELRIQGAQKLVF

SEQ ID NO: 550 - protein, 14 aa, synthetic construct
CVVIFTGTASKLTF

SEQ ID NO: 551 - protein, 15 aa, synthetic construct
CAVSGLGGGADGLTF

SEQ ID NO: 552 - protein, 15 aa, synthetic construct
CALPDSGGGADGLTF

SEQ ID NO: 553 - protein, 16 aa, synthetic construct
CAENIKGSSGYSTLTF

SEQ ID NO: 554 - protein, 13 aa, synthetic construct
CAGEGAGSYQLTF

SEQ ID NO: 555 - protein, 12 aa, synthetic construct
CAVGDSNYQLIW

SEQ ID NO: 556 - protein, 14 aa, synthetic construct
CAVQGALNNARLMF

SEQ ID NO: 557 - protein, 16 aa, synthetic construct
CAVSDPLGGSNYKLTF

SEQ ID NO: 558 - protein, 12 aa, synthetic construct
CAGHQAGTALIF

SEQ ID NO: 559 - protein, 13 aa, synthetic construct
CAVSGGATNKLIF

SEQ ID NO: 560 - protein, 16 aa, synthetic construct
CAASIELTGGGNKLTF

SEQ ID NO: 561 - protein, 14 aa, synthetic construct
CAAGMYSSASKIIF

SEQ ID NO: 562 - protein, 13 aa, synthetic construct
CALSLSGYSTLTF

SEQ ID NO: 563 - protein, 13 aa, synthetic construct
CAVINVDFQKLVF

SEQ ID NO: 564 - protein, 13 aa, synthetic construct
CASLTGGGNKLTF

SEQ ID NO: 565 - protein, 13 aa, synthetic construct
CAMGITSGYALNF

SEQ ID NO: 566 - protein, 14 aa, synthetic construct
CAVKGGGATNKLIF

SEQ ID NO: 567 - protein, 12 aa, synthetic construct
CAVLGYGNKLVF

SEQ ID NO: 568 - protein, 15 aa, synthetic construct
CAGQLAAGTASKLTF

SEQ ID NO: 569 - protein, 14 aa, synthetic construct
CARYSGGGADGLTF

SEQ ID NO: 570 - protein, 14 aa, synthetic construct
CAVSPSGGYQKVTF

SEQ ID NO: 571 - protein, 17 aa, synthetic construct
CAENRRAGGTSYGKLTF

SEQ ID NO: 572 - protein, 13 aa, synthetic construct
CAGYNSGTYKYIF

SEQ ID NO: 573 - protein, 12 aa, synthetic construct
CAASDAGNMLTF

SEQ ID NO: 574 - protein, 16 aa, synthetic construct
CAARGNSGGSNYKLTF

SEQ ID NO: 575 - protein, 13 aa, synthetic construct
CATVNSGNTPLVF

SEQ ID NO: 576 - protein, 13 aa, synthetic construct
CAVERGSQGNLIF

SEQ ID NO: 577 - protein, 12 aa, synthetic construct
CAVMDSNYQLIW

SEQ ID NO: 578 - protein, 12 aa, synthetic construct
CAGPGYGNKLVF

SEQ ID NO: 579 - protein, 14 aa, synthetic construct
CAAGVNFGNEKLTF

SEQ ID NO: 580 - protein, 12 aa, synthetic construct
CAASRGFNDMRF

SEQ ID NO: 581 - protein, 14 aa, synthetic construct
CAVSGLVGNEKLTF

SEQ ID NO: 582 - protein, 10 aa, synthetic construct
CAVVFNKFYF

SEQ ID NO: 583 - protein, 14 aa, synthetic construct
CATEGDSGYSTLTF

SEQ ID NO: 584 - protein, 12 aa, synthetic construct
CAVSGTGNQFYF

SEQ ID NO: 585 - protein, 14 aa, synthetic construct
CAASVGGSNYKLTF

SEQ ID NO: 586 - protein, 15 aa, synthetic construct
CAASGSDSGNTPLVF

SEQ ID NO: 587 - protein, 14 aa, synthetic construct
CAMSSRGSARQLTF

SEQ ID NO: 588 - protein, 12 aa, synthetic construct
CAVNGFGNVLHC

SEQ ID NO: 589 - protein, 12 aa, synthetic construct
CAAPSRDDKIIF

SEQ ID NO: 590 - protein, 17 aa, synthetic construct
CLVGDNAPSGSARQLTF

SEQ ID NO: 591 - protein, 12 aa, synthetic construct
CAENGSDYKLSF

SEQ ID NO: 592 - protein, 14 aa, synthetic construct
CAALSHQGAQKLVF

SEQ ID NO: 593 - protein, 15 aa, synthetic construct
CAVRVFSGGYNKLIF

SEQ ID NO: 594 - protein, 14 aa, synthetic construct
CAVGERGATNKLIF

SEQ ID NO: 595 - protein, 14 aa, synthetic construct
CAVKSNSGNTPLVF

SEQ ID NO: 596 - protein, 13 aa, synthetic construct
CAASEPGAQKLVF

SEQ ID NO: 597 - protein, 15 aa, synthetic construct
CAGAVTTDSWGKLQF

SEQ ID NO: 598 - protein, 16 aa, synthetic construct
CAVNVNSGAGSYQLTF

SEQ ID NO: 599 - protein, 15 aa, synthetic construct
CAMRERTGGSYIPTF

SEQ ID NO: 600 - protein, 14 aa, synthetic construct
CAIGRGSTLGRLYF

SEQ ID NO: 601 - protein, 16 aa, synthetic construct
CAVSPPGYSSASKIIF

SEQ ID NO: 602 - protein, 14 aa, synthetic construct
CAEELSGGYQKVTF

SEQ ID NO: 603 - protein, 14 aa, synthetic construct
CAVEFTEYGNKLVF

SEQ ID NO: 604 - protein, 13 aa, synthetic construct
CAVSYSSASKIIF

SEQ ID NO: 605 - protein, 13 aa, synthetic construct
CAEFYNQGGKLIF

SEQ ID NO: 606 - protein, 11 aa, synthetic construct
CAVNNGNKLVF

SEQ ID NO: 607 - protein, 11 aa, synthetic construct
CAGGNAGKSTF

SEQ ID NO: 608 - protein, 14 aa, synthetic construct
CAASRRGSQGNLIF

SEQ ID NO: 609 - protein, 14 aa, synthetic construct
CAGPMKTSYDKVIF

SEQ ID NO: 610 - protein, 12 aa, synthetic construct
CAVKDSNYQLIW

SEQ ID NO: 611 - protein, 14 aa, synthetic construct
CAASIVGSQGNLIF

SEQ ID NO: 612 - protein, 12 aa, synthetic construct
CAALPGNTPLVF

SEQ ID NO: 613 - protein, 13 aa, synthetic construct
CAPWRGSARQLTF

SEQ ID NO: 614 - protein, 13 aa, synthetic construct
CAVNPTGGFKTIF

SEQ ID NO: 615 - protein, 18 aa, synthetic construct
CIVRPSNAGGTSYGKLTF

SEQ ID NO: 616 - protein, 11 aa, synthetic construct
CAASRVGQLTF

SEQ ID NO: 617 - protein, 15 aa, synthetic construct
CATDAWTGANSKLTF

SEQ ID NO: 618 - protein, 17 aa, synthetic construct
CAAKWAYSGAGSYQLTF

SEQ ID NO: 619 - protein, 14 aa, synthetic construct
CAVRDNNQGGKLIF

SEQ ID NO: 620 - protein, 15 aa, synthetic construct
CAYRSQETSGSRLTF

SEQ ID NO: 621 - protein, 12 aa, synthetic construct
CAADTGRRALTF

SEQ ID NO: 622 - protein, 10 aa, synthetic construct
CAKYTDKLIF

SEQ ID NO: 623 - protein, 14 aa, synthetic construct
CAASIGSTLGRLYF

SEQ ID NO: 624 - protein, 15 aa, synthetic construct
CVVNGPPGGSYIPTF

SEQ ID NO: 625 - protein, 12 aa, synthetic construct
CAVTDSWGKLQF

SEQ ID NO: 626 - protein, 12 aa, synthetic construct
CAVVDSNYQLIW

SEQ ID NO: 627 - protein, 14 aa, synthetic construct
CAEELSGGYQKVTF

SEQ ID NO: 628 - protein, 14 aa, synthetic construct
CAVSFKAAGNKLTF

SEQ ID NO: 629 - protein, 13 aa, synthetic construct
CAASRGGYSTLTF

SEQ ID NO: 630 - protein, 13 aa, synthetic construct
CSAGGDSPGGYTF

SEQ ID NO: 631 - protein, 13 aa, synthetic construct
CASRRGNTGELFF

SEQ ID NO: 632 - protein, 13 aa, synthetic construct
CAGYNSGTYKYIF

SEQ ID NO: 633 - protein, 14 aa, synthetic construct
CASSSGGLNTEAFF

SEQ ID NO: 634 - protein, 14 aa, synthetic construct
CAASIVGSQGNLIF

(19) United States
(12) Patent Application Publication
(10) Pub. No.: US 2026/0166146 A1
(43) Pub. Date: Jun. 18, 2026

(54) MUTANT KRAS-SPECIFIC TCRS

(71) Applicant: The Johns Hopkins University, Baltimore, MD (US)

(72) Inventors: Neeha Zaidi, Baltimore, MD (US); Amanda Huff, Baltimore, MD (US); Elizabeth A. Jaffee, Baltimore, MD (US); Elana Fertig, Baltimore, MD (US); Alexander Gergis, Baltimore, MD (US)

(21) Appl. No.: 19/357,703

(22) Filed: Oct. 14, 2025

Related U.S. Application Data
(63) Continuation of application No. PCT/US24/24543, filed on Apr. 14, 2024.
(60) Provisional application No. 63/459,451, filed on Apr. 14, 2023.

(51) Int. Cl. A61K 40/42; A61K 40/11; A61K 40/32; A61P 35/00; C07K 14/725; C07K 16/22

(52) U.S. Cl. CPC A61K 40/4238; A61K 40/11; A61K 40/32; A61P 35/00; C07K 14/7051; C07K 16/22

(57) ABSTRACT

T cell receptors (TCRs) which specifically target tumor antigens such as KRAS and KRAS mutations are generated using gene-editing complexes. In vivo and ex vivo methods for altering the TCR specificity are provided. These TCRs are utilized for adoptive T cell therapies for a broad range of HLA types.

### CROSS REFERENCE TO RELATED APPLICATIONS

This application is a continuation of PCT Application No. PCT/US24/24543, filed Apr. 14, 2024, which claims the benefit of priority under 35 U.S.C. § 119(e) to U.S. Provisional Application No. 63/459,451 filed Apr. 14, 2023, the entire contents of which applications are incorporated herein by reference.

### STATEMENT REGARDING FEDERALLY SPONSORED RESEARCH

This invention was made with government support under grant nos. CA248624, CA062924 and CA247886, awarded by the National Institutes of Health. The government has certain rights in the invention.

### SEQUENCE LISTING STATEMENT

The instant application contains a Sequence Listing which has been submitted electronically in XML format and is hereby incorporated by reference in its entirety. Said XML copy, created on Aug. 15, 2024, is named 348358_16302_SL.xml and is 553,615 bytes in size.

### BACKGROUND

Kirsten rat sarcoma viral oncogene homologue (KRAS) is the best-known oncogene with the highest mutation rate among all cancers and is associated with a series of highly fatal cancers, including pancreatic ductal adenocarcinoma (PDAC), nonsmall-cell lung cancer (NSCLC), and colorectal cancer (CRC). The identification of tumor driver genes and the development of specific inhibitors have revolutionized cancer treatment strategies and clinical outcomes. Numerous clinical results have shown that targeted therapies significantly extend progression-free survival and are less toxic than standard chemotherapy (Sequist, L. V. et al. Phase III study of afatinib or cisplatin plus pemetrexed in patients with metastatic lung adenocarcinoma with EGFR mutations. J. Clin. Oncol. 31, 3327-3334 (2013). Katayama, R., et al. Therapeutic targeting of anaplastic lymphoma kinase in lung cancer: a paradigm for precision cancer medicine. Clin. Cancer Res 21, 2227-2235 (2015). Mok, T. S. et al. Gefitinib or carboplatin-paclitaxel in pulmonary adenocarcinoma. N. Engl. J. Med 361, 947-957 (2009)). For instance, targeted therapies in patients harboring epidermal growth factor receptor (EGFR)-sensitive mutation or anaplastic lymphoma kinase (ALK) gene fusion have markedly enhanced survival time, with a median overall survival of 3 years or more (Soria, J.-C. et al. Osimertinib in Untreated EGFR-Mutated Advanced Non-Small-Cell Lung Cancer. N. Engl. J Med 378, 113-125 (2018). Kwak, E. L. et al. Anaplastic lymphoma kinase inhibition in non-small-cell lung cancer. N. Engl. J. Med 363, 1693-1703 (2010). Shaw, A. T. et al. Crizotinib in ROS1-rearranged non-small-cell lung cancer. N. Engl. J. Med 371, 1963-1971 (2014)).

Unfortunately, despite 40 years of proprietary drug efforts, there is still a need for effective strategies targeting KRAS mutations. Due to the intrinsic characteristics of KRAS proteins, targeting KRAS has been considered to be quite challenging. Therefore, many efforts have focused on indirectly targeting KRAS, including targeting its downstream signaling effectors, epigenetic approaches such as telomerase inhibitors and RNA interference and synthetic lethality approaches, such as cyclin-dependent kinase inhibitors. However, most of these strategies have failed due to a lack of activity or selectivity (Huang, L., Guo, Z., Wang, F. et al. KRAS mutation: from undruggable to druggable in cancer. Sig Transduct Target Ther 6, 386 (2021). doi.org/10.1038/s41392-021-00780-4). In addition, patients with KRAS mutations usually have a poor response to current standard therapy (Gao, W. et al. KRAS and TP53 mutations in bronchoscopy samples from former lung cancer patients. Mol. Carcinog. 56, 381-388 (2017)). There has been an urgent and unmet need to target KRAS mutations in KRAS-driven cancer.

### SUMMARY

Embodiments are directed to unique mutant-KRAS T cell receptors (TCRs) and use in adoptive T cell therapies across a broad range of HLA types.

Accordingly, in certain aspects, a method of redirecting T cell specificity in vitro or in vivo. In some embodiments, a method disclosed herein comprises contacting isolated cells obtained from a biological sample from a subject with a gene editing agent comprising a Clustered Regularly Interspaced Short Palindromic Repeat (CRISPR)-associated endonuclease or a nucleic acid sequence encoding the CRISPR-associated endonuclease; and, at least one guide nucleic acid or a nucleic acid sequence encoding the guide nucleic acid, the guide nucleic acid being complementary to a target nucleic acid sequence within or near a T cell receptor α chain (TCRα) sequence and/or TCRβ chain sequence for knocking out an endogenous T cell receptor and introducing a TCR specific for a tumor antigen into the isolated cells; thereby redirecting the T cell specificity. In certain embodiments, the gene editing agent is introduced into a cell by a vector or by homology-directed repair (HDR). In certain embodiments, the CRISPR/Cas comprises class I or class II CRISPR system. In some embodiments, the CRICPR/Cas system is type I, II, III, IV, V, or VI, In certain embodiments, the CRISPR/Cas system comprises a Cas9, Cas3 or Cas 12a endonuclease. In certain embodiments, the CRISPR/Cas system is CRISPR/Cas12a. In certain embodiments, the CRISPR/Cas12a introduces the recombinant TCRα and TCRβ chain sequence at the endogenous T cell receptor α and β constant (TRAC/TRBC) locus of healthy donor T cells. In certain embodiments, the CRISPR-Cas system, is introduced in single and multiplex configurations. In certain embodiments, the CRISPR/Cas editing composition comprises a plurality of guide RNAs (gRNAs).

In certain aspects, a T cell receptor (TCR) comprises an antigen binding domain which specifically binds a tumor antigen. In certain embodiments, the tumor antigen is a Kirsten rat sarcoma viral (KRAS) tumor antigen. In certain embodiments, the KRAS tumor antigen comprises one or more mutations. In certain embodiments, the TCR comprises a TCRα chain variable domain and a TCRβ chain variable domain having complementary determining regions (CDRs) which specifically bind to mutant KRAS epitopes. In certain embodiments, the complementary determining region of the TCRα chain, CDR3α, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID Nos: 315-629, 632, or 634. In certain embodiments, the complementary determining region of the TCRα chain, CDR3α, comprises an amino acid sequence of SEQ ID Nos: 315-629, 632, or 634. In certain embodiments, the complementary determining region of the TCRβ chain, CDR3β, comprises an amino acid sequence having a sequence identity of at least 50%, at least 60%, at least 70%, at least 75%, at least 80%, at least 85%, at least 90%, at least 95%, at least 99%, or 100% amino acid sequence identity to SEQ ID Nos: 1-314, or 630, or 631 or 633. In certain embodiments, the complementary determining region of the TCRβ chain, CDR3β, comprises an amino acid sequence comprising SEQ ID Nos: 1-314, or 630, or 631 or 633. In certain embodiments, mutant KRAS epitopes comprise G12V, G12D, G12C, G12R, G12A, G13D or combinations thereof. In certain embodiments, the TCR is soluble. In certain embodiments, the TCR is single-stranded. In certain embodiments, the TCR is formed by linking an a chain variable domain and a p chain variable domain through a peptide linking sequence. In certain embodiments, the TCR comprises (a) all or part of the TCRα chain except a transmembrane domain; and (b) all or part of the TCRβ chain except a transmembrane domain. In certain embodiments, the TCR comprises (a) all or part of the TCRα chain and a transmembrane domain; and (b) all or part of the TCRβ chain and a transmembrane domain. In some embodiments, a TCR disclosed herein comprises a V gene CDR1 or CDR2. In some embodiments, a TCR disclosed herein comprises a C*01 or C*02 constant region.

In another aspect, the complementary determining region of the TCRα chain comprises the amino acid sequence of any one of SEQ ID NOs: 315-629, 632, or 634, comprise one or more amino acid mutations, amino acid deletions, amino acid substitutions, modified amino acids, amino acid variants or combinations thereof. In certain embodiments, any one of SEQ ID NOs: 315-629, 632, or 634 comprises one amino acid mutation, amino acid deletions amino acid substitution, modified amino acid or amino acid variant. In certain embodiments, one or more amino acids of any one of SEQ ID NOs: 315-629, 632, or 634 are substituted with conservative amino acids. In certain embodiments, the complementary determining region of the TCRα chain, CDR3α, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the complementary determining region of the TCRα chain, CDR3α, comprises an amino acid sequence of any one of SEQ ID NOs: 315-629, 632, or 634.

In another aspect, the complementary determining region of the TCRβ chain comprises amino acid sequences of any one of SEQ ID NOs: 1-314, or 630, or 631 or 633, comprise one or more amino acid mutations, amino acid deletions, amino acid substitutions, modified amino acids, amino acid variants or combinations thereof. In certain embodiments, SEQ ID NOs: 1-314, or 630, or 631 or 633 comprise one amino acid mutation, amino acid deletions amino acid substitution, modified amino acid or amino acid variant. In certain embodiments, one or more amino acids of SEQ ID NOs: 1-314, or 630, or 631 or 633 are substituted with conservative amino acids. In certain embodiments, the complementary determining region of the TCRβ chain, CDR3β, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the complementary determining region of the TCRβ chain, CDR3β, comprises an amino acid sequence of any one of SEQ ID NOs: 1-314, or 630, or 631 or 633. In certain embodiments, mutant KRAS epitopes comprise G12V, G12D, G12C, G12R, G12A, G13D or combinations thereof. In some embodiments, a TCR disclosed herein comprises a V gene CDR1 or CDR2. In some embodiments, a TCR disclosed herein comprises a C*01 or C*02 constant region.

In another aspect, an isolated cell comprises the T cell receptors (TCRs) and chimeric antigen receptors (CARs) embodied herein. In some embodiments, the isolated cell is an immune cell. In certain embodiments, the isolated cell comprises T cells, B cells, natural killer (NK) cells, macrophages, stem cells, induced pluripotent stem cells (iPSCs) or combinations thereof. In certain embodiments, the T cell is a CD8⁺ T cell, a CD4⁺ T cell, a regulatory T cell (Treg), gamma delta T cells (γδ T cells), or a tumor infiltrating T lymphocyte (TIL).

In another aspect, a chimeric antigen receptor (CAR) comprises an antigen specific binding domain, a transmembrane domain(s), a co-stimulatory domain(s), and a CD3ζ signaling domain, wherein the antigen specific binding domain specifically binds Kirsten rat sarcoma viral (KRAS) tumor antigens. In certain embodiments, the KRAS tumor antigen comprises one or more mutations. In certain embodiments, the antigen binding domain comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the antigen binding domain comprises an amino acid sequence of any one of SEQ ID NOs: 315-629, 632, or 634. In certain embodiments, the antigen binding domain comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the antigen binding domain comprises an amino acid sequence of any one of SEQ ID NOs: 1-314, or 630, or 631 or 633. In certain embodiments, the co-stimulatory domain comprises a cluster of differentiation antigen 28 (CD28), 41BB domain, an ICOS (Inducible T cell Co-stimulator) (CD278), OX40 (CD134), Glucocorticoid-induced Tumor Necrosis Factor Receptor (GITR), CD40 or CD27. In some embodiments, a TCR disclosed herein comprises a V gene CDR1 or CDR2. In some embodiments, a TCR disclosed herein comprises a C*01 or C*02 constant region.

In another aspect, an expression vector is provided, encoding the T cell receptors (TCRs) or the chimeric antigen receptors (CARs) embodied herein. In certain embodiments, the expression vector comprises adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus or vesicular stomatitis virus. In certain embodiments, the expression vector further comprises an inducible promoter, a cell specific promoter, a tissue specific promoter or a constitutive promoter. In certain embodiments, the expression vector further comprises one or more enhancer or regulatory sequences. In certain embodiments, the expression vector further comprises an inducible suicide gene. In certain embodiments, the expression vector further comprises a nucleic acid sequence that encodes one or more cytokines.

In another aspect, disclosed herein is a method of treating cancer in a subject diagnosed with cancer. In some embodiments, the method comprises isolating cells from a biological sample of the subject, culturing the isolated cells with one or more tumor antigens, isolating T cells and/or NK cells cultured with the one or more tumor antigens and expanding the T cells and/or NK cells to produce a therapeutically effective composition of tumor antigen specific T cells and NK cells. In some embodiments, the method comprises adoptively transferring the tumor antigen specific T cells and NK cells into the subject, thereby treating the subject diagnosed with cancer. In certain embodiments, the isolated cells are autologous cells. In certain embodiments, the T cells comprise a CD8⁺ T lymphocyte, a CD4⁺ T lymphocyte, a γδ T cell, a regulatory T cell (Treg), a tumor infiltrating T lymphocyte (TIL) and combinations thereof. In certain embodiments, the tumor antigen is a Kirsten rat sarcoma viral (KRAS) tumor antigen. In certain embodiments, the KRAS tumor antigen comprises one or more mutations.

In another aspect, disclosed herein is a method of treating a subject diagnosed with cancer. In some embodiments, the method comprises isolating T lymphocytes from a biological sample obtained from the subject; transducing the T lymphocytes with an expression vector encoding a chimeric antigen receptor (CAR) which specifically binds to a Kirsten rat sarcoma viral (KRAS) tumor antigen; expanding the transduced T lymphocytes at least once ex vivo to obtain expanded T lymphocytes specific for the KRAS tumor antigen; and reinfusing the T lymphocytes into the subject, thereby treating the subject. In certain embodiments, the CAR comprises an antigen binding domain linked to at least one co-stimulatory domain and a CD3 signaling domain In some embodiments, the antigen binding domain comprises a single chain variable fragment (scFv) which specifically binds to the KRAS tumor antigen. In certain embodiments, the co-stimulatory domain comprises a cluster of differentiation antigen 28 (CD28), 41BB domain, an ICOS (Inducible T cell Co-stimulator) (CD278), OX40 (CD134), Glucocorticoid-induced Tumor Necrosis Factor Receptor (GITR), CD40 or CD27polypeptide. In certain embodiments, the T cell is a CD8⁺ T cell, a CD4⁺ T cell, a γδ T cell, a T regulatory cell (Treg) or a tumor infiltrating T lymphocyte (TIL).

In another aspect, disclosed herein is a T cell receptor (TCR) isolated from a T cell, wherein the TCR specifically binds to a tumor antigen. In certain embodiments, the T cell comprises a co-stimulatory domain. In certain embodiments, the co-stimulatory domain comprises a cluster of differentiation antigen 28 (CD28), 41BB domain, an ICOS (Inducible T cell Co-stimulator) (CD278), OX40 (CD134), Glucocorticoid-induced Tumor Necrosis Factor Receptor (GITR), CD40 or CD27. In certain embodiments, the tumor antigen is a Kirsten rat sarcoma viral (KRAS) tumor antigen. In certain embodiments, the KRAS tumor antigen comprises one or more mutations. In certain embodiments, the TCR comprises a TCRα chain variable domain and a TCRβ chain variable domain having complementary determining regions (CDRs) which specifically bind to mutant KRAS epitopes. In certain embodiments, the TCR is soluble. In certain embodiments, the TCR is single-stranded. In certain embodiments, the TCR comprises (a) all or part of the TCRα chain except a transmembrane domain; and (b) all or part of the TCRβ chain except a transmembrane domain. In certain embodiments, the TCR comprises (a) all or part of the TCRα chain and a transmembrane domain; and (b) all or part of the TCRβ chain and a transmembrane domain.

In another aspect, therapies disclosed herein can be administered in combination with one or more other therapies such as pharmaceutical, chemotherapies, hormone therapies, surgeries, radiation therapies and the like.

In another aspect, disclosed herein is a kit comprising a T cell receptor (TCR) wherein the TCR comprises a TCRα chain variable domain and a TCRβ chain variable domain having complementary determining regions (CDRs) which specifically bind to mutant KRAS epitopes. In certain embodiments, the complementary determining region of the TCRα chain, CDR3α, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the complementary determining region of the TCRα chain, CDR3α, comprises an amino acid sequence of any one of SEQ ID NOs: 315-629, 632, or 634. In certain embodiments, the complementary determining region of the TCRβ chain, CDR3β, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the complementary determining region of the TCRβ chain, CDR3β, comprises an amino acid sequence having a sequence identity of at least 75%, 85%, 90%, 95%, 98%, or 99% to SEQ ID NOs: 1-314, or 630, or 631 or 633. In certain embodiments, the complementary determining region of the TCRβ chain, CDR3β, comprises an amino acid sequence of any one of SEQ ID NOs: 1-314, or 630, or 631 or 633. In certain embodiments, the TCR is soluble. In certain embodiments, the TCR is single-stranded. In certain embodiments, the TCR is formed by linking an a chain variable domain and a β chain variable domain through a peptide linking sequence. In certain embodiments, the TCR comprises (a) all or part of the TCRα chain except a transmembrane domain; and (b) all or part of the TCRβ chain except a transmembrane domain. In certain embodiments, the TCR comprises (a) all or part of the TCRα chain and a transmembrane domain; and (b) all or part of the TCRβ chain and a transmembrane domain. In some embodiments, a TCR disclosed herein comprises a V gene CDR1 or CDR2. In some embodiments, a TCR disclosed herein comprises a C*01 or C*02 constant region.

In another aspect, disclosed herein is an isolated cell comprises an expression vector encoding a T cell receptor (TCR). In certain embodiments, the TCR comprises a TCRα chain variable domain and a TCRβ chain variable domain having complementary determining regions (CDRs) which specifically bind to mutant KRAS epitopes. In certain embodiments, the expression vector comprises adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus or vesicular stomatitis virus. In certain embodiments, the expression vector further comprises an inducible promoter, a tissue specific promoter or a constitutive promoter. In certain embodiments, the expression vector further comprises one or more enhancer or regulatory sequences. In certain embodiments, the expression vector further comprises an inducible suicide gene. In certain embodiments, the expression vector further comprises a nucleic acid sequence encoding for one or more cytokines.

In another aspect, disclosed herein is an isolated nucleic acid that encodes a T cell receptor (TCR) comprising an antigen binding domain which specifically binds a tumor antigen. In certain embodiments, the tumor antigen is a Kirsten rat sarcoma viral (KRAS) tumor antigen. In certain embodiments, the TCR comprises a TCRα chain variable domain and a TCRβ chain variable domain having complementary determining regions (CDRs) which specifically bind to mutant KRAS epitopes. In certain embodiments, the complementary determining region of the TCRα chain, CDR3α, comprises a nucleic acid sequence encoding an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the complementary determining region of the TCRα chain, CDR3α, comprises a nucleic acid sequence encoding an amino acid sequence of any one of SEQ ID NOs: 315-629, 632, or 634. In certain embodiments, the complementary determining region of the TCRα chain, CDR3α, comprises a nucleic acid sequence encoding an amino acid sequence having a sequence identity of at least 75%, 85%, 90%, 95%, 98%, 99% to any one of SEQ ID NOs: 315-629, 632, or 634. In certain embodiments, the complementary determining region of the TCRα chain, CDR3α, comprises a nucleic acid sequence encoding an amino acid sequence of any one of SEQ ID NOs: 315-629, 632, or 634. In certain embodiments, the complementary determining region of the TCRβ chain, CDR3β, comprises a nucleic acid sequence encoding an amino acid sequence having a sequence identity of at least 50%, at least 60%, at least 70%, at least 75%, at least 80%, at least 85%, at least 90%, at least 95%, or at least 99%, or 100% amino acid sequence identity to any one of SEQ ID NOs: 1-314, or 630, or 631 or 633. In certain embodiments, the complementary determining region of the TCRβ chain, CDR3β, comprises a nucleic acid sequence encoding an amino acid sequence having a sequence identity of at least 75%, 85%, 90%, 95%, 98%, 99% to any one of SEQ ID NOs: 1-314, or 630, or 631 or 633. In certain embodiments, the complementary determining region of the TCRβ chain, CDR3β, comprises a nucleic acid sequence encoding an amino acid sequence to any one of SEQ ID NOs: 1-314, or 630, or 631 or 633. In some embodiments, a TCR disclosed herein comprises a V gene CDR1 or CDR2. In some embodiments, a TCR disclosed herein comprises a C*01 or C*02 constant region.

In another aspect, disclosed herein is an isolated nucleic acid encoding a chimeric antigen receptor (CAR) comprising an antigen specific binding domain, a transmembrane domain(s), a co-stimulatory domain(s), and a CD3ζ signaling domain, wherein the antigen specific binding domain specifically binds Kirsten rat sarcoma viral (KRAS) tumor antigens. In certain embodiments, the antigen binding domain comprises a nucleic acid sequence encoding an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the antigen binding domain comprises a nucleic acid sequence encoding an amino acid sequence of any one of SEQ ID NOs: 315-629, 632, or 634. In certain embodiments, the antigen binding domain comprises a nucleic acid sequence encoding an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the antigen binding domain comprises a nucleic acid sequence encoding an amino acid sequence any one of SEQ ID NOs: 1-314, or 630, or 631 or 633. In certain embodiments, an isolated nucleic acid encodes a co-stimulatory domain comprising a cluster of differentiation antigen 28 (CD28), 41BB domain, an ICOS (Inducible T cell Co-stimulator) (CD278), OX40 (CD134), Glucocorticoid-induced Tumor Necrosis Factor Receptor (GITR), CD40 or CD27. In certain embodiments, the isolated nucleic acid encodes the CAR and the co-stimulatory domain. In certain embodiments, a CAR is encoded by a first nucleic acid and the co-stimulatory domain is encoded by a second nucleic acid. In some embodiments, a TCR disclosed herein comprises a V gene CDR1 or CDR2. In some embodiments, a TCR disclosed herein comprises a C*01 or C*02 constant region.

Disclosed herein are T cell receptors comprising a CD3α chain and a CD3β chain. In certain embodiments, a T cell receptor comprises a CD3α chain and a CD3β chain comprising CAFILNNNDMRF (SEQ ID NO:315) and CAIAGPGQGARGYTF (SEQ ID NO:1) respectively. In certain embodiments, a T cell receptor comprises a CD3α chain and a CD3β chain comprising CALSEAVGGANNLFF (SEQ ID NO:316) and CAISEGSPEAFF (SEQ ID NO:2) respectively. In certain embodiments, a T cell receptor comprises a CD3α chain and a CD3β chain comprising CAFMKQSGGSQGNLIF (SEQ ID NO:317) and CASGLRLNEKLFF (SEQ ID NO:3) respectively. In certain embodiments, a T cell receptor comprises a CD3α chain and a CD3β chain comprising CAVRSYSGAGSYQLTF (SEQ ID NO:318) and CASGLVDYELFF (SEQ ID NO:4) respectively. In certain embodiments, a T cell receptor comprises a CD3α chain and a CD3β chain comprising CAACDRGSTLGRLYF (SEQ ID NO:319) and CASIHLVGGTGRQPQHF (SEQ ID NO:5) respectively. In certain embodiments, a T cell receptor comprises a CD3α chain and a CD3β chain comprising CAAGNNARLMF (SEQ ID NO:320) and CASKEATAASTNEKLFF (SEQ ID NO:6) respectively. In certain embodiments, a T cell receptor comprises a CD3α chain and a CD3β chain comprising CAGLSNDYKLSF (SEQ ID NO:321) and CASKQGNEQFF (SEQ ID NO:7) respectively. In certain embodiments, a T cell receptor comprises a CD3α chain and a CD3β chain comprising CIVKSWGKLQF (SEQ ID NO:322) and CASLLDAGAANTEAFF (SEQ ID NO:8) respectively. In certain embodiments, a T cell receptor comprises a CD3α chain and a CD3β chain comprising CATDAGNDMRF (SEQ ID NO:323) and CASLSRGLNEKLFF (SEQ ID NO:9) respectively. In certain embodiments, a T cell receptor comprises a CD3α chain and a CD3β chain comprising CAVGAGGTSYGKLTF (SEQ ID NO:324) and CASMLQGALNQPQHF (SEQ ID NO:10) respectively. In certain embodiments, a T cell receptor comprises a CD3α chain and a CD3β chain comprising CAGHSNTGNQFYF (SEQ ID NO:325) and CASNFGQGRYGYTF (SEQ ID NO:11) respectively. In certain embodiments, a T cell receptor comprises a CD3α chain and a CD3β chain comprising CVVSRVKAAGNKLTF (SEQ ID NO:326) and CASNPDNALDNSPLHF (SEQ ID NO:12) respectively. In certain embodiments, a T cell receptor comprises a CD3α chain and a CD3β chain comprising CAAPYPTGGTSYGKLTF (SEQ ID NO:327) and CASRDSYSNQPQHF (SEQ ID NO:13) respectively. In certain embodiments, a T cell receptor comprises a CD3α chain and a CD3β chain comprising CAVSERGFQKLVF (SEQ ID NO:328) and CASRDTQGGGADTQYF (SEQ ID NO:14) respectively.

Disclosed herein are T cell receptors. In certain embodiments, a T cell receptor comprises a CD3α chain and a CD3β chain comprising CALSESWGKLQF (SEQ ID NO:329) and CASREQGQGTGELFF (SEQ ID NO:15) respectively. In certain embodiments, a T cell receptor comprises a CD3α chain and a CD3β chain comprising CAVGASGAAGNKLTF (SEQ ID NO:330) and CASRGDSGFNYGYTF (SEQ ID NO:16) respectively. In certain embodiments, a T cell receptor comprises a CD3α chain and a CD3β chain comprising CAVSDNQGAQKLVF (SEQ ID NO:331) and CASRGQGAATDTQYF (SEQ ID NO:17) respectively. In certain embodiments, a T cell receptor comprises a CD3α chain and a CD3β chain comprising CAAPTSGGGADGLTF (SEQ ID NO:332) and CASRGQRRINYGYTF (SEQ ID NO:18) respectively. In certain embodiments, a T cell receptor comprises a CD3α chain and a CD3β chain comprising CAGQGYFGNEKLTF (SEQ ID NO:333) and CASRGTGVNQPQHF (SEQ ID NO:19) respectively. In certain embodiments, a T cell receptor comprises a CD3α chain and a CD3β chain comprising CALSYNQGGKLIF (SEQ ID NO:334) and CASRKDTGELFF (SEQ ID NO:20) respectively. In certain embodiments, a T cell receptor comprises a CD3α chain and a CD3β chain comprising CAVSEPGSGGSNYKLTF (SEQ ID NO:335) and CASRLDRSEAFF (SEQ ID NO:21) respectively. In certain embodiments, a T cell receptor comprises a CD3α chain and a CD3β chain comprising CVVSDVNRNQFYF (SEQ ID NO:336) and CASRPGQGYEKLFF (SEQ ID NO:22) respectively. In certain embodiments, a T cell receptor comprises a CD3α chain and a CD3β chain comprising CAVNAPFGNEKLTF (SEQ ID NO:337) and CASRRNGLYYTF (SEQ ID NO:23) respectively. In certain embodiments, a T cell receptor comprises a CD3α chain and a CD3β chain comprising CVVNWGGGYNKLIF (SEQ ID NO:338) and CASRSGTGGSGELFF (SEQ ID NO:24) respectively. In certain embodiments, a T cell receptor comprises a CD3α chain and a CD3β chain comprising CAEITRYGGSQGNLIF (SEQ ID NO:339) and CASRSPWTGANVLTF (SEQ ID NO:25) respectively. In certain embodiments, a T cell receptor comprises a CD3α chain and a CD3β chain comprising CALSTSGTYKYIF (SEQ ID NO:340) and CASRTGGNLDDTQYF (SEQ ID NO:26) respectively. In certain embodiments, a T cell receptor comprises a CD3α chain and a CD3β chain comprising CVVGVRGGGTSYGKLTF (SEQ ID NO:341) and CASRTLSGGDTQYF (SEQ ID NO:27) respectively. In certain embodiments, a T cell receptor comprises a CD3α chain and a CD3β chain comprising CALRAQNSGYSTLTF (SEQ ID NO:342) and CASSAFYEQYF (SEQ ID NO:28) respectively. In certain embodiments, a T cell receptor comprises a CD3α chain and a CD3β chain comprising CAFMKHMSGNNRKLIW (SEQ ID NO:343) and CASSALGSGNTIYF (SEQ ID NO:29) respectively. In certain embodiments, a T cell receptor comprises a CD3α chain and a CD3β chain comprising CAASGTYKYIF (SEQ ID NO:344) and CASSAQGTSYNEQFF (SEQ ID NO:30) respectively.

Disclosed herein are T cell receptors. In certain embodiments, a T cell receptor comprises a CD3α chain and a CD3β chain comprising CAEIAGNQFYF (SEQ ID NO:345) and CASSASFYQPQHF (SEQ ID NO:31) respectively. In certain embodiments, a T cell receptor comprises a CD3α chain and a CD3β chain comprising CAVSDSFQKLVF (SEQ ID NO:346) and CASSATGGNSPLHF (SEQ ID NO:32) respectively. In certain embodiments, a T cell receptor comprises a CD3α chain and a CD3β chain comprising CAVSSTYGNKLVF (SEQ ID NO:347) and CASSEGQGDYGYTF (SEQ ID NO:33) respectively. In certain embodiments, a T cell receptor comprises a CD3α chain and a CD3β chain comprising CAAIGSSNTGKLIF (SEQ ID NO:348) and CASSENRRREPQHF (SEQ ID NO:34) respectively. In certain embodiments, a T cell receptor comprises a CD3α chain and a CD3β chain comprising CAVSEDTGGFKTIF (SEQ ID NO:349) and CASSEQSGLTNSPLHF (SEQ ID NO:35) respectively. In certain embodiments, a T cell receptor comprises a CD3α chain and a CD3β chain comprising CAVLGTSGSRLTF (SEQ ID NO:350) and CASSFAAGLGYEQYF (SEQ ID NO:36) respectively. In certain embodiments, a T cell receptor comprises a CD3α chain and a CD3β chain comprising CAVRDKRSNDYKLSF (SEQ ID NO:351) and CASSFAGVYTGELFF (SEQ ID NO:37) respectively. In certain embodiments, a T cell receptor comprises a CD3α chain and a CD3β chain comprising CAASNVLTGGGNKLTF (SEQ ID NO:352) and CASSFPTGGLSSEQFF (SEQ ID NO:38) respectively. In certain embodiments, a T cell receptor comprises a CD3α chain and a CD3β chain comprising CAAISRTGSARQLTF (SEQ ID NO:353) and CASSFQETQYF (SEQ ID NO:39) respectively. In certain embodiments, a T cell receptor comprises a CD3α chain and a CD3β chain comprising CATDATGANSKLTF (SEQ ID NO:354) and CASSFRGGLQETQYF (SEQ ID NO:40) respectively. In certain embodiments, a T cell receptor comprises a CD3α chain and a CD3β chain comprising CALSDSGGGADGLTF (SEQ ID NO:355) and CASSFTDRRKDTF (SEQ ID NO:41) respectively. In certain embodiments, a T cell receptor comprises a CD3α chain and a CD3β chain comprising CALSEAGNAGNMLTF (SEQ ID NO:356) and CASSFYGTGGEGKPQHF (SEQ ID NO:42) respectively. In certain embodiments, a T cell receptor comprises a CD3α chain and a CD3β chain comprising CAVPGGTSYGKLTF (SEQ ID NO:357) and CASSGTDFYEQYF (SEQ ID NO:43) respectively.

Disclosed herein are T cell receptors. In certain embodiments, a T cell receptor comprises a CD3α chain and a CD3β chain comprising CAVGARQAGTALIF (SEQ ID NO:358) and CASSGTGDSGEAFF (SEQ ID NO:44) respectively. In certain embodiments, a T cell receptor comprises a CD3α chain and a CD3β chain comprising CAFMTINAGGTSYGKLTF (SEQ ID NO:359) and CASSGTGGAISNQPQHF (SEQ ID NO:45) respectively. In certain embodiments, a T cell receptor comprises a CD3α chain and a CD3β chain comprising CVVRVGFGNVLHC (SEQ ID NO:360) and CASSIGGTTGELFF (SEQ ID NO:46) respectively. In certain embodiments, a T cell receptor comprises a CD3α chain and a CD3β chain comprising CAATRRSNDYKLSF (SEQ ID NO:361) and CASSISTNTGELFF (SEQ ID NO:47) respectively. In certain embodiments, a T cell receptor comprises a CD3α chain and a CD3β chain comprising CIAPNMDSNYQLIW (SEQ ID NO:362) and CASSITGSSGQPQHF (SEQ ID NO:48) respectively. In certain embodiments, a T cell receptor comprises a CD3α chain and a CD3β chain comprising CATPKIYNQGGKLIF (SEQ ID NO:363) and CASSKDRGLALETQYF (SEQ ID NO:49) respectively. In certain embodiments, a T cell receptor comprises a CD3α chain and a CD3β chain comprising CAVRPPGANSKLTF (SEQ ID NO:364) and CASSKESANRYNEQFF (SEQ ID NO:50) respectively. In certain embodiments, a T cell receptor comprises a CD3α chain and a CD3β chain comprising CAVEDDNNARLMF (SEQ ID NO:365) and CASSLASNQPQHF (SEQ ID NO:51) respectively. In certain embodiments, a T cell receptor comprises a CD3α chain and a CD3β chain comprising CAASPRFNKFYF (SEQ ID NO:366) and CASSLDQTSNEQFF (SEQ ID NO:52) respectively. In certain embodiments, a T cell receptor comprises a CD3α chain and a CD3β chain comprising CAVNSNYQLIW (SEQ ID NO:367) and CASSLDRGLGNSPLHF (SEQ ID NO:53) respectively. In certain embodiments, a T cell receptor comprises a CD3α chain and a CD3β chain comprising CAVKGPAGNNRKLIW (SEQ ID NO:368) and CASSLDSGTNTGELFF (SEQ ID NO:54) respectively. In certain embodiments, a T cell receptor comprises a CD3α chain and a CD3β chain comprising CALSVGGAQKLVF (SEQ ID NO:369) and CASSLDSLATDTQYF (SEQ ID NO:55) respectively. In certain embodiments, a T cell receptor comprises a CD3α chain and a CD3β chain comprising CVVLERNTGGFKTIF (SEQ ID NO:370) and CASSLEGGLAKNIQYF (SEQ ID NO:56) respectively. In certain embodiments, a T cell receptor comprises a CD3α chain and a CD3β chain comprising CATDRDSNYQLIW (SEQ ID NO:371) and CASSLEGRGPTNEKLFF (SEQ ID NO:57) respectively. In certain embodiments, a T cell receptor comprises a CD3α chain and a CD3β chain comprising CAMREGSDYKLSF (SEQ ID NO:372) and CASSLESGNSPLHF (SEQ ID NO:58) respectively. In certain embodiments, a T cell receptor comprises a CD3α chain and a CD3β chain comprising CAASGGGADGLTF (SEQ ID NO:373) and CASSLETGVEQFF (SEQ ID NO:59) respectively. In certain embodiments, a T cell receptor comprises a CD3α chain and a CD3β chain comprising

Disclosed herein are T cell receptors comprising a CD3α chain and a CD3β chain. In certain embodiments, a T cell receptor comprises a CD3α chain and a CD3β chain comprising CAASLPGNTPLVF (SEQ ID NO:374) and CASSLFGGGGEKLFF (SEQ ID NO:60) respectively. In certain embodiments, a T cell receptor comprises a CD3α chain and a CD3β chain comprising CAYRSGATNKLIF (SEQ ID NO:375) and CASSLFLGSYEQYF (SEQ ID NO:61) respectively. In certain embodiments, a T cell receptor comprises a CD3α chain and a CD3β chain comprising CAVPPPNFGNEKLTF (SEQ ID NO:376) and CASSLGGGNQPQHF (SEQ ID NO:62) respectively. In certain embodiments, a T cell receptor comprises a CD3α chain and a CD3β chain comprising CATDSSNTGNQFYF (SEQ ID NO:377) and CASSLGGNTGELFF (SEQ ID NO:63) respectively. In certain embodiments, a T cell receptor comprises a CD3α chain and a CD3β chain comprising CIAHDRGSTLGRLYF (SEQ ID NO:378) and CASSLGGSGSFYHNEQFF (SEQ ID NO:64) respectively. In certain embodiments, a T cell receptor comprises a CD3α chain and a CD3β chain comprising CALSPQGTGGFKTIF (SEQ ID NO:379) and CASSLGLRPINEQFF (SEQ ID NO:65) respectively. In certain embodiments, a T cell receptor comprises a CD3α chain and a CD3β chain comprising CAALAGPGYALNF (SEQ ID NO:380) and CASSLGRGPTDTQYF (SEQ ID NO:66) respectively. In certain embodiments, a T cell receptor comprises a CD3α chain and a CD3β chain comprising CAVRGAGNNRKLIW (SEQ ID NO:381) and CASSLGVNTEAFF (SEQ ID NO:67) respectively. In certain embodiments, a T cell receptor comprises a CD3α chain and a CD3β chain comprising CADSYGGATNKLIF (SEQ ID NO:382) and CASSLGYRGEQYF (SEQ ID NO:68) respectively. In certain embodiments, a T cell receptor comprises a CD3α chain and a CD3β chain comprising CILSNVYSGAGSYQLTF (SEQ ID NO:383) and CASSLLDRGDSPLHF (SEQ ID NO:69) respectively. In certain embodiments, a T cell receptor comprises a CD3α chain and a CD3β chain comprising CAFDNNDMRF (SEQ ID NO:384) and CASSLLTGGQYF (SEQ ID NO:70) respectively.

In certain cases, a T cell receptor disclosed herein comprises a CD3α chain and a CD3β chain comprising CAFIGLLGIQGAQKLVF (SEQ ID NO:385) and CASSLNRGYEQYV (SEQ ID NO:71) respectively; CAASIRSGGSYIPTF (SEQ ID NO:386) and CASSLNTEAFF (SEQ ID NO:72) respectively; CAANAGNNRKLIW (SEQ ID NO:387) and CASSLQGRTEAFF (SEQ ID NO:73) respectively; CAAEFTGTASKLTF (SEQ ID NO:388) and CASSLQGSYGYTF (SEQ ID NO:74) respectively; CAEGRLTGGFKTIF (SEQ ID NO:389) and CASSLRGEAFF (SEQ ID NO:75) respectively; CAVRGGGGFKTIF (SEQ ID NO:390) and CASSLRGNEQFF (SEQ ID NO:76) respectively; CGTEEMNRDDKIIF (SEQ ID NO:391) and CASSLRTGGRMPQHF (SEQ ID NO:77) respectively; CAASNSGYALNF (SEQ ID NO:392) and CASSLRTNTGEKLFF (SEQ ID NO:78) respectively; CAAASGYSTLTF (SEQ ID NO:393) and CASSLSPGKSNQPQHF (SEQ ID NO:79) respectively; CVVSLLTGGGNKLTF (SEQ ID NO:394) and CASSLSYEQYF (SEQ ID NO:80) respectively; CAVRSSLGNNRLAF (SEQ ID NO:395) and CASSLTEGVRTEAFF (SEQ ID NO:81) respectively; CAAMGNRDDKIIF (SEQ ID NO:396) and CASSLVETQYF (SEQ ID NO:82) respectively; CAVDRARNSGGSNYKLTF (SEQ ID NO:397) and CASSLVGGNTIYF (SEQ ID NO:83) respectively; CALGEYGNKLVF (SEQ ID NO:398) and CASSLVGNTEAFF (SEQ ID NO:84) respectively; CAASGANSGYALNF (SEQ ID NO:399) and CASSLVSTAEQYF (SEQ ID NO:85) respectively; CAGPTNSGGYQKVTF (SEQ ID NO:400) and CASSLVVTGELFF (SEQ ID NO:86) respectively; CIVRVAYNNAGNMLTF (SEQ ID NO:401) and CASSLWGATDTQYF (SEQ ID NO:87) respectively; CAAGDTGRRALTF (SEQ ID NO:402) and CASSPDSSFGNQPQHF (SEQ ID NO:88) respectively; CASGRGSQGNLIF (SEQ ID NO:403) and CASSPDSYNEQFF (SEQ ID NO:89) respectively; CALIDRGSTLGRLYF (SEQ ID NO:404) and CASSPEETQYF (SEQ ID NO:90) respectively; CAAPPGGTSYGKLTF (SEQ ID NO:405) and CASSPGQAANSPLHF (SEQ ID NO:91) respectively; CAVQAAGGYQKVTF (SEQ ID NO:406) and CASSPGRVAFF (SEQ ID NO:92) respectively; CAERQGNTPLVF (SEQ ID NO:407) and CASSPGTDQPQHF (SEQ ID NO:93) respectively; CAASGDRDDKIIF (SEQ ID NO:408) and CASSPGTEAFF (SEQ ID NO:94) respectively; CATDPGANNLFF (SEQ ID NO:409) and CASSPMGTGNTEAFF (SEQ ID NO:95) respectively; CALPPGGGTSYGKLTF (SEQ ID NO:410) and CASSPPDRGRHEQFF (SEQ ID NO:96) respectively; CAVRDAGGYNKLIF (SEQ ID NO:411) and CASSPPSGSGELFF (SEQ ID NO:97) respectively or CAMRLGAAGNKLTF (SEQ ID NO:412) and CASSPPVLVSGNTIYF (SEQ ID NO:98) respectively.

In certain cases, a T cell receptor disclosed herein comprises a CD3α chain and a CD3β chain comprising CALSDYNQGGKLIF (SEQ ID NO:413) and CASSPQDRGQGNTEAFF (SEQ ID NO:99) respectively; CALSPRGGYQKVTF (SEQ ID NO:414) and CASSPRDRGLYQPQHF (SEQ ID NO:100) respectively; CAVRGRGYSTLTF (SEQ ID NO:415) and CASSPRGAGNTIYF (SEQ ID NO:101) respectively; CAGDFGGTSYGKLTF (SEQ ID NO:416) and CASSPRTGGNQPQHF (SEQ ID NO:102) respectively; CAASRGNNRLAF (SEQ ID NO:417) and CASSPSAGAGYEQYF (SEQ ID NO: 103) respectively; CAVRDGGGYNKLIF (SEQ ID NO:418) and CASSPSQGIDSGANVLTF (SEQ ID NO: 104) respectively; CAVQGYLGGATNKLIF (SEQ ID NO:419) and CASSPSRDRSYEQYF (SEQ ID NO: 105) respectively; CAVYSNTGKLIF (SEQ ID NO:420) and CASSPTDRIRAFF (SEQ ID NO: 106) respectively; CAMRVNNARLMF (SEQ ID NO:421) and CASSPYRGLNHSF (SEQ ID NO:107) respectively; CALSDRTGANSKLTF (SEQ ID NO:422) and CASSQDGGGTDTQYF (SEQ ID NO: 108) respectively; CALSDRGSARQLTF (SEQ ID NO:423) and CASSQDGVATDTQYF (SEQ ID NO: 109) respectively; CAAKGNTGNQFYF (SEQ ID NO:424) and CASSQDKGRDQPQHF (SEQ ID NO: 110) respectively; CAALRDRGSTLGRLYF (SEQ ID NO:425) and CASSQDRPSFTEAFF (SEQ ID NO:111) respectively; CAVSETGFQKLVF (SEQ ID NO:426) and CASSQDRQKLSGELFF (SEQ ID NO: 112) respectively; CATDATSGSRLTF (SEQ ID NO:427) and CASSQDRTSTRDEQFF (SEQ ID NO: 113) respectively; CAERNNNARLMF (SEQ ID NO:428) and CASSQDWVVGNQPQHF (SEQ ID NO: 114) respectively; CAASTGNQFYF (SEQ ID NO:429) and CASSQEDRGNQPQHF (SEQ ID NO:115) respectively; CVVTLNNAGNMLTF (SEQ ID NO:430) and CASSQGGVGETQYF (SEQ ID NO: 116) respectively; CAASGGEGGGADGLTF (SEQ ID NO:431) and CASSQGRGGYQPQHF (SEQ ID NO: 117) respectively; CAVGPWGDYKLSF (SEQ ID NO:432) and CASSQGTGGMRGYTF (SEQ ID NO: 118) respectively; CAASWGNTPLVF (SEQ ID NO:433) and CASSQQGSEQYV (SEQ ID NO: 119) respectively; CAVRRRGDSNYQLIW (SEQ ID NO:434) and CASSQSEVGGQFF (SEQ ID NO: 120) respectively; CAVSEKGAGGFKTIF (SEQ ID NO:435) and CASSRDSGRAGDTQYF (SEQ ID NO: 121) respectively; CAVSQMDSSYKLIF (SEQ ID NO:436) and CASSREGYGYTF (SEQ ID NO:122) respectively; CAVSGLNNARLMF (SEQ ID NO:437) and CASSRQSSGNTIYF (SEQ ID NO:123) respectively or CALSGGQAGTALIF (SEQ ID NO:438) and CASSRSGLFNTEGAFF (SEQ ID NO: 124) respectively.

In certain cases, a T cell receptor disclosed herein comprises a CD3α chain and a CD3β chain comprising CAVRRQGGKLIF (SEQ ID NO:439) and CASSRTALAANVLTF (SEQ ID NO: 125) respectively; CAVRPESNFGNEKLTF (SEQ ID NO:440) and CASSRTGDNSPLHF (SEQ ID NO: 126) respectively; CIVRVAGGTSYGKLTF (SEQ ID NO:441) and CASSRTGGGRGYTF (SEQ ID NO:127) respectively; CAEMNNAGNMLTF (SEQ ID NO:442) and CASSSENSPLHF (SEQ ID NO:128) respectively; CAASIVGSQGNLIF (SEQ ID NO:443) and CASSSGGLNTEAFF (SEQ ID NO:129) respectively; CIVRVGGISNFGNEKLTF (SEQ ID NO:444) and CASSSGNSPLHF (SEQ ID NO: 130) respectively; CAVETSGSRLTF (SEQ ID NO:445) and CASSSLQVNSGNTIYF (SEQ ID NO:131) respectively; CALRAGGTSYGKLTF (SEQ ID NO:446) and CASSSLTPGYGYTF (SEQ ID NO: 132) respectively; CAASTGGYNKLIF (SEQ ID NO:447) and CASSSPTLSTNEKLFF (SEQ ID NO:133) respectively; CAYSGDGYALNF (SEQ ID NO:448) and CASSSRTGYYEQYF (SEQ ID NO:134) respectively; CATDARGDFGNEKLTF (SEQ ID NO:449) and CASSSSQRTMDGYTF (SEQ ID NO: 135) respectively; CATDKGSNYQLIW (SEQ ID NO:450) and CASSSTGTGPFF (SEQ ID NO:136) respectively; CAASTGNQFYF (SEQ ID NO:451) and CASSSVWGQGGEQYF (SEQ ID NO:137) respectively; CLVGVDQTGANNLFF (SEQ ID NO:452) and CASSTGGWGPNSPLHF (SEQ ID NO: 138) respectively; CAVSVPGSNYQLIW (SEQ ID NO:453) and CASSTGPQETQYF (SEQ ID NO:139) respectively; CAANNNAGNMLTF (SEQ ID NO:454) and CASSTQENEKLFF (SEQ ID NO:140) respectively; CAGRNSGYALNF (SEQ ID NO:455) and CASSTRTNQHEKLFF (SEQ ID NO:141) respectively; CAVPYLSGAGSYQLTF (SEQ ID NO:456) and CASSTTAAGNTIYF (SEQ ID NO: 142) respectively; CAVSPNSGGYQKVTF (SEQ ID NO:457) and CASSVGGLASSYEQYF (SEQ ID NO: 143) respectively; CAASTPNNNARLMF (SEQ ID NO:458) and CASSVGLAGSQETQYF (SEQ ID NO:144) respectively or CVVEPGNYGQNFVF (SEQ ID NO:459) and CASSWGMPNEKLFF (SEQ ID NO: 145) respectively.

In certain cases, a T cell receptor disclosed herein comprises a CD3α chain and a CD3β chain comprising CAMSASTGGFKTIF (SEQ ID NO:460) and CASSWGSNQPQHF (SEQ ID NO: 146) respectively; CAENMMDSSYKLIF (SEQ ID NO:461) and CASSWTPAGETQYF (SEQ ID NO: 147) respectively; CAASGNFGNEKLTF (SEQ ID NO:462) and CASSYPSGAFGNEQFF (SEQ ID NO: 148) respectively; CAVPSNAGGTSYGKLTF (SEQ ID NO:463) and CASSYRGAGQPQHF (SEQ ID NO: 149) respectively; CAVRDGAGSYQLTF (SEQ ID NO:464) and CASSYSYEQYF (SEQ ID NO: 150) respectively; CALTGMMDSSYKLIF (SEQ ID NO:465) and CASSYTTEAFF (SEQ ID NO:151) respectively; CAYRTPPNDMRF (SEQ ID NO:466) and CASTPGSGANVLTF (SEQ ID NO:152) respectively; CAASATDSSYKLIF (SEQ ID NO:467) and CASTPSQGHNSPLHF (SEQ ID NO:153) respectively; CAVNAPFGNEKLTF (SEQ ID NO:468) and CASRRNGLYYTF (SEQ ID NO:154) respectively; CAASRTGRRALTF (SEQ ID NO:469) and CATSDDSGQGAEAFF (SEQ ID NO:155) respectively; CAHTSSGGSYIPTF (SEQ ID NO:470) and CATSDMGLADNEQFF (SEQ ID NO: 156) respectively; CAASDSNYQLIW (SEQ ID NO:471) and CATSDPSGPNYNEQFF (SEQ ID NO:157) respectively; CAVGAYNNNDMRF (SEQ ID NO:472) and CATSEGGQGGYGYTF (SEQ ID NO: 158) respectively; CAGFNSGYALNF (SEQ ID NO:473) and CATSGGGAYEQYF (SEQ ID NO:159) respectively; CAVSSTGANSKLTF (SEQ ID NO:474) and CATSQERRQVGSPLHF (SEQ ID NO: 160) respectively; CAFILPSGAGSYQLTF (SEQ ID NO:475) and CAWSALAGSWAGELFF (SEQ ID NO:161) respectively or CAMSEPNGQNFVF (SEQ ID NO:476) and CSAAGTGNTEAFF (SEQ ID NO: 162) respectively.

In certain cases, a T cell receptor disclosed herein comprises a CD3α chain and a CD3β chain comprising CAPRDSGYSTLTF (SEQ ID NO:477) and CSAARQRTNYGYTF (SEQ ID NO: 163) respectively; CAVRRNAGNMLTF (SEQ ID NO:478) and CSADRTSAKNIQYF (SEQ ID NO: 164) respectively; CAESHNTDKLIF (SEQ ID NO:479) and CSAFRLAAQGGSYEQYF (SEQ ID NO: 165) respectively; CVVGTGTASKLTF (SEQ ID NO:480) and CSAIRPGVGDYEQYF (SEQ ID NO: 166) respectively; CAMKTGGGNKLTF (SEQ ID NO:481) and CSAKSTGYDYEQYF (SEQ ID NO:167) respectively; CVVKLNSSASKIIF (SEQ ID NO:482) and CSALSQSGGTNIQYF (SEQ ID NO:168) respectively; CAASLNFNKFYF (SEQ ID NO:483) and CSALWSGDGEQFF (SEQ ID NO:169) respectively; CAVRDGGGYSTLTF (SEQ ID NO:484) and CSAMTREGGNQPQHF (SEQ ID NO: 170) respectively; CASRFSGGYNKLIF (SEQ ID NO:485) and CSANPLAGGGEQYF (SEQ ID NO:171) respectively; CAVSDPGGYNKLIF (SEQ ID NO:486) and CSAPGPAAAGELFF (SEQ ID NO:172) respectively; CAVSEPGGYQKVTF (SEQ ID NO:487) and CSAPGTSAGANVLTF (SEQ ID NO: 173) respectively; CAFRSNNNDMRF (SEQ ID NO:488) and CSAPKLVGSGNTIYF (SEQ ID NO:174) respectively; CAVWGVNQAGTALIF (SEQ ID NO:489) and CSAPQDRNNEQFF (SEQ ID NO: 175) respectively; CGTPSGGYQKVTF (SEQ ID NO:490) and CSAPSTDRVRGYTF (SEQ ID NO:176) respectively; CAASQAAGNKLTF (SEQ ID NO:491) and CSARDHTSGSGNEQFF (SEQ ID NO: 177) respectively; CAASMGTGNQFYF (SEQ ID NO:492) and CSARDPDRGSGNEQYF (SEQ ID NO: 178) respectively; CALSEVYNNDMRF (SEQ ID NO:493) and CSARDQGALLNSPLHF (SEQ ID NO: 179) respectively; CALHWRGAQKLVF (SEQ ID NO:494) and CSARDRGGNTEAFF (SEQ ID NO: 180) respectively; CAVRDQAGTALIF (SEQ ID NO:495) and CSARDRVGGEQFF (SEQ ID NO:181) respectively; CAASMAAGNQFYF (SEQ ID NO:496) and CSARDVRLNTEAFF (SEQ ID NO:182) respectively; CAASTGAGNMLTF (SEQ ID NO:497) and CSARDVWGTGNSQASGNEQFF (SEQ ID NO: 183) respectively; CAVRDTGNQFYF (SEQ ID NO:498) and CSARGLAGADTQYF (SEQ ID NO: 184) respectively; CAVKGSNTGKLIF (SEQ ID NO:499) and CSARGPGGNTEAFF (SEQ ID NO: 185) respectively or CATHPNSGYALNF (SEQ ID NO:500) and CSARGPGTDTQYF (SEQ ID NO: 186) respectively.

In certain cases, a T cell receptor disclosed herein comprises a CD3α chain and a CD3β chain comprising CAGLNTGNQFYF (SEQ ID NO:501) and CSARGRQDQPQHF (SEQ ID NO: 187) respectively; CALSDQNARLMF (SEQ ID NO:502) and CSARTITGSGYTF (SEQ ID NO:188) respectively; CIVRVSNSGNTPLVF (SEQ ID NO:503) and CSARTSGRGYNEQFF (SEQ ID NO: 189) respectively; CALSDPQAALTF (SEQ ID NO:504) and CSARVLGAGPNNEQFF (SEQ ID NO: 190) respectively; CVVSDRPGGGNKLTF (SEQ ID NO:505) and CSARVSAVSTDTQYF (SEQ ID NO:191) respectively; CAMRTGGGNKLTF (SEQ ID NO:506) and CSASPLAGGSYEQYF (SEQ ID NO: 192) respectively; CAVSEPGGYNKLIF (SEQ ID NO:507) and CSASPLKAGANVLTF (SEQ ID NO: 193) respectively; CIVKNTGTALIF (SEQ ID NO:508) and CSASRDSNQPQHF (SEQ ID NO:194) respectively; CAVGGRGSTLGRLYF (SEQ ID NO:509) and CSASSDRGGNQPQHF (SEQ ID NO: 195) respectively; CAMSNNFNKFYF (SEQ ID NO:510) and CSASSGTVGGYTF (SEQ ID NO:196) respectively; CAPPRGTGGYNKLIF (SEQ ID NO:511) and CSASSGVSSYNEQFF (SEQ ID NO:197) respectively; CAVSEPGGYQKVTF (SEQ ID NO:512) and CSATRFGQANTGELFF (SEQ ID NO: 198) respectively; CAVRDSGGYNKLIF (SEQ ID NO:513) and CSATTWTGGNTEAFF (SEQ ID NO: 199) respectively; CAVSESGGYQKVTF (SEQ ID NO:514) and CSAVDWTSGSSYEQYV (SEQ ID NO:200) respectively; CAVRGFSDGQKLLF (SEQ ID NO:515) and CSAVLGLAGVRDTQYF (SEQ ID NO:201) respectively; CARRGSSGSARQLTF (SEQ ID NO:516) and CSISPDRGGNQPQHF (SEQ ID NO:202) respectively; CAVPYLTNAGKSTF (SEQ ID NO:517) and CSLVPDRGGNQPQHF (SEQ ID NO:203) respectively; CAVEETSGSRLTF (SEQ ID NO:518) and CSRGGREGEQFF (SEQ ID NO:204) respectively; CAGQAAYKYIF (SEQ ID NO:519) and CSVEGQATYEQYF (SEQ ID NO:205) respectively; CAVSQAWGGKLIF (SEQ ID NO:520) and CSVEGQGNYGYTF (SEQ ID NO:206) respectively; CAGTNTDKLIF (SEQ ID NO:521) and CSVLGQGAPRSYEQYF (SEQ ID NO:207) respectively or CAESIGTDKLIF (SEQ ID NO:522) and CSVRGRANEQYF (SEQ ID NO:208) respectively.

In certain cases, a T cell receptor disclosed herein comprises a CD3α chain and a CD3β chain comprising CAVLFGNEKLTF (SEQ ID NO:523) and CASARTGQETQYF (SEQ ID NO:209) respectively; CAVDDSGGGADGLTF (SEQ ID NO:524) and CASNLPRSGELFF (SEQ ID NO:210) respectively; CAASWGGTSYGKLTF (SEQ ID NO:525) and CASRPELDSYEQYF (SEQ ID NO:211) respectively; CAPILQGAQKLVF (SEQ ID NO:526) and CASRRGGISNQPQHF (SEQ ID NO:212) respectively; CILRDNYGQNFVF (SEQ ID NO:527) and CASSEHGGNYGYTF (SEQ ID NO:213) respectively; CAVAGTASKLTF (SEQ ID NO:528) and CASSIFTLSNQPQHF (SEQ ID NO:214) respectively; CAVYSNTGKLIF (SEQ ID NO:529) and CASSKQGATEAFF (SEQ ID NO:215) respectively; CAVNTGNQFYF (SEQ ID NO:530) and CASSLGANYGYTF (SEQ ID NO:216) respectively; CAVLLTGGGNKLTF (SEQ ID NO:531) and CASSLTDLYEQYF (SEQ ID NO:217) respectively; CIVGNTGGFKTIF (SEQ ID NO:532) and CASSPDRVEQYF (SEQ ID NO:218) respectively; CAASVWGGSEKLVF (SEQ ID NO:533) and CASSPPGGTEVYEQYF (SEQ ID NO:219) respectively; CALSDRGGNKLVF (SEQ ID NO:534) and CASSPPPGRAETGELFF (SEQ ID NO:220) respectively; CAARETYNTDKLIF (SEQ ID NO:535) and CASSRGAGELFF (SEQ ID NO:221) respectively; CGSPGAGSYQLTF (SEQ ID NO:536) and CASSVGGDYGYTF (SEQ ID NO:222) respectively; CAGGNAGNNRKLIW (SEQ ID NO:537) and CASSYGTANTEAFF (SEQ ID NO:223) respectively; CAFMMLTGGGADGLTF (SEQ ID NO:538) and CASSYSTLAGGHSYEQYF (SEQ ID NO:224) respectively; CAVRDGAGSYQLTF (SEQ ID NO:539) and CASSYSYEQYF (SEQ ID NO:225) respectively or CIVRVEAGKSTF (SEQ ID NO:540) and CSVAGQGNSPLHF (SEQ ID NO:226) respectively.

In certain cases, a T cell receptor disclosed herein comprises a CD3α chain and a CD3β chain comprising CATIQTGANNLFF (SEQ ID NO:541) and CASGGTTDTQYF (SEQ ID NO:227) respectively; CAGYNSGTYKYIF (SEQ ID NO:542) and CASRRGNTGELFF (SEQ ID NO:228) respectively; CAVQAVNNNARLMF (SEQ ID NO:543) and CASRSTGTGEKLFF (SEQ ID NO:229) respectively; CAFMNTDKLIF (SEQ ID NO:544) and CASSFWAGVSTDTQYF (SEQ ID NO:230) respectively; CAMREGSGGYNKLIF (SEQ ID NO:545) and CASSGGRKLDTQYF (SEQ ID NO:231) respectively; CAMRPRSSNTGKLIF (SEQ ID NO:546) and CASSLNLLDRASLETQYF (SEQ ID NO:232) respectively; CATDFFGNEKLTF (SEQ ID NO:547) and CASSLTGYNSPLHF (SEQ ID NO:233) respectively; CAVSHTGNQFYF (SEQ ID NO:548) and CASSLVLEHEQFF (SEQ ID NO:234) respectively; CAELRIQGAQKLVF (SEQ ID NO:549) and CASSQDRITSGYGYTF (SEQ ID NO:235) respectively; CVVIFTGTASKLTF (SEQ ID NO:550) and CASSTGGRSNQPQHF (SEQ ID NO:236) respectively; CAVSGLGGGADGLTF (SEQ ID NO:551) and CASSVVPGAGGEQFF (SEQ ID NO:237) respectively; CALPDSGGGADGLTF (SEQ ID NO:552) and CASSVVPGGPGGELFF (SEQ ID NO:238) respectively; CAENIKGSSGYSTLTF (SEQ ID NO:553) and CASSWAPHTDEQFF (SEQ ID NO:239) respectively; CAGEGAGSYQLTF (SEQ ID NO:554) and CASSWTGNTGELFF (SEQ ID NO:240) respectively; CAVGDSNYQLIW (SEQ ID NO:555) and CASSYTQETQYF (SEQ ID NO:241) respectively; CAVQGALNNARLMF (SEQ ID NO:556) and CASSYTTSGGTYEQYF (SEQ ID NO:242) respectively; CAVSDPLGGSNYKLTF (SEQ ID NO:557) and CASTPSGGTQPQHF (SEQ ID NO:243) respectively; CAGHQAGTALIF (SEQ ID NO:558) and CATSDPGTREQFF (SEQ ID NO:244) respectively; CAVSGGATNKLIF (SEQ ID NO:559) and CATSYRAGGGYNEQFF (SEQ ID NO:245) respectively; CAASIELTGGGNKLTF (SEQ ID NO:560) and CSARGNEQFF (SEQ ID NO:246) respectively; CAAGMYSSASKIIF (SEQ ID NO:561) and CSASSSGTQYF (SEQ ID NO:247) respectively; CALSLSGYSTLTF (SEQ ID NO:562) and CAAEDLAKNIQYF (SEQ ID NO:248) respectively or CAVINVDFQKLVF (SEQ ID NO:563) and CAGRRRLGDSPLHF (SEQ ID NO:249) respectively.

In certain cases, a T cell receptor disclosed herein comprises a CD3α chain and a CD3β chain comprising CASLTGGGNKLTF (SEQ ID NO:564) and CASKQDLNTEAFF (SEQ ID NO:250) respectively; CAMGITSGYALNF (SEQ ID NO:565) and CASLSGPGYEQYF (SEQ ID NO:251) respectively; CAVKGGGATNKLIF (SEQ ID NO:566) and CASNAGYTSGELFF (SEQ ID NO:252) respectively; CAVLGYGNKLVF (SEQ ID NO:567) and CASQDRTALEQYF (SEQ ID NO:253) respectively; CAGQLAAGTASKLTF (SEQ ID NO:568) and CASRGGSSGANVLTF (SEQ ID NO:254) respectively; CARYSGGGADGLTF (SEQ ID NO:569) and CASRGTSGRTYEQYF (SEQ ID NO:255) respectively; CAVSPSGGYQKVTF (SEQ ID NO:570) and CASRLAGQEANYGYTF (SEQ ID NO:256) respectively; CAENRRAGGTSYGKLTF (SEQ ID NO:571) and CASRPSLLRELFF (SEQ ID NO:257) respectively; CAGYNSGTYKYIF (SEQ ID NO:572) and CASRRGNTGELFF (SEQ ID NO:258) respectively; CAASDAGNMLTF (SEQ ID NO:573) and CASRRNSGANVLTF (SEQ ID NO:259) respectively; CAARGNSGGSNYKLTF (SEQ ID NO:574) and CASSARDRYYGYTF (SEQ ID NO:260) respectively; CATVNSGNTPLVF (SEQ ID NO:575) and CASSDRDTDTQYF (SEQ ID NO:261) respectively; CAVERGSQGNLIF (SEQ ID NO:576) and CASSEGGTRHETQYF (SEQ ID NO:262) respectively; CAVMDSNYQLIW (SEQ ID NO:577) and CASSEGQGADTQYF (SEQ ID NO:263) respectively; CAGPGYGNKLVF (SEQ ID NO:578) and CASSEVSGNQPQHF (SEQ ID NO:264) respectively; CAAGVNFGNEKLTF (SEQ ID NO:579) and CASSFGLTNEKLFF (SEQ ID NO:265) respectively; CAASRGFNDMRF (SEQ ID NO:580) and CASSFGTGVYGYTF (SEQ ID NO:266) respectively or CAVSGLVGNEKLTF (SEQ ID NO:581) and CASSFMDRDNSPLHF (SEQ ID NO:267) respectively.

In certain cases, a T cell receptor disclosed herein comprises a CD3α chain and a CD3β chain comprising CAVVFNKFYF (SEQ ID NO:582) and CASSFSGDNEQFF (SEQ ID NO:268) respectively; CATEGDSGYSTLTF (SEQ ID NO:583) and CASSGQGGGYGYTF (SEQ ID NO:269) respectively; CAVSGTGNQFYF (SEQ ID NO:584) and CASSITRKETQYF (SEQ ID NO:270) respectively; CAASVGGSNYKLTF (SEQ ID NO:585) and CASSLAHYEQYF (SEQ ID NO:271) respectively; CAASGSDSGNTPLVF (SEQ ID NO:586) and CASSLAPHTDEQFF (SEQ ID NO:272) respectively; CAMSSRGSARQLTF (SEQ ID NO:587) and CASSLDEQGQNEQFF (SEQ ID NO:273) respectively; CAVNGFGNVLHC (SEQ ID NO:588) and CASSLEADYEQYF (SEQ ID NO:274) respectively; CAAPSRDDKIIF (SEQ ID NO:589) and CASSLEDNQPQHF (SEQ ID NO:275) respectively; CLVGDNAPSGSARQLTF (SEQ ID NO:590) and CASSLGGQVYGYTF (SEQ ID NO:276) respectively; CAENGSDYKLSF (SEQ ID NO:591) and CASSLGQGLNEKLFF (SEQ ID NO:277) respectively; CAALSHQGAQKLVF (SEQ ID NO:592) and CASSLGRNYGYTF (SEQ ID NO:278) respectively; CAVRVFSGGYNKLIF (SEQ ID NO:593) and CASSLGTSAYNEQFF (SEQ ID NO:279) respectively; CAVGERGATNKLIF (SEQ ID NO:594) and CASSLMQAANSPLHF (SEQ ID NO:280) respectively; CAVKSNSGNTPLVF (SEQ ID NO:595) and CASSLMSATNYGYTF (SEQ ID NO:281) respectively; CAASEPGAQKLVF (SEQ ID NO:596) and CASSLQGAREKLFF (SEQ ID NO:282) respectively; CAGAVTTDSWGKLQF (SEQ ID NO:597) and CASSLQGGTEAFF (SEQ ID NO:283) respectively; CAVNVNSGAGSYQLTF (SEQ ID NO:598) and CASSLSGSSYNEQFF (SEQ ID NO:284) respectively or CAMRERTGGSYIPTF (SEQ ID NO:599) and CASSLSGTGNGRNQPQHF (SEQ ID NO:285) respectively.

In certain cases, a T cell receptor disclosed herein comprises a CD3α chain and a CD3β chain comprising CAIGRGSTLGRLYF (SEQ ID NO:600) and CASSLSRDAVGGYTF (SEQ ID NO:286) respectively; CAVSPPGYSSASKIIF (SEQ ID NO:601) and CASSLTGTGGYEQYF (SEQ ID NO:287) respectively; CAEELSGGYQKVTF (SEQ ID NO:602) and CASSLVAGGYEQYF (SEQ ID NO:288) respectively; CAVEFTEYGNKLVF (SEQ ID NO:603) and CASSLYNEQFF (SEQ ID NO:289) respectively; CAVSYSSASKIIF (SEQ ID NO:604) and CASSPPFGSYEQYF (SEQ ID NO:290) respectively; CAEFYNQGGKLIF (SEQ ID NO:605) and CASSQADTQYF (SEQ ID NO:291) respectively; CAVNNGNKLVF (SEQ ID NO:606) and CASSQGQEFGKLFF (SEQ ID NO:292) respectively; CAGGNAGKSTF (SEQ ID NO:607) and CASSQTSGSYNEQFF (SEQ ID NO:293) respectively; CAASRRGSQGNLIF (SEQ ID NO:608) and CASSRTYEQYF (SEQ ID NO:294) respectively; CAGPMKTSYDKVIF (SEQ ID NO:609) and CASSSANYGYTF (SEQ ID NO:295) respectively; CAVKDSNYQLIW (SEQ ID NO:610) and CASSSGEGEAGELFF (SEQ ID NO:296) respectively; CAASIVGSQGNLIF (SEQ ID NO:611) and CASSSGGLNTEAFF (SEQ ID NO:297) respectively; CAALPGNTPLVF (SEQ ID NO:612) and CASSSGGRAWDTQYF (SEQ ID NO:298) respectively; CAPWRGSARQLTF (SEQ ID NO:613) and CASSSGLAAYEQYF (SEQ ID NO:299) respectively; CAVNPTGGFKTIF (SEQ ID NO:614) and CASSSQGSQETQYF (SEQ ID NO:300) respectively; CIVRPSNAGGTSYGKLTF (SEQ ID NO:615) and CASSSTGGNQPQHF (SEQ ID NO:301) respectively; CAASRVGQLTF (SEQ ID NO:616) and CASSVRQGSAGELFF (SEQ ID NO:302) respectively; CATDAWTGANSKLTF (SEQ ID NO:617) and CASSWGLADETQYF (SEQ ID NO:303) respectively; CAAKWAYSGAGSYQLTF (SEQ ID NO:618) and CASSYDSRYGYTF (SEQ ID NO:304) respectively; CAVRDNNQGGKLIF (SEQ ID NO:619) and CASSYSAGEQYF (SEQ ID NO:305) respectively; CAYRSQETSGSRLTF (SEQ ID NO:620) and CASSYSPSTKNIQYF (SEQ ID NO:306) respectively; CAADTGRRALTF (SEQ ID NO:621) and CATEGRGNTIYF (SEQ ID NO:307) respectively; CAKYTDKLIF (SEQ ID NO:622) and CATPPGGLANTGELFF (SEQ ID NO:308) respectively; CAASIGSTLGRLYF (SEQ ID NO:623) and CATSDSSGRYYNEQFF (SEQ ID NO:309) respectively; CVVNGPPGGSYIPTF (SEQ ID NO:624) and CAWSGMNTEAFF (SEQ ID NO:310) respectively; CAVTDSWGKLQF (SEQ ID NO:625) and CSARGGHSFEQYF (SEQ ID NO:311) respectively; CAVVDSNYQLIW (SEQ ID NO:626) and CSARNGDTEAFF (SEQ ID NO:312) respectively; CAEELSGGYQKVTF (SEQ ID NO:627) and CASSLVAGGYEQYF (SEQ ID NO:313) respectively; CAVSFKAAGNKLTF (SEQ ID NO:628) and CSVRVNTEAFF (SEQ ID NO:314) respectively; CAGYNSGTYKYIF (SEQ ID NO: 632) and CASRRGNTGELFF (SEQ ID NO: 631) respectively; CAASIVGSQGNLIF (SEQ ID NO: 634) and CASSSGGLNTEAFF (SEQ ID NO: 633) respectively.

Disclosed herein are T cell receptors comprising a CD3α chain and a CD3β chain. In certain embodiments, a T cell receptor comprises a CD3α chain comprising SEQ ID NO: 315 and a CD3β comprising any one of SEQ ID NOs: 1-314, or 630, or 631 or 633. In certain embodiments, a T cell receptor comprises a CD3α chain comprising SEQ ID NO: 316 and a CD3β comprising any one of SEQ ID NOs: 1-314, or 630, or 631 or 633. In certain embodiments, a T cell receptor comprises a CD3α chain comprising SEQ ID NO: 317 and a CD3β comprising any one of SEQ ID NOs: 1-314, or 630, or 631 or 633. In certain embodiments, a T cell receptor comprises a CD3α chain comprising SEQ ID NO: 318 and a CD3β comprising any one of SEQ ID NOs: 1-314, or 630, or 631 or 633. In certain embodiments, a T cell receptor comprises a CD3α chain comprising SEQ ID NO: 319 and a CD3β comprising any one of SEQ ID NOs: 1-314, or 630, or 631 or 633. In certain embodiments, a T cell receptor comprises a CD3α chain comprising SEQ ID NO: 320 and a CD3β comprising any one of SEQ ID NOs: 1-314, or 630, or 631 or 633. In certain embodiments, a T cell receptor comprises a CD3α chain comprising SEQ ID NO: 321 and a CD3β comprising any one of SEQ ID NOs: 1-314, or 630, or 631 or 633. In certain embodiments, a T cell receptor comprises a CD3α chain comprising SEQ ID NO: 332 and a CD3β comprising any one of SEQ ID NOs: 1-314, or 630, or 631 or 633. In certain embodiments, a T cell receptor comprises a CD3α chain comprising SEQ ID NO: 334 and a CD3β comprising any one of SEQ ID NOs: 1-314, or 630, or 631 or 633. In certain embodiments, a T cell receptor comprises a CD3α chain comprising SEQ ID NO: 335 and a CD3β comprising any one of SEQ ID NOs: 1-314, or 630, or 631 or 633. In certain embodiments, a T cell receptor comprises a CD3α chain comprising SEQ ID NO: 336 and a CD3β comprising any one of SEQ ID NOs: 1-314, or 630, or 631 or 633. In certain embodiments, a T cell receptor comprises a CD3α chain comprising SEQ ID NO: 337 and a CD3β comprising any one of SEQ ID NOs: 1-314, or 630, or 631 or 633. In certain embodiments, a T cell receptor comprises a CD3α chain comprising SEQ ID NO: 338 and a CD3β comprising any one of SEQ ID NOs: 1-314, or 630, or 631 or 633.

Disclosed herein are T cell receptors comprising a CD3α chain and a CD3β chain. In certain embodiments, a T cell receptor comprises a CD3α chain comprising SEQ ID NO: 339 and a CD3β comprising any one of SEQ ID NOs: 1-314, or 630, or 631 or 633. In certain embodiments, a T cell receptor comprises a CD3α chain comprising SEQ ID NO: 340 and a CD3β comprising any one of SEQ ID NOs: 1-314, or 630, or 631 or 633. In certain embodiments, a T cell receptor comprises a CD3α chain comprising SEQ ID NO: 341 and a CD3β comprising any one of SEQ ID NOs: 1-314, or 630, or 631 or 633. In certain embodiments, a T cell receptor comprises a CD3α chain comprising SEQ ID NO: 342 and a CD3β comprising any one of SEQ ID NOs: 1-314, or 630, or 631 or 633. In certain embodiments, a T cell receptor comprises a CD3α chain comprising SEQ ID NO: 343 and a CD3β comprising any one of SEQ ID NOs: 1-314, or 630, or 631 or 633. In certain embodiments, a T cell receptor comprises a CD3α chain comprising SEQ ID NO: 344 and a CD3β comprising any one of SEQ ID NOs: 1-314, or 630, or 631 or 633. In certain embodiments, a T cell receptor comprises a CD3α chain comprising SEQ ID NO: 345 and a CD3β comprising any one of SEQ ID NOs: 1-314, or 630, or 631 or 633. In certain embodiments, a T cell receptor comprises a CD3α chain comprising SEQ ID NO: 346 and a CD3β comprising any one of SEQ ID NOs: 1-314, or 630, or 631 or 633. In certain embodiments, a T cell receptor comprises a CD3α chain comprising SEQ ID NO: 347 and a CD3β comprising any one of SEQ ID NOs: 1-314, or 630, or 631 or 633. In certain embodiments, a T cell receptor comprises a CD3α chain comprising SEQ ID NO: 348 and a CD3β comprising any one of SEQ ID NOs: 1-314, or 630, or 631 or 633.

Disclosed herein are T cell receptors comprising a CD3α chain and a CD3β chain. In certain embodiments, a T cell receptor comprises a CD3α chain comprising SEQ ID NO: 349 and a CD3β comprising any one of SEQ ID NOs: 1-314, or 630, or 631 or 633. In certain embodiments, a T cell receptor comprises a CD3α chain comprising SEQ ID NO: 350 and a CD3β comprising any one of SEQ ID NOs: 1-314, or 630, or 631 or 633. In certain embodiments, a T cell receptor comprises a CD3α chain comprising SEQ ID NO: 351 and a CD3β comprising any one of SEQ ID NOs: 1-314, or 630, or 631 or 633. In certain embodiments, a T cell receptor comprises a CD3α chain comprising SEQ ID NO: 352 and a CD3β comprising any one of SEQ ID NOs: 1-314, or 630, or 631 or 633. In certain embodiments, a T cell receptor comprises a CD3α chain comprising SEQ ID NO: 353 and a CD3β comprising any one of SEQ ID NOs: 1-314, or 630, or 631 or 633.

In certain embodiments, a T cell receptor comprises a CD3α chain comprising SEQ ID NO: 354 and a CD3β comprising any one of SEQ ID NOs: 1-314, or 630, or 631 or 633. In certain embodiments, a T cell receptor comprises a CD3α chain comprising SEQ ID NO: 355 and a CD3β comprising any one of SEQ ID NOs: 1-314, or 630, or 631 or 633. In certain embodiments, a T cell receptor comprises a CD3α chain comprising SEQ ID NO: 356 and a CD3β comprising any one of SEQ ID NOs: 1-314, or 630, or 631 or 633. In certain embodiments, a T cell receptor comprises a CD3α chain comprising SEQ ID NO: 357 and a CD3β comprising any one of SEQ ID NOs: 1-314, or 630, or 631 or 633. In certain embodiments, a T cell receptor comprises a CD3α chain comprising SEQ ID NO: 358 and a CD3β comprising any one of SEQ ID NOs: 1-314, or 630, or 631 or 633. In certain embodiments, a T cell receptor comprises a CD3α chain comprising SEQ ID NO: 359 and a CD3β comprising any one of SEQ ID NOs: 1-314, or 630, or 631 or 633. In certain embodiments, a T cell receptor comprises a CD3α chain comprising SEQ ID NO: 360 and a CD3β comprising any one of SEQ ID NOs: 1-314, or 630, or 631 or 633. In certain embodiments, a T cell receptor comprises a CD3α chain comprising SEQ ID NO: 361 and a CD3β comprising any one of SEQ ID NOs: 1-314, or 630, or 631 or 633.

Disclosed herein are T cell receptors comprising a CD3α chain and a CD3β chain. In certain embodiments, a T cell receptor comprises a CD3α chain comprising SEQ ID NO: 362 and a CD3β comprising any one of SEQ ID NOs: 1-314, or 630, or 631 or 633. In certain embodiments, a T cell receptor comprises a CD3α chain comprising SEQ ID NO: 363 and a CD3β comprising any one of SEQ ID NOs: 1-314, or 630, or 631 or 633. In certain embodiments, a T cell receptor comprises a CD3α chain comprising SEQ ID NO: 364 and a CD3β comprising any one of SEQ ID NOs: 1-314, or 630, or 631 or 633. In certain embodiments, a T cell receptor comprises a CD3α chain comprising SEQ ID NO: 365 and a CD3β comprising any one of SEQ ID NOs: 1-314, or 630, or 631 or 633. In certain embodiments, a T cell receptor comprises a CD3α chain comprising SEQ ID NO: 366 and a CD3β comprising any one of SEQ ID NOs: 1-314, or 630, or 631 or 633.

In certain embodiments, a T cell receptor comprises a CD3α chain comprising SEQ ID NO: 367 and a CD3β comprising any one of SEQ ID NOs: 1-314, or 630, or 631 or 633. In certain embodiments, a T cell receptor comprises a CD3α chain comprising SEQ ID NO: 368 and a CD3β comprising any one of SEQ ID NOs: 1-314, or 630, or 631 or 633. In certain embodiments, a T cell receptor comprises a CD3α chain comprising SEQ ID NO: 369 and a CD3β comprising any one of SEQ ID NOs: 1-314, or 630, or 631 or 633. In certain embodiments, a T cell receptor comprises a CD3α chain comprising SEQ ID NO: 370 and a CD3β comprising any one of SEQ ID NOs: 1-314, or 630, or 631 or 633. In certain embodiments, a T cell receptor comprises a CD3α chain comprising SEQ ID NO: 371 and a CD3β comprising any one of SEQ ID NOs: 1-314, or 630, or 631 or 633. In certain embodiments, a T cell receptor comprises a CD3α chain comprising SEQ ID NO: 372 and a CD3β comprising any one of SEQ ID NOs: 1-314, or 630, or 631 or 633.

In certain embodiments, a T cell receptor comprises a CD3α chain comprising SEQ ID NO: 373 and a CD3β comprising any one of SEQ ID NOs: 1-314, or 630, or 631 or 633. In certain embodiments, a T cell receptor comprises a CD3α chain comprising SEQ ID NO: 374 and a CD3β comprising any one of SEQ ID NOs: 1-314, or 630, or 631 or 633. In certain embodiments, a T cell receptor comprises a CD3α chain comprising SEQ ID NO: 375 and a CD3β comprising any one of SEQ ID NOs: 1-314, or 630, or 631 or 633. In certain embodiments, a T cell receptor comprises a CD3α chain comprising SEQ ID NO: 376 and a CD3β comprising any one of SEQ ID NOs: 1-314, or 630, or 631 or 633. In certain embodiments, a T cell receptor comprises a CD3α chain comprising SEQ ID NO: 376 and a CD3β comprising any one of SEQ ID NOs: 1-314, or 630, or 631 or 633. In certain embodiments, a T cell receptor comprises a CD3α chain comprising SEQ ID NO: 377 and a CD3β comprising any one of SEQ ID NOs: 1-314, or 630, or 631 or 633. In certain embodiments, a T cell receptor comprises a CD3α chain comprising SEQ ID NO: 378 and a CD3β comprising any one of SEQ ID NOs: 1-314, or 630, or 631 or 633. In certain embodiments, a T cell receptor comprises a CD3α chain comprising SEQ ID NO: 379 and a CD3β comprising any one of SEQ ID NOs: 1-314, or 630, or 631 or 633. In certain embodiments, a T cell receptor comprises a CD3α chain comprising SEQ ID NO: 378 and a CD3β comprising any one of SEQ ID NOs: 1-314, or 630, or 631 or 633. In certain embodiments, a T cell receptor comprises a CD3α chain comprising SEQ ID NO: 379 and a CD3β comprising any one of SEQ ID NOs: 1-314, or 630, or 631 or 633. In certain embodiments, a T cell receptor comprises a CD3α chain comprising SEQ ID NO: 380 and a CD3β comprising any one of SEQ ID NOs: 1-314, or 630, or 631 or 633. In certain embodiments, a T cell receptor comprises a CD3α chain comprising any one of SEQ ID NO: 381-629, 632 or 634 and a CD3β comprising any one of SEQ ID NOs: 1-314, or 630, or 631 or 633.

Further disclosed herein an antigen binding protein comprising a binding domain having at least 75%, 75%, 90%, 95%, 96%, 98%, 99% or 100% amino acid sequence identity to any one of SEQ ID Nos: 1-314, or 630, or 631 or 633. In some embodiments, the antigen binding protein further comprises a binding domain having at least 75%, 75%, 90%, 95%, 96%, 98%, 99% or 100% amino acid sequence identity to any one of SEQ ID NOs. 315-629, 632, or 634. In some embodiments, the antigen binding protein comprises a CD3α chain disclosed herein. In some embodiments, the antigen binding protein comprises aa CD3β chain disclosed herein. In some embodiments, disclosed herein are pharmaceutical compositions comprising the TCRs, the isolated cell, the composition, the expression vector or the antigen binding protein disclosed herein. In some embodiments, disclosed herein are use of the TCR, the isolated cell, the composition, the expression vector, the antigen binding protein or the method disclosed herein for the treatment of a subject or for the manufacture of a medicament for the treatment of the subject.

### Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art.

Alternatively, “about” can mean a range of up to 20%, up to 10%, up to 5%, or up to 1% of a given value or range. Alternatively, particularly with respect to biological systems or processes, the term can mean within an order of magnitude, within 5-fold, and also within 2-fold, of a value. Where particular values are described in the application and claims, unless otherwise stated the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

As used herein, the term “alteration” or “alteration of genetic information” refers to any change in the genome of a cell. In the context of treating genetic disorders, alterations may include, but are not limited to, insertion, deletion and correction. As used herein, the term “insertion” refers to an addition of one or more nucleotides in a DNA sequence. Insertions can range from small insertions of a few nucleotides to insertions of large segments such as a cDNA or a gene. The term “deletion” refers to a loss or removal of one or more nucleotides in a DNA sequence or a loss or removal of the function of a gene. In some cases, a deletion can include, for example, a loss of a few nucleotides, an exon, an intron, a gene segment, or the entire sequence of a gene. In some cases, deletion of a gene refers to the elimination or reduction of the function or expression of a gene or its gene product. This can result from not only a deletion of sequences within or near the gene, but also other events (e.g., insertion, nonsense mutation) that disrupt the expression of the gene. The term “correction” or “corrected” as used herein, refers to a change of one or more nucleotides of a genome in a cell, whether by insertion, deletion or substitution. Such correction may result in a more favorable genotypic or phenotypic outcome, whether in structure or function, to the genomic site which was corrected. One non-limiting example of a “correction” includes the correction of a mutant or defective sequence to a wild-type sequence which restores structure or function to a gene or its gene product(s). Depending on the nature of the mutation, correction may be achieved via various strategies disclosed herein. In one non-limiting example, a missense mutation may be corrected by replacing the region containing the mutation with its wild-type counterpart. As another example, duplication mutations (e.g., repeat expansions) in a gene may be corrected by removing the extra sequences.

In some aspects, alterations may also include a gene knock-in, knock-out or knock-down. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a cDNA encoding the wild-type protein may be inserted into the genome of a cell carrying a mutant gene. Knock-in strategies need not replace the defective gene, in whole or in part. In some cases, a knock-in strategy may further involve substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. In some embodiments, the term “knock-down” as used herein refers to reduction in the expression of a gene or its gene product(s). As a result of a gene knock-down, the protein activity or function may be attenuated or the protein levels may be reduced or eliminated.

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C”, “one or more of A, B, and C” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)2 fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

By “ameliorate” is meant decrease, suppress, attenuate, diminish, arrest, or stabilize the development or progression of a disease.

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, p-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The amino acids can be non-naturally occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “amino acid sequence” is the order in which amino acid residues, connected by peptide bonds, lie in the chain in peptides and proteins.

By “cancer” as used herein is meant, a disease, condition, trait, genotype or phenotype characterized by unregulated cell growth or replication as is known in the art; including colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, Primary CNS Lymphoma, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype.

The term “chimeric antigen receptor” or “CAR” as used herein refers to an antigen-binding domain that is fused to an intracellular signaling domain capable of activating or stimulating an immune cell. In certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation” CARs include those that provide both co-stimulation (e.g., CD28 or CD137) and activation (CD3ζ). “Third-generation” CARs include those that provide multiple co-stimulation (e.g. CD28 and CD137) and activation (CD3ζ). “Fourth generation” of CARs have been described as CAR T cells redirected for cytokine killing (TRUCKS) where the vector containing the CAR construct possesses a cytokine cassette. In some embodiments, when the CAR is ligated, the CAR T cell deposits a pro-inflammatory cytokine into the tumor lesion. “Fifth generation” of CARs are mainly designed based on the second generation. However, these CAR-T cells can contain a truncated cytoplasmic receptor (IL-12) and a β-chain domain (IL-2Rβ truncated intracellular interleukin 2β chain receptor) along with the transcription factor STAT3/5 binding motif (Tokarew et al., 2019. Teaching an Old Dog New Tricks: Next-Generation CAR T Cells. Br. J. Cancer 120, 26-37. doi:10.1038/s41416-018-0325-1). In some embodiments, a CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.”

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart. Treatment of a subject also includes a variety of combination therapies with both physical, e.g. surgery, and radiation based treatments.

As used herein, the transitional term “comprising,” which is synonymous with “including,” “containing,” or “characterized by,” is inclusive or open-ended and does not exclude additional, unrecited elements or method steps. When used herein the term “comprising” can be substituted with the term “containing” or “including” or sometimes when used herein with the term “having.” By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

A “conservative amino acid substitution” is one in which the amino acid residue is replaced with an amino acid residue having a similar sidechain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate.

As used herein, the term “guide sequence,” “crRNA,” “guide RNA,” or “single guide RNA,” or “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the guide sequence and a CRISPR effector protein to the target nucleic acid sequence. In some example embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq. sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmic RNA (scRNA).

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises a naive CD4⁺ T cell. In some aspects, an immune cell comprises a memory CD4⁺ T cell. In some aspects, an immune cell comprises an effector CD4⁺ T cell. In some aspects, an immune cell comprises a naïve CD8⁺ T cell. In some aspects, an immune cell comprises a memory CD8⁺ T cell. In some aspects, an immune cell comprises an effector CD8⁺ T cell. In some aspects, an immune cell comprises a gamma delta T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “linker”, also referred to as a “spacer” or “spacer domain” as used herein, refers to an amino acid or sequence of amino acids that that is optionally located between two amino acid sequences in a fusion protein of the invention.

As used herein, the term “kit” refers to any delivery system for delivering materials. Inclusive of the term “kits” are kits for both research and clinical applications. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a sub portion of the total kit components. The containers may be delivered to the intended recipient together or separately. For example, a first container may contain an enzyme for use in an assay, while a second container contains oligonucleotides or liposomes. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a sub portion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, a “natural amino acid” refers to the twenty genetically encoded alpha-amino acids. See, e.g., Biochemistry by L. Stryer, 3^{rd} ed. 1988, Freeman and Company, New York for structures of the twenty natural amino acids.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise and should be understood to mean “either or both” of the elements so conjoined, e.g., elements that are conjunctively present in some cases and disjunctively present in other cases.

“Parenteral” administration of an immunogenic composition includes, e.g., subcutaneous (s.c.), intravenous (i.v.), intramuscular (i.m.), intravitreal (i.v.i.), intra-cisterna magna (i.e.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the helicases described herein, one or more amino acids of the helicase are substituted with one or more unnatural amino acids and/or one or more natural amino acids.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

As used herein, the term “virus” includes any type of virus or virus vector. For example, adenovirus, adeno-associated virus (AAV), recombinant adeno-associated virus (rAAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus. In various embodiments the virus is a chimeric virus, a synthetic virus, a recombinant virus, a mosaic virus or a pseudotyped virus.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Any compositions or methods provided herein can be combined with one or more of any of the other compositions and methods provided herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1D show functional validation of predicted mKRAS-specific TCRs. FIG. 1A. shows a schematic of CRISPR-based TCR knock-in strategy. FIG. 1B. shows a quantification of recombinant TCR knock-in into healthy donor T cells by expression of murine TCRβ and NGFR co-expression on healthy donor T cell populations. FIG. 1C. shows IFNγ production measured by ELISPOT of recombinant TCR expressing cells co-cultured with autologous moDCs pulsed with 2 μg/mL control peptide or pooled KRAS G12V, G12D, G12C, G12R, G12A, G13D peptides. FIG. 1D shows quantification of IFNγ ELISPOT per recombinant TCR of interest. Two-way ANOVA followed by Sidak's multiple comparisons test, ns=p>0.05, *p≤0.05, **p<0.005, ***p≤0.0005, ****p≤0.00001.

FIG. 2A. shows CD137 and CD25 activation marker expression on TCR8 expressing cells co-cultured with CD3− antigen-presenting cells pulsed with 2 μg/mL individual KRAS peptides. FIG. 2B. shows quantification of CD25 and CD137 upregulation of TCR8 in response to individual KRAS peptide recognition. FIG. 2C. shows CD137 and CD25 activation marker expression on TCR8 expressing cells co-cultured with HLA null K562s nucleofected with each patient 1 HLA-allele and pulsed with 2 μg/mL of KRAS G12V peptide. Two-way ANOVA followed by Dunnett's multiple comparisons test, ns=p>0.05, *p<0.05, **p<0.005, ***p<0.0005.

FIG. 3A-FIG. 3D are a series of plots demonstrating the in vitro T cell expansion of post-vaccine PBMCs with mutant KRAS peptide followed by TCRβ sequencing. 1×10⁶ PMBCs from post-vaccine timepoints of patient (FIG. 3A) J1994_12, (FIG. 3B) J1994_5, (FIG. 3C) J1994_1 or (FIG. 3D) J1995_2, were seeded in a 48 well plate and incubated with 2pg/mL KRAS G12V, G12A, G12R, G12C, G12D, G13D, or an irrelevant synthetic long peptide and 100IU/mL hIL-2 for 7 days. Genomic DNA was isolated from peptide expanded PBMCs and sent for TCRβ sequencing. Genomic DNA for baseline timepoint was collected immediately after thawing PBMCs. Frequency of TCRs in the control condition is shown relative to the frequency of each TCR in the peptide expansion conditions or baseline sample. Differential abundance analysis was performed to identify TCRs that expanded greater than 10-fold com pared to the control sample and was present at least 0.1% of the repertoire. Significantly enriched TCRs are shown in black and number of significantly enriched is shown above each condition plot.

FIG. 4A-FIG. 4C are a series of plots and graphs showing the phenotyping and validation of mutant KRAS-specific T cells identified from in vitro expansion assays. FIG. 4A shows single cell RNA/TCR sequencing was performed on pre- and post-vaccine PBMCs for patients J1994_12, J1994_5, J1994_1, and J1994_6. T cells were identified and phenotyped into CD4 (naïve, proliferating, central memory (TCM), and effector memory (TEM)), CD8 (naïve, proliferating, TCM, and TEM), double negative T (dnT), MAIT, and Tregs. FIG. 4B shows phenotype of T cells with TCRβ chains from each mKRAS expansion conditions or that were found in more than one expansion (XR). FIG. 4C shows that Jurkat-TCRKO-NFAT-GFP cells were transduced to express a putative mKRAS-specific TCR. TCR transduced cells were co-cultured with peptide-pulsed, patient-matched LCLs at the indicated peptide concentrations, overnight, 37 C. Cells were analyzed for activation by GFP expression and normalized to specific activity of minimal and maximal activation conditions. The monoreactive TCR transduced cells (TCR01, TCR02, TCR04) showed specific activity to their respective mKRAS epitope and no cross-reactivity with other epitopes (FIG. 4C). The cross reactive TCR03 transduced T cell was validated to respond to KRAS G12V, G12A, and G12C as predicted (FIG. 4D). Jurkat reporter line transduced to express Public TCR008 co-cultured with patient matched LCLs pulsed with control, KRAS G13D, or wild type peptide at the indicated concentrations (FIG. 4E). Public TCR008 Jurkats co-cultured with LCLs from a HLA-DQB*03:01-donor (left) or irrelevant HLA-DQB*03:01+ donor (right) pulsed with control, KRAS G13D, or wild type KRAS peptide (FIG. 4F). Cells were analyzed for activation by GFP expression and normalized to specific activity of minimal (unpulsed LCL only) and maximal (PMA+Ionomycin) activation conditions.

FIG. 5A-FIG. 5C shows validation of KRAS-specific CD8 T cell reactivity. Jurkat-TCR_{KO}-NFAT-GFP cells were transduced to express putative mono-reactive, mKRAS-specific TCRs and co-cultured with peptide-pulsed, patient-matched LCLs at 10 uM per peptide. Cells were analyzed for activation by GFP expression and normalized to specific activity of minimal (unpulsed LCL only) and maximal (PMA+Ionomycin) activation conditions (FIG. 5A—TCR05, FIG. 5B—TCR06, FIG. 5C—TCR07).

### DETAILED DESCRIPTION

The present disclosure provides strategies and techniques for the targeted, specific alteration of the genetic information (genome) of living organisms. This disclosure is based in part on the redirecting of T cells to specifically bind to tumor antigens. In certain embodiments, recombinant T cell and chimeric antigen receptors are provided.

CRISPR-based genetic engineering is a flexible way of introducing genomic mutations in cells. Double strand DNA (dsDNA) breaks can be induced at desired genomic loci through the use of “programmable”, user-defined short guide RNAs which complex with a nuclease. Frequently used nucleases include Cas proteins, for example Cas9, but can be variations thereof. Variations include altered nucleases with altered DNA binding specificities or fusion proteins which add distinct features such as transcriptional activation or repression or enzymatic activity to directly edit nucleotides. Cas nucleases can also be modified to induce single-stranded “nicks” to genomic DNA. The cellular response to these induced DNA breaks is the activation of the DNA repair machinery which mainly consists of the non-homologous end joining (NHEJ) pathway and the homology directed repair (HDR) pathway. NHEJ usually results in random insertions and deletions (indels) which can be exploited to delete genes. This can be useful for experimental purposes, but for clinical use the inherently stochastic NHEJ repair pathway bears significant risks. Targeted, precise gene editing is safer and therefore more desirable. The HDR pathway provides the opportunity to introduce precise mutations by repairing a (ds)DNA break based on a DNA template.

### T-Cell Receptors

In some embodiments, a T-cell antigen receptor (TCR) of a T-cell clone are each composed of a unique combination of domains designated variable (V), [diversity (D),] joining (J), and constant (C). In each T-cell clone, the combination of V, D, and J domains of both the alpha and the beta chains or of both the delta and gamma chains participates in antigen recognition in a manner which is uniquely characteristic of that T-cell clone and defines a unique binding site, also known as the idiotype of the T-cell clone. In contrast, the C domain does not participate in antigen binding.

In some embodiments, a TCR is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. In some embodiments, the extracellular portion of native heterodimeric αβTCR and γδTCR each contain two polypeptides, each of which has a membrane-proximal constant region, and a membrane-distal variable region. In some embodiments, each of the constant and variable regions include an intra-chain disulfide bond. In some embodiments, the variable regions contain the highly polymorphic loops analogous to the complementarity determining regions (CDRs), also known as hypervariable regions, of antibodies. The variable regions of both the TCRα and TCRβ chain each have three CDRs, numbered CDR1, CDR2, and CDR3 in the direction from the amino terminal end to the carboxy terminal end. CDR3 is the main CDR responsible for recognizing processed antigen. The TCRβ CDR3 has been recognized as more structurally diverse than the other CDRs.

In some embodiments, the variable regions of both the TCRα and TCRβ chain can further comprise framework regions (FRs). In some embodiments, each TCRβ chain and TCRα chain can includes three CDRs and four FRs, arranged from N-terminus to C-terminus in the following order: FR1, CDR1, FR2, CDR2, FR3, CDR3, FR4. The techniques for determining CDRs are generally known in the art. In some embodiments, the CDRs can be determined by approaches based on cross-species sequence variability. In some embodiments, the CDRs can be determined by approaches based on crystallographic studies of antigen-antibody complexes. In addition, combinations of these approaches are sometimes used in the art to determine CDRs. In certain embodiments, CDRs can be determined using sequence-based prediction tools. Such tools are generally available in the art, e.g., the Loupe V(D)J Browser provided by 10× Genomics® (Pleasanton, Calif.). For instance, in one embodiment, the single cell TCR sequencing of epitope reactive T-cell population can be conducted using the 10× Genomics® platform. Then the sequence can be processed using the Loupe V(D)J Browser to identify the clonotypes, V(D)J genes, and the CDR motifs, etc. More detailed information about the Loupe V(D)J Browser is available over the world-wide-web at site: support.10×genomics.com/single-cell-vdj/software/visualization/latest/tutorial-clonotypes, which is herein incorporated by reference in its entirety.

In some embodiments, the TCR comprises a constant domain and a variable region. In some embodiments, the variable region comprises a TCRα chain and a TCRβ chain.

In some embodiments, the constant domain comprises a TCRα constant domain (TRAC) and a TCRβ constant domain (TRBC).

In some embodiments, the TCRβ variable chain (TRBV) can be further classified into subgroups. The terms TCRBV, TCRVB, TRBV, TCRβV, TCRVβ or TRβV can be used interchangeably herein and refer to a TCRβ variable chain, e.g., as described herein.

The TRBV subgroups and subgroup members can be found in Table 3.

In some embodiments, the TCRα variable chain (TRAV) can be further classified into subgroups. The terms TCRAV, TCRVA, TRAV, TCRαV, TCRVα or TRαV can be used interchangeably herein and refer to a TCRα variable chain, e.g., as described herein.

The TRAV subgroups and subgroup members can be found in Table 4.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

In some embodiments, a TCRβ chain comprises an extracellular region, which comprises the antigen recognition domain of the TCR. In some embodiments, a TCRα chain comprises an extracellular region, which comprises the antigen binding domain of the TCR. In some embodiments, the antigen binding domain of the TCR comprises complementarity determining regions (CDRs). In some embodiments, the antigen binding domain comprises 1, 2, or 3 CDRs.

TABLE 3
TRBV Subgroups

| TRBV subgroup | TRBV gene name | TRBV allele name |
|---|---|---|
| TRBV1 | TRBV1 | TRBV1*01 |
| TRBV2 | TRBV2 | TRBV2*01 |
| | | TRBV2*02 |
| | | TRBV2*03 |
| TRBV3 | TRBV3-1 | TRBV3-1*01 |
| | | TRBV3-1*02 |
| | TRBV3-2 | TRBV3-2*01 |
| | | TRBV3-2*02 |
| | | TRBV3-2*03 |
| TRBV4 | TRBV4-1 | TRBV4-1*01 |
| | | TRBV4-1*02 |
| | TRBV4-2 | TRBV4-2*01 |
| | | TRBV4-2*02 |
| | TRBV7-3 | TRBV4-3*02 |
| | | TRBV4-3*03 |
| | | TRBV4-3*04 |
| TRBV5 | TRBV5-1 | TRBV5-1*01 |
| | | TRBV5-1*02 |
| | TRBV5-2 | TRBV5-2*01 |
| | TRBV5-3 | TRBV5-3*01 |
| | | TRBV5-3*02 |
| | TRBV5-4 | TRBV5-4*01 |
| | | TRBV5-4*02 |
| | | TRBV5-4*03 |
| | | TRBV5-4*04 |
| | TRBV5-5 | TRBV5-5*01 |
| | | TRBV5-5*02 |
| | | TRBV5-5*03 |
| | TRBV5-6 | TRBV5-6*01 |
| | TRBV5-7 | TRBV5-7*01 |
| | TRBV5-8 | TRBV5-8*01 |
| | | TRBV5-8*02 |
| TRBV6 | TRBV6-1 | TRBV6-1*01 |
| | TRBV6-2 | TRBV6-2*01 |
| | TRBV6-3 | TRBV6-3*01 |
| | TRBV6-4 | TRBV6-4*01 |
| | | TRBV6-4*02 |
| | TRBV6-5 | TRBV6-5*01 |
| | TRBV6-6 | TRBV6-6*01 |
| | | TRBV6-6*02 |
| | | TRBV6-6*03 |
| | | TRBV6-6*04 |
| | | TRBV6-6*05 |
| | TRBV6-7 | TRBV6-7*01 |
| | TRBV6-8 | TRBV6-8*01 |
| | TRBV6-9 | TRBV6-9*01 |
| TRBV7 | TRBV7-1 | TRBV7-1*01 |
| | TRBV7-2 | TRBV7-2*01 |
| | | TRBV7-2*02 |
| | | TRBV7-2*03 |
| | | TRBV7-2*04 |
| | TRBV7-3 | TRBV7-3*01 |
| | | TRBV7-3*02 |
| | | TRBV7-3*03 |
| | | TRBV7-3*04 |
| | | TRBV7-3*05 |
| | TRBV7-4 | TRBV7-4*01 |
| | | TRBV7-4*02 |
| | TRBV7-5 | TRBV7-5*01 |
| | | TRBV7-5*02 |
| | TRBV7-6 | TRBV7-6*01 |
| | | TRBV7-6*02 |
| | TRBV7-7 | TRBV7-7*01 |
| | | TRBV7-7*02 |
| | TRBV7-8 | TRBV7-8*01 |
| | | TRBV7-8*02 |
| | | TRBV7-8*03 |
| | TRBV7-9 | TRBV7-9*01 |
| | | TRBV7-9*02 |
| | | TRBV7-9*03 |
| | | TRBV7-9*04 |
| | | TRBV7-9*05 |
| | | TRBV7-9*06 |
| | | TRBV7-9*07 |
| TRBV8 | TRBV8-1 | TRBV8-1*01 |
| | | TRBV8-1*02 |
| | TRBV8-2 | TRBV8-2*01 |
| | | TRBV8-2*02 |
| TRBV9 | TRBV9 | TRBV9*01 |
| | | TRBV9*02 |
| | | TRBV9*03 |
| TRBV10 | TRBV10-1 | TRBV10-1*01 |
| | | TRBV10-1*02 |
| | | TRBV10-1*03 |
| | TRBV10-2 | TRBV10-2*01 |
| | | TRBV10-2*02 |
| | TRBV10-3 | TRBV10-3*01 |
| | | TRBV10-3*02 |
| | | TRBV10-3*03 |
| | | TRBV10-3*04 |
| TRBV11 | TRBV11-1 | TRBV11-1*01 |
| | TRBV11-2 | TRBV11-2*01 |
| | | TRBV11-2*02 |
| | | TRBV11-2*03 |
| | TRBV11-3 | TRBV11-3*01 |
| | | TRBV11-3*02 |
| | | TRBV11-3*03 |
| | | TRBV11-3*04 |
| TRBV12 | TRBV12-1 | TRBV12-1*01 |
| | TRBV12-2 | TRBV12-2*01 |
| | TRBV12-3 | TRBV12-3*01 |
| | TRBV12-4 | TRBV12-4*01 |
| | | TRBV12-4*02 |
| | TRBV12-5 | TRBV12-5*01 |
| TRBV13 | TRBV13 | TRBV13*01 |
| | | TRBV13*02 |
| TRBV14 | TRBV14 | TRBV14*01 |
| | | TRBV14*02 |
| TRBV15 | TRBV15 | TRBV15*01 |
| | | TRBV15*02 |
| | | TRBV15*03 |
| TRBV16 | TRBV16 | TRBV16*01 |
| | | TRBV16*02 |
| | | TRBV16*03 |
| TRBV17 | TRBV17 | TRBV17*01 |
| | | TRBV17*02 |
| TRBV18 | TRBV18 | TRBV18*01 |
| TRBV19 | TRBV19 | TRBV19*01 |
| | | TRBV19*02 |
| | | TRBV19*03 |
| TRBV20 | TRBV20-1 | TRBV20-1*01 |
| | | TRBV20-1*02 |
| | | TRBV20-1*03 |
| | | TRBV20-1*04 |
| | | TRBV20-1*05 |
| | | TRBV20-1*06 |
| | | TRBV20-1*07 |
| TRBV21 | TRBV21-1 | TRBV21-1*01 |
| | | TRBV21-1*02 |
| TRBV22 | TRBV22-1 | TRBV22-1*01 |
| TRBV23 | TRBV23-1 | TRBV23-1*01 |
| TRBV24 | TRBV24-1 | TRBV24-1*01 |
| | | TRBV24-1*02 |
| TRBV25 | TRBV25-1 | TRBV25-1*01 |
| TRBV26 | TRBV26 | TRBV26*01 |
| TRBV27 | TRBV27 | TRBV27*01 |
| TRBV28 | TRBV28 | TRBV28*01 |
| TRBV29 | TRBV29-1 | TRBV29-1*01 |
| | | TRBV29-1*02 |
| | | TRBV29-1*03 |
| TRBV30 | TRBV30 | TRBV30*01 |
| | | TRBV30*02 |
| | | TRBV30*03 |
| | | TRBV30*04 |
| | | TRBV30*05 |
| TRBVA | TRBVA | TRBVA*01 |
| | | TRBVA*02 |
| TRBVB | TRBVB | TRBVB*01 |
| | | TRBVB*02 |
| TRBVC | TRBVC | TRBVC*01 |
| | | TRBVC*02 |

TABLE 4
TRAV subgroups

| TRAV subgroup | TRAV gene name | TRAV allele name |
|---|---|---|
| TRAV1 | TRAV1-1 | TRAV1-1*01 |
| | | TRAV1-1*02 |
| | TRAV1-2 | TRAV1-2*01 |
| | | TRAV1-2*02 |
| | | TRAV1-2*03 |
| TRAV2 | TRAV2 | TRAV2*01 |
| | | TRAV2*02 |
| TRAV3 | TRAV3 | TRAV3*01 |
| | | TRAV3*02 |
| TRAV4 | TRAV4 | TRAV4*01 |
| TRAV5 | TRAV5 | TRAV5*01 |
| TRAV6 | TRAV6 | TRAV6*01 |
| | | TRAV6*02 |
| | | TRAV6*03 |
| | | TRAV6*04 |
| | | TRAV6*05 |
| | | TRAV6*06 |
| | | TRAV6*07 |
| TRAV7 | TRAV7 | TRAV7*01 |
| TRAV8 | TRAV8-1 | TRAV8-1*01 |
| | | TRAV8-1*02 |
| | TRAV8-2 | TRAV8-2*01 |
| | | TRAV8-2*02 |
| | | TRAV8-2*03 |
| | TRAV8-3 | TRAV8-3*01 |
| | | TRAV8-3*02 |
| | | TRAV8-3*03 |
| | TRAV8-4 | TRAV8-4*01 |
| | | TRAV8-4*02 |
| | | TRAV8-4*03 |
| | | TRAV8-4*04 |
| | | TRAV8-4*05 |
| | | TRAV8-4*06 |
| | | TRAV8-4*07 |
| | TRAV8-5 | TRAV8-5*01 |
| | TRAV8-6 | TRAV8-6*01 |
| | | TRAV8-6*02 |
| | TRAV8-6-1 | TRAV8-6-1*01 |
| | TRAV8-7 | TRAV8-7*01 |
| | | TRAV8-7*02 |
| TRAV9 | TRAV9-1 | TRAV9-1*01 |
| | TRAV9-2 | TRAV9-2*01 |
| | | TRAV9-2*02 |
| | | TRAV9-2*03 |
| | | TRAV9-2*04 |
| TRAV10 | TRAV10 | TRAV10*01 |
| | | TRAV10*02 |
| TRAV11 | TRAV11 | TRAV11*01 |
| | TRAV11-1 | TRAV11-1*01 |
| TRAV12 | TRAV12-1 | TRAV12-1*01 |
| | | TRAV12-1*02 |
| | TRAV12-2 | TRAV12-2*01 |
| | | TRAV12-2*02 |
| | | TRAV12-2*03 |
| | TRAV12-3 | TRAV12-3*01 |
| | | TRAV12-3*02 |
| TRAV13 | TRAV13-1 | TRAV13-1*01 |
| | | TRAV13-1*02 |
| | | TRAV13-1*03 |
| | TRAV13-2 | TRAV13-2*01 |
| | | TRAV13-2*02 |
| TRAV14 | TRAV14/DV4 | TRAV14/DV4*01 |
| | | TRAV14/DV4*02 |
| | | TRAV14/DV4*03 |
| | | TRAV14/DV4*04 |
| | TRAV14-1 | TRAV14-1*01 |
| | | TRAV14-1*02 |
| TRAV15 | TRAV15 | TRAV15*01 |
| TRAV16 | TRAV16 | TRAV16*01 |
| TRAV17 | TRAV17 | TRAV17*01 |
| TRAV18 | TRAV18 | TRAV18*01 |
| TRAV19 | TRAV19 | TRAV19*01 |
| TRAV20 | TRAV20 | TRAV20*01 |
| | | TRAV20*02 |
| | | TRAV20*03 |
| | | TRAV20*04 |
| TRAV21 | TRAV21 | TRAV21*01 |
| | | TRAV21*02 |
| TRAV22 | TRAV22 | TRAV22*01 |
| TRAV23 | TRAV23/DV6 | TRAV23/DV6*01 |
| | | TRAV23/DV6*02 |
| | | TRAV23/DV6*03 |
| | | TRAV23/DV6*04 |
| | | TRAV23/DV6*05 |
| TRAV24 | TRAV24 | TRAV24*01 |
| | | TRAV24*02 |
| TRAV25 | TRAV25 | TRAV25*01 |
| TRAV26 | TRAV26-1 | TRAV26-1*01 |
| | | TRAV26-1*02 |
| | | TRAV26-1*03 |
| | TRAV26-2 | TRAV26-2*01 |
| | | TRAV26-2*02 |
| TRAV27 | TRAV27 | TRAV27*01 |
| | | TRAV27*02 |
| | | TRAV27*03 |
| TRAV28 | TRAV28 | TRAV28*01 |
| | | TRAV28*02 |
| TRAV29 | TRAV29/DV5 | TRAV29/DV5*01 |
| | | TRAV29/DV5*02 |
| | | TRAV29/DV5*03 |
| | | TRAV29/DV5*04 |
| TRAV30 | TRAV30 | TRAV30*01 |
| | | TRAV30*02 |
| | | TRAV30*03 |
| | | TRAV30*04 |
| | | TRAV30*05 |
| TRAV31 | TRAV31 | TRAV31*01 |
| | | TRAV31*02 |
| TRAV32 | TRAV32 | TRAV32*01 |
| TRAV33 | TRAV33 | TRAV33*01 |
| | | TRAV33*02 |
| TRAV34 | TRAV34 | TRAV34*01 |
| TRAV35 | TRAV35 | TRAV35*01 |
| | | TRAV35*02 |
| | | TRAV35*03 |
| TRAV36 | TRAV36/DV7 | TRAV36/DV7*01 |
| | | TRAV36/DV7*02 |
| | | TRAV36/DV7*03 |
| | | TRAV36/DV7*04 |
| | | TRAV36/DV7*05 |
| TRAV37 | TRAV37 | TRAV37*01 |
| TRAV38 | TRAV38-1 | TRAV38-1*01 |
| | | TRAV38-1*02 |
| | | TRAV38-1*03 |
| | | TRAV38-1*04 |
| | TRAV38-2/DV8 | TRAV38-2/DV8*01 |
| TRAV39 | TRAV39 | TRAV39*01 |
| TRAV40 | TRAV40 | TRAV40*01 |
| TRAV41 | TRAV41 | TRAV41*01 |
| TRAV46 | TRAV46 | TRAV46*01 |
| TRAVA | TRAVA | TRAVA*01 |
| | | TRAVA*02 |
| TRAVB | TRAVB | TRAVB*01 |
| | | TRAVB*02 |
| TRAVC | TRAVC | TRAVC*01 |

In some embodiments, isolation of mKRAS-specific TCRs can be obtained from a subject vaccinated with a pooled mutant KRAS long peptide vaccine. In certain embodiments, T cells are isolated from a subject and cultured ex vivo with KRAS peptides or mutants (mKRAS) thereof. In certain embodiments, the mutant KRAS epitopes comprise G12V, G12D, G12C, G12R, G12A, G13D or combinations thereof. In certain embodiments, the KRAS peptides or mutants (mKRAS) thereof, comprise one or more modified amino acids, unnatural amino acids, substituted amino acids or combinations thereof. Accordingly, the KRAS peptides or mutants (mKRAS) thereof, further comprise one or more modified amino acids, unnatural amino acids, substituted amino acids or combinations thereof. Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a β-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof, a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

In certain embodiments, a T cell receptor (TCR) comprises a TCRα chain variable domain and a TCRβ chain variable domain having complementary determining regions (CDRs) which specifically bind to mutant KRAS epitopes. In certain embodiments, the TCRα chain variable domain comprises a CDR1α, a CDR2α, and a CDR3α. In certain embodiments, the TCRβ chain variable domain comprises a CDR1β, a CDR2α, and a CDR3β. In certain embodiments, the complementary determining region of the TCRα chain, CDR3α, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the complementary determining region of the TCRα chain, CDR3α, comprises an amino acid sequence of any one of SEQ ID NOs: 315-629, 632, or 634. In certain embodiments, the complementary determining region of the TCRβ chain, CDR3β, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the complementary determining region of the TCRβ chain, CDR3β, comprises an amino acid sequence having a sequence identity of at least 75%, at least 80%, at least 85%, at least 90%, at least 95%, at least 99%, or 100% to SEQ ID Nos: 1-314, or 630, or 631 or 633. In certain embodiments, the complementary determining region of the TCRβ chain, CDR3β, comprises an amino acid sequence of any one of SEQ ID Nos: 1-314, or 630, or 631 or 633. In certain embodiments, the TCR is soluble. In certain embodiments, the TCR is single-stranded. In certain embodiments, the TCR is formed by linking an a chain variable domain and a p chain variable domain through a peptide linking sequence. In certain embodiments, the TCR comprises (a) all or part of the TCRα chain except a transmembrane domain; and (b) all or part of the TCRβ chain except a transmembrane domain. In certain embodiments, the TCR comprises (a) all or part of the TCRα chain and a transmembrane domain; and (b) all or part of the TCRβ chain and a transmembrane domain. In some embodiments, a TCR disclosed herein comprises a V gene CDR1 or CDR2. In some embodiments, a TCR disclosed herein comprises a C*01 or C*02 constant region.

In some embodiments, the CDR1a comprises TRAV17, TRAV10, TRAV8-3, TRAV13-1, TRAV3, TRAV29/DV5, TRAV27, TRAV2, TRAV5, TRAV9-2, TRAV21, TRAV23/DV6, TRAV12-3, TRAV8-6, TRAV1-2, TRAV26-2, TRAV22, TRAV8-4, TRAV26-1, TRAV14/DV4, TRAV12-1, TRAV16, TRAV13-2, TRAV30, TRAV12-2, TRAV35, or TRAV38-2/DV8.

In some embodiments, the CDR2a comprises CDR2a of TRAV17, TRAV10, TRAV8-3, TRAV13-1, TRAV3, TRAV29/DV5, TRAV27, TRAV2, TRAV5, TRAV9-2, TRAV21, TRAV23/DV6, TRAV12-3, TRAV8-6, TRAV1-2, TRAV26-2, TRAV22, TRAV8-4, TRAV26-1, TRAV14/DV4, TRAV12-1, TRAV16, TRAV13-2, TRAV30, TRAV12-2, TRAV35, or TRAV38-2/DV8.

In some embodiments, the CDR1(3 comprises CDR1(3 of TRBV7-9, TRBV2, TRBV12-3, TRBV5-6, TRBV11-3, TRBV5-1, TRBV8, TRBV18, TRBV27, TRBV28, TRBV7-9, TRBV9, TRBV20-1, TRBV6-4, TRBV6-5, TRBV6-2, TRBV19, TRBV5-5, TRBV29-1, TRBV28, TRBV11-2, TRBV9, TRBV6-6, TRBV15, TRBV12-4, or TRBV3-1.

In some embodiments, the CDR20 comprises CDR20 of TRBV7-9, TRBV2, TRBV12-3, TRBV5-6, TRBV11-3, TRBV5-1, TRBV8, TRBV18, TRBV27, TRBV28, TRBV7-9, TRBV9, TRBV20-1, TRBV6-4, TRBV6-5, TRBV6-2, TRBV19, TRBV5-5, TRBV29-1, TRBV28, TRBV11-2, TRBV9, TRBV6-6, TRBV15, TRBV12-4, or TRBV3-1.

In some embodiments, the TRAC comprises TRAC*01. In some embodiments, the TRBC comprises TRBC1 or TRBC2. In some embodiments, the TRBC1 comprises TRBC1*01. In some embodiments, the TRBC2 comprises TRBC2*01

In certain embodiments, an isolated cell comprises an expression vector encoding a T cell receptor (TCR). In some embodiments, the TCR comprises a TCRα chain variable domain and a TCRβ chain variable domain having complementary determining regions (CDRs) which specifically bind to mutant KRAS epitopes. In certain embodiments, the vector comprises adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus or vesicular stomatitis virus. In certain embodiments, the expression vector further comprises an inducible promoter, a tissue specific promoter or a constitutive promoter. In certain embodiments, the expression vector further comprises one or more enhancer or regulatory sequences. In certain embodiments, the expression vector further comprises an inducible suicide gene. In certain embodiments, the expression vector further comprises a nucleic acid sequence encoding for one or more cytokines.

In another aspect, an isolated nucleic acid encodes a T cell receptor (TCR) comprising an antigen binding domain which specifically binds a tumor antigen. In certain embodiments, the tumor antigen is a Kirsten rat sarcoma viral (KRAS) tumor antigen. In certain embodiments, the TCR comprises a TCRα chain variable domain and a TCRβ chain variable domain having complementary determining regions (CDRs) which specifically bind to mutant KRAS epitopes. In certain embodiments, the complementary determining region of the TCRα chain, CDR3α, comprises a nucleic acid sequence encoding an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 315-629, 632, or 634. In certain embodiments, the complementary determining region of the TCRα chain, CDR3α, comprises a nucleic acid sequence encoding an amino acid sequence of any one of SEQ ID NOs: 315-629, 632, or 634. In certain embodiments, the complementary determining region of the TCRα chain, CDR3α, comprises a nucleic acid sequence encoding an amino acid sequence having a sequence identity of at least 75% to any one of SEQ ID NOs: 315-629, 632, or 634. In certain embodiments, the complementary determining region of the TCRα chain, CDR3α, comprises a nucleic acid sequence encoding an amino acid sequence of any one of SEQ ID NOs: 315-629, 632, or 634. In certain embodiments, the complementary determining region of the TCRβ chain, CDR3β, comprises a nucleic acid sequence encoding an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the complementary determining region of the TCRβ chain, CDR3β, comprises a nucleic acid sequence encoding an amino acid sequence having a sequence identity of at least 75% to any one of SEQ ID NOs: 1-314, or 630, or 631 or 633. In certain embodiments, the complementary determining region of the TCRβ chain, CDR3β, comprises a nucleic acid sequence encoding an amino acid sequence to any one of SEQ ID NOs: 1-314, or 630, or 631 or 633. In some embodiments, a TCR disclosed herein comprises a V gene CDR1 or CDR2. In some embodiments, a TCR disclosed herein comprises a C*01 or C*02 constant region.

The isolated cells can be transduced by a vector encoding the TCRs embodied herein. In certain embodiments, the vector comprises adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus or vesicular stomatitis virus. In certain embodiments, the vector further comprises an inducible promoter, a cell specific promoter, a tissue specific promoter or a constitutive promoter. In certain embodiments, the vector further comprises one or more enhancer or regulatory sequences. In certain embodiments, the vector further comprises an inducible suicide gene. In certain embodiments, the vector further comprises a nucleic acid sequence encoding for one or more cytokines.

In certain embodiments, the TCRs comprise a CD3α sequence comprising 60%, 70%, 75%, 80%, 85%, 86%, 87%, 88%, 89%, 90%, 91%, 92%, 93%, 94%, 95%, 96%, 97%, 98%, or 99%, sequence identity to any one of SEQ ID NOs: 315-629, 632, or 634. In certain embodiments, a CD3α sequence comprises at least a 75%, 80%, 85%, 86%, 87%, 88%, 89%, 90%, 91%, 92%, 93%, 94%, 95%, 96%, 97%, 98%, or 99%, sequence identity to any one of SEQ ID NOs: 315-629, 632, or 634.In certain embodiments a CD3α sequence comprises any one of SEQ ID NOs: 315-629, 632, or 634.

In certain embodiments, the TCRs comprise a CD3β sequence comprising 60%, 70%, 75%, 80%, 85%, 86%, 87%, 88%, 89%, 90%, 91%, 92%, 93%, 94%, 95%, 96%, 97%, 98%, or 99%, sequence identity to any one of SEQ ID NOs: 1-314, or 630, or 631 or 633. In certain embodiments, a CD3β sequence comprises at least a 75%, 80%, 85%, 86%, 87%, 88%, 89%, 90%, 91%, 92%, 93%, 94%, 95%, 96%, 97%, 98%, or 99%, sequence identity to any one of SEQ ID NOs: 1-314, or 630, or 631 or 633. In certain embodiments a CD3α sequence comprises any one of SEQ ID NOs: 1-314, or 630, or 631 or 633.

In certain embodiments, a TCR comprises a CD3α sequence comprising 60%, 70%, 75%, 80%, 85%, 86%, 87%, 88%, 89%, 90%, 91%, 92%, 93%, 94%, 95%, 96%, 97%, 98%, or 99%, sequence identity to any one of SEQ ID NOs: 315-629, 632, or 634 and a CD3β sequence comprising 60%, 70%, 75%, 80%, 85%, 86%, 87%, 88%, 89%, 90%, 91%, 92%, 93%, 94%, 95%, 96%, 97%, 98%, or 99%, sequence identity to any one of SEQ ID NOs: 1-314, or 630, or 631 or 633. In certain embodiments, a TCR comprises a CD3α sequence comprising any one of SEQ ID NOs: 315-629, 632, or 634 and a CD3β sequence comprising any one of SEQ ID NOs: 1-314, or 630, or 631 or 633.

### Genome Editing

Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as reviewed in Cox et al., Nature Medicine 21(2), 121-31 (2015). These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015). In some instances, it may be possible to predict likely repair outcomes based on analysis of potential microhomologies at the site of the DNA break.

Each of these genome editing mechanisms can be used to create desired genomic alterations. A step in the genome editing process can be to create one or two DNA breaks, the latter as double-strand breaks or as two single-stranded breaks, in the target locus as near the site of intended mutation. This can be achieved via the use of site-directed polypeptides, as described and illustrated herein.

Accordingly, in certain aspects, a method of redirecting T cell specificity in vitro or in vivo, comprises contacting isolated cells obtained from a biological sample from a subject with a gene editing agent comprising a Clustered Regularly Interspaced Short Palindromic Repeat (CRISPR)-associated endonuclease or a nucleic acid sequence encoding the CRISPR-associated endonuclease; and, at least one guide nucleic acid or a nucleic acid sequence encoding the guide nucleic acid, the guide nucleic acid being complementary to a target nucleic acid sequence within or near a T cell receptor α chain (TCRα) sequence and/or TCRβ chain sequence for knocking out an endogenous T cell receptor and introducing a TCR specific for a tumor antigen into the isolated cells; thereby redirecting the T cell specificity. In certain embodiments, the gene editing agent is introduced into a cell by a vector or by homology-directed repair (HDR). In certain embodiments, the CRISPR/Cas comprises a Cas9, Cas3 or Cas 12a endonuclease. In certain embodiments, the CRISPR/Cas is CRISPR/Cas12a. In certain embodiments, the CRISPR/Cas12a introduces the recombinant TCRα and TCRβ chain sequence at the endogenous T cell receptor α and β constant (TRAC/TRBC) locus of healthy donor T cells. In certain embodiments, the CRISPR-Cas system, is introduced in single and multiplex configurations. In certain embodiments, the CRISPR/Cas editing composition comprises a plurality of guide RNAs (gRNAs).

### CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. In some embodiments, there are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g., hairpins) and/or comprise unstructured single-stranded sequences. It some embodiments, The repeats usually occur in clusters and frequently diverge between species. In some embodiments, the repeats are regularly interspaced with unique intervening sequences referred to as “spacers,” resulting in a repeat-spacer-repeat locus architecture. In some embodiments, the spacers are identical to or have high homology with known foreign invader sequences. In some embodiments, a spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). In some embodiments, a spacer sequence is located at the 5′ or 3′ end of the crRNA.

In some embodiments, the CRISPR locus also comprises polynucleotide sequences encoding CRISPR Associated (Cas) genes. In some embodiments, Cas genes encode endonucleases involved in the biogenesis and the interference stages of crRNA function in prokaryotes. Some Cas genes comprise homologous secondary and/or tertiary structures.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8al, Cas8a2, Cas8b, Cas8c, Cas9, Casio, CaslOd, CasF, CasG, CasH, CasX, CasΦ, Csyl, Csy2, Csy3, Csel (or CasA), Cse2 (or CasB), Cse3 (or CasE), Cse4 (or CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csxl0, Csxl6, CsaX, Csx3, Cszl, Csxl5, Csfl, Csf2, Csf3, Csf4, and Cui 966. By way of further example, in some embodiments, the CRISPR-Cas protein is a Casl, Cas1B, Cas2, Cas3, Cas4, Cas5, Cash, Cas7, Cas8, Casio, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, Csxl6, CsaX, Csx3, Csxl, Csxl5, Csfl, Csf2, Csf3, Csf4, Cas9, Cas12 (e.g., Cas12a, Cas12b, Cas12c, Cas12d, Cas12k, Cas12j/CasΦ, Cas12L etc.), Cas 13 (e.g., Cas13a, Cas13b (such as Cas13b-tl, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas 12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is Cas(.

In some embodiments, the composition comprises a CRISPR-associated (Cas) protein, or functional fragment or derivative thereof. In some embodiments, the Cas protein is an endonuclease, including but not limited to the Cas9, Cas3 or Cas12a nuclease. In some embodiments, the Cas protein comprises the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. In some embodiments, the Cas protein comprises a modified amino acid sequence, as compared to its natural source. In some embodiments, CRISPR/Cas proteins comprise at least one RNA recognition and/or RNA binding domain. In some embodiments, RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein. The disclosed CRISPR-Cas compositions should also be construed to include any form of a protein having substantial homology to a Cas protein (e.g., Cas12 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein.

### Type II CRISPR Systems:

In some embodiments, crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a site-directed polypeptide (e.g., Cas9, Cas12a). The crRNA of the crRNA-tracrRNA-Cas complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM can be essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096): 816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

### Type V CRISPR Systems:

Type V CRISPR systems have several important differences from Type II systems. For example, Cas12a (formerly Cas12a) is a single RNA-guided endonuclease that, in contrast to Type II systems, lacks tracrRNA. In fact, Cas12a-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also, Cas12a can utilize a T-rich protospacer-adjacent motif such that Cas12a-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cas12a cleaves DNA via a staggered DNA double-stranded break with a 4 or 5 nucleotide 5′ overhang. Type II systems cleave via a blunt double-stranded break. Similar to Type II systems, Cas12a contains a predicted RuvC-like endonuclease domain, but lacks a second HNH endonuclease domain, which is in contrast to Type II systems.

Unlike Cas9, Cas12a has intrinsic RNase activity that allows processing of its own crRNA array, enabling multigene editing from a single RNA transcript. This established characteristic of Cas12a makes it suited to multiplex editing. (Gier, R. A., et al. High-performance CRISPR-Cas12a genome editing for combinatorial genetic screening. Nat Commun 11, 3455 (2020). doi.org/10.1038/s41467-020-17209-1).

### Cas Genes/Polypeptides and Protospacer Adjacent Motifs:

Exemplary CRISPR/Cas polypeptides include the Cas9 polypeptides as published in Fonfara et al., Nucleic Acids Research, 42: 2577-2590 (2014). The CRISPR/Cas gene naming system has undergone extensive rewriting since the Cas genes were discovered.

The disclosure should also be construed to include any form of a peptide having substantial homology to a Cas peptide (e.g., Cas12a) disclosed herein. Preferably, a peptide which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a Cas peptide disclosed herein.

The peptide may alternatively be made by recombinant means or by cleavage from a longer polypeptide. The composition of a peptide may be confirmed by amino acid analysis or sequencing. The variants of the peptides according to the present disclosure may be (i) one in which one or more of the amino acid residues are substituted with a conserved or nonconserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). In some embodiments, the fragments include peptides generated via proteolytic cleavage (including multi-site proteolysis) of an original sequence. In some embodiments, variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants can include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. The peptides of the disclosure may include unnatural amino acids formed by post-translational modification or by introducing unnatural amino acids during translation. A variety of approaches are available for introducing unnatural amino acids during protein translation.

A peptide or protein of the disclosure may be conjugated with other molecules, such as proteins, to prepare fusion proteins. This may be accomplished, for example, by the synthesis of N-terminal or C-terminal fusion proteins provided that the resulting fusion protein retains the functionality of the Cas peptide.

### Editing Strategy

Provided herein are cellular, ex vivo and in vivo methods for using genome engineering tools to create permanent changes to the genome. In certain aspects these changes may include: (i) knocking out the endogenous receptor and replacing it with a TCR or interest or by editing the endogenous TCR to reprogram the T cell to specifically recognize certain tumor antigens, e.g. mutant KRAS.

In the first editing strategy, a wild-type or corrected TCR, a cDNA or a minigene (comprised of one or more exons and optionally one or more introns, including natural or synthetic introns) can be inserted into a locus of the endogenous T cell receptor α and β constant (TRAC/TRBC) locus of healthy donor T cells. In certain aspects, this can be achieved by delivering into the cell one or more CRISPR endonucleases, one or more gRNAs (e.g., crRNA+tracrRNA, or sgRNA) targeting upstream, downstream, or within an intron or exon of the TCR gene and a donor DNA that contains the desired sequence and homology arms to the flanking regions of the target locus. In certain aspects, this can be achieved by delivering into the cell one or more CRISPR endonucleases, one or more gRNAs (e.g., crRNA+tracrRNA, or sgRNA) targeting T cell receptor α and β constant (TRAC/TRBC) locus and a donor DNA that contains the desired sequence and homology arms to the flanking regions of the target locus. The donor DNA can be single or double stranded DNA.

In the second and third editing strategies, wild-type TCR sequences can be replaced by inducing one single stranded break or double stranded break in the TCR gene with one or more CRISPR endonucleases and a gRNA (e.g., crRNA+tracrRNA, or sgRNA), or two or more single stranded breaks or double stranded breaks in the TCR a and R genes, e.g. TRAC/TRBC loci with one or more CRISPR endonucleases and two or more gRNAs, in the presence of a donor DNA template introduced exogenously to direct the cellular DSB response to Homology-Directed Repair (the donor DNA template can be a short single stranded oligonucleotide, a short double stranded oligonucleotide, a long single or double stranded DNA molecule). This strategy can be used to replace or correct one or more mutations in one or more exons, introns, intron:exon junctions, or other DNA sequences encoding regulatory elements of the TCR a and R genes, e.g., TRAC/TRBC loci or combination thereof.

In addition to the editing options listed above, Cas9, Cas12 or similar proteins can be used to target effector domains to the same target sites that can be identified for editing, or additional target sites within range of the effector domain. A range of chromatin modifying enzymes, methylases, or demethylases can be used to alter expression of the target gene.

A number of types of genomic target sites can be present. The regulation of transcription and translation implicates a number of different classes of sites that interact with cellular proteins or nucleotides. Often the DNA binding sites of transcription factors or other proteins can be targeted for mutation or deletion to study the role of the site, though they can also be targeted to change gene expression. Sites can be added through non-homologous end joining NHEJ or direct genome editing by homology directed repair (HDR). Increased use of genome sequencing, RNA expression and genome-wide studies of transcription factor binding have increased the ability to identify how the sites lead to developmental or temporal gene regulation. These control systems can be direct or can involve extensive cooperative regulation that can require the integration of activities from multiple enhancers. In some embodiments, transcription factors can bind 6-12 bp-long degenerate DNA sequences. In some embodiments, binding sites with less degeneracy can provide simpler means of regulation. Artificial transcription factors can be designed to specify longer sequences that have less similar sequences in the genome and have lower potential for off-target cleavage. Any of these types of binding sites can be mutated, deleted or even created to enable changes in gene regulation or expression (Canver, M. C. et al., Nature (2015)).

### Site-Directed Polypeptides (Endonucleases, Enzymes):

A site-directed polypeptide is can be a nuclease used in genome editing to cleave DNA. The site-directed polypeptide can be administered to a cell or a patient as either: one or more polypeptides, or one or more mRNAs encoding the polypeptide. Single molecule guide RNA can be pre-complexed with a site-directed polypeptide. The site-directed polypeptide can be any of the DNA endonuclease disclosed herein.

In the context of a CRISPR/Cas9 or CRISPR/Cas12a system, the site-directed polypeptide can bind to a guide RNA that, in turn, specifies the site in the target DNA to which the polypeptide is directed. In the CRISPR/Cas9 or CRISPR/Cas12a systems disclosed herein, the site-directed polypeptide can be an endonuclease, such as a DNA endonuclease.

A site-directed polypeptide can comprise a plurality of nucleic acid-cleaving (i.e., nuclease) domains. Two or more nucleic acid-cleaving domains can be linked together via a linker. For example, the linker can comprise a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Site-directed polypeptides can introduce double-strand breaks or single-strand breaks in nucleic acids, e.g., genomic DNA. The double-strand break can stimulate a cell's endogenous DNA-repair pathways (e.g., homology-dependent repair (HDR) or NHEJ or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. HDR can occur when a homologous repair template, or donor, is available. The homologous donor template can comprise sequences that are homologous to sequences flanking the target nucleic acid cleavage site. The sister chromatid can be used by the cell as the repair template. In some embodiments, the repair template can be supplied as an exogenous nucleic acid, such as a plasmid, duplex oligonucleotide, single-strand oligonucleotide or viral nucleic acid. With exogenous donor templates, an additional nucleic acid sequence (such as a transgene) or modification (such as a single or multiple base change or a deletion) can be introduced between the flanking regions of homology so that the additional or altered nucleic acid sequence also becomes incorporated into the target locus. In some embodiments, MMEJ can result in a genetic outcome that is similar to NHEJ in that small deletions and insertions can occur at the cleavage site. MMEJ can make use of homologous sequences of a few base pairs flanking the cleavage site to drive a favored end-joining DNA repair outcome. In some instances, it may be possible to predict likely repair outcomes based on analysis of potential microhomologies in the nuclease target regions.

Thus, in some cases, homologous recombination can be used to insert an exogenous polynucleotide sequence into the target nucleic acid cleavage site. An exogenous polynucleotide sequence is termed a “donor polynucleotide” (or donor or donor sequence) herein. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

The site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed and various other site-directed polypeptides. The site-directed polypeptide can comprise at least 70, 75, 80, 85, 90, 95, 97, 99, or 100% identity to a wild-type site-directed polypeptide (e.g., Cas12a) over 10 contiguous amino acids.

In some embodiments, the site-directed polypeptide can comprise at most: 70, 75, 80, 85, 90, 95, 97, 99, or 100% identity to a wild-type site-directed polypeptide (e.g., Cas12a) over 10 contiguous amino acids. The site-directed polypeptide can comprise at least: 70, 75, 80, 85, 90, 95, 97, 99, or 100% identity to a wild-type site-directed polypeptide over 10 contiguous amino acids in a HNH nuclease domain of the site-directed polypeptide. The site-directed polypeptide can comprise at most: 70, 75, 80, 85, 90, 95, 97, 99, or 100% identity to a wild-type site-directed polypeptide over 10 contiguous amino acids in a HNH nuclease domain of the site-directed polypeptide. The site-directed polypeptide can comprise at least: 70, 75, 80, 85, 90, 95, 97, 99, or 100% identity to a wild-type site-directed polypeptide over 10 contiguous amino acids in a RuvC nuclease domain of the site-directed polypeptide. The site-directed polypeptide can comprise at most: 70, 75, 80, 85, 90, 95, 97, 99, or 100% identity to a wild-type site-directed polypeptide over 10 contiguous amino acids in a RuvC nuclease domain or HNH domain of the site-directed polypeptide.

In some embodiments, the site-directed polypeptide can comprise a modified form of a wild-type exemplary site-directed polypeptide. The modified form of the wild-type exemplary site-directed polypeptide can comprise a mutation that reduces the nucleic acid-cleaving activity of the site-directed polypeptide. The modified form of the wild-type exemplary site-directed polypeptide can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide. The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

The modified form of the site-directed polypeptide can comprise a mutation such that it can induce a single-strand break (SSB) on a target nucleic acid (e.g., by cutting only one of the sugar-phosphate backbones of a double-strand target nucleic acid). In some aspects, the mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide. In some aspects, the mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid.

In some embodiments, nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it may be necessary for a pair of nickases to bind in close proximity and on opposite strands of the target nucleic acid, thereby creating a pair of nicks, which is the equivalent of a double-strand break. This may require that two separate guide RNAs—one for each nickase—bind in close proximity and on opposite strands of the target nucleic acid. This doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes.

Mutations contemplated can include substitutions, additions, and deletions, or any combination thereof. In some embodiments, the mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). In some embodiments, the mutation converts the mutated amino acid to a non-natural amino acid (e.g., selenomethionine). In some embodiments, the mutation converts the mutated amino acid to amino acid mimics (e.g., phosphomimics). In some embodiments, the mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). In some embodiments, the mutation can cause a shift in reading frame and/or the creation of a premature stop codon. In some embodiments, mutations can cause changes to regulatory regions of genes or loci that affect expression of one or more genes.

The site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target DNA. The site-directed polypeptide (e.g., variant, mutated, enzymatically inactive and/or conditionally enzymatically inactive endoribonuclease) can target RNA. The site-directed polypeptide can comprise one or more non-native sequences (e.g., the site-directed polypeptide is a fusion protein).

### Genome-Targeting Nucleic Acids:

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. In some embodiments, a guide RNA can comprise at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In some embodiments, in the Type V guide RNA (gRNA), the crRNA forms a duplex. In some embodiments, in both systems, the duplex can bind a site-directed polypeptide, such that the guide RNA and site-direct polypeptide form a complex. The genome-targeting nucleic acid can provide target specificity to the complex by virtue of its association with the site-directed polypeptide. In some embodiments, the genome-targeting nucleic acid thus can direct the activity of the site-directed polypeptide. The genome-targeting nucleic acid can be a double-molecule guide RNA. The genome-targeting nucleic acid can be a single-molecule guide RNA.

In some embodiments, a double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

In some embodiments, a single-molecule guide RNA (sgRNA) in a Type II system can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

The sgRNA can comprise a 20 nucleotide spacer sequence at the 5′ end of the sgRNA sequence. The sgRNA can comprise a less than a 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence. The sgRNA can be unmodified or modified. For example, modified sgRNAs can comprise one or more 2′-O-methyl phosphorothioate nucleotides.

In some embodiments, the composition comprises at least one isolated guide nucleic acid, or fragment thereof, where the guide nucleic acid comprises a nucleotide sequence that is complementary to one or more target sequences in the T cell receptor α and β constant (TRAC/TRBC) locus. In some embodiments, the guide nucleic acid is a guide RNA (gRNA).

In some embodiments, the gRNA comprises a crRNA:tracrRNA duplex. In some embodiments, the gRNA comprises a stem-loop that mimics the natural duplex between the crRNA and tracrRNA. In some embodiments, the stem-loop comprises a nucleotide sequence comprising AGAAAU. For example, in some embodiments, the composition comprises a synthetic or chimeric guide RNA comprising a crRNA, stem, and tracrRNA.

In certain embodiments, the composition disclosed herein comprises an isolated crRNA and/or an isolated tracrRNA which hybridize to form a natural duplex. For example, in some embodiments, the gRNA comprises a crRNA or crRNA precursor (pre-crRNA) comprising a targeting sequence. In some embodiments, the gRNA comprises a nucleotide sequence that is substantially complementary to a target sequence in a TCR gene. The target sequence in the genome may be any sequence in any coding or non-coding region where CRISPR/Cas-mediated gene editing would result in the editing of the genome. In certain embodiments, the target sequence, to which the gRNA is substantially complementary, is the T cell receptor α and β constant (TRAC/TRBC) locus.

Further, the disclosure encompasses an isolated nucleic acid (e.g., gRNA) having substantial homology to a nucleic acid disclosed herein. In certain embodiments, the isolated nucleic acid has at least 75%, 80%, 85%, 90%, 95%, 96%, 97%, 98%, or 99% sequence homology with a nucleotide sequence of a gRNA described herein.

In some embodiments, the guide RNA sequence can be a sense or anti-sense sequence. In some embodiments, in the CRISPR-Cas system derived from S. pyogenes, the target DNA immediately precedes a 5′-NGG proto-spacer adjacent motif (PAM). Other Cas9 orthologs may have different PAM specificities. For example, Cas9 from S. thermophilus requires 5′-NNAGAA for CRISPR 1 and 5′-NGGNG for CRISPR3) and Neisseria meningiditis requires 5′-NNNNGATT). The specific sequence of the guide RNA may vary, but, regardless of the sequence, useful guide RNA sequences will be those that minimize off-target effects while achieving high efficiency editing of the TCR target sequence(s). The specific sequence of the guide RNA may vary, but, regardless of the sequence, useful guide RNA sequences will be those that minimize off-target effects while achieving high efficiency editing of the TCR genome. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome, include bioinformatic screening using target sequence+NGG target selection criteria to exclude off-target human transcriptome or (even rarely) untranslated-genomic sites, and WGS, Sanger sequencing and SURVEYOR assay, to identify and exclude potential off-target effects. Algorithms, such as CRISPR Design Tool (CRISPR Genome Engineering Resources; Broad Institute) can be used to identify target sequences with or near requisite PAM sequences as defined by the type of Cas peptide (i.e. Cas9, Cas9 variant, Cas12a) used.

In certain embodiments, the composition disclosed herein comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In certain embodiments, the RNA (e.g., crRNA, tracrRNA, gRNA) may be engineered to comprise one or more modified nucleobases. For example, known modifications of RNA can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxy carbonylmethyluridine; 5-methoxycarbonylmethyl-2′-O-methyluridine; 5-methoxy carbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N⁶, N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1; 2′-O-dimethyl inosine; 2′-O-methyl guanosine; 1-methyl guanosine; N²-methyl guanosine; N²,N²-dimethyl guanosine; N², 2′-O-dimethyl guanosine; N², N², 2′-O-trimethyl guanosine; 2′-O-ribosyl guanosine (phosphate); 7-methyl guanosine; N²; 7-dimethyl guanosine; N²; N²; 7-trimethyl guanosine; wyosine; methylwyosine; undermodified hydroxywybutosine; wybutosine; hydroxywybutosine; peroxywybutosine; queuosine; epoxy queuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The methods of the present disclosure or others in the art can be used to identify additional modified RNA. In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises Sulphur (S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region (e.g, the TCR genes) and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence. In some embodiments, gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable doublestranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Drechsler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid (e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion crRNA, tracrRNA, RNA-encoding DNA, or of a Cas12a-encoding DNA.

In certain embodiments, the isolated RNAs are synthesized from an expression vector encoding the RNA molecule, as described in detail elsewhere herein.

### Nucleic Acids Encoding System Components

The present disclosure also provides a nucleic acid comprising a nucleotide sequence encoding a genome-targeting nucleic acid of the disclosure, a site-directed polypeptide of the disclosure, and/or any nucleic acid or proteinaceous molecule necessary to carry out the aspects of the methods of the disclosure.

The nucleic acid encoding a genome-targeting nucleic acid of the disclosure, a site-directed polypeptide of the disclosure, and/or any nucleic acid or proteinaceous molecule necessary to carry out the aspects of the methods of the disclosure can comprise a vector (e.g., a recombinant expression vector).

The term “vector” refers to a nucleic acid molecule capable of transporting another nucleic acid to which it has been linked. One type of vector is a “plasmid”, which refers to a circular double-stranded DNA loop into which additional nucleic acid segments can be ligated.

Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operatively linked. Such vectors are referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas such as RNA polymerase III promoters, including for example U6 and H1, can be advantageous.

Descriptions of and parameters for enhancing the use of such promoters are known in art, and additional information and approaches are regularly being described; see, e.g., Ma, H. et al., Molecular Therapy—Nucleic Acids 3, e161 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter, etc.).

The nucleic acid encoding a genome-targeting nucleic acid of the disclosure and/or a site-directed polypeptide can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### Ex Vivo Based Therapy

Another aspect of such method is an ex vivo cell-based therapy. For example, a T cells cell can be isolated from the patient's peripheral blood or bone marrow. Then, the chromosomal DNA of these cells can be edited using the materials and methods described herein. Finally, the cells are implanted into the patient. Any source or type of cell may be used as the progenitor cell.

One advantage of an ex vivo cell therapy approach is the ability to conduct a comprehensive analysis of the therapeutic prior to administration. Nuclease-based therapeutics can have some level of off-target effects. Performing gene editing ex vivo allows one to characterize the cell population prior to implantation. The present disclosure includes sequencing the entire genome of the corrected cells to ensure that the off-target effects, if any, can be in genomic locations associated with minimal risk to the patient. Furthermore, populations of specific cells, including clonal populations, can be isolated prior to implantation.

Another advantage of ex vivo cell therapy relates to genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability. Thus, manipulation of iPSCs for the treatment of cancer can be much easier and can shorten the amount of time needed to make the desired genetic correction.

### In Vivo Based Therapy

Methods can also include an in vivo based therapy. Chromosomal DNA of the cells in the patient is edited using the materials and methods described herein.

Although certain cells present an attractive target for ex vivo treatment and therapy, increased efficacy in delivery may permit direct in vivo delivery to such cells. Ideally the targeting and editing would be directed to the relevant cells. Cleavage in other cells can also be prevented by the use of promoters only active in certain cells and or developmental stages. Additional promoters are inducible, and therefore can be temporally controlled if the nuclease is delivered as a plasmid. The amount of time that delivered RNA and protein remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy will have the potential to be used to treat more than one patient, for example a number of patients who share the same or similar genotype or allele. In contrast, ex vivo cell therapy typically requires using a patient's own cells, which are isolated, manipulated and returned to the same patient.

### Genome Engineering Strategies

A step of the ex vivo methods of the present disclosure can comprise editing/correcting a T cell isolated from the patient using genome engineering. For example, if there are small or large deletions or multiple mutations, a wild-type gene, a cDNA or a minigene (comprised of one or more exons and introns or natural or synthetic introns) can be knocked into the gene locus or a heterologous location in the genome. Pairs of nucleases can be used to delete gene regions, and a donor is provided to restore function. In this case two gRNAs and one donor sequence would be supplied. A full length cDNA can be knocked into any locus, but must use a supplied or other promoter. If this construct is knocked into the correct location, it will have physiological control, similar to the normal gene.

Alternatively, in some embodiments, the donor for correction by HDR contains the corrected sequence with small or large flanking homology arms to allow for annealing. HDR is essentially an error-free mechanism that uses a supplied homologous DNA sequence as a template during DSB repair. The rate of homology directed repair (HDR) is a function of the distance between the targeted region and the cut site so choosing overlapping or nearest target sites is important. Templates can include extra sequences flanked by the homologous regions or can contain a sequence that differs from the genomic sequence, thus allowing sequence editing.

Homology directed repair is a cellular mechanism for repairing double-stranded breaks (DSBs). The most common form is homologous recombination. There are additional pathways for HDR, including single-strand annealing and alternative-HDR. Genome engineering tools allow researchers to manipulate the cellular homologous recombination pathways to create site-specific modifications to the genome. It has been found that cells can repair a double-stranded break using a synthetic donor molecule provided in trans. Therefore, by introducing a double-stranded break near a specific mutation and providing a suitable donor, targeted changes can be made in the genome. Specific cleavage increases the rate of HDR more than 1,000 fold above the rate of 1 in 10⁶ cells receiving a homologous donor alone. The rate of homology directed repair (HDR) at a particular nucleotide is a function of the distance to the cut site, so choosing overlapping or nearest target sites is important. Gene editing offers the advantage over gene addition, as correcting in situ leaves the rest of the genome unperturbed.

Supplied donors for editing by HDR vary markedly but can contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors have been used. These oligonucleotides range in size from less than 100 nt to over many kb, though longer ssDNA can also be generated and used. Double-stranded donors can be used, including PCR amplicons, plasmids, and mini-circles. In general, it has been found that an AAV vector can be a very effective means of delivery of a donor template, though the packaging limits for individual donors is <5 kb. Active transcription of the donor can increase HDR three-fold, indicating the inclusion of promoter may increase conversion. Conversely, CpG methylation of the donor decreased gene expression and HDR.

In addition to wild-type endonucleases, such as Cas9, nickase variants exist that have one or the other nuclease domain inactivated resulting in cutting of only one DNA strand. HDR can be directed from individual Cas nickases or using pairs of nickases that flank the target area. Donors can be single-stranded, nicked, or dsDNA.

The donor DNA can be supplied with the nuclease or independently by a variety of different methods, for example by transfection, nano-particle, micro-injection, or viral transduction. A range of tethering options has been proposed to increase the availability of the donors for HDR. Examples include attaching the donor to the nuclease, attaching to DNA binding proteins that bind nearby, or attaching to proteins that are involved in DNA end binding or repair.

The repair pathway choice can be guided by a number of culture conditions, such as those that influence cell cycling, or by targeting of DNA repair and associated proteins. For example, to increase HDR, key NHEJ molecules can be suppressed, such as KU70, KU80 or DNA ligase IV.

Without a donor present, the ends from a DNA break or ends from different breaks can be joined using the several non-homologous repair pathways in which the DNA ends are joined with little or no base-pairing at the junction. In addition to canonical NHEJ, there are similar repair mechanisms, such as alt-NHEJ. If there are two breaks, the intervening segment can be deleted or inverted. NHEJ repair pathways can lead to insertions, deletions or mutations at the joints.

In addition to genome editing by NHEJ or HDR, site-specific gene insertions have been conducted that use both the NHEJ pathway and HDR. A combination approach may be applicable in certain settings, possibly including intron/exon borders. NHEJ may prove effective for ligation in the intron, while the error-free HDR may be better suited in the coding region.

In some embodiments, the methods can provide gRNA pairs that make a deletion by cutting the gene twice, one gRNA cutting at the 5′ end of one or more mutations and the other gRNA cutting at the 3′ end of one or more nucleic acid bases that facilitates insertion of a new sequence from a polynucleotide donor template to replace the one or more mutations. The cutting can be accomplished by a pair of DNA endonucleases that each makes a DSB in the genome, or by multiple nickases that together make a DSB in the genome.

Alternatively, the methods can provide one gRNA to make one double-strand cut around one or more mutations that facilitates insertion of a new sequence from a polynucleotide donor template to replace the one or more mutations. The double-strand cut can be made by a single DNA endonuclease or multiple nickases that together make a DSB in the genome.

Numerous variations of the replacements including without limitation larger as well as smaller deletions, are contemplated. Such variations can include replacements that are larger in the 5′ and/or 3′ direction than the specific nucleic acid in question, or smaller in either direction.

Accordingly, by “near” or “proximal” with respect to specific replacements, it is intended that the SSB or DSB locus associated with a desired replacement boundary (also referred to herein as an endpoint) can be within a region that is less than about 3 kb from the reference locus noted. The SSB or DSB locus can be more proximal and within 2 kb, within 1 kb, within 0.5 kb, or within 0.1 kb. In the case of small replacement, the desired endpoint can be at or “adjacent to” the reference locus, by which it is intended that the endpoint can be within 100 bp, within 50 bp, within 25 bp, or less than about 10 bp to 5 bp from the reference locus.

In order to ensure that the pre-mRNA is properly processed following deletion, the surrounding splicing signals can be deleted. Splicing donor and acceptors are generally within 100 base pairs of the neighboring intron. In some examples, methods can provide all gRNAs that cut approximately ±100-3100 bp with respect to each exon/intron junction of interest.

For any of the genome editing strategies, gene editing can be confirmed by sequencing or PCR analysis.

### Pharmaceutical Compositions

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. In some embodiments, the liposomes can be targeted to and taken up selectively by the organ. The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

Liquid suspensions may be prepared using conventional methods to achieve suspension the composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Methods of Treatment

The present disclosure provides a method of treating or preventing cancer. In some embodiments, the method comprises administering to a subject in need thereof, an effective amount of a composition comprising a KRAS peptide or mutants thereof. In certain embodiments, mutant KRAS peptides comprise epitopes comprising G12V, G12D, G12C, G12R, G12A, G13D or combinations thereof. In certain embodiments, T cells are isolated from a subject and cultured ex vivo with KRAS peptides or mutants (mKRAS) thereof. In certain embodiments, the mutant KRAS epitopes comprise G12V, G12D, G12C, G12R, G12A, G13D or combinations thereof. In certain embodiments, the KRAS peptides or mutants (mKRAS) thereof, comprise one or more modified amino acids, unnatural amino acids, substituted amino acids or combinations thereof. Accordingly, the KRAS peptides or mutants (mKRAS) thereof, further comprise one or more modified amino acids, unnatural amino acids, substituted amino acids or combinations thereof. Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAco-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

In certain embodiments, the KRAS and/or mKRAS peptides are administered in combination with an adjuvant. As used herein, an “adjuvant” refers to a substance that enhances the body's immune response to an antigen or a vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers comprise nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC™ brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In a preferred embodiment the adjuvant is at a concentration of about 0.01 to about 50%, at a concentration of about 2% to 30%, at a concentration of about 5% to about 25%, at a concentration of about 7% to about 22%, and at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

In certain embodiments, a method of treating a subject requiring immunotherapy such as for example, cancer, comprises isolating T lymphocytes and/or NK cells from a biological sample obtained from the subject; transducing the T lymphocytes and/or NK cells with an expression vector encoding a T cell receptor or chimeric antigen receptor (CAR) which specifically binds to tumor antigens, e.g., KRAS and mutants thereof, stimulating the transduced T lymphocytes and/or NK cells with KRAS and mutants thereof at least once ex vivo to obtain cells specific for the KRAS and mutants thereof; and reinfusing the cells into the subject, thereby treating the subject. In certain embodiments, the cells are autologous cells. The T cells may be obtained from any suitable source of T cells known in the art including, but not limited to, T cells collected from a subject. The collected cells may be expanded ex vivo using methods commonly known in the art before transduction with a T cell receptor embodied herein.

In some embodiments, the CAR comprises an antigen specific binding domain, a transmembrane domain, a costimulatory domain, and an intracellular signaling domain.

In some embodiments, the intracellular signaling domain comprises at least a portion of an intracellular signaling domain from FcRγ, FcRβ, CD3γ, CD3δ, CD3ε, CD3ζ, CD22, CD79a, CD79b, or CD66d.

In some embodiments, the costimulatory domain comprises at least a portion of CD28, 4-1BB, OX-40, HVEM, BTLA, B7-H3, ICOS, GITR, CD80, CD86, TNFR, CD40L, CD27, or TLR.

In some embodiments, the transmembrane domain comprises or can be derived from at least a portion of a transmembrane domain from a TCRα chain, TCRβ chain, TCRγ chain, TCRδ chain, CD3ζ subunit, CD3ε subunit, CD3γ subunit, or CD3δ subunit.

In some embodiments, a method of treating cancer in a subject diagnosed with cancer comprises isolating cells from a biological sample subject; culturing the isolated cells with one or more tumor antigens, isolating T cells and/or NK cells cultured with the one or more tumor antigens and expanding the T cells and/or NK cells to produce a therapeutically effective composition of tumor antigen specific T cells and NK cells; adoptively transferring the tumor antigen specific T cells and NK cells into the subject, thereby treating the subject diagnosed with cancer. In certain embodiments, the isolated cells comprise autologous cells. In certain embodiments, the isolated cells comprise allogeneic cells. In certain embodiments, the T cells comprise a CD8⁺ T lymphocyte, a CD4⁺ T lymphocyte, a γδ T cell, a regulatory T cell (Treg), a tumor infiltrating T lymphocyte (TIL) and combinations thereof. In certain embodiments, the tumor antigen is a Kirsten rat sarcoma viral (KRAS) tumor antigen. In certain embodiments, the KRAS tumor antigen comprises one or more mutations.

In another aspect, isolated cells are transformed with an expression vector encoding the T cell receptors (TCRs) or the chimeric antigen receptors (CARs) embodied herein. In certain embodiments, the vector comprises adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus or vesicular stomatitis virus. In certain embodiments, the expression vector further comprises an inducible promoter, a cell specific promoter, a tissue specific promoter or a constitutive promoter. In certain embodiments, the expression vector further comprises one or more enhancer or regulatory sequences. In certain embodiments, the expression vector further comprises an inducible suicide gene. In certain embodiments, the vector further comprises a nucleic acid sequence encoding for one or more cytokines. In certain embodiments the isolated cells comprise autologous, allogeneic, haplotype matched, haplotype mismatched, haplo-identical, xenogeneic, cell lines or combinations thereof. In certain embodiments, the isolated cells comprise autologous cells. The isolated cells expressing T cell receptors (TCRs) or the chimeric antigen receptors (CARs) embodied herein, are expanded ex vivo to produce a therapeutically effective composition of tumor antigen specific T cells and NK cells; adoptively transferring the tumor antigen specific T cells and NK cells into the subject, thereby treating the subject diagnosed with cancer. In certain embodiments, the T cells comprise a CD8⁺ T lymphocyte, a CD4⁺ T lymphocyte, a γδ T cell, a regulatory T cell (Treg), a tumor infiltrating T lymphocyte (TIL) and combinations thereof.

Methods for CAR design, delivery and expression in T cells, and the manufacturing of clinical-grade CAR-T cell populations are known in the art. See, for example, Lee et al., Clin. Cancer Res. 2012, 18(10): 2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. The CAR-T cells, once they have been expanded ex vivo in response to, for example, a tumor antigen, can be reinfused into the subject in a therapeutically effective amount. The term “therapeutically effective amount” as used herein means the amount of CAR-T or CAR-NK cells when administered to a mammal, in particular a human, in need of such treatment, is sufficient to treat cancer. In certain embodiments, administration of any of the compositions embodied herein, can be combined with other cell-based therapies, for example, stem cells, antigen presenting cells, etc.

The compositions of the present disclosure may be prepared in a manner known in the art and in a manner suitable for parenteral administration to mammals, particularly humans, comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the transduced T cells expressing a T cell receptor embodied herein, CAR-T cells and/or CAR-NK cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

In other embodiments, the compositions comprise a cell which has been transformed or transfected with one or more vectors or nucleic acids encoding one or more T cell receptors embodied herein. In some embodiments, the methods of the disclosure can be applied ex vivo. That is, a subject's cells can be removed from the body and transduced with the compositions in culture with a desired target antigen, expand target-antigen specific, e.g. T cells and the expanded cells returned to the subject's body. The cells can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The T cell receptors can be easily delivered to a subject by methods known in the art, for example, methods which deliver siRNA. Thus, the T cell receptor molecules can be used clinically, similar to the approaches taken by current gene therapy. In particular, a T cell receptor stable expression stem cell or iPS cells for cell transplantation therapy as well as vaccination can be developed for use in subjects.

The transduced cells, once they have been expanded ex vivo in response to a tumor antigen, are reinfused into the subject in a therapeutically effective amount. The term “therapeutically effective amount” as used herein means the amount of T cells encoding the T cell receptors embodied herein, when administered to a mammal, in particular a human, in need of such treatment, is sufficient to treat the disease.

The precise amount of transduced T cells to be administered can be determined by a physician with consideration of individual differences in age, weight, extent of disease and condition of the subject. Typically, administration of T cell therapies is defined by number of cells per kilogram of body weight. However, because T cells will replicate and expand after transfer, the administered cell dose will not resemble the final steady-state number of cells.

In an embodiment, a pharmaceutical composition comprising the T cells encoding the T cell receptors embodied herein, may be administered at a dosage of 10⁴ to 10⁹ cells/kg body weight. In another embodiment, a pharmaceutical composition comprising the T cells encoding the T cell receptors embodied herein, may be administered at a dosage of 10⁵ to 10⁶ cells/kg body weight, including all integer values within those ranges.

Compositions comprising the T cells encoding the T cell receptors embodied herein, may also be administered multiple times at these dosages. The cells can be administered by using infusion techniques that are known in the art (see, for example, Rosenberg et al., 1988, New England Journal of Medicine, 319: 1676). The optimal dosage and treatment regimen for a particular subject can be readily determined by one skilled in the art by monitoring the patient for signs of disease and adjusting the treatment accordingly.

### Combination Therapies

The disclosure also contemplates the combination of the compositions of the present disclosure with other drugs and/or in addition to other treatment regimens or modalities such as surgery. When the compositions of the present disclosure are used in combination with known therapeutic agents, the combination may be administered either in sequence (either continuously or broken up by periods of no treatment) or concurrently or as an admixture. For example, in the case of cancer chemotherapeutic agents may be administered as part of the combination therapy.

In certain embodiments, the T cells encoding the T cell receptors embodied herein, are administered in conjunction with one or more cytokines, e.g., IL-2.

In certain embodiments, the T cells encoding the T cell receptors embodied herein, are administered in conjunction with a cancer therapy. As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™ Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin 71 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

In various embodiments, the cancer therapeutic is an immunotherapy selected from the group comprising oncolytic virus, bacteria, oncolytic bacteria or other bacterial compositions, Bacillus Calmette-Guerin (BCG), a microbiome modulator, and/or a toll-like receptor (TLR) agonist. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, plants, bacteria and/or made synthetically. In various embodiments, the immunotherapy is a is a stimulator of interferon genes (STING) pathway modulator.

It will be appreciated by those skilled in the art of cancer immunotherapy that other complementary immune therapies may be added to the regimens described above to further enhance their efficacy including but not limited to GM-CSF to increase the number of myeloid derived innate immune system cells, low dose cyclophosphamide or PI3K inhibitors (e.g., PI3K delta inhibitors) to eliminate T regulatory cells that inhibit innate and adaptive immunity and 5FU (e.g., capecitabine), PI3K inhibitors or histone deacetylase inhibitors to remove inhibitory myeloid derived suppressor cells. For example, PI3K inhibitors include, but are not limited to, LY294002, Perifosine, BKM120, Duvelisib, PX-866, BAY 80-6946, BEZ235, SF1126, GDC-0941, XL147, XL765, Palomid 529, GSK1059615, PWT33597, IC87114, TG100-15, CAL263, PI-103, GNE-477, CUDC-907, and AEZS-136. In some aspects, the PI3K inhibitor is a PI3K delta inhibitor such as, but not limited to, Idelalisib, RP6530, TGR1202, and RP6503. Additional PI3K inhibitors are disclosed in U.S. Patent Application Nos. US20150291595, US20110190319, and International Patent Application Nos. WO2012146667, WO2014164942, WO2012062748, and WO2015082376. The immunotherapy may also comprise the administration of an interleukin such as IL-2, or an interferon such as INFα.

In certain embodiments, the CAR-T cells and/or CAR-NK cells are administered with one or more immune checkpoint modulators. Immune checkpoints refer to inhibitory pathways of the immune system that are responsible for maintaining self-tolerance and modulating the duration and amplitude of physiological immune responses. Examples of checkpoint inhibitor include, without limitation an inhibitor of. PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

The term “checkpoint inhibitor” means a group of molecules on the cell surface of CD4⁺ and/or CD8⁺ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPα (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

In some embodiments, such therapy involves blockade of programmed cell death 1 (PD-1). In some embodiments, such therapy involves treatment with an agent that interferes with an interaction involving PD-1 (e.g., with PD-L1). In some embodiments, such therapy involves administration of an antibody agent that specifically interacts with PD-1 or with PD-L1. In some embodiments, such therapy involves administration of one or more of nivolumab (BMS-936558, MDX-1106, ONO-4538, a fully human Immunoglobulin G4 (IgG4) monoclonal PD-1 antibody), pembrolizumab (MK-3475, a humanized monoclonal IgG4 anti-PD-1 antibody), BMS-936559 (a fully human IgG4 PD-L1 antibody), MPDL3280A (a humanized engineered IgG1 monoclonal PD-L1 antibody) and/or MEDI4736 (a humanized engineered IgG1 monoclonal PD-L1 antibody).

### Kits

Kits are also contemplated herein. In one aspect, a kit comprises a T cell receptor (TCR) wherein the TCR comprises a TCRα chain variable domain and a TCRβ chain variable domain having complementary determining regions (CDRs) which specifically bind to mutant KRAS epitopes. In certain embodiments, the complementary determining region of the TCRα chain, CDR3α, comprises an amino acid sequence having a sequence identity of at least 50%, at least 60%, at least 70%, at least 75%, at least 80%, at least 85%, at least 90%, at least 95%, at least 99%, or 100% amino acid sequence identity any one of SEQ ID NOs: 315-629, 632, or 634. In certain embodiments, the complementary determining region of the TCRα chain, CDR3α, comprises an amino acid sequence of any one of SEQ ID NOs: 315-629, 632, or 634. In certain embodiments, the complementary determining region of the TCRβ chain, CDR3β, comprises an amino acid sequence having a sequence identity of at least 50%, at least 60%, at least 70%, at least 75%, at least 80%, at least 85%, at least 90%, at least 95%, at least 99%, or 100% amino acid sequence identity to any one of SEQ ID NOs: 1-314, or 630, or 631 or 633. In certain embodiments, the complementary determining region of the TCRβ chain, CDR3β, comprises an amino acid sequence having a sequence identity of at least 75% to any one of SEQ ID NOs: 1-314, or 630, or 631 or 633. In certain embodiments, the complementary determining region of the TCRβ chain, CDR3β, comprises an amino acid sequence to any one of SEQ ID NOs: 1-314, or 630, or 631 or 633. In certain embodiments, the TCR is soluble. In certain embodiments, the TCR is single-stranded. In certain embodiments, the TCR is formed by linking an a chain variable domain and a p chain variable domain through a peptide linking sequence. In certain embodiments, the TCR comprises (a) all or part of the TCRα chain except a transmembrane domain; and (b) all or part of the TCRβ chain except a transmembrane domain. In certain embodiments, the TCR comprises (a) all or part of the TCRα chain and a transmembrane domain; and (b) all or part of the TCRβ chain and a transmembrane domain. In some embodiments, a T cell receptor disclosed herein comprises a CD3α chain and a CD3β chain comprising a sequence disclosed in Table 1 or Table 2. In some embodiments, a T cell receptor disclosed herein comprises a CD3α chain sequence disclosed in Table 1 and a CD3β chain comprising a sequence disclosed Table 2. In some embodiments, a T cell receptor disclosed herein comprises a CD3α chain sequence disclosed in Table 2 and a CD3β chain comprising a sequence disclosed Table 1.

In some embodiments, the kit can further comprise at least one reagent for use in one or more embodiments of the methods described herein. Reagents that can be provided in the kit can include at least one or more of the following: a hybridization reagent, a purification reagent, an immobilization reagent, an imaging agent, a cell permeabilization agent, a blocking agent, a cleaving agent for the cleavable linker, and any combinations thereof.

In some embodiments, the kit can further include a computer-readable (non transitory) storage medium in accordance with one or more embodiments described herein. For example, in one embodiment, the computer-readable (non-transitory) storage medium included in the kit can provide instructions to determine the presence or expression levels of one or more target molecules in a sample. The computer-readable (non-transitory) storage medium can be in a CD, DVD, and/or USB drive.

In some embodiments of the aspect, the kit includes the necessary packaging materials and informational material therein to store and use said kits. The informational material can be descriptive, instructional, marketing or other material that relates to the methods described herein and/or the use of an agent(s) described herein for the methods described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In embodiments of the aspects described herein, the kit can be provided with its various elements included in one package, e.g., a fiber-based, e.g., a cardboard, or polymeric, e.g., a styrofoam box. The enclosure can be configured so as to maintain a temperature differential between the interior and the exterior, e.g., it can provide insulating properties to keep the reagents at a preselected temperature for a preselected time. The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit usage forms of target probes described herein. For example, the kit can include a plurality of syringes, ampules, foil packets, or blister packs, each containing a single unit usage of target probes described herein. The containers of the kits can be airtight, waterproof (e.g., impermeable to changes in moisture or evaporation), and/or lighttight.

In some aspects, embodiments disclosed herein is contemplated as being applicable to each of the other disclosed embodiments. Thus, all combinations of the various elements described herein are within the scope of the disclosure.

This disclosure is further illustrated by the following examples which should not be construed as limiting. The contents of all references, patents, and published patent applications cited throughout this application, as well as the figures and the sequence listing, are hereby incorporated by reference.

### EXAMPLES

### Example 1: Isolation of Mutant KRAS-Specific TCRs from Vaccinated Patients

mKRAS-specific TCRs have been isolated and validated. These TCRs have been obtained from the peripheral blood of patients vaccinated with a pooled mutant KRAS long peptide vaccine that the inventors have developed. From the first round of experiments, a novel TCR that recognizes a KRAS G12V epitope was isolated and validated from a vaccinated patient.

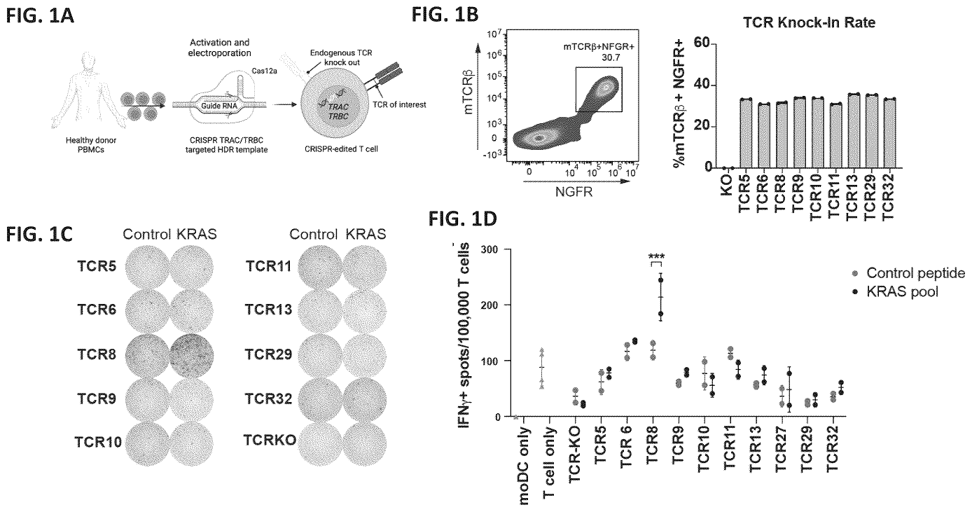

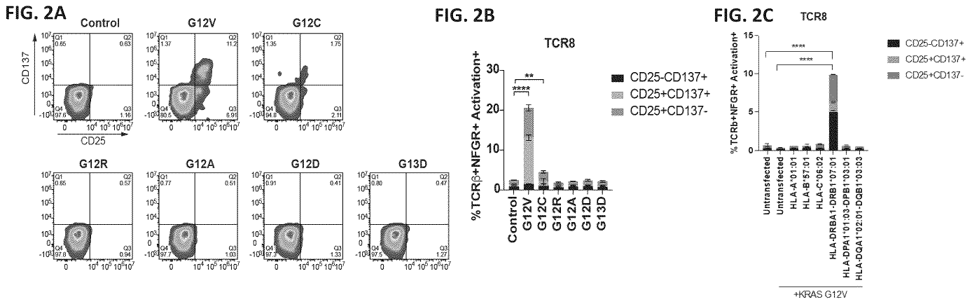

Single-cell RNA/TCR sequencing from pre- and post-vaccination PBMCs was performed and several potential mKRAS-specific TCRs were identified for further validation. CRISPR-Cas12a-based genome editing was used to introduce a recombinant TCRα and TCRβ chain sequence at the endogenous T cell receptor α and β constant (TRAC/TRBC) locus of healthy donor T cells (FIG. 1A). Number of recombinant TCR knock-in into healthy donor T cells was quantified by measuring murine TCRβ+ and NGFR+ T-cells (FIG. 1B). T cell co-cultures were performed with autologous monocyte-derived dendritic cells (moDCs) pulsed with the pooled mutant KRAS peptides. Several TCRs showed a minor, but not significant, increase in reactivities against peptide pools by IFNγ ELISPOT (FIG. 1C, FIG. 1D). However, TCR8 showed a significant increase in IFNγ production compared to co-culture with moDCs pulsed with the control peptide (FIG. 1D). Further co-culture of TCR8 with CD3− antigen-presenting cells isolated from PBMCs and pulsed individually with each mutant KRAS peptide resulted in significant activation and upregulation of CD137 and CD25 after recognition of the KRAS G12V peptide (FIG. 2A, FIG. 2B).

A weaker, but significant response over APCs pulsed with control peptide was observed against the KRAS G12C peptide. Furthermore, HLA restriction to patient's HLA allele was confirmed by nucleofecting HLA null K562s with each HLA allele from this patient which were then pulsed with KRAS G12V SLP and co-cultured with TCR8 expressing cells. These data demonstrated that TCR8 recognized KRAS G12V in the context of HLA-DRB1*07:01 (FIG. 2C). The result demonstrates the cross-reactive potential of KRAS-reactive TCRs.

TABLE 1

| TCR | | | | | | | |
|---|---|---|---|---|---|---|---|
| ID | CDR3A | TRAV | TRAJ | CDR3B | TRBV | TRBJ | Specificity |
| TCR8 | CAASR | TRAV13- | TRAJ11 | CSAG | TRBV20- | TRBJ1- | KRAS |
| | GGYST | 1*01 | | GDSP | 1*01 | 2 | G12V |
| | LTF | | | GGYT | | | |
| | (SEQ | | | F | | | |
| | ID | | | (SEQ | | | |
| | NO: | | | ID | | | |
| | 629) | | | NO: | | | |
| | | | | 630) | | | |

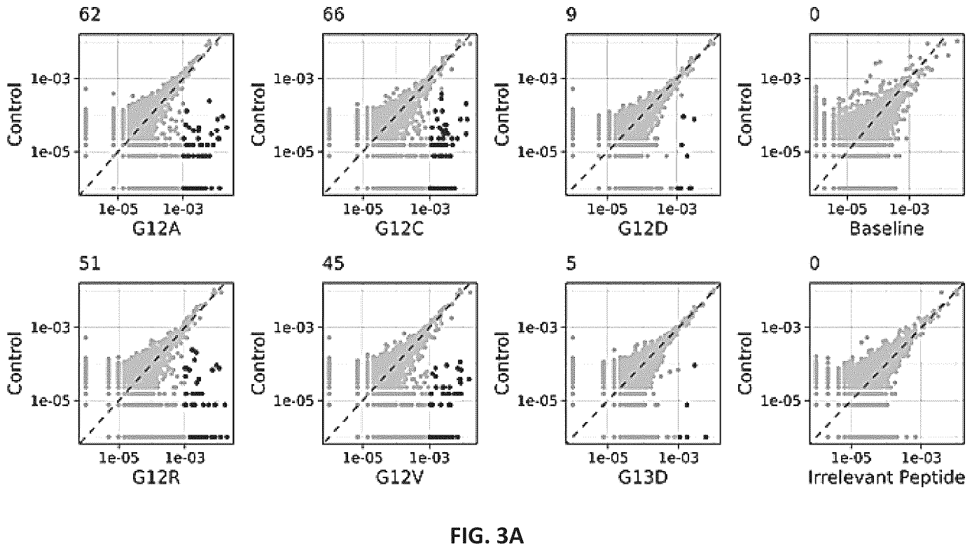

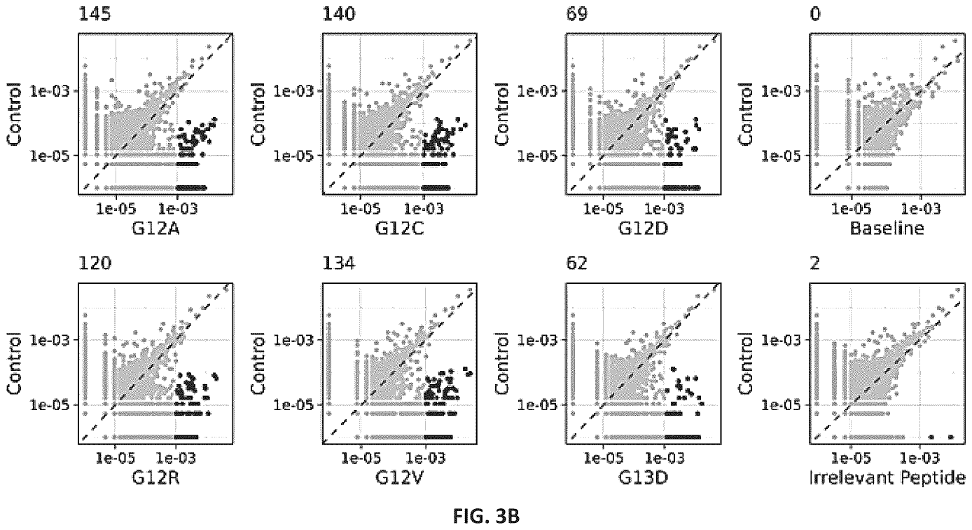

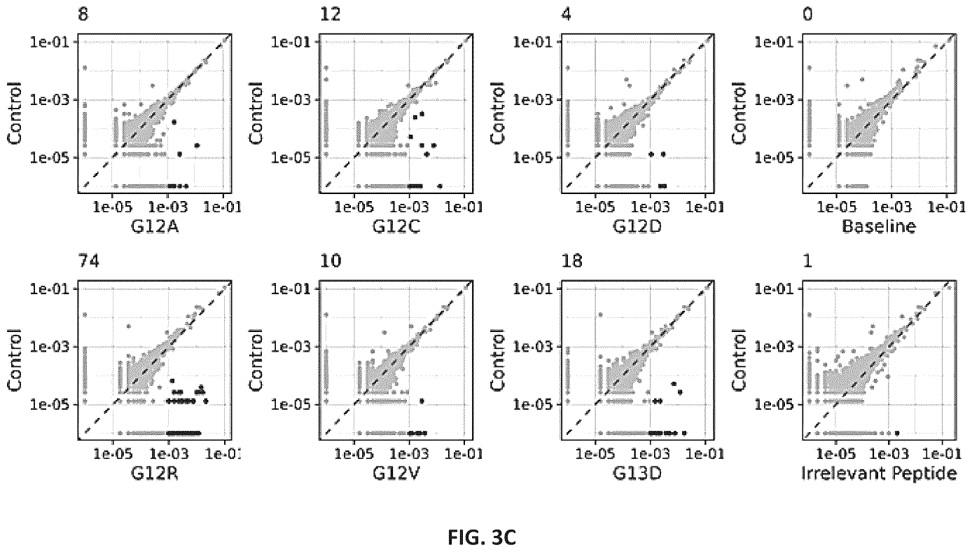

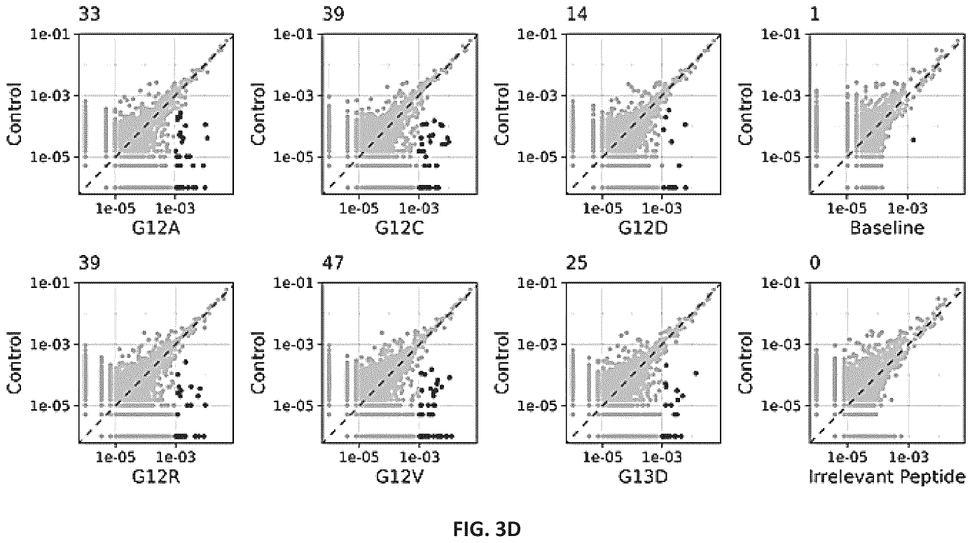

Further TCRs have been isolated and validated. The identification of a library of mKRAS-specific T cells by in vitro peptide expansion and TCRβ sequencing for 4 patients vaccinated with the pooled mutant KRAS peptide vaccine (FIGS. 1A-1D). 1×10⁶ PMBCs from post-vaccine timepoints of patient (FIG. 3A) J1994_12, (FIG. 3B) J1994_5, (FIG. 3C) J1994_2 or (FIG. 3D) J1994_6, were seeded in a 48 well plate and incubated with 2 μg/mL KRAS G12V, G12A, G12R, G12C, G12D, G13D, or an irrelevant synthetic long peptide and 100IU/mL hIL-2 for 7 days. Genomic DNA was isolated from peptide expanded PBMCs and sent for TCRβ sequencing. Genomic DNA for baseline timepoint was collected immediately after thawing PBMCs. Frequency of TCRs in the control condition is shown relative to the frequency of each TCR in the peptide expansion conditions or baseline sample. Differential abundance analysis was performed to identify TCRs that expanded greater than 10-fold compared to the control sample and was present at least 0.1% of the repertoire. Significantly enriched TCRs are shown in black and number of significantly enriched is shown above each condition plot (FIGS. 3A-3D).

Paired alpha-beta TCRs were identified, and phenotypic characterization of mKRAS-specific T cells was conducted by mapping peptide-expanded TCRβ chains onto single cell RNA/TCR sequencing of unstimulated PBMCs for each patient (FIGS. 2A, 2B, Table 2). mKRAS-reactivity of 4 mKRAS-specific TCRs were identified in peptide expansion assays (FIG. 2C).

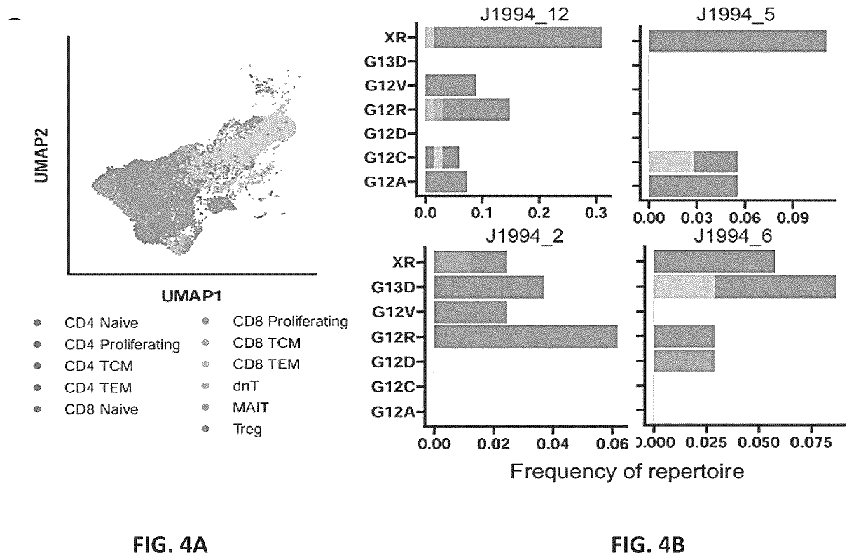

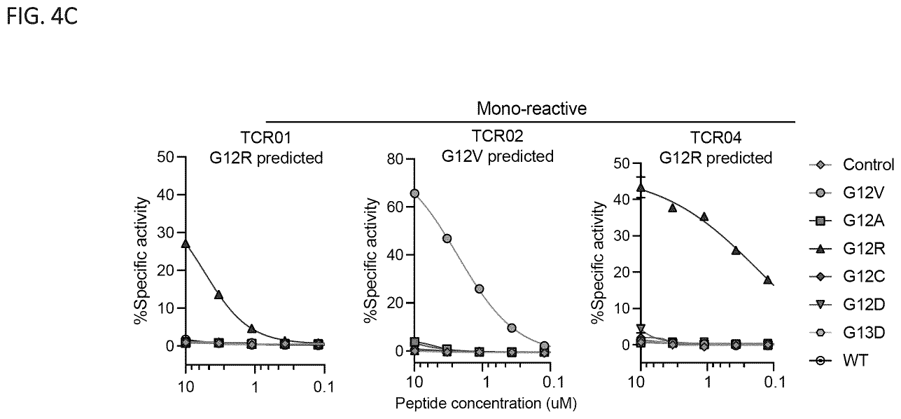

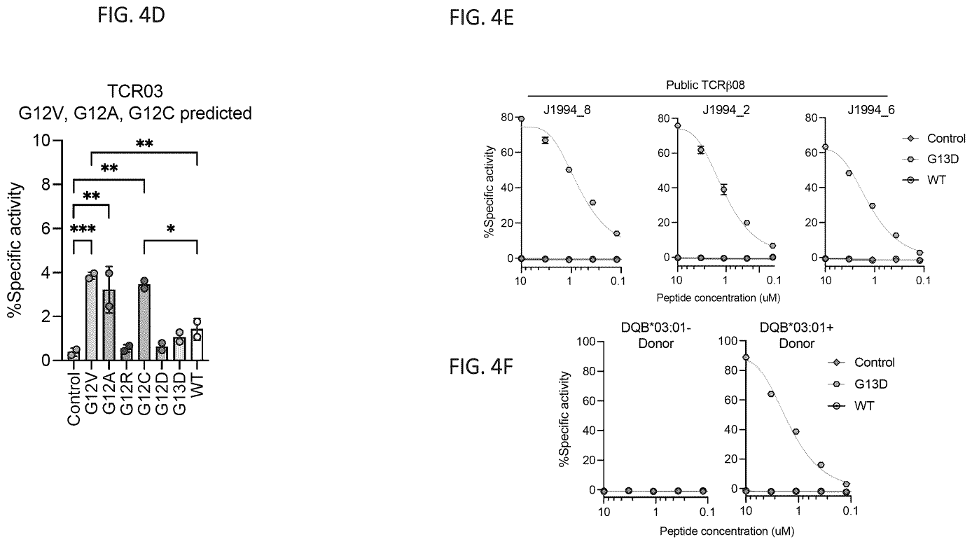

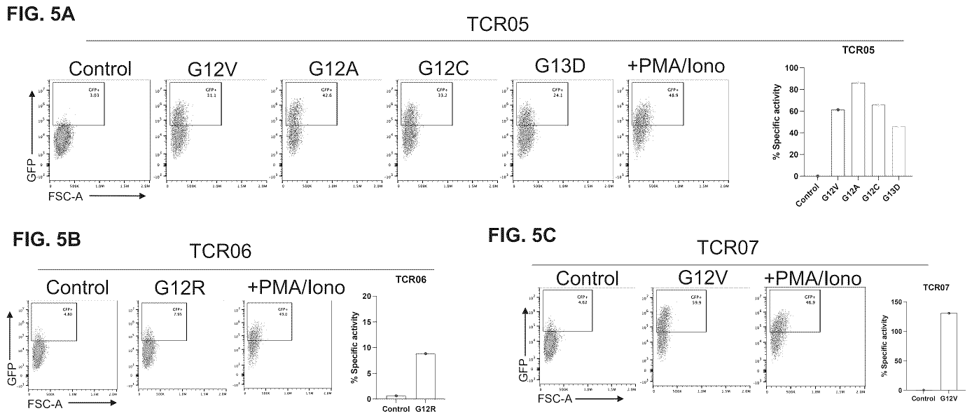

Single cell RNA/TCR sequencing was performed on pre- and post-vaccine PBMCs for patients J1994_12, J1994_5, J1994_1, and J1994_6. T cells were identified and phenotyped into CD4 (naïve, proliferating, central memory (TCM), and effector memory (TEM)), CD8 (naïve, proliferating, TCM, and TEM), double negative T (dnT), MAIT, and Tregs (FIG. 4A). Phenotype of T cells with TCRβ chains from each mKRAS expansion conditions or that were found in more than one expansion (XR) (FIG. 4B). Jurkat-TCRKO-NFAT-GFP cells were transduced to express a putative mKRAS-specific TCR (FIG. 4C). TCR transduced cells were co-cultured with peptide-pulsed, patient-matched LCLs at the indicated peptide concentrations, overnight, 37° C. Cells were analyzed for activation by GFP expression and normalized to specific activity of minimal and maximal activation conditions. The TCR transduced cells showed specific activity to their respective mKRAS epitope and no cross-reactivity with other epitopes (FIG. 4C). The cross reactive TCR03 transduced T cell was validated to respond to KRAS G12V, G12A, and G12C as predicted (FIG. 4D). Jurkat reporter line transduced to express Public TCRβ08 co-cultured with patient matched LCLs pulsed with control, KRAS G13D, or wild type peptide at the indicated concentrations (FIG. 4E). Public TCRβ08 Jurkats co-cultured with LCLs from a HLA-DQB*03:01-donor (left) or irrelevant HLA-DQB*03:01+ donor (right) pulsed with control, KRAS G13D, or wild type KRAS peptide (FIG. 4F). Cells were analyzed for activation by GFP expression and normalized to specific activity of minimal (unpulsed LCL only) and maximal (PMA+Ionomycin) activation conditions. FIG. 5A-FIG. 5C shows validation of KRAS-specific CD8 T cell reactivity. Jurkat-TCR_{KO}-NFAT-GFP cells were transduced to express putative mono-reactive, mKRAS-specific TCRs and co-cultured with peptide-pulsed, patient-matched LCLs at 10 uM per peptide. Cells were analyzed for activation by GFP expression and normalized to specific activity of minimal (unpulsed LCL only) and maximal (PMA+Ionomycin) activation conditions (FIG. 5A—TCR05, FIG. 5B—TCR06, FIG. 5C—TCR07).

TABLE 2

| TCR | | CDR3. | | | CDR3. | | | |
|---|---|---|---|---|---|---|---|---|
| ID | TRBV | beta.aa | TRBJ | TRAV | alpha.aa | TRAJ | Antigen | Phenotype |
| | TRBV28 | CAIAG | TRBJ1-2 | TRAV38-1 | CAFIL | TRAJ43 | G12D | NA |
| | | PGQGA | | | NNNDM | | | |
| | | RGYTF | | | RF | | | |
| | | (SEQ ID | | | (SEQ ID | | | |
| | | NO: 1) | | | NO: 315) | | | |
| | TRBV10-3 | CAISE | TRBJ1-1 | TRAV19 | CALSE | TRAJ36 | G12V; | CD8 |
| | | GSPEA | | | AVGGA | | G13D | TCM |
| | | FF | | | NNLFF | | | |
| | | (SEQ ID | | | (SEQ ID | | | |
| | | NO: 2) | | | NO: 316) | | | |
| | TRBV4-1 | CASGL | TRBJ1-4 | TRAV38-1 | CAFMK | TRAJ42 | G12R | CD4 |
| | | RLNEK | | | QSGGS | | | TCM |
| | | LFF | | | QGNLI | | | |
| | | (SEQ ID | | | F | | | |
| | | NO: 3) | | | (SEQ ID | | | |
| | | | | | NO: 317) | | | |
| | TRBV7-9 | CASGL | TRBJ2-2 | TRAV21 | CAVRS | TRAJ28 | G12R | NA |
| | | VDYEL | | | YSGAG | | | |
| | | FF | | | SYQLT | | | |
| | | (SEQ ID | | | F | | | |
| | | NO: 4) | | | (SEQ ID | | | |
| | | | | | NO: 318) | | | |
| | TRBV6-1 | CASIH | TRBJ1-5 | TRAV23/ | CAACD | TRAJ18 | G12A | CD4 |
| | | LVGGT | | DV6 | RGSTL | | | TCM |
| | | GRQPQ | | | GRLYF | | | |
| | | HF | | | (SEQ ID | | | |
| | | (SEQ ID | | | NO: 319) | | | |
| | | NO: 5) | | | | | | |
| | TRBV2 | CASKE | TRBJ1-4 | TRAV21 | CAAGN | TRAJ31 | G12V | CD4 |
| | | ATAAS | | | NARLM | | | TCM |
| | | TNEKL | | | F | | | |
| | | FF | | | (SEQ ID | | | |
| | | (SEQ ID | | | NO: 320) | | | |
| | | NO: 6) | | | | | | |
| | TRBV5-1 | CASKQ | TRBJ2-1 | TRAV27 | CAGLS | TRAJ20 | G12A; | CD4 |
| | | GNEQF | | | NDYKL | | G13D | Prolif- |
| | | F | | | SF | | | erating |
| | | (SEQ ID | | | (SEQ ID | | | |
| | | NO: 7) | | | NO: 321) | | | |
| | TRBV2 | CASLL | TRBJ1-1 | TRAV26-1 | CIVKS | TRAJ24 | G12A | NA |
| | | DAGAA | | | WGKLQ | | | |
| | | NTEAF | | | F | | | |
| | | F | | | (SEQ ID | | | |
| | | (SEQ ID | | | NO: 322) | | | |
| | | NO: 8) | | | | | | |
| | TRBV7-9 | CASLS | TRBJ1-4 | TRAV17 | CATDA | TRAJ43 | G12A; | CD4 |
| | | RGLNE | | | GNDMR | | G12C; | TCM |
| | | KLFF | | | F | | G12D; | |
| | | (SEQ ID | | | (SEQ ID | | G12R; | |
| | | NO: 9) | | | NO: 323) | | G12V; | |
| | | | | | | | G13D | |
| | TRBV2 | CASML | TRBJ1-5 | TRAV8-3 | CAVGA | TRAJ52 | G12R | CD4 |
| | | QGALN | | | GGTSY | | | TCM |
| | | QPQHF | | | GKLTF | | | |
| | | (SEQ ID | | | (SEQ ID | | | |
| | | NO: 10) | | | NO: 324) | | | |
| | TRBV28 | CASNF | TRBJ1-2 | TRAV13-1 | CAGHS | TRAJ49 | G12V | CD4 |
| | | GQGRY | | | NTGNQ | | | TCM |
| | | GYTF | | | FYF | | | |
| | | (SEQ ID | | | (SEQ ID | | | |
| | | NO: 11) | | | NO: 325) | | | |
| | TRBV2 | CASNP | TRBJ1-6 | TRAV10 | CVVSR | TRAJ17 | G12A | CD4 |
| | | DNALD | | | VKAAG | | | TCM |
| | | NSPLH | | | NKLTF | | | |
| | | F | | | (SEQ ID | | | |
| | | (SEQ ID | | | NO: 326) | | | |
| | | NO: 12) | | | | | | |
| | TRBV19 | CASRD | TRBJ1-5 | TRAV23/ | CAAPY | TRAJ18 | G12R | NA |
| | | SYSNQ | | DV6 | PTGGT | | | |
| | | PQHF | | | SYGKL | | | |
| | | (SEQ ID | | | TF | | | |
| | | NO: 13) | | | (SEQ ID | | | |
| | | | | | NO: 327) | | | |
| | TRBV25-1 | CASRD | TRBJ2-3 | TRAV8-6 | CAVSE | TRAJ8 | G12R | CD4 |
| | | TQGGG | | | RGFQK | | | TCM |
| | | ADTQY | | | LVF | | | |
| | | F | | | (SEQ ID | | | |
| | | (SEQ ID | | | NO: 328) | | | |
| | | NO: 14) | | | | | | |
| | TRBV6-6 | CASRE | TRBJ2-2 | TRAV19 | CALSE | TRAJ24 | G12V | CD4 |
| | | QGQGT | | | SWGKL | | | TCM |
| | | GELFF | | | QF | | | |
| | | (SEQ ID | | | (SEQ ID | | | |
| | | NO: 15) | | | NO: 329) | | | |
| TCR01 | TRBV12-3 | CASRG | TRBJ1-2 | TRAV8-3 | CAVGA | TRAJ17 | G12R | CD4 |
| (Validated) | | DSGFN | | | SGAAG | | | TCM |
| | | YGYTF | | | NKLTF | | | |
| | | (SEQ ID | | | (SEQ ID | | | |
| | | NO: 16) | | | NO: 330) | | | |
| | TRBV2 | CASRG | TRBJ2-3 | TRAV8-4 | CAVSD | TRAJ54 | G12A; | CD4 |
| | | QGAAT | | | NQGAQ | | G12C; | TCM |
| | | DTQYF | | | KLVF | | G12D; | |
| | | (SEQ ID | | | (SEQ ID | | G12R; | |
| | | NO: 17) | | | NO: 331) | | G12V | |
| TCR05 | TRBV2 | CASRG | TRBJ1-2 | TRAV13-1 | CAAPT | TRAJ45 | G12A; | CD8 |
| (Validated) | | QRRIN | | | SGGGA | | G12C; | TEM |
| | | YGYTF | | | DGLTF | | G12V; | |
| | | (SEQ ID | | | (SEQ ID | | G13D | |
| | | NO: 18) | | | NO: 332) | | | |
| | TRBV11-2 | CASRG | TRBJ1-5 | TRAV19 | CAGQG | TRAJ40 | G12C | CD4 |
| | | TGVNQ | | | YFGNE | | | TCM |
| | | PQHF | | | KLTF | | | |
| | | (SEQ ID | | | (SEQ ID | | | |
| | | NO: 19) | | | NO: 333) | | | |
| | TRBV2 | CASRK | TRBJ2-2 | TRAV19 | CALSY | TRAJ23 | G13D | NA |
| | | DTGEL | | | NQGGK | | | |
| | | FF | | | LIF | | | |
| | | (SEQ ID | | | (SEQ ID | | | |
| | | NO: 20) | | | NO: 334) | | | |
| | TRBV2 | CASRL | TRBJ1-1 | TRAV8-6 | CAVSE | TRAJ53 | G12A; | CD4 |
| | | DRSEA | | | PGSGG | | G12C; | TCM |
| | | FF | | | SNYKL | | G12D; | |
| | | (SEQ ID | | | TF | | G12V; | |
| | | NO: 21) | | | (SEQ ID | | G13D | |
| | | | | | NO: 335) | | | |
| | TRBV19 | CASRP | TRBJ1-4 | TRAV8-2 | CVVSD | TRAJ49 | G12R; | CD4 |
| | | GQGYE | | | VNRNQ | | G13D | TCM |
| | | KLFF | | | FYF | | | |
| | | (SEQ ID | | | (SEQ ID | | | |
| | | NO: 22) | | | NO: 336) | | | |
| | TRBV2 | CASRR | TRBJ1-2 | TRAV8-1 | CAVNA | TRAJ48 | G12R | CD4 |
| | | NGLYY | | | PFGNE | | | Prolif- |
| | | TF | | | KLTF | | | erating |
| | | (SEQ ID | | | (SEQ ID | | | |
| | | NO: 23) | | | NO: 337) | | | |
| | TRBV12-3 | CASRS | TRBJ2-2 | TRAV12-1 | CVVNW | TRAJ4 | G12R | NA |
| | | GTGGS | | | GGGYN | | | |
| | | GELFF | | | KLIF | | | |
| | | (SEQ ID | | | (SEQ ID | | | |
| | | NO: 24) | | | NO: 338) | | | |
| | TRBV28 | CASRS | TRBJ2-6 | TRAV17 | CAEIT | TRAJ28 | G12D | CD4 |
| | | PWTGA | | | RYGGS | | | TCM |
| | | NVLTF | | | QGNLI | | | |
| | | (SEQ ID | | | F | | | |
| | | NO: 25) | | | (SEQ ID | | | |
| | | | | | NO: 339) | | | |
| | TRBV28 | CASRT | TRBJ2-3 | TRAV9-2 | CALST | TRAJ40 | G12A; | CD4 |
| | | GGNLD | | | SGTYK | | G12C; | TCM |
| | | DTQYF | | | YIF | | G12D; | |
| | | (SEQ ID | | | (SEQ ID | | G12V | |
| | | NO: 26) | | | NO: 340) | | | |
| | TRBV7-9 | CASRT | TRBJ2-3 | TRAV8-2 | CVVGV | TRAJ52 | G12D | CD4 |
| | | LSGGD | | | RGGGT | | | TCM |
| | | TQYF | | | SYGKL | | | |
| | | (SEQ ID | | | TF | | | |
| | | NO: 27) | | | (SEQ ID | | | |
| | | | | | NO: 341) | | | |
| | TRBV28 | CASSA | TRBJ2-7 | TRAV16 | CALRA | TRAJ11 | G12V | CD8 |
| | | FYEQY | | | QNSGY | | | TCM |
| | | F | | | STLTF | | | |
| | | (SEQ ID | | | (SEQ ID | | | |
| | | NO: 28) | | | NO: 342) | | | |
| | TRBV11-2 | CASSA | TRBJ1-3 | TRAV38-1 | CAFMK | TRAJ38 | G12R | CD4 |
| | | LGSGN | | | HMSGN | | | TCM |
| | | TIYF | | | NRKLI | | | |
| | | (SEQ ID | | | W | | | |
| | | NO: 29) | | | (SEQ ID | | | |
| | | | | | NO: 343) | | | |
| | TRBV6-4 | CASSA | TRBJ2-1 | TRAV29/ | CAASG | TRAJ40 | G12A; | CD4 |
| | | QGTSY | | DV5 | TYKYI | | G12C; | TCM |
| | | NEQFF | | | F | | G12V | |
| | | (SEQ ID | | | (SEQ ID | | | |
| | | NO: 30) | | | NO: 344) | | | |
| | TRBV6-5 | CASSA | TRBJ1-5 | TRAV13-2 | CAEIA | TRAJ49 | G13D | CD4 |
| | | SFYQP | | | GNQFY | | | Prolif- |
| | | QHF | | | F | | | erating |
| | | (SEQ ID | | | (SEQ ID | | | |
| | | NO: 31) | | | NO: 345) | | | |
| | TRBV12-4 | CASSA | TRBJ1-6 | TRAV8-4 | CAVSD | TRAJ8 | G12R | CD4 |
| | | TGGNS | | | SFQKL | | | TCM |
| | | PLHF | | | VF | | | |
| | | (SEQ ID | | | (SEQ ID | | | |
| | | NO: 32) | | | NO: 346) | | | |
| | TRBV7-9 | CASSE | TRBJ1-2 | TRAV21 | CAVSS | TRAJ47 | G12C | CD8 |
| | | GQGDY | | | TYGNK | | | TCM |
| | | GYTF | | | LVF | | | |
| | | (SEQ ID | | | (SEQ ID | | | |
| | | NO: 33) | | | NO: 347) | | | |
| | TRBV2 | CASSE | TRBJ1-5 | TRAV29/ | CAAIG | TRAJ37 | G12A; | CD4 |
| | | NRRRE | | DV5 | SSNTG | | G12V; | TCM |
| | | PQHF | | | KLIF | | G13D | |
| | | (SEQ ID | | | (SEQ ID | | | |
| | | NO: 34) | | | NO: 348) | | | |
| | TRBV6-1 | CASSE | TRBJ1-6 | TRAV8-4 | CAVSE | TRAJ9 | G12C; | CD4 |
| | | QSGLT | | | DTGGF | | G12D; | TCM |
| | | NSPLH | | | KTIF | | G12V | |
| | | F | | | (SEQ ID | | | |
| | | (SEQ ID | | | NO: 349) | | | |
| | | NO: 35) | | | | | | |
| | TRBV7-3 | CASSF | TRBJ2-7 | TRAV21 | CAVLG | TRAJ58 | G12C | NA |
| | | AAGLG | | | TSGSR | | | |
| | | YEQYF | | | LTF | | | |
| | | (SEQ ID | | | (SEQ ID | | | |
| | | NO: 36) | | | NO: 350) | | | |
| TCR02 | TRBV5-6 | CASSF | TRBJ2-2 | TRAV3 | CAVRD | TRAJ20 | G12V | CD4 |
| (Validated) | | AGVYT | | | KRSND | | | Prolif- |
| | | GELFF | | | YKLSF | | | erating |
| | | (SEQ ID | | | (SEQ ID | | | |
| | | NO: 37) | | | NO: 351) | | | |
| | TRBV27 | CASSF | TRBJ2-1 | TRAV23/ | CAASN | TRAJ10 | G12R; | CD8 |
| | | PTGGL | | DV6 | VLTGG | | G13D | TCM |
| | | SSEQF | | | GNKLT | | | |
| | | F | | | F | | | |
| | | (SEQ ID | | | (SEQ ID | | | |
| | | NO: 38) | | | NO: 352) | | | |
| | TRBV5-1 | CASSF | TRBJ2-5 | TRAV13-1 | CAAIS | TRAJ20 | G12R | CD4 |
| | | QETQY | | | RTGSA | | | TCM |
| | | F | | | RQLTF | | | |
| | | (SEQ ID | | | (SEQ ID | | | |
| | | NO: 39) | | | NO: 353) | | | |
| | TRBV7-9 | CASSF | TRBJ2-5 | TRAV17 | CATDA | TRAJ56 | G12A | CD4 |
| | | RGGLQ | | | TGANS | | | TCM |
| | | ETQYF | | | KLTF | | | |
| | | (SEQ ID | | | (SEQ ID | | | |
| | | NO: 40) | | | NO: 354) | | | |
| | TRBV28 | CASSF | TRBJ1-2 | TRAV9-2 | CALSD | TRAJ45 | G12C; | NA |
| | | TDRRK | | | SGGGA | | G12R; | |
| | | DTF | | | DGLTF | | G13D | |
| | | (SEQ ID | | | (SEQ ID | | | |
| | | NO: 41) | | | NO: 355) | | | |
| | TRBV7-9 | CASSF | TRBJ1-5 | TRAV19 | CALSE | TRAJ39 | G12R; | CD4 |
| | | YGTGG | | | AGNAG | | G13D | TCM |
| | | EGKPQ | | | NMLTF | | | |
| | | HF | | | (SEQ ID | | | |
| | | (SEQ ID | | | NO: 356) | | | |
| | | NO: 42) | | | | | | |
| | TRBV28 | CASSG | TRBJ2-7 | TRAV22 | CAVPG | TRAJ52 | G12D | CD4 |
| | | TDFYE | | | GTSYG | | | TCM |
| | | QYF | | | KLTF | | | |
| | | (SEQ ID | | | (SEQ ID | | | |
| | | NO: 43) | | | NO: 357) | | | |
| | TRBV28 | CASSG | TRBJ1-1 | TRAV8-3 | CAVGA | TRAJ15 | G12V | CD4 |
| | | TGDSG | | | RQAGT | | | Prolif- |
| | | EAFF | | | ALIF | | | erating |
| | | (SEQ ID | | | (SEQ ID | | | |
| | | NO: 44) | | | NO: 358) | | | |
| | TRBV6-2 | CASSG | TRBJ1-5 | TRAV38-1 | CAFMT | TRAJ52 | G12C; | NA |
| | | TGGAI | | | INAGG | | G12R; | |
| | | SNQPQ | | | TSYGK | | G12V | |
| | | HF | | | LTF | | | |
| | | (SEQ ID | | | (SEQ ID | | | |
| | | NO: 45) | | | NO: 359) | | | |
| | TRBV19 | CASSI | TRBJ2-2 | TRAV10 | CVVRV | TRAJ35 | G12A; | CD4 |
| | | GGTTG | | | GFGNV | | G12C | TEM |
| | | ELFF | | | LHC | | | |
| | | (SEQ ID | | | (SEQ ID | | | |
| | | NO: 46) | | | NO: 360) | | | |
| | TRBV19 | CASSI | TRBJ2-2 | TRAV13-1 | CAATR | TRAJ20 | G12V | CD4 |
| | | STNTG | | | RSNDY | | | TCM |
| | | ELFF | | | KLSF | | | |
| | | (SEQ ID | | | (SEQ ID | | | |
| | | NO: 47) | | | NO: 361) | | | |
| | TRBV12-3 | CASSI | TRBJ1-5 | TRAV26-1 | CIAPN | TRAJ33 | G12V | CD4 |
| | | TGSSG | | | MDSNY | | | TCM |
| | | QPQHF | | | QLIW | | | |
| | | (SEQ ID | | | (SEQ ID | | | |
| | | NO: 48) | | | NO: 362) | | | |
| | TRBV2 | CASSK | TRBJ2-5 | TRAV17 | CATPK | TRAJ23 | G12A; | CD4 |
| | | DRGLA | | | IYNQG | | G12C; | TCM |
| | | LETQY | | | GKLIF | | G12D; | |
| | | F | | | (SEQ ID | | G12V | |
| | | (SEQ ID | | | NO: 363) | | | |
| | | NO: 49) | | | | | | |
| | TRBV2 | CASSK | TRBJ2-1 | TRAV21 | CAVRP | TRAJ56 | G12A; | CD4 |
| | | ESANR | | | PGANS | | G12C; | Prolif- |
| | | YNEQF | | | KLTF | | G12D; | erating |
| | | F | | | (SEQ ID | | G12V | |
| | | (SEQ ID | | | NO: 364) | | | |
| | | NO: 50) | | | | | | |
| | TRBV12-3 | CASSL | TRBJ1-5 | TRAV2 | CAVED | TRAJ31 | G12R | NA |
| | | ASNQP | | | DNNAR | | | |
| | | QHF | | | LMF | | | |
| | | (SEQ ID | | | (SEQ ID | | | |
| | | NO: 51) | | | NO: 365) | | | |
| | TRBV11-2 | CASSL | TRBJ2-1 | TRAV13-1 | CAASP | TRAJ21 | G12D | NA |
| | | DQTSN | | | RFNKF | | | |
| | | EQFF | | | YF | | | |
| | | (SEQ ID | | | (SEQ ID | | | |
| | | NO: 52) | | | NO: 366) | | | |
| | TRBV6-6 | CASSL | TRBJ1-6 | TRAV12-2 | CAVNS | TRAJ33 | G12D; | CD4 |
| | | DRGLG | | | NYQLI | | G12R | TCM |
| | | NSPLH | | | W | | | |
| | | F | | | (SEQ ID | | | |
| | | (SEQ ID | | | NO: 367) | | | |
| | | NO: 53) | | | | | | |
| | TRBV7-8 | CASSL | TRBJ2-2 | TRAV8-1 | CAVKG | TRAJ38 | G12A | CD4 |
| | | DSGTN | | | PAGNN | | | Prolif- |
| | | TGELF | | | RKLIW | | | erating |
| | | F | | | (SEQ ID | | | |
| | | (SEQ ID | | | NO: 368) | | | |
| | | NO: 54) | | | | | | |
| | TRBV5-1 | CASSL | TRBJ2-3 | TRAV9-2 | CALSV | TRAJ54 | G12R | CD4 |
| | | DSLAT | | | GGAQK | | | Prolif- |
| | | DTQYF | | | LVF | | | erating |
| | | (SEQ ID | | | (SEQ ID | | | |
| | | NO: 55) | | | NO: 369) | | | |
| | TRBV7-8 | CASSL | TRBJ2-4 | TRAV12-1 | CVVLE | TRAJ9 | G12A | dnT |
| | | EGGLA | | | RNTGG | | | |
| | | KNIQY | | | FKTIF | | | |
| | | F | | | (SEQ ID | | | |
| | | (SEQ ID | | | NO: 370) | | | |
| | | NO: 56) | | | | | | |
| | TRBV7-8 | CASSL | TRBJ1-4 | TRAV17 | CATDR | TRAJ33 | G13D | CD8 |
| | | EGRGP | | | DSNYQ | | | TCM |
| | | TNEKL | | | LIW | | | |
| | | FF | | | (SEQ ID | | | |
| | | (SEQ ID | | | NO: 371) | | | |
| | | NO: 57) | | | | | | |
| | TRBV7-9 | CASSL | TRBJ1-6 | TRAV14/ | CAMRE | TRAJ20 | G12D | CD4 |
| | | ESGNS | | DV4 | GSDYK | | | TCM |
| | | PLHF | | | LSF | | | |
| | | (SEQ ID | | | (SEQ ID | | | |
| | | NO: 58) | | | NO: 372) | | | |
| | TRBV7-9 | CASSL | TRBJ2-1 | TRAV13-1 | CAASG | TRAJ45 | G12R | NA |
| | | ETGVE | | | GGADG | | | |
| | | QFF | | | LTF | | | |
| | | (SEQ ID | | | (SEQ ID | | | |
| | | NO: 59) | | | NO: 373) | | | |
| | TRBV11-2 | CASSL | TRBJ1-4 | TRAV13-1 | CAASL | TRAJ29 | G12R | CD4 |
| | | FGGGG | | | PGNTP | | | Prolif- |
| | | EKLFF | | | LVF | | | erating |
| | | (SEQ ID | | | (SEQ ID | | | |
| | | NO: 60) | | | NO: 374) | | | |
| | TRBV5-5 | CASSL | TRBJ2-7 | TRAV38-2/ | CAYRS | TRAJ32 | G12D | CD4 |
| | | FLGSY | | DV8 | GATNK | | | TCM |
| | | EQYF | | | LIF | | | |
| | | (SEQ ID | | | (SEQ ID | | | |
| | | NO: 61) | | | NO: 375) | | | |
| | TRBV7-9 | CASSL | TRBJ1-5 | TRAV21 | CAVPP | TRAJ48 | G12R | NA |
| | | GGGNQ | | | PNFGN | | | |
| | | PQHF | | | EKLTF | | | |
| | | (SEQ ID | | | (SEQ ID | | | |
| | | NO: 62) | | | NO: 376) | | | |
| | TRBV7-3 | CASSL | TRBJ2-2 | TRAV17 | CATDS | TRAJ49 | G12V | CD4 |
| | | GGNTG | | | SNTGN | | | TCM |
| | | ELFF | | | QFYF | | | |
| | | (SEQ ID | | | (SEQ ID | | | |
| | | NO: 63) | | | NO: 377) | | | |
| | TRBV28 | CASSL | TRBJ2-1 | TRAV26-1 | CIAHD | TRAJ18 | G12V | CD4 |
| | | GGSGS | | | RGSTL | | | TCM |
| | | FYHNE | | | GRLYF | | | |
| | | QFF | | | (SEQ ID | | | |
| | | (SEQ ID | | | NO: 378) | | | |
| | | NO: 64) | | | | | | |
| | TRBV7-9 | CASSL | TRBJ2-1 | TRAV9-2 | CALSP | TRAJ9 | G12R | CD4 |
| | | GLRPI | | | QGTGG | | | TCM |
| | | NEQFF | | | FKTIF | | | |
| | | (SEQ ID | | | (SEQ ID | | | |
| | | NO: 65) | | | NO: 379) | | | |
| | TRBV7-9 | CASSL | TRBJ2-3 | TRAV29/ | CAALA | TRAJ41 | G12A; | CD4 |
| | | GRGPT | | DV5 | GPGYA | | G12C; | TCM |
| | | DTQYF | | | LNF | | G12D; | |
| | | (SEQ ID | | | (SEQ ID | | G12V; | |
| | | NO: 66) | | | NO: 380) | | G13D | |
| | TRBV14 | CASSL | TRBJ1-1 | TRAV21 | CAVRG | TRAJ38 | G12R | CD4 |
| | | GVNTE | | | AGNNR | | | TCM |
| | | AFF | | | KLIW | | | |
| | | (SEQ ID | | | (SEQ ID | | | |
| | | NO: 67) | | | NO: 381) | | | |
| | TRBV7-2 | CASSL | TRBJ2-7 | TRAV25 | CADSY | TRAJ32 | G12R | CD8 |
| | | GYRGE | | | GGATN | | | TEM |
| | | QYF | | | KLIF | | | |
| | | (SEQ ID | | | (SEQ ID | | | |
| | | NO: 68) | | | NO: 382) | | | |
| | TRBV6-6 | CASSL | TRBJ1-6 | TRAV26-2 | CILSN | TRAJ28 | G12C | CD8 |
| | | LDRGD | | | VYSGA | | | TCM |
| | | SPLHF | | | GSYQL | | | |
| | | (SEQ ID | | | TF | | | |
| | | NO: 69) | | | (SEQ ID | | | |
| | | | | | NO: 383) | | | |
| | TRBV7-9 | CASSL | TRBJ2-7 | TRAV24 | CAFDN | TRAJ43 | G13D | NA |
| | | LTGGQ | | | NDMRF | | | |
| | | YF | | | (SEQ ID | | | |
| | | (SEQ ID | | | NO: 384) | | | |
| | | NO: 70) | | | | | | |
| | TRBV28 | CASSL | TRBJ2-7 | TRAV38-1 | CAFIG | TRAJ54 | G12R | NA |
| | | NRGYE | | | LLGIQ | | | |
| | | QYV | | | GAQKL | | | |
| | | (SEQ ID | | | VF | | | |
| | | NO: 71) | | | (SEQ ID | | | |
| | | | | | NO: 385) | | | |
| | TRBV3-1 | CASSL | TRBJ1-1 | TRAV13-1 | CAASI | TRAJ52 | G12R | CD4 |
| | | NTEAF | | | RSGGS | | | TCM |
| | | F | | | YIPTF | | | |
| | | (SEQ ID | | | (SEQ ID | | | |
| | | NO: 72) | | | NO: 386) | | | |
| | TRBV12-3 | CASSL | TRBJ1-1 | TRAV23/ | CAANA | TRAJ38 | G12R | CD4 |
| | | QGRTE | | DV6 | GNNRK | | | TCM |
| | | AFF | | | LIW | | | |
| | | (SEQ ID | | | (SEQ ID | | | |
| | | NO: 73) | | | NO: 387) | | | |
| | TRBV5-5 | CASSL | TRBJ1-2 | TRAV23/ | CAAEF | TRAJ44 | G12A; | NA |
| | | QGSYG | | DV6 | TGTAS | | G12C; | |
| | | YTF | | | KLTF | | G12D; | |
| | | (SEQ ID | | | (SEQ ID | | G12R; | |
| | | NO: 74) | | | NO: 388) | | G12V; | |
| | | | | | | | G13D | |
| | TRBV6-6 | CASSL | TRBJ1-1 | TRAV13-2 | CAEGR | TRAJ9 | G12R | CD4 |
| | | RGEAF | | | LTGGF | | | TCM |
| | | F | | | KTIF | | | |
| | | (SEQ ID | | | (SEQ ID | | | |
| | | NO: | | | NO: | | | |
| | | 75) | | | 389) | | | |
| | TRBV5-6 | CASSL | TRBJ2-1 | TRAV1-2 | CAVRG | TRAJ9 | G12R | NA |
| | | RGNEQ | | | GGGFK | | | |
| | | FF | | | TIF | | | |
| | | (SEQ ID | | | (SEQ ID | | | |
| | | NO: | | | NO: | | | |
| | | 76) | | | 390) | | | |
| | TRBV12-3 | CASSL | TRBJ1-5 | TRAV30 | CGTEE | TRAJ30 | G12A; | NA |
| | | RTGGR | | | MNRDD | | G12C; | |
| | | MPQHF | | | KIIF | | G12D; | |
| | | (SEQ ID | | | (SEQ ID | | G12V | |
| | | NO: | | | NO: | | | |
| | | 77) | | | 391) | | | |
| | TRBV12-3 | CASSL | TRBJ1-4 | TRAV29/ | CAASN | TRAJ41 | G12A; | NA |
| | | RTNTG | | DV5 | SGYAL | | G12C; | |
| | | EKLFF | | | NF | | G12D; | |
| | | (SEQ ID | | | (SEQ ID | | G12V | |
| | | NO: | | | NO: | | | |
| | | 78) | | | 392) | | | |
| | TRBV12-3 | CASSL | TRBJ1-5 | TRAV13-1 | CAAAS | TRAJ11 | G12C; | NA |
| | | SPGKS | | | GYSTL | | G12D; | |
| | | NQPQH | | | TF | | G12R | |
| | | F | | | (SEQ ID | | | |
| | | (SEQ ID | | | NO: | | | |
| | | NO: | | | 393) | | | |
| | | 79) | | | | | | |
| | TRBV28 | CASSL | TRBJ2-7 | TRAV12-1 | CVVSL | TRAJ10 | G12C | CD4 |
| | | SYEQY | | | LTGGG | | | TCM |
| | | F | | | NKLTF | | | |
| | | (SEQ ID | | | (SEQ ID | | | |
| | | NO: | | | NO: | | | |
| | | 80) | | | 394) | | | |
| | TRBV12-4 | CASSL | TRBJ1-1 | TRAV21 | CAVRS | TRAJ7 | G12C | NA |
| | | TEGVR | | | SLGNN | | | |
| | | TEAFF | | | RLAF | | | |
| | | (SEQ ID | | | (SEQ ID | | | |
| | | NO: | | | NO: | | | |
| | | 81) | | | 395) | | | |
| | TRBVI | CASSL | TRBJ2-5 | TRAV21 | CAAMG | TRAJ30 | G12R | NA |
| | 1-2 | VETQY | | | NRDDK | | | |
| | | F | | | IIF | | | |
| | | (SEQ ID | | | (SEQ ID | | | |
| | | NO: | | | NO: | | | |
| | | 82) | | | 396) | | | |
| | TRBV7-9 | CASSL | TRBJ1-3 | TRAV39 | CAVDR | TRAJ53 | G12C; | NA |
| | | VGGNT | | | ARNSG | | G12R; | |
| | | IYF | | | GSNYK | | G12V; | |
| | | (SEQ ID | | | LTF | | G13D | |
| | | NO: | | | (SEQ ID | | | |
| | | 83) | | | NO: | | | |
| | | | | | 397) | | | |
| | TRBV7-9 | CASSL | TRBJ1-1 | TRAV24 | CALGE | TRAJ26 | G13D | NA |
| | | VGNTE | | | YGNKL | | | |
| | | AFF | | | VF | | | |
| | | (SEQ ID | | | (SEQ ID | | | |
| | | NO: | | | NO: | | | |
| | | 84) | | | 398) | | | |
| | TRBV11-3 | CASSL | TRBJ2-7 | TRAV29/ | CAASG | TRAJ41 | G12A; | NA |
| | | VSTAE | | DV5 | ANSGY | | G12C; | |
| | | QYF | | | ALNF | | G12D; | |
| | | (SEQ ID | | | (SEQ ID | | G12R; | |
| | | NO: | | | NO: | | G12V; | |
| | | 85) | | | 399) | | G13D | |
| | TRBV5-1 | CASSL | TRBJ2-2 | TRAV27 | CAGPT | TRAJ13 | G12R; | NA |
| | | VVTGE | | | NSGGY | | G13D | |
| | | LFF | | | QKVTF | | | |
| | | (SEQ ID | | | (SEQ ID | | | |
| | | NO: | | | NO: | | | |
| | | 86) | | | 400) | | | |
| | TRBV28 | CASSL | TRBJ2-3 | TRAV26-1 | CIVRV | TRAJ39 | G12A | NA |
| | | WGATD | | | AYNNA | | | |
| | | TQYF | | | GNMLT | | | |
| | | (SEQ ID | | | F | | | |
| | | NO: | | | (SEQ ID | | | |
| | | 87) | | | NO: | | | |
| | | | | | 401) | | | |
| TCR03 | TRBV2 | CASSP | TRBJ1-5 | TRAV2 | CAAGD | TRAJ5 | G12A; | CD4 |
| (Validated) | | DSSFG | | | TGRRA | | G12C; | TCM |
| | | NQPQH | | | LTF | | G12D; | |
| | | F | | | (SEQ ID | | G12V | |
| | | (SEQ ID | | | NO: | | | |
| | | NO: | | | 402) | | | |
| | | 88) | | | | | | |
| | TRBV7-9 | CASSP | TRBJ2-1 | TRAV12-1 | CASGR | TRAJ42 | G12R | MAIT |
| | | DSYNE | | | GSQGN | | | |
| | | QFF | | | LIF | | | |
| | | (SEQ ID | | | (SEQ ID | | | |
| | | NO: | | | NO: | | | |
| | | 89) | | | 403) | | | |
| | TRBV6-6 | CASSP | TRBJ2-5 | TRAV10 | CALID | TRAJ18 | G12D | NA |
| | | EETQY | | | RGSTL | | | |
| | | F | | | GRLYF | | | |
| | | (SEQ ID | | | (SEQ ID | | | |
| | | NO: | | | NO: | | | |
| | | 90) | | | 404) | | | |
| | TRBV27 | CASSP | TRBJ1-6 | TRAV12-2 | CAAPP | TRAJ52 | G12C; | CD8 |
| | | GQAAN | | | GGTSY | | G12D; | TCM |
| | | SPLHF | | | GKLTF | | G13D | |
| | | (SEQ ID | | | (SEQ ID | | | |
| | | NO: | | | NO: | | | |
| | | 91) | | | 405) | | | |
| | TRBV7-8 | CASSP | TRBJ1-1 | TRAV20 | CAVQA | TRAJ13 | G13D | NA |
| | | GRVAF | | | AGGYQ | | | |
| | | F | | | KVTF | | | |
| | | (SEQ ID | | | (SEQ ID | | | |
| | | NO: | | | NO: | | | |
| | | 92) | | | 406) | | | |
| | TRBV18 | CASSP | TRBJ1-5 | TRAV5 | CAERQ | TRAJ29 | G12A; | CD4 |
| | | GTDQP | | | GNTPL | | G12C | TCM |
| | | QHF | | | VF | | | |
| | | (SEQ ID | | | (SEQ ID | | | |
| | | NO: | | | NO: | | | |
| | | 93) | | | 407) | | | |
| | TRBV18 | CASSP | TRBJ1-1 | TRAV23/ | CAASG | TRAJ30 | G13D | CD4 |
| | | GTEAF | | DV6 | DRDDK | | | TCM |
| | | F | | | IIF | | | |
| | | (SEQ ID | | | (SEQ ID | | | |
| | | NO: | | | NO: | | | |
| | | 94) | | | 408) | | | |
| | TRBV18 | CASSP | TRBJ1-1 | TRAV17 | CATDP | TRAJ36 | G12R | CD4 |
| | | MGTGN | | | GANNL | | | TCM |
| | | TEAFF | | | FF | | | |
| | | (SEQ ID | | | (SEQ ID | | | |
| | | NO: | | | NO: | | | |
| | | 95) | | | 409) | | | |
| | TRBV18 | CASSP | TRBJ2-1 | TRAV9-2 | CALPP | TRAJ52 | G12C; | NA |
| | | PDRGR | | | GGGTS | | G12R | |
| | | HEQFF | | | YGKLT | | | |
| | | (SEQ ID | | | F | | | |
| | | NO: | | | (SEQ ID | | | |
| | | 96) | | | NO: | | | |
| | | | | | 410) | | | |
| | TRBV18 | CASSP | TRBJ2-2 | TRAV3 | CAVRD | TRAJ4 | G12C; | CD4 |
| | | PSGSG | | | AGGYN | | G12V | TCM |
| | | ELFF | | | KLIF | | | |
| | | (SEQ ID | | | (SEQ ID | | | |
| | | NO: | | | NO: | | | |
| | | 97) | | | 411) | | | |
| | TRBV6-1 | CASSP | TRBJ1-3 | TRAV12-3 | CAMRL | TRAJ17 | G12D | CD8 |
| | | PVLVS | | | GAAGN | | | TCM |
| | | GNTIY | | | KLTF | | | |
| | | F | | | (SEQ ID | | | |
| | | (SEQ ID | | | NO: | | | |
| | | NO: | | | 412) | | | |
| | | 98) | | | | | | |
| | TRBV5-1 | CASSP | TRBJ1-1 | TRAV9-2 | CALSD | TRAJ23 | G12V | CD4 |
| | | QDRGQ | | | YNQGG | | | Prolif- |
| | | GNTEA | | | KLIF | | | erating |
| | | FF | | | (SEQ ID | | | |
| | | (SEQ ID | | | NO: | | | |
| | | NO: | | | 413) | | | |
| | | 99) | | | | | | |
| | TRBV27 | CASSP | TRBJ1-5 | TRAV9-2 | CALSP | TRAJ13 | G12V | NA |
| | | RDRGL | | | RGGYQ | | | |
| | | YQPQH | | | KVTF | | | |
| | | F | | | (SEQ ID | | | |
| | | (SEQ ID | | | NO: | | | |
| | | NO: | | | 414) | | | |
| | | 100) | | | | | | |
| | TRBV7-8 | CASSP | TRBJ1-3 | TRAV3 | CAVRG | TRAJ11 | G12D | CD4 |
| | | RGAGN | | | RGYST | | | TCM |
| | | TIYF | | | LTF | | | |
| | | (SEQ ID | | | (SEQ ID | | | |
| | | NO: | | | NO: | | | |
| | | 101) | | | 415) | | | |
| | TRBV6-2 | CASSP | TRBJ1-5 | TRAV25 | CAGDF | TRAJ52 | G12V | CD4 |
| | | RTGGN | | | GGTSY | | | TCM |
| | | QPQHF | | | GKLTF | | | |
| | | (SEQ ID | | | (SEQ ID | | | |
| | | NO: | | | NO: | | | |
| | | 102) | | | 416) | | | |
| | TRBV5-1 | CASSP | TRBJ2-7 | TRAV13-1 | CAASR | TRAJ7 | G12A | CD4 |
| | | SAGAG | | | GNNRL | | | TCM |
| | | YEQYF | | | AF | | | |
| | | (SEQ ID | | | (SEQ ID | | | |
| | | NO: | | | NO: | | | |
| | | 103) | | | 417) | | | |
| | TRBV18 | CASSP | TRBJ2-6 | TRAV3 | CAVRD | TRAJ4 | G12A | CD4 |
| | | SQGID | | | GGGYN | | | TCM |
| | | SGANV | | | KLIF | | | |
| | | LTF | | | (SEQ ID | | | |
| | | (SEQ ID | | | NO: | | | |
| | | NO: | | | 418) | | | |
| | | 104) | | | | | | |
| | TRBV27 | CASSP | TRBJ2-7 | TRAV21 | CAVQG | TRAJ32 | G12A | CD8 |
| | | SRDRS | | | YLGGA | | | TCM |
| | | YEQYF | | | TNKLI | | | |
| | | (SEQ ID | | | F | | | |
| | | NO: | | | (SEQ ID | | | |
| | | 105) | | | NO: | | | |
| | | | | | 419) | | | |
| | TRBV2 | CASSP | TRBJ1-1 | TRAV21 | CAVYS | TRAJ22 | G12A; | CD4 |
| | | TDRIR | | | NTGKL | | G12C; | TCM |
| | | AFF | | | IF | | G12D; | |
| | | (SEQ ID | | | (SEQ ID | | G12V; | |
| | | NO: | | | NO: | | G13D | |
| | | 106) | | | 420) | | | |
| | TRBV7-2 | CASSP | TRBJ2-5 | TRAV12-3 | CAMRV | TRAJ31 | G12D | NA |
| | | YRGLN | | | NNARL | | | |
| | | HSF | | | MF | | | |
| | | (SEQ ID | | | (SEQ ID | | | |
| | | NO: | | | NO: | | | |
| | | 107) | | | 421) | | | |
| | TRBV4-2 | CASSQ | TRBJ2-3 | TRAV9-2 | CALSD | TRAJ56 | G12R | NA |
| | | DGGGT | | | RTGAN | | | |
| | | DTQYF | | | SKLTF | | | |
| | | (SEQ ID | | | (SEQ ID | | | |
| | | NO: | | | NO: | | | |
| | | 108) | | | 422) | | | |
| | TRBV3-1 | CASSQ | TRBJ2-3 | TRAV9-2 | CALSD | TRAJ22 | G12R | NA |
| | | DGVAT | | | RGSAR | | | |
| | | DTQYF | | | QLTF | | | |
| | | (SEQ ID | | | (SEQ ID | | | |
| | | NO: | | | NO: | | | |
| | | 109) | | | 423) | | | |
| | TRBV4-2 | CASSQ | TRBJ1-5 | TRAV29/ | CAAKG | TRAJ49 | G12R | CD4 |
| | | DKGRD | | DV5 | NTGNQ | | | TCM |
| | | QPQHF | | | FYF | | | |
| | | (SEQ ID | | | (SEQ ID | | | |
| | | NO: | | | NO: | | | |
| | | 110) | | | 424) | | | |
| | TRBV14 | CASSQ | TRBJ1-1 | TRAV12-2 | CAALR | TRAJ18 | G12R | CD4 |
| | | DRPSF | | | DRGST | | | Prolif- |
| | | TEAFF | | | LGRLY | | | erating |
| | | (SEQ ID | | | F | | | |
| | | NO: | | | (SEQ ID | | | |
| | | 111) | | | NO: | | | |
| | | | | | 425) | | | |
| | TRBV11-2 | CASSQ | TRBJ2-2 | TRAV8-6 | CAVSE | TRAJ8 | G12A | NA |
| | | DRQKL | | | TGFQK | | | |
| | | SGELF | | | LVF | | | |
| | | F | | | (SEQ ID | | | |
| | | (SEQ ID | | | NO: | | | |
| | | NO: | | | 426) | | | |
| | | 112) | | | | | | |
| | TRBV4-1 | CASSQ | TRBJ2-1 | TRAV17 | CATDA | TRAJ58 | G12D | NA |
| | | DRTST | | | TSGSR | | | |
| | | RDEQF | | | LTF | | | |
| | | F | | | (SEQ ID | | | |
| | | (SEQ ID | | | NO: | | | |
| | | NO: | | | 427) | | | |
| | | 113) | | | | | | |
| | TRBV4-1 | CASSQ | TRBJ1-5 | TRAV3 | CAERN | TRAJ31 | G12R | dnT |
| | | DWVVG | | | NNARL | | | |
| | | NQPQH | | | MF | | | |
| | | F | | | (SEQ ID | | | |
| | | (SEQ ID | | | NO: | | | |
| | | NO: | | | 428) | | | |
| | | 114) | | | | | | |
| | TRBV4-1 | CASSQ | TRBJ1-5 | TRAV29/ | CAAST | TRAJ49 | G13D | CD4 |
| | | EDRGN | | DV5 | GNQFY | | | TCM |
| | | QPQHF | | | F | | | |
| | | (SEQ ID | | | (SEQ ID | | | |
| | | NO: | | | NO: | | | |
| | | 115) | | | 429) | | | |
| | TRBV14 | CASSQ | TRBJ2-5 | TRAV12-1 | CVVTL | TRAJ39 | G12V | NA |
| | | GGVGE | | | NNAGN | | | |
| | | TQYF | | | MLTF | | | |
| | | (SEQ ID | | | (SEQ ID | | | |
| | | NO: | | | NO: | | | |
| | | 116) | | | 430) | | | |
| | TRBV3-1 | CASSQ | TRBJ1-5 | TRAV29/ | CAASG | TRAJ45 | G12C; | CD8 |
| | | GRGGY | | DV5 | GEGGG | | G12R | TCM |
| | | QPQHF | | | ADGLT | | | |
| | | (SEQ ID | | | F | | | |
| | | NO: | | | (SEQ ID | | | |
| | | 117) | | | NO: | | | |
| | | | | | 431) | | | |
| | TRBV4-3 | CASSQ | TRBJ1-2 | TRAV21 | CAVGP | TRAJ20 | G12C; | NA |
| | | GTGGM | | | WGDYK | | G12D; | |
| | | RGYTF | | | LSF | | G12R; | |
| | | (SEQ ID | | | (SEQ ID | | G12V; | |
| | | NO: | | | NO: | | G13D | |
| | | 118) | | | 432) | | | |
| | TRBV18 | CASSQ | TRBJ2-7 | TRAV23/ | CAASW | TRAJ29 | G13D | NA |
| | | QGSEQ | | DV6 | GNTPL | | | |
| | | YV | | | VF | | | |
| | | (SEQ ID | | | (SEQ ID | | | |
| | | NO: | | | NO: | | | |
| | | 119) | | | 433) | | | |
| | TRBV3-1 | CASSQ | TRBJ2-1 | TRAV21 | CAVRR | TRAJ33 | G12A; | CD4 |
| | | SEVGG | | | RGDSN | | G12C; | Prolif- |
| | | QFF | | | YQLIW | | G12R; | erating |
| | | (SEQ ID | | | (SEQ ID | | G12V | |
| | | NO: | | | NO: | | | |
| | | 120) | | | 434) | | | |
| | TRBV6-2 | CASSR | TRBJ2-3 | TRAV8-6 | CAVSE | TRAJ9 | G12C | CD4 |
| | | DSGRA | | | KGAGG | | | TCM |
| | | GDTQY | | | FKTIF | | | |
| | | F | | | (SEQ ID | | | |
| | | (SEQ ID | | | NO: | | | |
| | | NO: | | | 435) | | | |
| | | 121) | | | | | | |
| | TRBV6-2 | CASSR | TRBJ1-2 | TRAV40 | CAVSQ | TRAJ12 | G12C | CD4 |
| | | EGYGY | | | MDSSY | | | TCM |
| | | TF | | | KLIF | | | |
| | | (SEQ ID | | | (SEQ ID | | | |
| | | NO: | | | NO: | | | |
| | | 122) | | | 436) | | | |
| | TRBV12-3 | CASSR | TRBJ1-3 | TRAV8-6 | CAVSG | TRAJ31 | G12A | CD4 |
| | | QSSGN | | | LNNAR | | | TCM |
| | | TIYF | | | LMF | | | |
| | | (SEQ ID | | | (SEQ ID | | | |
| | | NO: | | | NO: | | | |
| | | 123) | | | 437) | | | |
| | TRBV28 | CASSR | TRBJ1-1 | TRAV1-2 | CALSG | TRAJ15 | G12R | CD4 |
| | | SGLFN | | | GQAGT | | | TCM |
| | | TEGAF | | | ALIF | | | |
| | | F | | | (SEQ ID | | | |
| | | (SEQ ID | | | NO: | | | |
| | | NO: | | | 438) | | | |
| | | 124) | | | | | | |
| | TRBV28 | CASSR | TRBJ2-6 | TRAV1-1 | CAVRR | TRAJ23 | G12V | CD4 |
| | | TALAA | | | QGGKL | | | Prolif- |
| | | NVLTF | | | IF | | | erating |
| | | (SEQ ID | | | (SEQ ID | | | |
| | | NO: | | | NO: | | | |
| | | 125) | | | 439) | | | |
| | TRBV5-1 | CASSR | TRBJ1-6 | TRAV21 | CAVRP | TRAJ48 | G12D | CD4 |
| | | TGDNS | | | ESNFG | | | TCM |
| | | PLHF | | | NEKLT | | | |
| | | (SEQ ID | | | F | | | |
| | | NO: | | | (SEQ ID | | | |
| | | 126) | | | NO: | | | |
| | | | | | 440) | | | |
| | TRBV3-1 | CASSR | TRBJ1-2 | TRAV26-1 | CIVRV | TRAJ52 | G13D | CD8 |
| | | TGGGR | | | AGGTS | | | TEM |
| | | GYTF | | | YGKLT | | | |
| | | (SEQ ID | | | F | | | |
| | | NO: | | | (SEQ ID | | | |
| | | 127) | | | NO: | | | |
| | | | | | 441) | | | |
| | TRBV5-6 | CASSS | TRBJ1-6 | TRAV13-2 | CAEMN | TRAJ39 | G12R | CD4 |
| | | ENSPL | | | NAGNM | | | TCM |
| | | HF | | | LTF | | | |
| | | (SEQ ID | | | (SEQ ID | | | |
| | | NO: | | | NO: | | | |
| | | 128) | | | 442) | | | |
| | TRBV28 | CASSS | TRBJ1-1 | TRAV13-1 | CAASI | TRAJ42 | G12C; | NA |
| | | GGLNT | | | VGSQG | | G12V | |
| | | EAFF | | | NLIF | | | |
| | | (SEQ ID | | | (SEQ ID | | | |
| | | NO: | | | NO: | | | |
| | | 129) | | | 443) | | | |
| | TRBV5-1 | CASSS | TRBJ1-6 | TRAV26-1 | CIVRV | TRAJ48 | G13D | CD4 |
| | | GNSPL | | | GGISN | | | TCM |
| | | HF | | | FGNEK | | | |
| | | (SEQ ID | | | LTF | | | |
| | | NO: | | | (SEQ ID | | | |
| | | 130) | | | NO: | | | |
| | | | | | 444) | | | |
| | TRBV7-9 | CASSS | TRBJ1-3 | TRAV21 | CAVET | TRAJ58 | G13D | NA |
| | | LQVNS | | | SGSRL | | | |
| | | GNTIY | | | TF | | | |
| | | F | | | (SEQ ID | | | |
| | | (SEQ ID | | | NO: | | | |
| | | NO: | | | 445) | | | |
| | | 131) | | | | | | |
| | TRBV12-4 | CASSS | TRBJ1-2 | TRAV9-2 | CALRA | TRAJ52 | G12D | CD4 |
| | | LTPGY | | | GGTSY | | | TCM |
| | | GYTF | | | GKLTF | | | |
| | | (SEQ ID | | | (SEQ ID | | | |
| | | NO: | | | NO: | | | |
| | | 132) | | | 446) | | | |
| | TRBV28 | CASSS | TRBJ1-4 | TRAV13-1 | CAAST | TRAJ4 | G12A; | CD4 |
| | | PTLST | | | GGYNK | | G12C; | TCM |
| | | NEKLF | | | LIF | | G12D; | |
| | | F | | | (SEQ ID | | G13D | |
| | | (SEQ ID | | | NO: | | | |
| | | NO: | | | 447) | | | |
| | | 133) | | | | | | |
| | TRBV12-3 | CASSS | TRBJ2-7 | TRAV38-2/ | CAYSG | TRAJ41 | G12D | CD8 |
| | | RTGYY | | DV8 | DGYAL | | | TCM |
| | | EQYF | | | NF | | | |
| | | (SEQ ID | | | (SEQ ID | | | |
| | | NO: | | | NO: | | | |
| | | 134) | | | 448) | | | |
| | TRBV7-6 | CASSS | TRBJ1-2 | TRAV17 | CATDA | TRAJ48 | G12R | NA |
| | | SQRTM | | | RGDFG | | | |
| | | DGYTF | | | NEKLT | | | |
| | | (SEQ ID | | | F | | | |
| | | NO: | | | (SEQ ID | | | |
| | | 135) | | | NO: | | | |
| | | | | | 449) | | | |
| | TRBV28 | CASSS | TRBJ2-1 | TRAV17 | CATDK | TRAJ33 | G12V | NA |
| | | TGTGP | | | GSNYQ | | | |
| | | FF | | | LIW | | | |
| | | (SEQ ID | | | (SEQ ID | | | |
| | | NO: | | | NO: | | | |
| | | 136) | | | 450) | | | |
| | TRBV5-1 | CASSS | TRBJ2-7 | TRAV23/ | CAAST | TRAJ49 | G12A; | CD4 |
| | | VWGQG | | DV6 | GNQFY | | G12C; | TCM |
| | | GEQYF | | | F | | G12D; | |
| | | (SEQ ID | | | (SEQ ID | | G12R; | |
| | | NO: | | | NO: | | G12V; | |
| | | 137) | | | 451) | | G13D | |
| | TRBV3-1 | CASST | TRBJ1-6 | TRAV4 | CLVGV | TRAJ36 | G12A; | NA |
| | | GGWGP | | | DQTGA | | G13D | |
| | | NSPLH | | | NNLFF | | | |
| | | F | | | (SEQ ID | | | |
| | | (SEQ ID | | | NO: | | | |
| | | NO: | | | 452) | | | |
| | | 138) | | | | | | |
| | TRBV5-5 | CASST | TRBJ2-5 | TRAV8-4 | CAVSV | TRAJ33 | G12A; | CD4 |
| | | GPQET | | | PGSNY | | G12D | TCM |
| | | QYF | | | QLIW | | | |
| | | (SEQ ID | | | (SEQ ID | | | |
| | | NO: | | | NO: | | | |
| | | 139) | | | 453) | | | |
| | TRBV18 | CASST | TRBJ1-4 | TRAV17 | CAANN | TRAJ39 | G12A | CD4 |
| | | QENEK | | | NAGNM | | | TCM |
| | | LFF | | | LTF | | | |
| | | (SEQ ID | | | (SEQ ID | | | |
| | | NO: | | | NO: | | | |
| | | 140) | | | 454) | | | |
| | TRBV12-3 | CASST | TRBJ1-4 | TRAV29/ | CAGRN | TRAJ41 | G12A; | CD4 |
| | | RTNQH | | DV5 | SGYAL | | G12C; | Prolif- |
| | | EKLFF | | | NF | | G12D; | erating |
| | | (SEQ ID | | | (SEQ ID | | G12R; | |
| | | NO: | | | NO: | | G12V | |
| | | 141) | | | 455) | | | |
| | TRBV28 | CASST | TRBJ1-3 | TRAV21 | CAVPY | TRAJ23 | G12A; | CD4 |
| | | TAAGN | | | LSGAG | | G12C | Prolif- |
| | | TIYF | | | SYQLT | | | erating |
| | | (SEQ ID | | | F | | | |
| | | NO: | | | (SEQ ID | | | |
| | | 142) | | | NO: | | | |
| | | | | | 456) | | | |
| | TRBV9 | CASSV | TRBJ2-7 | TRAV8-4 | CAVSP | TRAJ13 | G12A; | CD4 |
| | | GGLAS | | | NSGGY | | G12V | TCM |
| | | SYEQY | | | QKVTF | | | |
| | | F | | | (SEQ ID | | | |
| | | (SEQ ID | | | NO: | | | |
| | | NO: | | | 457) | | | |
| | | 143) | | | | | | |
| | TRBV9 | CASSV | TRBJ2-5 | TRAV23/ | CAAST | TRAJ31 | G12A; | NA |
| | | GLAGS | | DV6 | PNNNA | | G12C; | |
| | | QETQY | | | RLMF | | G12D; | |
| | | F | | | (SEQ ID | | G12V | |
| | | (SEQ ID | | | NO: | | | |
| | | NO: | | | 458) | | | |
| | | 144) | | | | | | |
| | TRBV28 | CASSW | TRBJ1-4 | TRAV8-3 | CVVEP | TRAJ26 | G12A; | CD4 |
| | | GMPNE | | | GNYGQ | | G12C | Prolif- |
| | | KLFF | | | NFVF | | | erating |
| | | (SEQ ID | | | (SEQ ID | | | |
| | | NO: | | | NO: | | | |
| | | 145) | | | 459) | | | |
| | TRBV6-2 | CASSW | TRBJ1-5 | TRAV12-3 | CAMSA | TRAJ9 | G12A | CD4 |
| | | GSNQP | | | STGGF | | | TCM |
| | | QHF | | | KTIF | | | |
| | | (SEQ ID | | | (SEQ ID | | | |
| | | NO: | | | NO: | | | |
| | | 146) | | | 460) | | | |
| | TRBV5-1 | CASSW | TRBJ2-5 | TRAV13-2 | CAENM | TRAJ12 | G12R; | CD4 |
| | | TPAGE | | | MDSSY | | G13D | TCM |
| | | TQYF | | | KLIF | | | |
| | | (SEQ ID | | | (SEQ ID | | | |
| | | NO: | | | NO: | | | |
| | | 147) | | | 461) | | | |
| | TRBV27 | CASSY | TRBJ2-1 | TRAV29/ | CAASG | TRAJ33 | G12D | CD8 |
| | | PSGAF | | DV5 | NFGNE | | | TCM |
| | | GNEQF | | | KLTF | | | |
| | | F | | | (SEQ ID | | | |
| | | (SEQ ID | | | NO: | | | |
| | | NO: | | | 462) | | | |
| | | 148) | | | | | | |
| | TRBV6-5 | CASSY | TRBJ1-5 | TRAV21 | CAVPS | TRAJ52 | G12A; | CD4 |
| | | RGAGQ | | | NAGGT | | G12C | TCM |
| | | PQHF | | | SYGKL | | | |
| | | (SEQ ID | | | TF | | | |
| | | NO: | | | (SEQ ID | | | |
| | | 149) | | | NO: | | | |
| | | | | | 463) | | | |
| | TRBV28 | CASSY | TRBJ2-7 | TRAV1-2 | CAVRD | TRAJ23 | G12C | CD4 |
| | | SYEQY | | | GAGSY | | | TCM |
| | | F | | | QLTF | | | |
| | | (SEQ ID | | | (SEQ ID | | | |
| | | NO: | | | NO: | | | |
| | | 150) | | | 464) | | | |
| | TRBV6-6 | CASSY | TRBJ1-1 | TRAV19 | CALTG | TRAJ12 | G13D | CD8 |
| | | TTEAF | | | MMDSS | | | TCM |
| | | F | | | YKLIF | | | |
| | | (SEQ ID | | | (SEQ ID | | | |
| | | NO: | | | NO: | | | |
| | | 151) | | | 465) | | | |
| | TRBV19 | CASTP | TRBJ2-6 | TRAV38-2/ | CAYRT | TRAJ43 | G12A | CD8 |
| | | GSGAN | | DV8 | PPNDM | | | Naive |
| | | VLTF | | | RF | | | |
| | | (SEQ ID | | | (SEQ ID | | | |
| | | NO: | | | NO: | | | |
| | | 152) | | | 466) | | | |
| | TRBV10-2 | CASTP | TRBJ1-6 | TRAV29/ | CAASA | TRAJ12 | G12A; | NA |
| | | SQGHN | | DV5 | TDSSY | | G12V | |
| | | SPLHF | | | KLIF | | | |
| | | (SEQ ID | | | (SEQ ID | | | |
| | | NO: | | | NO: | | | |
| | | 153) | | | 467) | | | |
| | TRBV2 | CASRR | TRBJ1-2 | TRAV8-1 | CAVNA | TRAJ48 | G12R | CD4 |
| | | NGLYY | | | PFGNE | | | Prolif- |
| | | TF | | | KLTF | | | erating |
| | | (SEQ ID | | | (SEQ ID | | | |
| | | NO: | | | NO: | | | |
| | | 154) | | | 468) | | | |
| | TRBV24-1 | CATSD | TRBJ1-1 | TRAV13-1 | CAASR | TRAJ5 | G12R | NA |
| | | DSGQG | | | TGRRA | | | |
| | | AEAFF | | | LTF | | | |
| | | (SEQ ID | | | (SEQ ID | | | |
| | | NO: | | | NO: | | | |
| | | 155) | | | 469) | | | |
| | TRBV24-1 | CATSD | TRBJ2-1 | TRAV12-3 | CAHTS | TRAJ6 | G12A; | NA |
| | | MGLAD | | | SGGSY | | G12C; | |
| | | NEQFF | | | IPTF | | G12V | |
| | | (SEQ ID | | | (SEQ ID | | | |
| | | NO: | | | NO: | | | |
| | | 156) | | | 470) | | | |
| | TRBV24-1 | CATSD | TRBJ2-1 | TRAV13-1 | CAASD | TRAJ33 | G12R; | NA |
| | | PSGPN | | | SNYQL | | G12V | |
| | | YNEQF | | | IW | | | |
| | | F | | | (SEQ ID | | | |
| | | (SEQ ID | | | NO: | | | |
| | | NO: | | | 471) | | | |
| | | 157) | | | | | | |
| | TRBV24-1 | CATSE | TRBJ1-2 | TRAV8-3 | CAVGA | TRAJ43 | G12A | CD4 |
| | | GGQGG | | | YNNND | | | TCM |
| | | YGYTF | | | MRF | | | |
| | | (SEQ ID | | | (SEQ ID | | | |
| | | NO: | | | NO: | | | |
| | | 158) | | | 472) | | | |
| | TRBV24-1 | CATSG | TRBJ2-7 | TRAV25 | CAGFN | TRAJ41 | G12R | NA |
| | | GGAYE | | | SGYAL | | | |
| | | QYF | | | NF | | | |
| | | (SEQ ID | | | (SEQ ID | | | |
| | | NO: | | | NO: | | | |
| | | 159) | | | 473) | | | |
| | TRBV15 | CATSQ | TRBJ1-6 | TRAV8-6 | CAVSS | TRAJ56 | G12A; | NA |
| | | ERRQV | | | TGANS | | G12V | |
| | | GSPLH | | | KLTF | | | |
| | | F | | | (SEQ ID | | | |
| | | (SEQ ID | | | NO: | | | |
| | | NO: | | | 474) | | | |
| | | 160) | | | | | | |
| | TRBV30 | CAWSA | TRBJ2-2 | TRAV21 | CAFIL | TRAJ28 | G12A | CD4 |
| | | LAGSW | | | PSGAG | | | Prolif- |
| | | AGELF | | | SYQLT | | | erating |
| | | F | | | F | | | |
| | | (SEQ ID | | | (SEQ ID | | | |
| | | NO: | | | NO: | | | |
| | | 161) | | | 475) | | | |
| | TRBV29-1 | CSAAG | TRBJ1-1 | TRAV12-3 | CAMSE | TRAJ26 | G12R | CD4 |
| | | TGNTE | | | PNGQN | | | TCM |
| | | AFF | | | FVF | | | |
| | | (SEQ ID | | | (SEQ ID | | | |
| | | NO: | | | NO: | | | |
| | | 162) | | | 476) | | | |
| | TRBV20-1 | CSAAR | TRBJ1-2 | TRAV12-2 | CAPRD | TRAJ11 | G12C | NA |
| | | QRTNY | | | SGYST | | | |
| | | GYTF | | | LTF | | | |
| | | (SEQ ID | | | (SEQ ID | | | |
| | | NO: | | | NO: | | | |
| | | 163) | | | 477) | | | |
| | TRBV20-1 | CSADR | TRBJ2-4 | TRAV20 | CAVRR | TRAJ39 | G12A; | CD4 |
| | | TSAKN | | | NAGNM | | G12C | TCM |
| | | IQYF | | | LTF | | | |
| | | (SEQ ID | | | (SEQ ID | | | |
| | | NO: | | | NO: | | | |
| | | 164) | | | 478) | | | |
| | TRBV20-1 | CSAFR | TRBJ2-7 | TRAV5 | CAESH | TRAJ34 | G12R | NA |
| | | LAAQG | | | NTDKL | | | |
| | | GSYEQ | | | IF | | | |
| | | YF | | | (SEQ ID | | | |
| | | (SEQ ID | | | NO: | | | |
| | | NO: | | | 479) | | | |
| | | 165) | | | | | | |
| | TRBV20-1 | CSAIR | TRBJ2-7 | TRAV10 | CVVGT | TRAJ44 | G12R | NA |
| | | PGVGD | | | GTASK | | | |
| | | YEQYF | | | LTF | | | |
| | | (SEQ ID | | | (SEQ ID | | | |
| | | NO: | | | NO: | | | |
| | | 166) | | | 480) | | | |
| TCR06 | TRBV20-1 | CSAKS | TRBJ2-7 | TRAV12-3 | CAMKT | TRAJ10 | G12R | CD8 |
| (Validated) | | TGYDY | | | GGGNK | | | Prolif- |
| | | EQYF | | | LTF | | | erating |
| | | (SEQ ID | | | (SEQ ID | | | |
| | | NO: | | | NO: | | | |
| | | 167) | | | 481) | | | |
| | TRBV20-1 | CSALS | TRBJ2-4 | TRAV12-1 | CVVKL | TRAJ3 | G12A; | CD4 |
| | | QSGGT | | | NSSAS | | G12C; | TCM |
| | | NIQYF | | | KIIF | | G12D; | |
| | | (SEQ ID | | | (SEQ ID | | G12V | |
| | | NO: | | | NO: | | | |
| | | 168) | | | 482) | | | |
| | TRBV20-1 | CSALW | TRBJ2-1 | TRAV23/ | CAASL | TRAJ21 | G12A; | NA |
| | | SGDGE | | DV6 | NFNKF | | G12C; | |
| | | QFF | | | YF | | G12R; | |
| | | (SEQ ID | | | (SEQ ID | | G12V; | |
| | | NO: | | | NO: | | G13D | |
| | | 169) | | | 483) | | | |
| | TRBV20-1 | CSAMT | TRBJ1-5 | TRAV3 | CAVRD | TRAJ11 | G12C | NA |
| | | REGGN | | | GGGYS | | | |
| | | QPQHF | | | TLTF | | | |
| | | (SEQ ID | | | (SEQ ID | | | |
| | | NO: | | | NO: | | | |
| | | 170) | | | 484) | | | |
| | TRBV20-1 | CSANP | TRBJ2-7 | TRAV9-2 | CASRF | TRAJ4 | G12V | CD4 |
| | | LAGGG | | | SGGYN | | | Prolif- |
| | | EQYF | | | KLIF | | | erating |
| | | (SEQ ID | | | (SEQ ID | | | |
| | | NO: | | | NO: | | | |
| | | 171) | | | 485) | | | |
| | TRBV20-1 | CSAPG | TRBJ2-2 | TRAV8-4 | CAVSD | TRAJ4 | G13D | NA |
| | | PAAAG | | | PGGYN | | | |
| | | ELFF | | | KLIF | | | |
| | | (SEQ ID | | | (SEQ ID | | | |
| | | NO: | | | NO: | | | |
| | | 172) | | | 486) | | | |
| | TRBV20-1 | CSAPG | TRBJ2-6 | TRAV38-2/ | CAVSE | TRAJ13 | G12A; | CD4 |
| | | TSAGA | | DV8 | PGGYQ | | G12C; | TCM |
| | | NVLTF | | | KVTF | | G12D; | |
| | | (SEQ ID | | | (SEQ ID | | G12V; | |
| | | NO: | | | NO: | | G13D | |
| | | 173) | | | 487) | | | |
| | TRBV20-1 | CSAPK | TRBJ1-3 | TRAV3 | CAFRS | TRAJ23 | G12A | CD4 |
| | | LVGSG | | | NNNDM | | | TCM |
| | | NTIYF | | | RF | | | |
| | | (SEQ ID | | | (SEQ ID | | | |
| | | NO: | | | NO: | | | |
| | | 174) | | | 488) | | | |
| | TRBV20-1 | CSAPQ | TRBJ2-1 | TRAV12-2 | CAVWG | TRAJ15 | G13D | CD8 |
| | | DRNNE | | | VNQAG | | | TEM |
| | | QFF | | | TALIF | | | |
| | | (SEQ ID | | | (SEQ ID | | | |
| | | NO: | | | NO: | | | |
| | | 175) | | | 489) | | | |
| | TRBV20-1 | CSAPS | TRBJ1-2 | TRAV30 | CGTPS | TRAJ13 | G12D; | CD4 |
| | | TDRVR | | | GGYQK | | G13D | TCM |
| | | GYTF | | | VTF | | | |
| | | (SEQ ID | | | (SEQ ID | | | |
| | | NO: | | | NO: | | | |
| | | 176) | | | 490) | | | |
| | TRBV20-1 | CSARD | TRBJ2-1 | TRAV13-1 | CAASQ | TRAJ17 | G12R | NA |
| | | HTSGS | | | AAGNK | | | |
| | | GNEQF | | | LTF | | | |
| | | F | | | (SEQ ID | | | |
| | | (SEQ ID | | | NO: | | | |
| | | NO: | | | 491) | | | |
| | | 177) | | | | | | |
| | TRBV20-1 | CSARD | TRBJ2-7 | TRAV13-1 | CAASM | TRAJ49 | G12C; | NA |
| | | PDRGS | | | GTGNQ | | G13D | |
| | | GNEQY | | | FYF | | | |
| | | F | | | (SEQ ID | | | |
| | | (SEQ ID | | | NO: | | | |
| | | NO: | | | 492) | | | |
| | | 178) | | | | | | |
| | TRBV20-1 | CSARD | TRBJ1-6 | TRAV19 | CALSE | TRAJ33 | G12D | CD4 |
| | | QGALL | | | VYNND | | | TEM |
| | | NSPLH | | | MRF | | | |
| | | F | | | (SEQ ID | | | |
| | | (SEQ ID | | | NO: | | | |
| | | NO: | | | 493) | | | |
| | | 179) | | | | | | |
| | TRBV20-1 | CSARD | TRBJ1-1 | TRAV8-2 | CALHW | TRAJ52 | G13D | CD4 |
| | | RGGNT | | | RGAQK | | | TCM |
| | | EAFF | | | LVF | | | |
| | | (SEQ ID | | | (SEQ ID | | | |
| | | NO: | | | NO: | | | |
| | | 180) | | | 494) | | | |
| | TRBV20-1 | CSARD | TRBJ2-1 | TRAV3 | CAVRD | TRAJ15 | G12R; | CD4 |
| | | RVGGE | | | QAGTA | | G12V | Prolif- |
| | | QFF | | | LIF | | | erating |
| | | (SEQ ID | | | (SEQ ID | | | |
| | | NO: | | | NO: | | | |
| | | 181) | | | 495) | | | |
| | TRBV20-1 | CSARD | TRBJ1-1 | TRAV13-1 | CAASM | TRAJ49 | G12V | CD4 |
| | | VRLNT | | | AAGNQ | | | TCM |
| | | EAFF | | | FYF | | | |
| | | (SEQ ID | | | (SEQ ID | | | |
| | | NO: | | | NO: | | | |
| | | 182) | | | 496) | | | |
| | TRBV20-1 | CSARD | TRBJ2-1 | TRAV13-1 | CAAST | TRAJ39 | G12R; | CD4 |
| | | VWGTG | | | GAGNM | | G12V | Prolif- |
| | | NSQAS | | | LTF | | | erating |
| | | GNEQF | | | (SEQ ID | | | |
| | | F | | | NO: | | | |
| | | (SEQ ID | | | 497) | | | |
| | | NO: | | | | | | |
| | | 183) | | | | | | |
| | TRBV20-1 | CSARG | TRBJ2-3 | TRAV21 | CAVRD | TRAJ49 | G12A; | CD4 |
| | | LAGAD | | | TGNQF | | G12V | TCM |
| | | TQYF | | | YF | | | |
| | | (SEQ ID | | | (SEQ ID | | | |
| | | NO: | | | NO: | | | |
| | | 184) | | | 498) | | | |
| | TRBV20-1 | CSARG | TRBJ1-1 | TRAV8-1 | CAVKG | TRAJ12 | G12R | CD4 |
| | | PGGNT | | | SNTGK | | | TCM |
| | | EAFF | | | LIF | | | |
| | | (SEQ ID | | | (SEQ ID | | | |
| | | NO: | | | NO: | | | |
| | | 185) | | | 499) | | | |
| | TRBV20-1 | CSARG | TRBJ2-3 | TRAV12-2 | CATHP | TRAJ20 | G12A; | NA |
| | | PGTDT | | | NSGYA | | G12C; | |
| | | QYF | | | LNF | | G12R; | |
| | | (SEQ ID | | | (SEQ ID | | G12V | |
| | | NO: | | | NO: | | | |
| | | 186) | | | 500) | | | |
| | TRBV20-1 | CSARG | TRBJ1-5 | TRAV35 | CAGLN | TRAJ49 | G13D | CD4 |
| | | RQDQP | | | TGNQF | | | TCM |
| | | QHF | | | YF | | | |
| | | (SEQ ID | | | (SEQ ID | | | |
| | | NO: | | | NO: | | | |
| | | 187) | | | 501) | | | |
| | TRBV20-1 | CSART | TRBJ1-2 | TRAV9-2 | CALSD | TRAJ31 | G12V | NA |
| | | ITGSG | | | QNARL | | | |
| | | YTF | | | MF | | | |
| | | (SEQ ID | | | (SEQ ID | | | |
| | | NO: | | | NO: | | | |
| | | 188) | | | 502) | | | |
| | TRBV20-1 | CSART | TRBJ2-1 | TRAV26-1 | CIVRV | TRAJ29 | G12D | CD4 |
| | | SGRGY | | | SNSGN | | | TCM |
| | | NEQFF | | | TPLVF | | | |
| | | (SEQ ID | | | (SEQ ID | | | |
| | | NO: | | | NO: | | | |
| | | 189) | | | 503) | | | |
| | TRBV20-1 | CSARV | TRBJ2-1 | TRAV9-2 | CALSD | TRAJ11 | G12A; | CD4 |
| | | LGAGP | | | PQAAL | | G12C; | Prolif- |
| | | NNEQF | | | TF | | G12D; | erating |
| | | F | | | (SEQ ID | | G12V; | |
| | | (SEQ ID | | | NO: | | G13D | |
| | | NO: | | | 504) | | | |
| | | 190) | | | | | | |
| | TRBV20-1 | CSARV | TRBJ2-3 | TRAV8-2 | CVVSD | TRAJ10 | G12V | CD4 |
| | | SAVST | | | RPGGG | | | TCM |
| | | DTQYF | | | NKLTF | | | |
| | | (SEQ ID | | | (SEQ ID | | | |
| | | NO: | | | NO: | | | |
| | | 191) | | | 505) | | | |
| | TRBV20-1 | CSASP | TRBJ2-7 | TRAV12-3 | CAMRT | TRAJ10 | G12R | CD4 |
| | | LAGGS | | | GGGNK | | | Prolif- |
| | | YEQYF | | | LTF | | | erating |
| | | (SEQ ID | | | (SEQ ID | | | |
| | | NO: | | | NO: | | | |
| | | 192) | | | 506) | | | |
| | TRBV20-1 | CSASP | TRBJ2-6 | TRAV8-6 | CAVSE | TRAJ4 | G12C; | NA |
| | | LKAGA | | | PGGYN | | G12V | |
| | | NVLTF | | | KLIF | | | |
| | | (SEQ ID | | | (SEQ ID | | | |
| | | NO: | | | NO: | | | |
| | | 193) | | | 507) | | | |
| | TRBV20-1 | CSASR | TRBJ1-5 | TRAV26-1 | CIVKN | TRAJ15 | G12R | NA |
| | | DSNQP | | | TGTAL | | | |
| | | QHF | | | IF | | | |
| | | (SEQ ID | | | (SEQ ID | | | |
| | | NO: | | | NO: | | | |
| | | 194) | | | 508) | | | |
| | TRBV20-1 | CSASS | TRBJ1-5 | TRAV8-3 | CAVGG | TRAJ18 | G12A; | NA |
| | | DRGGN | | | RGSTL | | G12C; | |
| | | QPQHF | | | GRLYF | | G12V | |
| | | (SEQ ID | | | (SEQ ID | | | |
| | | NO: | | | NO: | | | |
| | | 195) | | | 509) | | | |
| | TRBV20-1 | CSASS | TRBJ1-2 | TRAV12-3 | CAMSN | TRAJ21 | G12D | CD4 |
| | | GTVGG | | | NFNKF | | | TCM |
| | | YTF | | | YF | | | |
| | | (SEQ ID | | | (SEQ ID | | | |
| | | NO: | | | NO: | | | |
| | | 196) | | | 510) | | | |
| | TRBV20-1 | CSASS | TRBJ2-1 | TRAV19 | CAPPR | TRAJ4 | G12C | CD4 |
| | | GVSSY | | | GTGGY | | | TCM |
| | | NEQFF | | | NKLIF | | | |
| | | (SEQ ID | | | (SEQ ID | | | |
| | | NO: | | | NO: | | | |
| | | 197) | | | 511) | | | |
| | TRBV20-1 | CSATR | TRBJ2-2 | TRAV8-4 | CAVSE | TRAJ13 | G12A; | NA |
| | | FGQAN | | | PGGYQ | | G12C; | |
| | | TGELF | | | KVTF | | G12D; | |
| | | F | | | (SEQ ID | | G12V; | |
| | | (SEQ ID | | | NO: | | G13D | |
| | | NO: | | | 512) | | | |
| | | 198) | | | | | | |
| | TRBV20-1 | CSATT | TRBJ1-1 | TRAV3 | CAVRD | TRAJ4 | G12A; | NA |
| | | WTGGN | | | SGGYN | | G12V | |
| | | TEAFF | | | KLIF | | | |
| | | (SEQ ID | | | (SEQ ID | | | |
| | | NO: | | | NO: | | | |
| | | 199) | | | 513) | | | |
| TCR04 | TRBV20-1 | CSAVD | TRBJ2-7 | TRAV8-6 | CAVSE | TRAJ13 | G12R | CD4 |
| (Validated) | | WTSGS | | | SGGYQ | | | TCM |
| | | SYEQY | | | KVTF | | | |
| | | V | | | (SEQ ID | | | |
| | | (SEQ ID | | | NO: | | | |
| | | NO: | | | 514) | | | |
| | | 200) | | | | | | |
| | TRBV29-1 | CSAVL | TRBJ2-3 | TRAV21 | CAVRG | TRAJ16 | G12C; | NA |
| | | GLAGV | | | FSDGQ | | G12R | |
| | | RDTQY | | | KLLF | | | |
| | | F | | | (SEQ ID | | | |
| | | (SEQ ID | | | NO: | | | |
| | | NO: | | | 515) | | | |
| | | 201) | | | | | | |
| | TRBV20-1 | CSISP | TRBJ1-5 | TRAV1-2 | CARRG | TRAJ22 | G12A; | CD4 |
| | | DRGGN | | | SSGSA | | G12C; | TEM |
| | | QPQHF | | | RQLTF | | G12V; | |
| | | (SEQ ID | | | (SEQ ID | | G13D | |
| | | NO: | | | NO: | | | |
| | | 202) | | | 516) | | | |
| | TRBV20-1 | CSLVP | TRBJ1-5 | TRAV1-2 | CAVPY | TRAJ27 | G12A; | CD4 |
| | | DRGGN | | | LTNAG | | G12C; | TCM |
| | | QPQHF | | | KSTF | | G12D | |
| | | (SEQ ID | | | (SEQ ID | | | |
| | | NO: | | | NO: | | | |
| | | 203) | | | 517) | | | |
| | TRBV29-1 | CSRGG | TRBJ2-1 | TRAV21 | CAVEE | TRAJ43 | G12C | NA |
| | | REGEQ | | | TSGSR | | | |
| | | FF | | | LTF | | | |
| | | (SEQ ID | | | (SEQ ID | | | |
| | | NO: | | | NO: | | | |
| | | 204) | | | 518) | | | |
| | TRBV29-1 | CSVEG | TRBJ2-7 | TRAV35 | CAGQA | TRAJ40 | G12V | NA |
| | | QATYE | | | AYKYI | | | |
| | | QYF | | | F | | | |
| | | (SEQ ID | | | (SEQ ID | | | |
| | | NO: | | | NO: | | | |
| | | 205) | | | 519) | | | |
| | TRBV29-1 | CSVEG | TRBJ1-2 | TRAV21 | CAVSQ | TRAJ23 | G12V; | CD4 |
| | | QGNYG | | | AWGGK | | G13D | TCM |
| | | YTF | | | LIF | | | |
| | | (SEQ ID | | | (SEQ ID | | | |
| | | NO: | | | NO: | | | |
| | | 206) | | | 520) | | | |
| | TRBV29-1 | CSVLG | TRBJ2-7 | TRAV13-1 | CAGTN | TRAJ29 | G12A; | CD4 |
| | | QGAPR | | | TDKLI | | G12C; | TCM |
| | | SYEQY | | | F | | G12V | |
| | | F | | | (SEQ ID | | | |
| | | (SEQ ID | | | NO: | | | |
| | | NO: | | | 521) | | | |
| | | 207) | | | | | | |
| | TRBV20-1 | CSVRG | TRBJ2-7 | TRAV5 | CAESI | TRAJ34 | G12A | NA |
| | | RANEQ | | | GTDKL | | | |
| | | YF | | | IF | | | |
| | | (SEQ ID | | | (SEQ ID | | | |
| | | NO: | | | NO: | | | |
| | | 208) | | | 522) | | | |
| | TRBV18 | CASAR | TRBJ2-5 | TRAV21 | CAVLF | TRAJ48 | G12R | NA |
| | | TGQET | | | GNEKL | | | |
| | | QYF | | | TF | | | |
| | | (SEQ ID | | | (SEQ ID | | | |
| | | NO: | | | NO: | | | |
| | | 209) | | | 523) | | | |
| | TRBV6-6 | CASNL | TRBJ2-2 | TRAV36/ | CAVDD | TRAJ45 | G12A | CD4 |
| | | PRSGE | | DV7 | SGGGA | | | TCM |
| | | LFF | | | DGLTF | | | |
| | | (SEQ ID | | | (SEQ ID | | | |
| | | NO: | | | NO: | | | |
| | | 210) | | | 524) | | | |
| | TRBV5-1 | CASRP | TRBJ2-7 | TRAV13-1 | CAASW | TRAJ52 | G12A | CD4 |
| | | ELDSY | | | GGTSY | | | TCM |
| | | EQYF | | | GKLTF | | | |
| | | (SEQ ID | | | (SEQ ID | | | |
| | | NO: | | | NO: | | | |
| | | 211) | | | 525) | | | |
| | TRBV2 | CASRR | TRBJ1-5 | TRAV23/ | CAPIL | TRAJ23 | G12A; | CD4 |
| | | GGISN | | DV6 | QGAQK | | G12C; | TCM |
| | | QPQHF | | | LVF | | G12V | |
| | | (SEQ ID | | | (SEQ ID | | | |
| | | NO: | | | NO: | | | |
| | | 212) | | | 526) | | | |
| | TRBV6-4 | CASSE | TRBJ1-2 | TRAV26-2 | CILRD | TRAJ26 | G12C | CD8 |
| | | HGGNY | | | NYGQN | | | TEM |
| | | GYTF | | | FVF | | | |
| | | (SEQ ID | | | (SEQ ID | | | |
| | | NO: | | | NO: | | | |
| | | 213) | | | 527) | | | |
| | TRBV19 | CASSI | TRBJ1-5 | TRAV39 | CAVAG | TRAJ44 | G12A | CD4 |
| | | FTLSN | | | TASKL | | | TCM |
| | | QPQHF | | | TF | | | |
| | | (SEQ ID | | | (SEQ ID | | | |
| | | NO: | | | NO: | | | |
| | | 214) | | | 528) | | | |
| TCR07 | TRBV6-5 | CASSK | TRBJ1-1 | TRAV21 | CAVYS | TRAJ37 | G12V | CD8 |
| (Validated) | | QGATE | | | NTGKL | | | TEM |
| | | AFF | | | IF | | | |
| | | (SEQ ID | | | (SEQ ID | | | |
| | | NO: | | | NO: | | | |
| | | 215) | | | 529) | | | |
| | TRBV7-2 | CASSL | TRBJ1-2 | TRAV21 | CAVNT | TRAJ49 | G12C | CD4 |
| | | GANYG | | | GNQFY | | | TCM |
| | | YTF | | | F | | | |
| | | (SEQ ID | | | (SEQ ID | | | |
| | | NO: | | | NO: | | | |
| | | 216) | | | 530) | | | |
| | TRBV18 | CASSL | TRBJ2-7 | TRAV22 | CAVLL | TRAJ10 | G12A; | CD4 |
| | | TDLYE | | | TGGGN | | G12C | TCM |
| | | QYF | | | KLTF | | | |
| | | (SEQ ID | | | (SEQ ID | | | |
| | | NO: | | | NO: | | | |
| | | 217) | | | 531) | | | |
| | TRBV5-4 | CASSP | TRBJ2-7 | TRAV26-1 | CIVGN | TRAJ9 | G12A; | CD4 |
| | | DRVEQ | | | TGGFK | | G12C; | TCM |
| | | YF | | | TIF | | G12D; | |
| | | (SEQ ID | | | (SEQ ID | | G12R; | |
| | | NO: | | | NO: | | G12V; | |
| | | 218) | | | 532) | | G13D | |
| | TRBV6-2 | CASSP | TRBJ2-7 | TRAV29/ | CAASV | TRAJ57 | G12D; | CD8 |
| | | PGGTE | | DV5 | WGGSE | | G12V | TEM |
| | | VYEQY | | | KLVF | | | |
| | | F | | | (SEQ ID | | | |
| | | (SEQ ID | | | NO: | | | |
| | | NO: | | | 533) | | | |
| | | 219) | | | | | | |
| | TRBV19 | CASSP | TRBJ2-2 | TRAV9-2 | CALSD | TRAJ47 | G12A; | CD4 |
| | | PPGRA | | | RGGNK | | G12C; | TCM |
| | | ETGEL | | | LVF | | G12R; | |
| | | FF | | | (SEQ ID | | G12V | |
| | | (SEQ ID | | | NO: | | | |
| | | NO: | | | 534) | | | |
| | | 220) | | | | | | |
| | TRBV6-5 | CASSR | TRBJ2-2 | TRAV12-1 | CAARE | TRAJ34 | G12A; | CD4 |
| | | GAGEL | | | TYNTD | | G12C; | TCM |
| | | FF | | | KLIF | | G12V | |
| | | (SEQ ID | | | (SEQ ID | | | |
| | | NO: | | | NO: | | | |
| | | 221) | | | 535) | | | |
| | TRBV9 | CASSV | TRBJ1-2 | TRAV8-3 | CGSPG | TRAJ28 | G12A | CD8 |
| | | GGDYG | | | AGSYQ | | | TEM |
| | | YTF | | | LTF | | | |
| | | (SEQ ID | | | (SEQ ID | | | |
| | | NO: | | | NO: | | | |
| | | 222) | | | 536) | | | |
| | TRBV6-2 | CASSY | TRBJ1-1 | TRAV12-1 | CAGGN | TRAJ38 | G12A; | CD4 |
| | | GTANT | | | AGNNR | | G12C; | TCM |
| | | EAFF | | | KLIW | | G12R | |
| | | (SEQ ID | | | (SEQ ID | | | |
| | | NO: | | | NO: | | | |
| | | 223) | | | 537) | | | |
| | TRBV6-2 | CASSY | TRBJ2-7 | TRAV38-1 | CAFMM | TRAJ45 | G12A | CD4 |
| | | STLAG | | | LTGGG | | | TCM |
| | | GHSYE | | | ADGLT | | | |
| | | QYF | | | F | | | |
| | | (SEQ ID | | | (SEQ ID | | | |
| | | NO: | | | NO: | | | |
| | | 224) | | | 538) | | | |
| | TRBV28 | CASSY | TRBJ2-7 | TRAV1-2 | CAVRD | TRAJ23 | G12C | CD4 |
| | | SYEQY | | | GAGSY | | | TCM |
| | | F | | | QLTF | | | |
| | | (SEQ ID | | | (SEQ ID | | | |
| | | NO: | | | NO: | | | |
| | | 225) | | | 539) | | | |
| | TRBV29-1 | CSVAG | TRBJ1-6 | TRAV26-1 | CIVRV | TRAJ27 | G12A; | CD4 |
| | | QGNSP | | | EAGKS | | G12C; | TCM |
| | | LHF | | | TF | | G12D | |
| | | (SEQ ID | | | (SEQ ID | | | |
| | | NO: | | | NO: | | | |
| | | 226) | | | 540) | | | |
| | TRBV28 | CASGG | TRBJ2-3 | TRAV12-3 | CATIQ | TRAJ36 | G12R | CD4 |
| | | TTDTQ | | | TGANN | | | TCM |
| | | YF | | | LFF | | | |
| | | (SEQ ID | | | (SEQ ID | | | |
| | | NO: | | | NO: | | | |
| | | 227) | | | 541) | | | |
| | TRBV28 | CASRR | TRBJ2-2 | TRAV10 | CAGYN | TRAJ12 | G13D | CD4 |
| | | GNTGE | | | SGTYK | | | TCM |
| | | LFF | | | YIF | | | |
| | | (SEQ ID | | | (SEQ ID | | | |
| | | NO: | | | NO: | | | |
| | | 228) | | | 542) | | | |
| | TRBV28 | CASRS | TRBJ1-4 | TRAV20 | CAVQA | TRAJ31 | G12R | CD4 |
| | | TGTGE | | | VNNNA | | | TCM |
| | | KLFF | | | RLMF | | | |
| | | (SEQ ID | | | (SEQ ID | | | |
| | | NO: | | | NO: | | | |
| | | 229) | | | 543) | | | |
| | TRBV12-3 | CASSF | TRBJ2-3 | TRAV26-1 | CAFMN | TRAJ34 | G12R | NA |
| | | WAGVS | | | TDKLI | | | |
| | | TDTQY | | | F | | | |
| | | F | | | (SEQ ID | | | |
| | | (SEQ ID | | | NO: | | | |
| | | NO: | | | 544) | | | |
| | | 230) | | | | | | |
| | TRBV28 | CASSG | TRBJ2-3 | TRAV14/ | CAMRE | TRAJ4 | G13D | CD4 |
| | | GRKLD | | DV4 | GSGGY | | | TCM |
| | | TQYF | | | NKLIF | | | |
| | | (SEQ ID | | | (SEQ ID | | | |
| | | NO: | | | NO: | | | |
| | | 231) | | | 545) | | | |
| | TRBV11-2 | CASSL | TRBJ2-5 | TRAV14/ | CAMRP | TRAJ37 | G12R | CD4 |
| | | NLLDR | | DV4 | RSSNT | | | TCM |
| | | ASLET | | | GKLIF | | | |
| | | QYF | | | (SEQ ID | | | |
| | | (SEQ ID | | | NO: | | | |
| | | NO: | | | 546) | | | |
| | | 232) | | | | | | |
| | TRBV27 | CASSL | TRBJ1-6 | TRAV17 | CATDF | TRAJ48 | G13D | NA |
| | | TGYNS | | | FGNEK | | | |
| | | PLHF | | | LTF | | | |
| | | (SEQ ID | | | (SEQ ID | | | |
| | | NO: | | | NO: | | | |
| | | 233) | | | 547) | | | |
| | TRBV7-9 | CASSL | TRBJ2-1 | TRAV8-6 | CAVSH | TRAJ49 | G12A | CD4 |
| | | VLEHE | | | TGNQF | | | TCM |
| | | QFF | | | YF | | | |
| | | (SEQ ID | | | (SEQ ID | | | |
| | | NO: | | | NO: | | | |
| | | 234) | | | 548) | | | |
| | TRBV54 | CASSQ | TRBJ1-2 | TRAV36/ | CAELR | TRAJ54 | G12R | CD4 |
| | | DRITS | | DV7 | IQGAQ | | | TCM |
| | | GYGYT | | | KLVF | | | |
| | | F | | | (SEQ ID | | | |
| | | (SEQ ID | | | NO: | | | |
| | | NO: | | | 549) | | | |
| | | 235) | | | | | | |
| | TRBV28 | CASST | TRBJ1-5 | TRAV12-1 | CVVIF | TRAJ44 | G12R | CD4 |
| | | GGRSN | | | TGTAS | | | TCM |
| | | QPQHF | | | KLTF | | | |
| | | (SEQ ID | | | (SEQ ID | | | |
| | | NO: | | | NO: | | | |
| | | 236) | | | 550) | | | |
| | TRBV9 | CASSV | TRBJ2-1 | TRAV8-4 | CAVSG | TRAJ45 | G12A; | CD4 |
| | | VPGAG | | | LGGGA | | G12C; | TCM |
| | | GEQFF | | | DGLTF | | G12R; | |
| | | (SEQ ID | | | (SEQ ID | | G12V; | |
| | | NO: | | | NO: | | G13D | |
| | | 237) | | | 551) | | | |
| | TRBV9 | CASSV | TRBJ2-2 | TRAV16 | CALPD | TRAJ45 | G12A; | CD4 |
| | | VPGGP | | | SGGGA | | G12V | TCM |
| | | GGELF | | | DGLTF | | | |
| | | F | | | (SEQ ID | | | |
| | | (SEQ ID | | | NO: | | | |
| | | NO: | | | 552) | | | |
| | | 238) | | | | | | |
| | TRBV5-1 | CASSW | TRBJ2-1 | TRAV13-2 | CAENI | TRAJ11 | G12A; | CD4 |
| | | APHTD | | | KGSSG | | G12C; | CTL |
| | | EQFF | | | YSTLT | | G12R; | |
| | | (SEQ ID | | | F | | G12V; | |
| | | NO: | | | (SEQ ID | | G13D | |
| | | 239) | | | NO: | | | |
| | | | | | 553) | | | |
| | TRBV5-4 | CASSW | TRBJ2-2 | TRAV25 | CAGEG | TRAJ28 | G12R | CD4 |
| | | TGNTG | | | AGSYQ | | | TCM |
| | | ELFF | | | LTF | | | |
| | | (SEQ ID | | | (SEQ ID | | | |
| | | NO: | | | NO: | | | |
| | | 240) | | | 554) | | | |
| | TRBV6-6 | CASSY | TRBJ2-5 | TRAV8-3 | CAVGD | TRAJ33 | G12V | CD8 |
| | | TQETQ | | | SNYQL | | | TEM |
| | | YF | | | IW | | | |
| | | (SEQ ID | | | (SEQ ID | | | |
| | | NO: | | | NO: | | | |
| | | 241) | | | 555) | | | |
| | TRBV6-6 | CASSY | TRBJ2-7 | TRAV20 | CAVQG | TRAJ31 | G12C | CD4 |
| | | TTSGG | | | ALNNA | | | TCM |
| | | TYEQY | | | RLMF | | | |
| | | F | | | (SEQ ID | | | |
| | | (SEQ ID | | | NO: | | | |
| | | NO: | | | 556) | | | |
| | | 242) | | | | | | |
| | TRBV6-6 | CASTP | TRBJ1-5 | TRAV8-4 | CAVSD | TRAJ53 | G12C; | CD8 |
| | | SGGTQ | | | PLGGS | | G13D | TEM |
| | | PQHF | | | NYKLT | | | |
| | | (SEQ ID | | | F | | | |
| | | NO: | | | (SEQ ID | | | |
| | | 243) | | | NO: | | | |
| | | | | | 557) | | | |
| | TRBV24-1 | CATSD | TRBJ2-1 | TRAV13-1 | CAGHQ | TRAJ15 | G12C; | CD4 |
| | | PGTRE | | | AGTAL | | G12R | TCM |
| | | QFF | | | IF | | | |
| | | (SEQ ID | | | (SEQ ID | | | |
| | | NO: | | | NO: | | | |
| | | 244) | | | 558) | | | |
| | TRBV15 | CATSY | TRBJ2-1 | TRAV2 | CAVSG | TRAJ32 | G12A | CD4 |
| | | RAGGG | | | GATNK | | | TEM |
| | | YNEQF | | | LIF | | | |
| | | F | | | (SEQ ID | | | |
| | | (SEQ ID | | | NO: | | | |
| | | NO: | | | 559) | | | |
| | | 245) | | | | | | |
| | TRBV20-1 | CSARG | TRBJ2-1 | TRAV13-1 | CAASI | TRAJ10 | G12V | NA |
| | | NEQFF | | | ELTGG | | | |
| | | (SEQ ID | | | GNKLT | | | |
| | | NO: | | | F | | | |
| | | 246) | | | (SEQ ID | | | |
| | | | | | NO: | | | |
| | | | | | 560) | | | |
| | TRBV20-1 | CSASS | TRBJ2-7 | TRAV13-1 | CAAGM | TRAJ3 | G12R | NA |
| | | SGTQY | | | YSSAS | | | |
| | | F | | | KIIF | | | |
| | | (SEQ ID | | | (SEQ ID | | | |
| | | NO: | | | NO: | | | |
| | | 247) | | | 561) | | | |
| | TRBV28 | CAAED | TRBJ2-4 | TRAV9-2 | CALSL | TRAJ11 | G12A; | NA |
| | | LAKNI | | | SGYST | | G12C; | |
| | | QYF | | | LTF | | G12R; | |
| | | (SEQ ID | | | (SEQ ID | | G12V | |
| | | NO: | | | NO: | | | |
| | | 248) | | | 562) | | | |
| | TRBV28 | CAGRR | TRBJ1-6 | TRAV8-1 | CAVIN | TRAJ8 | G13D | CD8 |
| | | RLGDS | | | VDFQK | | | TEM |
| | | PLHF | | | LVF | | | |
| | | (SEQ ID | | | (SEQ ID | | | |
| | | NO: | | | NO: | | | |
| | | 249) | | | 563) | | | |
| | TRBV6-4 | CASKQ | TRBJ1-1 | TRAV1-1 | CASLT | TRAJ10 | G12D | CD4 |
| | | DLNTE | | | GGGNK | | | CTL |
| | | AFF | | | LTF | | | |
| | | (SEQ ID | | | (SEQ ID | | | |
| | | NO: | | | NO: | | | |
| | | 250) | | | 564) | | | |
| | TRBV28 | CASLS | TRBJ2-7 | TRAV12-3 | CAMGI | TRAJ41 | G12R; | CD8 |
| | | GPGYE | | | TSGYA | | G12V | TEM |
| | | QYF | | | LNF | | | |
| | | (SEQ ID | | | (SEQ ID | | | |
| | | NO: | | | NO: | | | |
| | | 251) | | | 565) | | | |
| | TRBV19 | CASNA | TRBJ2-2 | TRAV12-1 | CAVKG | TRAJ32 | G12A | CD4 |
| | | GYTSG | | | GGATN | | | TCM |
| | | ELFF | | | KLIF | | | |
| | | (SEQ ID | | | (SEQ ID | | | |
| | | NO: | | | NO: | | | |
| | | 252) | | | 566) | | | |
| | TRBV19 | CASQD | TRBJ2-7 | TRAV21 | CAVLG | TRAJ47 | G12D | CD4 |
| | | RTALE | | | YGNKL | | | TCM |
| | | QYF | | | VF | | | |
| | | (SEQ ID | | | (SEQ ID | | | |
| | | NO: | | | NO: | | | |
| | | 253) | | | 567) | | | |
| | TRBV28 | CASRG | TRBJ2-6 | TRAV35 | CAGQL | TRAJ44 | G12C | NA |
| | | GSSGA | | | AAGTA | | | |
| | | NVLTF | | | SKLTF | | | |
| | | (SEQ ID | | | (SEQ ID | | | |
| | | NO: | | | NO: | | | |
| | | 254) | | | 568) | | | |
| | TRBV6-6 | CASRG | TRBJ2-7 | TRAV13-1 | CARYS | TRAJ45 | G12A | CD8 |
| | | TSGRT | | | GGGAD | | | TEM |
| | | YEQYF | | | GLTF | | | |
| | | (SEQ ID | | | (SEQ ID | | | |
| | | NO: | | | NO: | | | |
| | | 255) | | | 569) | | | |
| | TRBV28 | CASRL | TRBJ1-2 | TRAV8-1 | CAVSP | TRAJ13 | G13D | CD4 |
| | | AGQEA | | | SGGYQ | | | TCM |
| | | NYGYT | | | KVTF | | | |
| | | F | | | (SEQ ID | | | |
| | | (SEQ ID | | | NO: | | | |
| | | NO: | | | 570) | | | |
| | | 256) | | | | | | |
| | TRBV12-4 | CASRP | TRBJ2-2 | TRAV13-2 | CAENR | TRAJ52 | G12A; | CD8 |
| | | SLLRE | | | RAGGT | | G12R | TEM |
| | | LFF | | | SYGKL | | | |
| | | (SEQ ID | | | TF | | | |
| | | NO: | | | (SEQ ID | | | |
| | | 257) | | | NO: | | | |
| | | | | | 571) | | | |
| | TRBV28 | CASRR | TRBJ2-2 | TRAV10 | CAGYN | TRAJ12 | G13D | CD4 |
| | | GNTGE | | | SGTYK | | | TCM |
| | | LFF | | | YIF | | | |
| | | (SEQ ID | | | (SEQ ID | | | |
| | | NO: | | | NO: | | | |
| | | 258) | | | 572) | | | |
| | TRBV6-1 | CASRR | TRBJ2-6 | TRAV29/ | CAASD | TRAJ39 | G12R | CD4 |
| | | NSGAN | | DV5 | AGNML | | | TCM |
| | | VLTF | | | TF | | | |
| | | (SEQ ID | | | (SEQ ID | | | |
| | | NO: | | | NO: | | | |
| | | 259) | | | 573) | | | |
| | TRBV13 | CASSA | TRBJ1-2 | TRAV14/ | CAARG | TRAJ27 | G12R | CD4 |
| | | RDRYY | | DV4 | NSGGS | | | TCM |
| | | GYTF | | | NYKLT | | | |
| | | (SEQ ID | | | F | | | |
| | | NO: | | | (SEQ ID | | | |
| | | 260) | | | NO: | | | |
| | | | | | 574) | | | |
| | TRBV6-4 | CASSD | TRBJ2-3 | TRAV1-2 | CATVN | TRAJ12 | G13D | CD4 |
| | | RDTDT | | | SGNTP | | | TCM |
| | | QYF | | | LVF | | | |
| | | (SEQ ID | | | (SEQ ID | | | |
| | | NO: | | | NO: | | | |
| | | 261) | | | 575) | | | |
| | TRBV2 | CASSE | TRBJ2-5 | TRAV2 | CAVER | TRAJ42 | G12D | CD4 |
| | | GGTRH | | | GSQGN | | | TCM |
| | | ETQYF | | | LIF | | | |
| | | (SEQ ID | | | (SEQ ID | | | |
| | | NO: | | | NO: | | | |
| | | 262) | | | 576) | | | |
| | TRBV6-1 | CASSE | TRBJ2-3 | TRAV1-2 | CAVMD | TRAJ33 | G12R | CD4 |
| | | GQGAD | | | SNYQL | | | TCM |
| | | TQYF | | | IW | | | |
| | | (SEQ ID | | | (SEQ ID | | | |
| | | NO: | | | NO: | | | |
| | | 263) | | | 577) | | | |
| | TRBV2 | CASSE | TRBJ1-5 | TRAV25 | CAGPG | TRAJ47 | G13D | CD8 |
| | | VSGNQ | | | YGNKL | | | Prolif- |
| | | PQHF | | | VF | | | erating |
| | | (SEQ ID | | | (SEQ ID | | | |
| | | NO: | | | NO: | | | |
| | | 264) | | | 578) | | | |
| | TRBV28 | CASSF | TRBJ1-4 | TRAV12-3 | CAAGV | TRAJ26 | G12C | CD4 |
| | | GLTNE | | | NFGNE | | | TCM |
| | | KLFF | | | KLTF | | | |
| | | (SEQ ID | | | (SEQ ID | | | |
| | | NO: | | | NO: | | | |
| | | 265) | | | 579) | | | |
| | TRBV27 | CASSF | TRBJ1-2 | TRAV29/ | CAASR | TRAJ43 | G13D | CD4 |
| | | GTGVY | | DV5 | GFNDM | | | TCM |
| | | GYTF | | | RF | | | |
| | | (SEQ ID | | | (SEQ ID | | | |
| | | NO: | | | NO: | | | |
| | | 266) | | | 580) | | | |
| | TRBV5-1 | CASSF | TRBJ1-6 | TRAV12-1 | CAVSG | TRAJ40 | G12A; | CD4 |
| | | MDRDN | | | LVGNE | | G12C; | TCM |
| | | SPLHF | | | KLTF | | G12R; | |
| | | (SEQ ID | | | (SEQ ID | | G12V | |
| | | NO: | | | NO: | | | |
| | | 267) | | | 581) | | | |
| | TRBV27 | CASSF | TRBJ2-1 | TRAV2 | CAVVF | TRAJ21 | G13D | CD8 |
| | | SGDNE | | | NKFYF | | | TEM |
| | | QFF | | | (SEQ ID | | | |
| | | (SEQ ID | | | NO: | | | |
| | | NO: | | | 582) | | | |
| | | 268) | | | | | | |
| | TRBV6-5 | CASSG | TRBJ1-2 | TRAV17 | CATEG | TRAJ11 | G12D | CD8 |
| | | QGGGY | | | DSGYS | | | TCM |
| | | GYTF | | | TLTF | | | |
| | | (SEQ ID | | | (SEQ ID | | | |
| | | NO: | | | NO: | | | |
| | | 269) | | | 583) | | | |
| | TRBV2 | CASSI | TRBJ2-5 | TRAV38-2/ | CAVSG | TRAJ49 | G12D | CD4 |
| | | TRKET | | DV8 | TGNQF | | | TCM |
| | | QYF | | | YF | | | |
| | | (SEQ ID | | | (SEQ ID | | | |
| | | NO: | | | NO: | | | |
| | | 270) | | | 584) | | | |
| | TRBV7-9 | CASSL | TRBJ2-7 | TRAV29/ | CAASV | TRAJ53 | G12A | NA |
| | | AHYEQ | | DV5 | GGSNY | | | |
| | | YF | | | KLTF | | | |
| | | (SEQ ID | | | (SEQ ID | | | |
| | | NO: | | | NO: | | | |
| | | 271) | | | 585) | | | |
| | TRBV5-1 | CASSL | TRBJ2-1 | TRAV29/ | CAASG | TRAJ29 | G12A; | CD4 |
| | | APHTD | | DV5 | SDSGN | | G12C; | TCM |
| | | EQFF | | | TPLVF | | G12V; | |
| | | (SEQ ID | | | (SEQ ID | | G13D | |
| | | NO: | | | NO: | | | |
| | | 272) | | | 586) | | | |
| | TRBV7-6 | CASSL | TRBJ2-1 | TRAV14/ | CAMSS | TRAJ22 | G13D | NA |
| | | DEQGQ | | DV4 | RGSAR | | | |
| | | NEQFF | | | QLTF | | | |
| | | (SEQ ID | | | (SEQ ID | | | |
| | | NO: | | | NO: | | | |
| | | 273) | | | 587) | | | |
| | TRBV5-5 | CASSL | TRBJ2-7 | TRAV8-1 | CAVNG | TRAJ35 | G12V | CD8 |
| | | EADYE | | | FGNVL | | | TCM |
| | | QYF | | | HC | | | |
| | | (SEQ ID | | | (SEQ ID | | | |
| | | NO: | | | NO: | | | |
| | | 274) | | | 588) | | | |
| | TRBV5-5 | CASSL | TRBJ1-5 | TRAV12-2 | CAAPS | TRAJ30 | G12R | CD4 |
| | | EDNQP | | | RDDKI | | | TCM |
| | | QHF | | | IF | | | |
| | | (SEQ ID | | | (SEQ ID | | | |
| | | NO: | | | NO: | | | |
| | | 275) | | | 589) | | | |
| | TRBV5-1 | CASSL | TRBJ1-2 | TRAV4 | CLVGD | TRAJ22 | G13D | CD8 |
| | | GGQVY | | | NAPSG | | | TCM |
| | | GYTF | | | SARQL | | | |
| | | (SEQ ID | | | TF | | | |
| | | NO: | | | (SEQ ID | | | |
| | | 276) | | | NO: | | | |
| | | | | | 590) | | | |
| | TRBV3-1 | CASSL | TRBJ1-4 | TRAV13-2 | CAENG | TRAJ20 | G12V | NA |
| | | GQGLN | | | SDYKL | | | |
| | | EKLFF | | | SF | | | |
| | | (SEQ ID | | | (SEQ ID | | | |
| | | NO: | | | NO: | | | |
| | | 277) | | | 591) | | | |
| | TRBV12-3 | CASSL | TRBJ1-2 | TRAV29/ | CAALS | TRAJ36 | G12A | CD4 |
| | | GRNYG | | DV5 | HQGAQ | | | TCM |
| | | YTF | | | KLVF | | | |
| | | (SEQ ID | | | (SEQ ID | | | |
| | | NO: | | | NO: | | | |
| | | 278) | | | 592) | | | |
| | TRBV5-1 | CASSL | TRBJ2-1 | TRAV3 | CAVRV | TRAJ4 | G12C | CD8 |
| | | GTSAY | | | FSGGY | | | TEM |
| | | NEQFF | | | NKLIF | | | |
| | | (SEQ ID | | | (SEQ ID | | | |
| | | NO: | | | NO: | | | |
| | | 279) | | | 593) | | | |
| | TRBV5-8 | CASSL | TRBJ1-6 | TRAV8-3 | CAVGE | TRAJ32 | G12R | NA |
| | | MQAAN | | | RGATN | | | |
| | | SPLHF | | | KLIF | | | |
| | | (SEQ ID | | | (SEQ ID | | | |
| | | NO: | | | NO: | | | |
| | | 280) | | | 594) | | | |
| | TRBV7-9 | CASSL | TRBJ1-2 | TRAV8-6 | CAVKS | TRAJ29 | G12R | CD4 |
| | | MSATN | | | NSGNT | | | TCM |
| | | YGYTF | | | PLVF | | | |
| | | (SEQ ID | | | (SEQ ID | | | |
| | | NO: | | | NO: | | | |
| | | 281) | | | 595) | | | |
| | TRBV5-1 | CASSL | TRBJ1-4 | TRAV29/ | CAASE | TRAJ54 | G13D | CD4 |
| | | QGARE | | DV5 | PGAQK | | | TCM |
| | | KLFF | | | LVF | | | |
| | | (SEQ ID | | | (SEQ ID | | | |
| | | NO: | | | NO: | | | |
| | | 282) | | | 596) | | | |
| | TRBV7-9 | CASSL | TRBJ1-1 | TRAV1-2 | CAGAV | TRAJ24 | G12D; | NA |
| | | OGGTE | | | TTDSW | | G12R | |
| | | AFF | | | GKLQF | | | |
| | | (SEQ ID | | | (SEQ ID | | | |
| | | NO: | | | NO: | | | |
| | | 283) | | | 597) | | | |
| | TRBV12-3 | CASSL | TRBJ2-1 | TRAV12-1 | CAVNV | TRAJ27 | G12D | NA |
| | | SGSSY | | | NSGAG | | | |
| | | NEQFF | | | SYQLT | | | |
| | | (SEQ ID | | | F | | | |
| | | NO: | | | (SEQ ID | | | |
| | | 284) | | | NO: | | | |
| | | | | | 598) | | | |
| | TRBV28 | CASSL | TRBJ1-5 | TRAV14/ | CAMRE | TRAJ6 | G13D | CD4 |
| | | SGTGN | | DV4 | RTGGS | | | TCM |
| | | GRNQP | | | YIPTF | | | |
| | | QHF | | | (SEQ ID | | | |
| | | (SEQ ID | | | NO: | | | |
| | | NO: | | | 599) | | | |
| | | 285) | | | | | | |
| | TRBV27 | CASSL | TRBJ1-2 | TRAV12-3 | CAIGR | TRAJ18 | G12C; | NA |
| | | SRDAV | | | GSTLG | | G12V | |
| | | GGYTF | | | RLYF | | | |
| | | (SEQ ID | | | (SEQ ID | | | |
| | | NO: | | | NO: | | | |
| | | 286) | | | 600) | | | |
| | TRBV28 | CASSL | TRBJ2-7 | TRAV8-1 | CAVSP | TRAJ3 | G12V | CD4 |
| | | TGTGG | | | PGYSS | | | TCM |
| | | YEQYF | | | ASKII | | | |
| | | (SEQ ID | | | F | | | |
| | | NO: | | | (SEQ ID | | | |
| | | 287) | | | NO: | | | |
| | | | | | 601) | | | |
| | TRBV12-4 | CASSL | TRBJ1-5 | TRAV13-2 | CAEEL | TRAJ13 | G12R | CD4 |
| | | VAGGY | | | SGGYQ | | | TEM |
| | | EQYF | | | KVTF | | | |
| | | (SEQ ID | | | (SEQ ID | | | |
| | | NO: | | | NO: | | | |
| | | 288) | | | 602) | | | |
| | TRBV27 | CASSL | TRBJ2-1 | TRAV22 | CAVEF | TRAJ47 | G12C; | NA |
| | | YNEQF | | | TEYGN | | G12R | |
| | | F | | | KLVF | | | |
| | | (SEQ ID | | | (SEQ ID | | | |
| | | NO: | | | NO: | | | |
| | | 289) | | | 603) | | | |
| | TRBV12-4 | CASSP | TRBJ2-7 | TRAV12-2 | CAVSY | TRAJ3 | G12A; | CD4 |
| | | PFGSY | | | SSASK | | G12C; | TCM |
| | | EQYF | | | IIF | | G12D; | |
| | | (SEQ ID | | | (SEQ ID | | G12V; | |
| | | NO: | | | NO: | | G13D | |
| | | 290) | | | 604) | | | |
| | TRBV11-2 | CASSQ | TRBJ2-3 | TRAV13-2 | CAEFY | TRAJ23 | G12D | CD4 |
| | | ADTQY | | | NQGGK | | | TCM |
| | | F | | | LIF | | | |
| | | (SEQ ID | | | (SEQ ID | | | |
| | | NO: | | | NO: | | | |
| | | 291) | | | 605) | | | |
| | TRBV3-1 | CASSQ | TRBJ1-4 | TRAV21 | CAVNN | TRAJ47 | G12V | CD4 |
| | | GQEFG | | | GNKLV | | | TCM |
| | | KLFF | | | F | | | |
| | | (SEQ ID | | | (SEQ ID | | | |
| | | NO: | | | NO: | | | |
| | | 292) | | | 606) | | | |
| | TRBV7-9 | CASSQ | TRBJ2-1 | TRAV21 | CAGGN | TRAJ27 | G12D | CD4 |
| | | TSGSY | | | AGKST | | | TCM |
| | | NEQFF | | | F | | | |
| | | (SEQ ID | | | (SEQ ID | | | |
| | | NO: | | | NO: | | | |
| | | 293) | | | 607) | | | |
| | TRBV2 | CASSR | TRBJ2-7 | TRAV12-2 | CAASR | TRAJ3 | G12D | Treg |
| | | TYEQY | | | RGSQG | | | |
| | | F | | | NLIF | | | |
| | | (SEQ ID | | | (SEQ ID | | | |
| | | NO: | | | NO: | | | |
| | | 294) | | | 608) | | | |
| | TRBV12-4 | CASSS | TRBJ1-2 | TRAV35 | CAGPM | TRAJ50 | G12A; | CD8 |
| | | ANYGY | | | KTSYD | | G12V | TEM |
| | | TF | | | KVIF | | | |
| | | (SEQ ID | | | (SEQ ID | | | |
| | | NO: | | | NO: | | | |
| | | 295) | | | 609) | | | |
| | TRBV19 | CASSS | TRBJ2-2 | TRAV1-2 | CAVKD | TRAJ33 | G12A; | NA |
| | | GEGEA | | | SNYQL | | G12R; | |
| | | GELFF | | | IW | | G12V | |
| | | (SEQ ID | | | (SEQ ID | | | |
| | | NO: | | | NO: | | | |
| | | 296) | | | 610) | | | |
| | TRBV28 | CASSS | TRBJ1-1 | TRAV13-1 | CAASI | TRAJ42 | G12CG12V | NA |
| | | GGLNT | | | VGSQG | | | |
| | | EAFF | | | NLIF | | | |
| | | (SEQ ID | | | (SEQ ID | | | |
| | | NO: | | | NO: | | | |
| | | 297) | | | 611) | | | |
| | TRBV6-2 | CASSS | TRBJ2-3 | TRAV21 | CAALP | TRAJ29 | G13D | CD8 |
| | | GGRAW | | | GNTPL | | | TEM |
| | | DTQYF | | | VF | | | |
| | | (SEQ ID | | | (SEQ ID | | | |
| | | NO: | | | NO: | | | |
| | | 298) | | | 612) | | | |
| | TRBV5-6 | CASSS | TRBJ2-7 | TRAV1-1 | CAPWR | TRAJ22 | G12A | CD8 |
| | | GLAAY | | | GSARQ | | | TCM |
| | | EQYF | | | LTF | | | |
| | | (SEQ ID | | | (SEQ ID | | | |
| | | NO: | | | NO: | | | |
| | | 299) | | | 613) | | | |
| | TRBV7-2 | CASSS | TRBJ2-5 | TRAV12-2 | CAVNP | TRAJ9 | G12V | CD4 |
| | | QGSQE | | | TGGFK | | | TCM |
| | | TQYF | | | TIF | | | |
| | | (SEQ ID | | | (SEQ ID | | | |
| | | NO: | | | NO: | | | |
| | | 300) | | | 614) | | | |
| | TRBV18 | CASSS | TRBJ1-5 | TRAV26-1 | CIVRP | TRAJ52 | G12R | CD4 |
| | | TGGNQ | | | SNAGG | | | TCM |
| | | PQHF | | | TSYGK | | | |
| | | (SEQ ID | | | LTF | | | |
| | | NO: | | | (SEQ ID | | | |
| | | 301) | | | NO: | | | |
| | | | | | 615) | | | |
| | TRBV5-1 | CASSV | TRBJ2-2 | TRAV23/ | CAASR | TRAJ22 | G12R | CD4 |
| | | RQGSA | | DV6 | VGQLT | | | TCM |
| | | GELFF | | | F | | | |
| | | (SEQ ID | | | (SEQ ID | | | |
| | | NO: | | | NO: | | | |
| | | 302) | | | 616) | | | |
| | TRBV7-9 | CASSW | TRBJ2-5 | TRAV17 | CATDA | TRAJ56 | G13D | CD8 |
| | | GLADE | | | WTGAN | | | TEM |
| | | TQYF | | | SKLTF | | | |
| | | (SEQ ID | | | (SEQ ID | | | |
| | | NO: | | | NO: | | | |
| | | 303) | | | 617) | | | |
| | TRBV6-5 | CASSY | TRBJ1-2 | TRAV13-1 | CAAKW | TRAJ28 | G12R | CD4 |
| | | DSRYG | | | AYSGA | | | Prolif- |
| | | YTF | | | GSYQL | | | erating |
| | | (SEQ ID | | | TF | | | |
| | | NO: | | | (SEQ ID | | | |
| | | 304) | | | NO: | | | |
| | | | | | 618) | | | |
| | TRBV6-2 | CASSY | TRBJ2-7 | TRAV1-2 | CAVRD | TRAJ23 | G12D; | CD8 |
| | | SAGEQ | | | NNQGG | | G12R | TEM |
| | | YF | | | KLIF | | | |
| | | (SEQ ID | | | (SEQ ID | | | |
| | | NO: | | | NO: | | | |
| | | 305) | | | 619) | | | |
| | TRBV6-6 | CASSY | TRBJ2-4 | TRAV38-2/ | CAYRS | TRAJ58 | G12C | CD4 |
| | | SPSTK | | DV8 | QETSG | | | TCM |
| | | NIQYF | | | SRLTF | | | |
| | | (SEQ ID | | | (SEQ ID | | | |
| | | NO: | | | NO: | | | |
| | | 306) | | | 620) | | | |
| | TRBV11-3 | CATEG | TRBJ1-3 | TRAV12-1 | CAADT | TRAJ5 | G12D | CD4 |
| | | RGNTI | | | GRRAL | | | TCM |
| | | YF | | | TF | | | |
| | | (SEQ ID | | | (SEQ ID | | | |
| | | NO: | | | NO: | | | |
| | | 307) | | | 621) | | | |
| | TRBV7-9 | CATPP | TRBJ2-2 | TRAV24 | CAKYT | TRAJ34 | G12C | CD8 |
| | | GGLAN | | | DKLIF | | | TEM |
| | | TGELF | | | (SEQ ID | | | |
| | | F | | | NO: | | | |
| | | (SEQ ID | | | 622) | | | |
| | | NO: | | | | | | |
| | | 308) | | | | | | |
| | TRBV24-1 | CATSD | TRBJ2-1 | TRAV23/ | CAASI | TRAJ18 | G12D | CD4 |
| | | SSGRY | | DV6 | GSTLG | | | TCM |
| | | YNEQF | | | RLYF | | | |
| | | F | | | (SEQ ID | | | |
| | | (SEQ ID | | | NO: | | | |
| | | NO: | | | 623) | | | |
| | | 309) | | | | | | |
| | TRBV30 | CAWSG | TRBJ1-1 | TRAV12-1 | CVVNG | TRAJ6 | G12A; | CD4 |
| | | MNTEA | | | PPGGS | | G12V; | TCM |
| | | FF | | | YIPTF | | G13D | |
| | | (SEQ ID | | | (SEQ ID | | | |
| | | NO: | | | NO: | | | |
| | | 310) | | | 624) | | | |
| | TRBV20-1 | CSARG | TRBJ2-7 | TRAV12-2 | CAVTD | TRAJ24 | G12C | CD4 |
| | | GHSFE | | | SWGKL | | | TEM |
| | | QYF | | | QF | | | |
| | | (SEQ ID | | | (SEQ ID | | | |
| | | NO: | | | NO: | | | |
| | | 311) | | | 625) | | | |
| | TRBV20-1 | CSARN | TRBJ1-1 | TRAV1-2 | CAVVD | TRAJ33 | G12C | MAIT |
| | | GDTEA | | | SNYQL | | | |
| | | FF | | | IW | | | |
| | | (SEQ ID | | | (SEQ ID | | | |
| | | NO: | | | NO: | | | |
| | | 312) | | | 626) | | | |
| | TRBV12-4 | CASSL | TRBJ1-5 | TRAV13-2 | CAEEL | TRAJ13 | G12R | CD4 |
| | | VAGGY | | | SGGYQ | | | TEM |
| | | EQYF | | | KVTF | | | |
| | | (SEQ ID | | | (SEQ ID | | | |
| | | NO: | | | NO: | | | |
| | | 313) | | | 627) | | | |
| | TRBV29-1 | CSVRV | TRBJ1-1 | TRAV8-4 | CAVSF | TRAJ17 | G12V | CD4 |
| | | NTEAF | | | KAAGN | | | Naive |
| | | F | | | KLTF | | | |
| | | (SEQ ID | | | (SEQ ID | | | |
| | | NO: | | | NO: | | | |
| | | 314) | | | 628) | | | |
| Public | TRBV28* | CASRR | TRBJ2-2*01 | TRAV35* | CAGYN | TRAJ40* | G13D | CD TCM |
| TCRβ08 | 01 | GNTGE | | 01 | SGTYK | 01 | | |
| (Validated) | | LFF | | | YIF | | | |
| | | (SEQ ID | | | (SEQ ID | | | |
| | | NO: | | | NO: | | | |
| | | 631) | | | 632) | | | |
| Public | TRBV2 | CASSS | TRBJ1- | TRAV13- | CAASI | TRAJ42 | G12V | NA |
| TCRβ10 | 8*01 | GGLNT | 1*01 | 1*01 | VGSQG | *01 | G12C | |
| | | EAFF | | | NLIF | | G12A | |
| | | (SEQ | | | (SEQ ID | | G13D | |
| | | ID | | | NO: | | | |
| | | NO: | | | 634) | | | |
| | | 633) | | | | | | |

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1.-98. (canceled)

99. A T cell receptor (TCR) comprising:
(i) a TCRα chain variable domain (TRAV) comprising a CDR3 α, wherein the CDR3α comprises an amino acid sequence having at least 75% sequence identity to any one of SEQ ID NOs: 315-629, 632, or 634; and
(ii) a TCRβ chain variable domain (TRBV) comprising a CDR3β, wherein the CDR3β comprises an amino acid sequence having at least 75% sequence identity to any one of SEQ ID NOs: 1-314, 630, 631, or 633.

100. The TCR of claim 99, wherein the TRAV and the TRBV are:
(i) TRAV13-1 and TRBV20-1, respectively;
(ii) TRAV17 and TRBV7-9, respectively;
(iii) TRAV10 and TRBV2, respectively;
(iv) TRAV8-3 and TRBV12-3, respectively;
(v) TRAV13-1 and TRBV2, respectively;
(vi) TRAV3 and TRBV5-6, respectively;
(vii) TRAV13-1 and TRBV12-3, respectively;
(viii) TRAV29/DV5 and TRBV11-3, respectively;
(ix) TRAV27 and TRBV5-1, respectively;
(x) TRAV2 and TRBV2, respectively;
(xi) TRAV5 and TRBV18, respectively;
(xii) TRAV9-2 and TRBV27, respectively;
(xiii) TRAV3 and TRBV18, respectively;
(xiv) TRAV21 and TRBV2, respectively;
(xv) TRAV1-2 and TRBV28, respectively;
(xvi) TRAV21 and TRBV7-9, respectively;
(xvii) TRAV23/DV6 and TRBV5-1, respectively;
(xviii) TRAV23/DV6 and TRBV9, respectively;
(xix) TRAV12-1 and TRBV20-1, respectively;
(xx) TRAV21 and TRBV20-1, respectively;
(xxi) TRAV12-3 and TRBV20-1, respectively;
(xxii) TRAV8-6 and TRBV20-1, respectively;
(xxiii) TRAV1-2 and TRBV20-1, respectively;
(xxiv) TRAV23/DV6 and TRBV2, respectively;
(xxv) TRAV26-2 and TRBV6-4, respectively;
(xxvi) TRAV21 and TRBV6-5, respectively;
(xxvii) TRAV22 and TRBV18, respectively;
(xxviii)TRAV29/DV5 and TRBV6-2, respectively;
(xxix) TRAV9-2 and TRBV19, respectively;
(xxx) TRAV8-4 and TRBV5-5, respectively;
(xxxi) TRAV26-1 and TRBV29-1, respectively;
(xxxii) TRAV14/DV4 and TRBV11-2, respectively;
(xxxiii)TRAV12-1 and TRBV28, respectively;
(xxxiv)TRAV8-4 and TRBV9, respectively;
(xxxv) TRAV16 and TRBV9, respectively;
(xxxvi)TRAV13-2 and TRBV5-1, respectively;
(xxxvii) TRAV20 and TRBV6-6, respectively;
(xxxviii) TRAV13-1 and TRBV24-1, respectively;
(xxxix)TRAV2 and TRBV15, respectively;
(xl) TRAV9-2 and TRBV28, respectively;
(xli) TRAV12-3 and TRBV28, respectively;
(xlii) TRAV13-1 and TRBV6-6, respectively;
(xliii) TRAV13-2 and TRBV12-4, respectively;
(xliv) TRAV29/DV5 and TRBV27, respectively;
(xlv) TRAV12-1 and TRBV5-1, respectively;
(xlvi) TRAV2 and TRBV27, respectively;
(xlvii) TRAV17 and TRBV6-5, respectively;
(xlviii) TRAV29/DV5 and TRBV5-1, respectively;
(xlix) TRAV13-2 and TRBV3-1, respectively;
(1) TRAV14/DV4 and TRBV28, respectively;
(li) TRAV12-2 and TRBV12-4, respectively;
(lii) TRAV35 and TRBV12-4, respectively;
(liii) TRAV13-1 and TRBV6-5, respectively;
(liv) TRAV1-2 and TRBV6-2, respectively; or
(lv) TRAV38-2/DV8 and TRBV6-6, respectively.

101. The TCR of claim 100, wherein the TRAV and the TRBV are TRAV13-1*01 and TRBV 20-1*01, respectively.

102. The TCR of claim 99, wherein the T cell receptor has binding specificity to KRAS tumor antigens.

103. The TCR of claim 99, wherein the TCR has binding specifically to mutant KRAS epitopes.

104. The TCR of claim 103, wherein the mutant KRAS epitopes comprise G12V, G12D, G12C, G12R, G12A, or G13D or a combination thereof.

105. The TCR of claim 99, wherein the CDR3α comprises an amino acid sequence of any one of SEQ ID NOs: 315-629, 632, or 634.

106. The TCR of claim 105, wherein the CDR3α comprises the amino acid sequence of SEQ ID NO: 629.

107. The TCR of claim 105, wherein the CDR30 comprises an amino acid sequence of any one of SEQ ID NOs: 1-314, 630, 631, or 633.

108. The TCR of claim 106, wherein the CDR30 comprises the amino acid sequence of SEQ ID NO: 630.

109. The TCR of claim 99, wherein the CDR30 comprises an amino acid sequence of any one of SEQ ID NOs: 1-314, 630, 631, or 633.

110. The TCR of claim 99, wherein the CDR3α and the CDR30 comprise amino acid sequences of:
(i) CAFILNNNDMRF (SEQ ID NO: 315) and CAIAGPGQGARGYTF (SEQ ID NO: 001) respectively;
(ii) CALSEAVGGANNLFF (SEQ ID NO: 316) and CAISEGSPEAFF (SEQ ID NO: 002) respectively;
(iii) CAFMKQSGGSQGNLIF (SEQ ID NO: 317) and CASGLRLNEKLFF (SEQ ID NO: 003) respectively;
(iv) CAVRSYSGAGSYQLTF (SEQ ID NO: 318) and CASGLVDYELFF (SEQ ID NO: 004) respectively;
(v) CAACDRGSTLGRLYF (SEQ ID NO: 319) and CASIHLVGGTGRQPQHF (SEQ ID NO: 005) respectively;
(vi) CAAGNNARLMF (SEQ ID NO: 320) and CASKEATAASTNEKLFF (SEQ ID NO: 006) respectively;
(vii) CAGLSNDYKLSF (SEQ ID NO: 321) and CASKQGNEQFF (SEQ ID NO: 007) respectively;
(viii) CIVKSWGKLQF (SEQ ID NO: 322) and CASLLDAGAANTEAFF (SEQ ID NO: 008) respectively;
(ix) CATDAGNDMRF (SEQ ID NO: 323) and CASLSRGLNEKLFF (SEQ ID NO: 009) respectively;
(x) CAVGAGGTSYGKLTF (SEQ ID NO: 324) and CASMLQGALNQPQHF (SEQ ID NO: 010) respectively;
(xi) CAGHSNTGNQFYF (SEQ ID NO: 325) and CASNFGQGRYGYTF (SEQ ID NO: 011) respectively;
(xii) CVVSRVKAAGNKLTF (SEQ ID NO: 326) and CASNPDNALDNSPLHF (SEQ ID NO: 012) respectively;
(xiii) CAAPYPTGGTSYGKLTF (SEQ ID NO: 327) and CASRDSYSNQPQHF (SEQ ID NO: 013) respectively;
(xiv) CAVSERGFQKLVF (SEQ ID NO: 328) and CASRDTQGGGADTQYF (SEQ ID NO: 014) respectively;
(xv) CALSESWGKLQF (SEQ ID NO: 329) and CASREQGQGTGELFF (SEQ ID NO: 015) respectively;
(xvi) CAVGASGAAGNKLTF (SEQ ID NO: 330) and CASRGDSGFNYGYTF (SEQ ID NO: 016) respectively;
(xvii) CAVSDNQGAQKLVF (SEQ ID NO: 331) and CASRGQGAATDTQYF (SEQ ID NO: 017) respectively;
(xviii) CAAPTSGGGADGLTF (SEQ ID NO: 332) and CASRGQRRINYGYTF (SEQ ID NO: 018) respectively;
(xix) CAGQGYFGNEKLTF (SEQ ID NO: 333) and CASRGTGVNQPQHF (SEQ ID NO: 019) respectively;
(xx) CALSYNQGGKLIF (SEQ ID NO: 334) and CASRKDTGELFF (SEQ ID NO: 020) respectively;
(xxi) CAVSEPGSGGSNYKLTF (SEQ ID NO: 335) and CASRLDRSEAFF (SEQ ID NO: 021) respectively;
(xxii) CVVSDVNRNQFYF (SEQ ID NO: 336) and CASRPGQGYEKLFF (SEQ ID NO: 022) respectively;
(xxiii) CAVNAPFGNEKLTF (SEQ ID NO: 337) and CASRRNGLYYTF (SEQ ID NO: 023) respectively;
(xxiv) CVVNWGGGYNKLIF (SEQ ID NO: 338) and CASRSGTGGSGELFF (SEQ ID NO: 024) respectively;
(xxv) CAEITRYGGSQGNLIF (SEQ ID NO: 339) and CASRSPWTGANVLTF (SEQ ID NO: 025) respectively;
(xxvi) CALSTSGTYKYIF (SEQ ID NO: 340) and CASRTGGNLDDTQYF (SEQ ID NO: 026) respectively;
(xxvii) CVVGVRGGGTSYGKLTF (SEQ ID NO: 341) and CASRTLSGGDTQYF (SEQ ID NO: 027) respectively;
(xxviii)CALRAQNSGYSTLTF (SEQ ID NO: 342) and CASSAFYEQYF (SEQ ID NO: 028) respectively;
(xxix) CAFMKHMSGNNRKLIW (SEQ ID NO: 343) and CASSALGSGNTIYF (SEQ ID NO: 029) respectively;
(xxx) CAASGTYKYIF (SEQ ID NO: 344) and CASSAQGTSYNEQFF (SEQ ID NO: 030) respectively;
(xxxi) CAEIAGNQFYF (SEQ ID NO: 345) and CASSASFYQPQHF (SEQ ID NO: 031) respectively;
(xxxii) CAVSDSFQKLVF (SEQ ID NO: 346) and CASSATGGNSPLHF (SEQ ID NO: 032) respectively;
(xxxiii) CAVSSTYGNKLVF (SEQ ID NO: 347) and CASSEGQGDYGYTF (SEQ ID NO: 033) respectively;
(xxxiv) CAAIGSSNTGKLIF (SEQ ID NO: 348) and CASSENRRREPQHF (SEQ ID NO: 034) respectively;
(xxxv) CAVSEDTGGFKTIF (SEQ ID NO: 349) and CASSEQSGLTNSPLHF (SEQ ID NO: 035) respectively;
(xxxvi) CAVLGTSGSRLTF (SEQ ID NO: 350) and CASSFAAGLGYEQYF (SEQ ID NO: 036) respectively;
(xxxvii) CAVRDKRSNDYKLSF (SEQ ID NO: 351) and CASSFAGVYTGELFF (SEQ ID NO: 037) respectively;
(xxxviii) CAASNVLTGGGNKLTF (SEQ ID NO: 352) and CASSFPTGGLSSEQFF (SEQ ID NO: 038) respectively;
(xxxix) CAAISRTGSARQLTF (SEQ ID NO: 353) and CASSFQETQYF (SEQ ID NO: 039) respectively;
(xl) CATDATGANSKLTF (SEQ ID NO: 354) and CASSFRGGLQETQYF (SEQ ID NO: 040) respectively;
(xli) CALSDSGGGADGLTF (SEQ ID NO: 355) and CASSFTDRRKDTF (SEQ ID NO: 041) respectively;
(xlii) CALSEAGNAGNMLTF (SEQ ID NO: 356) and CASSFYGTGGEGKPQHF (SEQ ID NO: 042) respectively;
(xliii) CAVPGGTSYGKLTF (SEQ ID NO: 357) and CASSGTDFYEQYF (SEQ ID NO: 043) respectively;
(xliv) CAVGARQAGTALIF (SEQ ID NO: 358) and CASSGTGDSGEAFF (SEQ ID NO: 044) respectively;
(xlv) CAFMTINAGGTSYGKLTF (SEQ ID NO: 359) and CASSGTGGAISNQPQHF (SEQ ID NO: 045) respectively;
(xlvi) CVVRVGFGNVLHC (SEQ ID NO: 360) and CASSIGGTTGELFF (SEQ ID NO: 046) respectively;
(xlvii) CAATRRSNDYKLSF (SEQ ID NO: 361) and CASSISTNTGELFF (SEQ ID NO: 047) respectively;
(xlviii) CIAPNMDSNYQLIW (SEQ ID NO: 362) and CASSITGSSGQPQHF (SEQ ID NO: 048) respectively;
(xlix) CATPKIYNQGGKLIF (SEQ ID NO: 363) and CASSKDRGLALETQYF (SEQ ID NO: 049) respectively;
(1) CAVRPPGANSKLTF (SEQ ID NO: 364) and CASSKESANRYNEQFF (SEQ ID NO: 050) respectively;
(li) CAVEDDNNARLMF (SEQ ID NO: 365) and CASSLASNQPQHF (SEQ ID NO: 051) respectively;
(lii) CAASPRFNKFYF (SEQ ID NO: 366) and CASSLDQTSNEQFF (SEQ ID NO: 052) respectively;
(liii) CAVNSNYQLIW (SEQ ID NO: 367) and CASSLDRGLGNSPLHF (SEQ ID NO: 053) respectively;
(liv) CAVKGPAGNNRKLIW (SEQ ID NO: 368) and CASSLDSGTNTGELFF (SEQ ID NO: 054) respectively;
(lv) CALSVGGAQKLVF (SEQ ID NO: 369) and CASSLDSLATDTQYF (SEQ ID NO: 055) respectively;
(lvi) CVVLERNTGGFKTIF (SEQ ID NO: 370) and CASSLEGGLAKNIQYF (SEQ ID NO: 056) respectively;
(lvii) CATDRDSNYQLIW (SEQ ID NO: 371) and CASSLEGRGPTNEKLFF (SEQ ID NO: 057) respectively;
(lviii) CAMREGSDYKLSF (SEQ ID NO: 372) and CASSLESGNSPLHF (SEQ ID NO: 058) respectively;
(lix) CAASGGGADGLTF (SEQ ID NO: 373) and CASSLETGVEQFF (SEQ ID NO: 059) respectively;
(lx) CAASLPGNTPLVF (SEQ ID NO: 374) and CASSLFGGGGEKLFF (SEQ ID NO: 060) respectively;
(lxi) CAYRSGATNKLIF (SEQ ID NO: 375) and CASSLFLGSYEQYF (SEQ ID NO: 061) respectively;
(lxii) CAVPPPNFGNEKLTF (SEQ ID NO: 376) and CASSLGGGNQPQHF (SEQ ID NO: 062) respectively;
(lxiii) CATDSSNTGNQFYF (SEQ ID NO: 377) and CASSLGGNTGELFF (SEQ ID NO: 063) respectively;
(lxiv) CIAHDRGSTLGRLYF (SEQ ID NO: 378) and CASSLGGSGSFYHNEQFF (SEQ ID NO: 064) respectively;
(lxv) CALSPQGTGGFKTIF (SEQ ID NO: 379) and CASSLGLRPINEQFF (SEQ ID NO: 065) respectively;
(lxvi) CAALAGPGYALNF (SEQ ID NO: 380) and CASSLGRGPTDTQYF (SEQ ID NO: 066) respectively;
(lxvii) CAVRGAGNNRKLIW (SEQ ID NO: 381) and CASSLGVNTEAFF (SEQ ID NO: 067) respectively;
(lxviii) CADSYGGATNKLIF (SEQ ID NO: 382) and CASSLGYRGEQYF (SEQ ID NO: 068) respectively;
(lxix) CILSNVYSGAGSYQLTF (SEQ ID NO: 383) and CASSLLDRGDSPLHF (SEQ ID NO: 069) respectively;
(lxx) CAFDNNDMRF (SEQ ID NO: 384) and CASSLLTGGQYF (SEQ ID NO: 070) respectively;
(lxxi) CAFIGLLGIQGAQKLVF (SEQ ID NO: 385) and CASSLNRGYEQYV (SEQ ID NO: 071) respectively;
(lxxii) CAASIRSGGSYIPTF (SEQ ID NO: 386) and CASSLNTEAFF (SEQ ID NO: 072) respectively;
(lxxiii) CAANAGNNRKLIW (SEQ ID NO: 387) and CASSLQGRTEAFF (SEQ ID NO: 073) respectively;
(lxxiv) CAAEFTGTASKLTF (SEQ ID NO: 388) and CASSLQGSYGYTF (SEQ ID NO: 074) respectively;
(lxxv) CAEGRLTGGFKTIF (SEQ ID NO: 389) and CASSLRGEAFF (SEQ ID NO: 075) respectively;
(lxxvi) CAVRGGGGFKTIF (SEQ ID NO: 390) and CASSLRGNEQFF (SEQ ID NO: 076) respectively;
(lxxvii) CGTEEMNRDDKIIF (SEQ ID NO: 391) and CASSLRTGGRMPQHF (SEQ ID NO: 077) respectively;
(lxxviii) CAASNSGYALNF (SEQ ID NO: 392) and CASSLRTNTGEKLFF (SEQ ID NO: 078) respectively;
(lxxix) CAAASGYSTLTF (SEQ ID NO: 393) and CASSLSPGKSNQPQHF (SEQ ID NO: 079) respectively;
(lxxx) CVVSLLTGGGNKLTF (SEQ ID NO: 394) and CASSLSYEQYF (SEQ ID NO: 080) respectively;
(lxxxi) CAVRSSLGNNRLAF (SEQ ID NO: 395) and CASSLTEGVRTEAFF (SEQ ID NO: 081) respectively;
(lxxxii) CAAMGNRDDKIIF (SEQ ID NO: 396) and CASSLVETQYF (SEQ ID NO: 082) respectively;
(lxxxiii) CAVDRARNSGGSNYKLTF (SEQ ID NO: 397) and CASSLVGGNTIYF (SEQ ID NO: 083) respectively;
(lxxxiv) CALGEYGNKLVF (SEQ ID NO: 398) and CASSLVGNTEAFF (SEQ ID NO: 084) respectively;
(lxxxv) CAASGANSGYALNF (SEQ ID NO: 399) and CASSLVSTAEQYF (SEQ ID NO: 085) respectively;
(lxxxvi) CAGPTNSGGYQKVTF (SEQ ID NO: 400) and CASSLVVTGELFF (SEQ ID NO: 086) respectively;
(lxxxvii) CIVRVAYNNAGNMLTF (SEQ ID NO: 401) and CASSLWGATDTQYF (SEQ ID NO: 087) respectively;
(lxxxviii) CAAGDTGRRALTF (SEQ ID NO: 402) and CASSPDSSFGNQPQHF (SEQ ID NO: 088) respectively;
(lxxxix) CASGRGSQGNLIF (SEQ ID NO: 403) and CASSPDSYNEQFF (SEQ ID NO: 089) respectively;
(xc) CALIDRGSTLGRLYF (SEQ ID NO: 404) and CASSPEETQYF (SEQ ID NO: 090) respectively;
(xci) CAAPPGGTSYGKLTF (SEQ ID NO: 405) and CASSPGQAANSPLHF (SEQ ID NO: 091) respectively;
(xcii) CAVQAAGGYQKVTF (SEQ ID NO: 406) and CASSPGRVAFF (SEQ ID NO: 092) respectively;
(xciii) CAERQGNTPLVF (SEQ ID NO: 407) and CASSPGTDQPQHF (SEQ ID NO: 093) respectively;
(xciv) CAASGDRDDKIIF (SEQ ID NO: 408) and CASSPGTEAFF (SEQ ID NO: 094) respectively;
(xcv) CATDPGANNLFF (SEQ ID NO: 409) and CASSPMGTGNTEAFF (SEQ ID NO: 095) respectively;
(xcvi) CALPPGGGTSYGKLTF (SEQ ID NO: 410) and CASSPPDRGRHEQFF (SEQ ID NO: 096) respectively;
(xcvii) CAVRDAGGYNKLIF (SEQ ID NO: 411) and CASSPPSGSGELFF (SEQ ID NO: 097) respectively;
(xcviii) CAMRLGAAGNKLTF (SEQ ID NO: 412) and CASSPPVLVSGNTIYF (SEQ ID NO: 098) respectively;
(xcix) CALSDYNQGGKLIF (SEQ ID NO: 413) and CASSPQDRGQGNTEAFF (SEQ ID NO: 099) respectively;
(c) CALSPRGGYQKVTF (SEQ ID NO: 414) and CASSPRDRGLYQPQHF (SEQ ID NO: 100) respectively;
(ci) CAVRGRGYSTLTF (SEQ ID NO: 415) and CASSPRGAGNTIYF (SEQ ID NO: 101) respectively;
(cii) CAGDFGGTSYGKLTF (SEQ ID NO: 416) and CASSPRTGGNQPQHF (SEQ ID NO: 102) respectively;
(ciii) CAASRGNNRLAF (SEQ ID NO: 417) and CASSPSAGAGYEQYF (SEQ ID NO: 103) respectively;
(civ) CAVRDGGGYNKLIF (SEQ ID NO: 418) and CASSPSQGIDSGANVLTF (SEQ ID NO: 104) respectively;
(cv) CAVQGYLGGATNKLIF (SEQ ID NO: 419) and CASSPSRDRSYEQYF (SEQ ID NO: 105) respectively;
(cvi) CAVYSNTGKLIF (SEQ ID NO: 420) and CASSPTDRIRAFF (SEQ ID NO: 106) respectively;
(cvii) CAMRVNNARLMF (SEQ ID NO: 421) and CASSPYRGLNHSF (SEQ ID NO: 107) respectively;
(cviii) CALSDRTGANSKLTF (SEQ ID NO: 422) and CASSQDGGGTDTQYF (SEQ ID NO: 108) respectively;
(cix) CALSDRGSARQLTF (SEQ ID NO: 423) and CASSQDGVATDTQYF (SEQ ID NO: 109) respectively;
(cx) CAAKGNTGNQFYF (SEQ ID NO: 424) and CASSQDKGRDQPQHF (SEQ ID NO: 110) respectively;
(cxi) CAALRDRGSTLGRLYF (SEQ ID NO: 425) and CASSQDRPSFTEAFF (SEQ ID NO: 111) respectively;
(cxii) CAVSETGFQKLVF (SEQ ID NO: 426) and CASSQDRQKLSGELFF (SEQ ID NO: 112) respectively;
(cxiii) CATDATSGSRLTF (SEQ ID NO: 427) and CASSQDRTSTRDEQFF (SEQ ID NO: 113) respectively;
(cxiv) CAERNNNARLMF (SEQ ID NO: 428) and CASSQDWVVGNQPQHF (SEQ ID NO: 114) respectively;
(cxv) CAASTGNQFYF (SEQ ID NO: 429) and CASSQEDRGNQPQHF (SEQ ID NO: 115) respectively;
(cxvi) CVVTLNNAGNMLTF (SEQ ID NO: 430) and CASSQGGVGETQYF (SEQ ID NO: 116) respectively;
(cxvii) CAASGGEGGGADGLTF (SEQ ID NO: 431) and CASSQGRGGYQPQHF (SEQ ID NO: 117) respectively;
(cxviii) CAVGPWGDYKLSF (SEQ ID NO:432) and CASSQGTGGMRGYTF (SEQ ID NO:118) respectively;
(cxix) CAASWGNTPLVF (SEQ ID NO: 433) and CASSQQGSEQYV (SEQ ID NO: 119) respectively;
(cxx) CAVRRRGDSNYQLIW (SEQ ID NO: 434) and CASSQSEVGGQFF (SEQ ID NO: 120) respectively;
(cxxi) CAVSEKGAGGFKTIF (SEQ ID NO: 435) and CASSRDSGRAGDTQYF (SEQ ID NO: 121) respectively;
(cxxii) CAVSQMDSSYKLIF (SEQ ID NO: 436) and CASSREGYGYTF (SEQ ID NO: 122) respectively;
(cxxiii) CAVSGLNNARLMF (SEQ ID NO: 437) and CASSRQSSGNTIYF (SEQ ID NO: 123) respectively;
(cxxiv) CALSGGQAGTALIF (SEQ ID NO: 438) and CASSRSGLFNTEGAFF (SEQ ID NO: 124) respectively;
(cxxv) CAVRRQGGKLIF (SEQ ID NO: 439) and CASSRTALAANVLTF (SEQ ID NO: 125) respectively;
(cxxvi) CAVRPESNFGNEKLTF (SEQ ID NO: 440) and CASSRTGDNSPLHF (SEQ ID NO: 126) respectively;
(cxxvii) CIVRVAGGTSYGKLTF (SEQ ID NO: 441) and CASSRTGGGRGYTF (SEQ ID NO: 127) respectively;
(cxxviii) CAEMNNAGNMLTF (SEQ ID NO: 442) and CASSSENSPLHF (SEQ ID NO: 128) respectively;
(cxxix) CAASIVGSQGNLIF (SEQ ID NO: 443) and CASSSGGLNTEAFF (SEQ ID NO: 129) respectively;
(cxxx) CIVRVGGISNFGNEKLTF (SEQ ID NO: 444) and CASSSGNSPLHF (SEQ ID NO: 130) respectively;
(cxxxi) CAVETSGSRLTF (SEQ ID NO: 445) and CASSSLQVNSGNTIYF (SEQ ID NO: 131) respectively;
(cxxxii) CALRAGGTSYGKLTF (SEQ ID NO: 446) and CASSSLTPGYGYTF (SEQ ID NO: 132) respectively;
(cxxxiii) CAASTGGYNKLIF (SEQ ID NO: 447) and CASSSPTLSTNEKLFF (SEQ ID NO: 133) respectively;
(cxxxiv) CAYSGDGYALNF (SEQ ID NO: 448) and CASSSRTGYYEQYF (SEQ ID NO: 134) respectively;
(cxxxv) CATDARGDFGNEKLTF (SEQ ID NO: 449) and CASSSSQRTMDGYTF (SEQ ID NO: 135) respectively;
(cxxxvi) CATDKGSNYQLIW (SEQ ID NO: 450) and CASSSTGTGPFF (SEQ ID NO: 136) respectively;
(cxxxvii) CAASTGNQFYF (SEQ ID NO:451) and CASSSVWGQGGEQYF (SEQ ID NO:137) respectively;
(cxxxviii) CLVGVDQTGANNLFF (SEQ ID NO: 452) and CASSTGGWGPNSPLHF (SEQ ID NO: 138) respectively;
(cxxxix) CAVSVPGSNYQLIW (SEQ ID NO: 453) and CASSTGPQETQYF (SEQ ID NO: 139) respectively;
(cxl) CAANNNAGNMLTF (SEQ ID NO: 454) and CASSTQENEKLFF (SEQ ID NO: 140) respectively;
(cxli) CAGRNSGYALNF (SEQ ID NO: 455) and CASSTRTNQHEKLFF (SEQ ID NO: 141) respectively;
(cxlii) CAVPYLSGAGSYQLTF (SEQ ID NO: 456) and CASSTTAAGNTIYF (SEQ ID NO: 142) respectively;
(cxliii) CAVSPNSGGYQKVTF (SEQ ID NO: 457) and CASSVGGLASSYEQYF (SEQ ID NO: 143) respectively;
(cxliv) CAASTPNNNARLMF (SEQ ID NO: 458) and CASSVGLAGSQETQYF (SEQ ID NO: 144) respectively;
(cxlv) CVVEPGNYGQNFVF (SEQ ID NO: 459) and CASSWGMPNEKLFF (SEQ ID NO: 145) respectively;
(cxlvi) CAMSASTGGFKTIF (SEQ ID NO: 460) and CASSWGSNQPQHF (SEQ ID NO: 146) respectively;
(cxlvii) CAENMMDSSYKLIF (SEQ ID NO: 461) and CASSWTPAGETQYF (SEQ ID NO: 147) respectively;
(cxlviii) CAASGNFGNEKLTF (SEQ ID NO: 462) and CASSYPSGAFGNEQFF (SEQ ID NO: 148) respectively;
(cxlix) CAVPSNAGGTSYGKLTF (SEQ ID NO: 463) and CASSYRGAGQPQHF (SEQ ID NO: 149) respectively;
(cl) CAVRDGAGSYQLTF (SEQ ID NO: 464) and CASSYSYEQYF (SEQ ID NO: 150) respectively;
(cli) CALTGMMDSSYKLIF (SEQ ID NO: 465) and CASSYTTEAFF (SEQ ID NO: 151) respectively;
(clii) CAYRTPPNDMRF (SEQ ID NO: 466) and CASTPGSGANVLTF (SEQ ID NO: 152) respectively;
(cliii) CAASATDSSYKLIF (SEQ ID NO: 467) and CASTPSQGHNSPLHF (SEQ ID NO: 153) respectively;
(cliv) CAVNAPFGNEKLTF (SEQ ID NO: 468) and CASRRNGLYYTF (SEQ ID NO: 154) respectively;
(clv) CAASRTGRRALTF (SEQ ID NO: 469) and CATSDDSGQGAEAFF (SEQ ID NO: 155) respectively;
(clvi) CAHTSSGGSYIPTF (SEQ ID NO: 470) and CATSDMGLADNEQFF (SEQ ID NO: 156) respectively;
(clvii) CAASDSNYQLIW (SEQ ID NO: 471) and CATSDPSGPNYNEQFF (SEQ ID NO: 157) respectively;
(clviii) CAVGAYNNNDMRF (SEQ ID NO: 472) and CATSEGGQGGYGYTF (SEQ ID NO: 158) respectively;
(clix) CAGFNSGYALNF (SEQ ID NO: 473) and CATSGGGAYEQYF (SEQ ID NO: 159) respectively;
(clx) CAVSSTGANSKLTF (SEQ ID NO: 474) and CATSQERRQVGSPLHF (SEQ ID NO: 160) respectively;
(clxi) CAFILPSGAGSYQLTF (SEQ ID NO: 475) and CAWSALAGSWAGELFF (SEQ ID NO: 161) respectively;
(clxii) CAMSEPNGQNFVF (SEQ ID NO: 476) and CSAAGTGNTEAFF (SEQ ID NO: 162) respectively;
(clxiii) CAPRDSGYSTLTF (SEQ ID NO: 477) and CSAARQRTNYGYTF (SEQ ID NO: 163) respectively;
(clxiv) CAVRRNAGNMLTF (SEQ ID NO: 478) and CSADRTSAKNIQYF (SEQ ID NO: 164) respectively;
(clxv) CAESHNTDKLIF (SEQ ID NO: 479) and CSAFRLAAQGGSYEQYF (SEQ ID NO: 165) respectively;
(clxvi) CVVGTGTASKLTF (SEQ ID NO: 480) and CSAIRPGVGDYEQYF (SEQ ID NO: 166) respectively;
(clxvii) CAMKTGGGNKLTF (SEQ ID NO: 481) and CSAKSTGYDYEQYF (SEQ ID NO: 167) respectively;
(clxviii) CVVKLNSSASKIIF (SEQ ID NO: 482) and CSALSQSGGTNIQYF (SEQ ID NO: 168) respectively;
(clxix) CAASLNFNKFYF (SEQ ID NO: 483) and CSALWSGDGEQFF (SEQ ID NO: 169) respectively;
(clxx) CAVRDGGGYSTLTF (SEQ ID NO: 484) and CSAMTREGGNQPQHF (SEQ ID NO: 170) respectively;
(clxxi) CASRFSGGYNKLIF (SEQ ID NO: 485) and CSANPLAGGGEQYF (SEQ ID NO: 171) respectively;
(clxxii) CAVSDPGGYNKLIF (SEQ ID NO: 486) and CSAPGPAAAGELFF (SEQ ID NO: 172) respectively;
(clxxiii) CAVSEPGGYQKVTF (SEQ ID NO: 487) and CSAPGTSAGANVLTF (SEQ ID NO: 173) respectively;
(clxxiv) CAFRSNNNDMRF (SEQ ID NO:488) and CSAPKLVGSGNTIYF (SEQ ID NO: 174) respectively;
(clxxv) CAVWGVNQAGTALIF (SEQ ID NO: 489) and CSAPQDRNNEQFF (SEQ ID NO: 175) respectively;
(clxxvi) CGTPSGGYQKVTF (SEQ ID NO: 490) and CSAPSTDRVRGYTF (SEQ ID NO: 176) respectively;
(clxxvii) CAASQAAGNKLTF (SEQ ID NO: 491) and CSARDHTSGSGNEQFF (SEQ ID NO: 177) respectively;
(clxxviii) CAASMGTGNQFYF (SEQ ID NO: 492) and CSARDPDRGSGNEQYF (SEQ ID NO: 178) respectively;
(clxxix) CALSEVYNNDMRF (SEQ ID NO: 493) and CSARDQGALLNSPLHF (SEQ ID NO: 179) respectively;
(clxxx) CALHWRGAQKLVF (SEQ ID NO: 494) and CSARDRGGNTEAFF (SEQ ID NO: 180) respectively;
(clxxxi) CAVRDQAGTALIF (SEQ ID NO: 495) and CSARDRVGGEQFF (SEQ ID NO: 181) respectively;
(clxxxii) CAASMAAGNQFYF (SEQ ID NO: 496) and CSARDVRLNTEAFF (SEQ ID NO: 182) respectively;
(clxxxiii) CAASTGAGNMLTF (SEQ ID NO: 497) and CSARDVWGTGNSQASGNEQFF (SEQ ID NO: 183) respectively;
(clxxxiv) CAVRDTGNQFYF (SEQ ID NO: 498) and CSARGLAGADTQYF (SEQ ID NO: 184) respectively;
(clxxxv) CAVKGSNTGKLIF (SEQ ID NO: 499) and CSARGPGGNTEAFF (SEQ ID NO: 185) respectively;
(clxxxvi) CATHPNSGYALNF (SEQ ID NO: 500) and CSARGPGTDTQYF (SEQ ID NO: 186) respectively;
(clxxxvii) CAGLNTGNQFYF (SEQ ID NO: 501) and CSARGRQDQPQHF (SEQ ID NO: 187) respectively;
(clxxxviii) CALSDQNARLMF (SEQ ID NO: 502) and CSARTITGSGYTF (SEQ ID NO: 188) respectively;
(clxxxix) CIVRVSNSGNTPLVF (SEQ ID NO: 503) and CSARTSGRGYNEQFF (SEQ ID NO: 189) respectively;
(cxc) CALSDPQAALTF (SEQ ID NO: 504) and CSARVLGAGPNNEQFF (SEQ ID NO: 190) respectively;
(cxci) CVVSDRPGGGNKLTF (SEQ ID NO: 505) and CSARVSAVSTDTQYF (SEQ ID NO: 191) respectively;
(cxcii) CAMRTGGGNKLTF (SEQ ID NO: 506) and CSASPLAGGSYEQYF (SEQ ID NO: 192) respectively;
(cxciii) CAVSEPGGYNKLIF (SEQ ID NO: 507) and CSASPLKAGANVLTF (SEQ ID NO: 193) respectively;
(cxciv) CIVKNTGTALIF (SEQ ID NO: 508) and CSASRDSNQPQHF (SEQ ID NO: 194) respectively;
(cxcv) CAVGGRGSTLGRLYF (SEQ ID NO: 509) and CSASSDRGGNQPQHF (SEQ ID NO: 195) respectively;
(cxcvi) CAMSNNFNKFYF (SEQ ID NO: 510) and CSASSGTVGGYTF (SEQ ID NO: 196) respectively;
(cxcvii) CAPPRGTGGYNKLIF (SEQ ID NO: 511) and CSASSGVSSYNEQFF (SEQ ID NO: 197) respectively;
(cxcviii) CAVSEPGGYQKVTF (SEQ ID NO: 512) and CSATRFGQANTGELFF (SEQ ID NO: 198) respectively;
(cxcix) CAVRDSGGYNKLIF (SEQ ID NO: 513) and CSATTWTGGNTEAFF (SEQ ID NO: 199) respectively;
(cc) CAVSESGGYQKVTF (SEQ ID NO: 514) and CSAVDWTSGSSYEQYV (SEQ ID NO: 200) respectively;
(cci) CAVRGFSDGQKLLF (SEQ ID NO: 515) and CSAVLGLAGVRDTQYF (SEQ ID NO: 201) respectively;
(ccii) CARRGSSGSARQLTF (SEQ ID NO: 516) and CSISPDRGGNQPQHF (SEQ ID NO: 202) respectively;
(cciii) CAVPYLTNAGKSTF (SEQ ID NO: 517) and CSLVPDRGGNQPQHF (SEQ ID NO: 203) respectively;
(cciv) CAVEETSGSRLTF (SEQ ID NO: 518) and CSRGGREGEQFF (SEQ ID NO: 204) respectively;
(ccv) CAGQAAYKYIF (SEQ ID NO: 519) and CSVEGQATYEQYF (SEQ ID NO: 205) respectively;
(ccvi) CAVSQAWGGKLIF (SEQ ID NO: 520) and CSVEGQGNYGYTF (SEQ ID NO: 206) respectively;
(ccvii) CAGTNTDKLIF (SEQ ID NO: 521) and CSVLGQGAPRSYEQYF (SEQ ID NO: 207) respectively;
(ccviii) CAESIGTDKLIF (SEQ ID NO: 522) and CSVRGRANEQYF (SEQ ID NO: 208) respectively;
(ccix) CAVLFGNEKLTF (SEQ ID NO: 523) and CASARTGQETQYF (SEQ ID NO: 209) respectively;
(ccx) CAVDDSGGGADGLTF (SEQ ID NO: 524) and CASNLPRSGELFF (SEQ ID NO: 210) respectively;
(ccxi) CAASWGGTSYGKLTF (SEQ ID NO: 525) and CASRPELDSYEQYF (SEQ ID NO: 211) respectively;
(ccxii) CAPILQGAQKLVF (SEQ ID NO: 526) and CASRRGGISNQPQHF (SEQ ID NO: 212) respectively;
(ccxiii) CILRDNYGQNFVF (SEQ ID NO: 527) and CASSEHGGNYGYTF (SEQ ID NO: 213) respectively;
(ccxiv) CAVAGTASKLTF (SEQ ID NO: 528) and CASSIFTLSNQPQHF (SEQ ID NO: 214) respectively;
(ccxv) CAVYSNTGKLIF (SEQ ID NO: 529) and CASSKQGATEAFF (SEQ ID NO: 215) respectively;
(ccxvi) CAVNTGNQFYF (SEQ ID NO: 530) and CASSLGANYGYTF (SEQ ID NO: 216) respectively;
(ccxvii) CAVLLTGGGNKLTF (SEQ ID NO: 531) and CASSLTDLYEQYF (SEQ ID NO: 217) respectively;
(ccxviii) CIVGNTGGFKTIF (SEQ ID NO: 532) and CASSPDRVEQYF (SEQ ID NO: 218) respectively;
(ccxix) CAASVWGGSEKLVF (SEQ ID NO: 533) and CASSPPGGTEVYEQYF (SEQ ID NO: 219) respectively;
(ccxx) CALSDRGGNKLVF (SEQ ID NO: 534) and CASSPPPGRAETGELFF (SEQ ID NO: 220) respectively;
(ccxxi) CAARETYNTDKLIF (SEQ ID NO: 535) and CASSRGAGELFF (SEQ ID NO: 221) respectively;
(ccxxii) CGSPGAGSYQLTF (SEQ ID NO: 536) and CASSVGGDYGYTF (SEQ ID NO: 222) respectively;
(ccxxiii) CAGGNAGNNRKLIW (SEQ ID NO: 537) and CASSYGTANTEAFF (SEQ ID NO: 223) respectively;
(ccxxiv) CAFMMLTGGGADGLTF (SEQ ID NO: 538) and CASSYSTLAGGHSYEQYF (SEQ ID NO: 224) respectively;
(ccxxv) CAVRDGAGSYQLTF (SEQ ID NO: 539) and CASSYSYEQYF (SEQ ID NO: 225) respectively;
(ccxxvi) CIVRVEAGKSTF (SEQ ID NO: 540) and CSVAGQGNSPLHF (SEQ ID NO: 226) respectively;
(ccxxvii) CATIQTGANNLFF (SEQ ID NO: 541) and CASGGTTDTQYF (SEQ ID NO: 227) respectively;
(ccxxviii) CAGYNSGTYKYIF (SEQ ID NO: 542) and CASRRGNTGELFF (SEQ ID NO: 228) respectively;
(ccxxix) CAVQAVNNNARLMF (SEQ ID NO: 543) and CASRSTGTGEKLFF (SEQ ID NO: 229) respectively;
(ccxxx) CAFMNTDKLIF (SEQ ID NO: 544) and CASSFWAGVSTDTQYF (SEQ ID NO: 230) respectively;
(ccxxxi) CAMREGSGGYNKLIF (SEQ ID NO: 545) and CASSGGRKLDTQYF (SEQ ID NO: 231) respectively;
(ccxxxii) CAMRPRSSNTGKLIF (SEQ ID NO: 546) and CASSLNLLDRASLETQYF (SEQ ID NO: 232) respectively;
(ccxxxiii) CATDFFGNEKLTF (SEQ ID NO: 547) and CASSLTGYNSPLHF (SEQ ID NO: 233) respectively;
(ccxxxiv) CAVSHTGNQFYF (SEQ ID NO: 548) and CASSLVLEHEQFF (SEQ ID NO: 234) respectively;
(ccxxxv) CAELRIQGAQKLVF (SEQ ID NO: 549) and CASSQDRITSGYGYTF (SEQ ID NO: 235) respectively;
(ccxxxvi) CVVIFTGTASKLTF (SEQ ID NO: 550) and CASSTGGRSNQPQHF (SEQ ID NO: 236) respectively;
(ccxxxvii) CAVSGLGGGADGLTF (SEQ ID NO: 551) and CASSVVPGAGGEQFF (SEQ ID NO: 237) respectively;
(ccxxxviii) CALPDSGGGADGLTF (SEQ ID NO: 552) and CASSVVPGGPGGELFF (SEQ ID NO: 238) respectively;
(ccxxxix) CAENIKGSSGYSTLTF (SEQ ID NO: 553) and CASSWAPHTDEQFF (SEQ ID NO: 239) respectively;
(ccxl) CAGEGAGSYQLTF (SEQ ID NO: 554) and CASSWTGNTGELFF (SEQ ID NO: 240) respectively;
(ccxli) CAVGDSNYQLIW (SEQ ID NO: 555) and CASSYTQETQYF (SEQ ID NO: 241) respectively;
(ccxlii) CAVQGALNNARLMF (SEQ ID NO: 556) and CASSYTTSGGTYEQYF (SEQ ID NO: 242) respectively;
(ccxliii) CAVSDPLGGSNYKLTF (SEQ ID NO: 557) and CASTPSGGTQPQHF (SEQ ID NO: 243) respectively;
(ccxliv) CAGHQAGTALIF (SEQ ID NO: 558) and CATSDPGTREQFF (SEQ ID NO: 244) respectively;
(ccxlv) CAVSGGATNKLIF (SEQ ID NO: 559) and CATSYRAGGGYNEQFF (SEQ ID NO: 245) respectively;
(ccxlvi) CAASIELTGGGNKLTF (SEQ ID NO: 560) and CSARGNEQFF (SEQ ID NO: 246) respectively;
(ccxlvii) CAAGMYSSASKIIF (SEQ ID NO: 561) and CSASSSGTQYF (SEQ ID NO: 247) respectively;
(ccxlviii) CALSLSGYSTLTF (SEQ ID NO: 562) and CAAEDLAKNIQYF (SEQ ID NO: 248) respectively;
(ccxlix) CAVINVDFQKLVF (SEQ ID NO: 563) and CAGRRRLGDSPLHF (SEQ ID NO: 249) respectively;
(ccl) CASLTGGGNKLTF (SEQ ID NO: 564) and CASKQDLNTEAFF (SEQ ID NO: 250) respectively;
(ccli) CAMGITSGYALNF (SEQ ID NO: 565) and CASLSGPGYEQYF (SEQ ID NO: 251) respectively;
(cclii) CAVKGGGATNKLIF (SEQ ID NO: 566) and CASNAGYTSGELFF (SEQ ID NO: 252) respectively;
(ccliii) CAVLGYGNKLVF (SEQ ID NO: 567) and CASQDRTALEQYF (SEQ ID NO: 253) respectively;
(ccliv) CAGQLAAGTASKLTF (SEQ ID NO: 568) and CASRGGSSGANVLTF (SEQ ID NO: 254) respectively;
(cclv) CARYSGGGADGLTF (SEQ ID NO: 569) and CASRGTSGRTYEQYF (SEQ ID NO: 255) respectively;
(cclvi) CAVSPSGGYQKVTF (SEQ ID NO: 570) and CASRLAGQEANYGYTF (SEQ ID NO: 256) respectively;
(cclvii) CAENRRAGGTSYGKLTF (SEQ ID NO: 571) and CASRPSLLRELFF (SEQ ID NO: 257) respectively;
(cclviii) CAGYNSGTYKYIF (SEQ ID NO: 572) and CASRRGNTGELFF (SEQ ID NO: 258) respectively;
(cclix) CAASDAGNMLTF (SEQ ID NO: 573) and CASRRNSGANVLTF (SEQ ID NO: 259) respectively;
(cclx) CAARGNSGGSNYKLTF (SEQ ID NO: 574) and CASSARDRYYGYTF (SEQ ID NO: 260) respectively;
(cclxi) CATVNSGNTPLVF (SEQ ID NO: 575) and CASSDRDTDTQYF (SEQ ID NO: 261) respectively;
(cclxii) CAVERGSQGNLIF (SEQ ID NO: 576) and CASSEGGTRHETQYF (SEQ ID NO: 262) respectively;
(cclxiii) CAVMDSNYQLIW (SEQ ID NO: 577) and CASSEGQGADTQYF (SEQ ID NO: 263) respectively;
(cclxiv) CAGPGYGNKLVF (SEQ ID NO: 578) and CASSEVSGNQPQHF (SEQ ID NO: 264) respectively;
(cclxv) CAAGVNFGNEKLTF (SEQ ID NO: 579) and CASSFGLTNEKLFF (SEQ ID NO: 265) respectively;
(cclxvi) CAASRGFNDMRF (SEQ ID NO: 580) and CASSFGTGVYGYTF (SEQ ID NO: 266) respectively;
(cclxvii) CAVSGLVGNEKLTF (SEQ ID NO: 581) and CASSFMDRDNSPLHF (SEQ ID NO: 267) respectively;
(cclxviii) CAVVFNKFYF (SEQ ID NO: 582) and CASSFSGDNEQFF (SEQ ID NO: 268) respectively;
(cclxix) CATEGDSGYSTLTF (SEQ ID NO: 583) and CASSGQGGGYGYTF (SEQ ID NO: 269) respectively;
(cclxx) CAVSGTGNQFYF (SEQ ID NO: 584) and CASSITRKETQYF (SEQ ID NO: 270) respectively;
(cclxxi) CAASVGGSNYKLTF (SEQ ID NO: 585) and CASSLAHYEQYF (SEQ ID NO: 271) respectively;
(cclxxii) CAASGSDSGNTPLVF (SEQ ID NO: 586) and CASSLAPHTDEQFF (SEQ ID NO: 272) respectively;
(cclxxiii) CAMSSRGSARQLTF (SEQ ID NO: 587) and CASSLDEQGQNEQFF (SEQ ID NO: 273) respectively;
(cclxxiv) CAVNGFGNVLHC (SEQ ID NO: 588) and CASSLEADYEQYF (SEQ ID NO: 274) respectively;
(cclxxv) CAAPSRDDKIIF (SEQ ID NO: 589) and CASSLEDNQPQHF (SEQ ID NO: 275) respectively;
(cclxxvi) CLVGDNAPSGSARQLTF (SEQ ID NO: 590) and CASSLGGQVYGYTF (SEQ ID NO: 276) respectively;
(cclxxvii) CAENGSDYKLSF (SEQ ID NO: 591) and CASSLGQGLNEKLFF (SEQ ID NO: 277) respectively;
(cclxxviii) CAALSHQGAQKLVF (SEQ ID NO: 592) and CASSLGRNYGYTF (SEQ ID NO: 278) respectively;
(cclxxix) CAVRVFSGGYNKLIF (SEQ ID NO: 593) and CASSLGTSAYNEQFF (SEQ ID NO: 279) respectively;
(cclxxx) CAVGERGATNKLIF (SEQ ID NO: 594) and CASSLMQAANSPLHF (SEQ ID NO: 280) respectively;
(cclxxxi) CAVKSNSGNTPLVF (SEQ ID NO: 595) and CASSLMSATNYGYTF (SEQ ID NO: 281) respectively;
(cclxxxii) CAASEPGAQKLVF (SEQ ID NO: 596) and CASSLQGAREKLFF (SEQ ID NO: 282) respectively;
(cclxxxiii) CAGAVTTDSWGKLQF (SEQ ID NO: 597) and CASSLQGGTEAFF (SEQ ID NO: 283) respectively;
(cclxxxiv) CAVNVNSGAGSYQLTF (SEQ ID NO: 598) and CASSLSGSSYNEQFF (SEQ ID NO: 284) respectively;
(cclxxxv) CAMRERTGGSYIPTF (SEQ ID NO: 599) and CASSLSGTGNGRNQPQHF (SEQ ID NO: 285) respectively;
(cclxxxvi) CAIGRGSTLGRLYF (SEQ ID NO: 600) and CASSLSRDAVGGYTF (SEQ ID NO: 286) respectively;
(cclxxxvii) CAVSPPGYSSASKIIF (SEQ ID NO: 601) and CASSLTGTGGYEQYF (SEQ ID NO: 287) respectively;
(cclxxxviii) CAEELSGGYQKVTF (SEQ ID NO: 602) and CASSLVAGGYEQYF (SEQ ID NO: 288) respectively;
(cclxxxix) CAVEFTEYGNKLVF (SEQ ID NO: 603) and CASSLYNEQFF (SEQ ID NO: 289) respectively;
(ccxc) CAVSYSSASKIIF (SEQ ID NO: 604) and CASSPPFGSYEQYF (SEQ ID NO: 290) respectively;
(ccxci) CAEFYNQGGKLIF (SEQ ID NO: 605) and CASSQADTQYF (SEQ ID NO: 291) respectively;
(ccxcii) CAVNNGNKLVF (SEQ ID NO: 606) and CASSQGQEFGKLFF (SEQ ID NO: 292) respectively;
(ccxciii) CAGGNAGKSTF (SEQ ID NO: 607) and CASSQTSGSYNEQFF (SEQ ID NO: 293) respectively;
(ccxciv) CAASRRGSQGNLIF (SEQ ID NO: 608) and CASSRTYEQYF (SEQ ID NO: 294) respectively;
(ccxcv) CAGPMKTSYDKVIF (SEQ ID NO: 609) and CASSSANYGYTF (SEQ ID NO: 295) respectively;
(ccxcvi) CAVKDSNYQLIW (SEQ ID NO: 610) and CASSSGEGEAGELFF (SEQ ID NO: 296) respectively;
(ccxcvii) CAASIVGSQGNLIF (SEQ ID NO: 611) and CASSSGGLNTEAFF (SEQ ID NO: 297) respectively;
(ccxcviii) CAALPGNTPLVF (SEQ ID NO: 612) and CASSSGGRAWDTQYF (SEQ ID NO: 298) respectively;
(ccxcix) CAPWRGSARQLTF (SEQ ID NO: 613) and CASSSGLAAYEQYF (SEQ ID NO: 299) respectively;
(ccc) CAVNPTGGFKTIF (SEQ ID NO: 614) and CASSSQGSQETQYF (SEQ ID NO: 300) respectively;
(ccci) CIVRPSNAGGTSYGKLTF (SEQ ID NO: 615) and CASSSTGGNQPQHF (SEQ ID NO: 301) respectively;
(cccii) CAASRVGQLTF (SEQ ID NO: 616) and CASSVRQGSAGELFF (SEQ ID NO: 302) respectively;
(ccciii) CATDAWTGANSKLTF (SEQ ID NO: 617) and CASSWGLADETQYF (SEQ ID NO: 303) respectively;
(ccciv) CAAKWAYSGAGSYQLTF (SEQ ID NO: 618) and CASSYDSRYGYTF (SEQ ID NO: 304) respectively;
(cccv) CAVRDNNQGGKLIF (SEQ ID NO: 619) and CASSYSAGEQYF (SEQ ID NO: 305) respectively;
(cccvi) CAYRSQETSGSRLTF (SEQ ID NO: 620) and CASSYSPSTKNIQYF (SEQ ID NO: 306) respectively;
(cccvii) CAADTGRRALTF (SEQ ID NO: 621) and CATEGRGNTIYF (SEQ ID NO: 307) respectively;
(cccviii) CAKYTDKLIF (SEQ ID NO: 622) and CATPPGGLANTGELFF (SEQ ID NO: 308) respectively;
(cccix) CAASIGSTLGRLYF (SEQ ID NO: 623) and CATSDSSGRYYNEQFF (SEQ ID NO: 309) respectively;
(cccx) CVVNGPPGGSYIPTF (SEQ ID NO: 624) and CAWSGMNTEAFF (SEQ ID NO: 310) respectively;
(cccxi) CAVTDSWGKLQF (SEQ ID NO: 625) and CSARGGHSFEQYF (SEQ ID NO: 311) respectively;
(cccxii) CAVVDSNYQLIW (SEQ ID NO: 626) and CSARNGDTEAFF (SEQ ID NO: 312) respectively;
(cccxiii) CAEELSGGYQKVTF (SEQ ID NO: 627) and CASSLVAGGYEQYF (SEQ ID NO: 313) respectively;
(cccxiv) CAVSFKAAGNKLTF (SEQ ID NO: 628) and CSVRVNTEAFF (SEQ ID NO: 314) respectively;
(cccxv) CAGYNSGTYKYIF (SEQ ID NO: 632) and CASRRGNTGELFF (SEQ ID NO: 631) respectively; or
(cccxvi) CAASIVGSQGNLIF (SEQ ID NO: 634) and CASSSGGLNTEAFF (SEQ ID NO: 633) respectively.

111. A pharmaceutical composition comprising the TCR of claim 99.

112. A method of treating a subject in need thereof with a T cell receptor (TCR), the TCR comprising:
(i) a TCRα chain variable domain (TRAV) comprising a CDR1α, a CDR2 α, and a CDR3 α, wherein the CDR3α comprises an amino acid sequence having at least 75% sequence identity to any one of SEQ ID NOs: 315-629, 632, or 634; and
(ii) a TCRβ chain variable domain (TRBV) comprising a CDR1β, a CDR2 β, and a CDR3β, wherein the CDR3β comprises an amino acid sequence having at least 75% sequence identity to any one of SEQ ID NOs: 1-314, 630, 631, or 633.

113. The method of claim 112, wherein the TRAV comprises an amino acid sequence of any one of SEQ ID NOs: 315-629, 632, or 634.

114. The method of claim 113, wherein the TRBV comprises an amino acid sequence of any one of SEQ ID NOs: 1-314, 630, 631, or 633.

115. The method of claim 114, wherein the TRAV comprises the amino acid sequence of SEQ ID NO: 629.

116. The method of claim 115, wherein the TRBV comprises the amino acid sequence of SEQ ID NO: 630.

117. The method of claim 112, wherein the treating comprises administering the TCR to the subject.

118. The method of claim 112, wherein the subject has a cancer.
